(19) United States
(12) Patent Application Publication
(10) Pub. No.: US 2023/0203594 A1
(43) Pub. Date: Jun. 29, 2023

(54) METHODS OF DIAGNOSING CANCER AND PREDICTING RESPONSIVENESS TO THERAPY

(71) Applicant: Yeda Research and Development Co. Ltd., Rehovot (IL)

(72) Inventors: Ravid STRAUSSMAN, Mazkeret Batia (IL); Noam SHENTAL, Rehovot (IL); Ilana LIVYATAN, Rehovot (IL); Deborah GITTA ROSENBERG NEJMAN, Rehovot (IL); Garold FUKS, Rehovot (IL)

(73) Assignee: Yeda Research and Development Co. Ltd., Rehovot (IL)

(21) Appl. No.: 17/958,516

(22) Filed: Oct. 3, 2022

Related U.S. Application Data
(63) Continuation of application No. PCT/IL2021/050388, filed on Apr. 6, 2021.
(60) Provisional application No. 63/005,540, filed on Apr. 6, 2020.

(51) Int. Cl. C12Q 1/6886; C12Q 1/689

(52) U.S. Cl. CPC C12Q 1/6886; C12Q 1/689; C12Q 1/6869

(57) ABSTRACT

A method of treating cancer in a subject is disclosed. The method comprises:
(a) analyzing for the abundance of at least one bacteria in the tumor microbiome of the subject; and
(b) administering to the subject a therapeutically effective amount of an immunotherapy treatment on the basis of the abundance of said at least one bacteria.

### RELATED APPLICATIONS

This application is a Continuation of PCT Pat. Application No. PCT/IL2021/050388, having international filing date of Apr. 6, 2021 which claims the benefit of priority under 35 USC § 119(e) of U.S. Provisional Pat. Application No. 63/005,540 filed on Apr. 6, 2020. The contents of the above applications are all incorporated by reference as if fully set forth herein in their entirety.

### SEQUENCE LISTING STATEMENT

The XML file, entitled 93676SequenceListing.xml, created on Oct. 3, 2022, comprising 723,942 bytes, submitted concurrently with the filing of this application is incorporated herein by reference.

### FIELD AND BACKGROUND OF THE INVENTION

The present invention, in some embodiments thereof, relates to methods of diagnosing cancer and predicting responsiveness to therapy based on tumor microbiome profiles.

More than 16 % of cancer incidence worldwide is estimated to be attributed to infectious agents. Over the years, intra-tumor bacteria have been reported in many tumor types, but a comprehensive characterization of these bacteria is still far from complete.

Background art includes US Pat. No. 20190365830, Gopalakrishnan V et al, Science.2018 Jan 5; 359(6371): 97-103; Geller et al., Science, Vol 357, Issue 635615 Sep. 2017; Riquelme E et al Cell. 2019 Aug 8;178(4):795-806.e12. doi:10.1016/j.cell.2019.07.008; Straussman R et al., Nature. 2012 Jul 26;487(7408):500-4. doi:10.1038/nature11183.

### SUMMARY OF THE INVENTION

According to an aspect of the present invention there is provided a method of treating cancer in a subject in need thereof the method comprising:
(a) analyzing for the abundance of at least one bacteria in the tumor microbiome of the subject;
(b) administering to the subject a therapeutically effective amount of an immunotherapy treatment on the basis of the abundance of the at least one bacteria.

According to an aspect of the present invention there is provided a method of determining whether a subject having been diagnosed with cancer will respond to an immunotherapy treatment comprising analyzing the abundance of at least one bacteria set forth in Table 4 in the tumor microbiome of the subject, wherein the abundance of the at least one bacteria is indicative whether a subject will respond to the immunotherapy treatment.

According to an aspect of the present invention there is provided a method of diagnosing a cancer in a subject, comprising analyzing the abundance of a bacteria of at least one family, order, genus or species set forth in any of Tables 1-3 in a tumor sample of the subject, wherein an abundance of the bacteria above a predetermined level is indicative of the cancer.

According to an aspect of the present invention there is provided a method of delivering an agent to a tumor of a subject, the method comprising administering to the subject bacteria which comprise or are linked to the agent, wherein the bacteria is of a family, order, genus or species set forth in Tables 1-3, thereby delivering the agent to a tumor of the subject.

According to an aspect of the present invention there is provided a composition of matter comprising a bacteria of a family, order, genus or species set forth in Tables 1-3 which comprises a therapeutic or diagnostic agent.

Accordance to embodiments of the present invention, the abundance of a bacteria in the tumor microbiome set forth in Table 5 is above a predetermined amount, the subject is deemed a suitable candidate for therapy using the immunotherapy treatment.

Accordance to embodiments of the present invention, when the abundance of a bacteria in the tumor microbiome set forth in Table 6 is below a predetermined amount, the subject is deemed a suitable candidate for therapy using the immunotherapy treatment.

Accordance to embodiments of the present invention, the immunotherapy treatment comprises an immune checkpoint inhibitor.

Accordance to embodiments of the present invention, the cancer is melanoma.

Accordance to embodiments of the present invention, the at least one bacteria is set forth in Table 4.

Accordance to embodiments of the present invention, the at least one bacteria is set forth in Table 5, the abundance above a predetermined level is indicative that the subject will respond to the immune checkpoint inhibitor.

Accordance to embodiments of the present invention, when the at least one bacteria is set forth in Table 6, the abundance below a predetermined level is indicative that the subject will respond to the immune checkpoint inhibitor.

Accordance to embodiments of the present invention, the at least one bacteria comprises each of the bacteria set forth in Table 4.

Accordance to embodiments of the present invention, the tumor is a metastasized tumor.

Accordance to embodiments of the present invention, the tumor is a non-metastasized tumor.

Accordance to embodiments of the present invention, the at least one family, order, genus or species comprises at least three family, order, genus or species.

Accordance to embodiments of the present invention, the cancer is selected from the group consisting of breast, melanoma, pancreatic cancer, ovarian cancer, bone cancer and brain cancer.

Accordance to embodiments of the present invention, the brain cancer comprises glioblastoma.

Accordance to embodiments of the present invention, the tumor sample is a non-metastasized tumor sample.

Accordance to embodiments of the present invention, the tumor sample is a metastasized tumor sample.

Accordance to embodiments of the present invention, the agent is a therapeutic agent.

Accordance to embodiments of the present invention, the therapeutic agent is a cytotoxic agent.

Accordance to embodiments of the present invention, the agent is a diagnostic agent.

Accordance to embodiments of the present invention, the bacteria are genetically modified to express the agent.

Accordance to embodiments of the present invention, the tumor is selected from the group consisting of a breast tumor, a lung tumor, a skin tumor, a pancreas tumor, an ovarian tumor, a bone tumor and a brain tumor.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D. Bacterial components are detected in human tumors. (A) Number of human samples analyzed in the study. Normal samples include both normal and normal adjacent to tumor (NAT) samples. (B) The presence of bacterial DNA in human tumors was assessed by bacterial 16S rDNA qPCR. A calibration curve, generated by spiking bacterial DNA into human DNA, was used to estimate bacterial load which was then normalized against batch-specific qPCR no-template controls (NTC). Negative values were floored to 0.1. Red bars represent the median. The proportion of samples of each cancer type that had more bacteria than the 99th percentile of the negative control samples (black bar) is depicted above each cancer type. (C) Heatmap representing the proportion of tumors that stained positively for 16S rRNA, LPS or LTA. n= 40 to 101/tumor type. (D) Consecutive slices from four human tumor types were stained with hematoxylin and eosin (H&E), anti-LPS antibody, or with FISH probes against bacterial 16S rRNA. Scale bars represent 200 µm.

FIGS. 2A-E. Intra-tumor bacteria are found inside both cancer and immune cells. (A) Summary of the staining patterns of LPS, LTA, and bacterial 16S rRNA in different cell types across 459, 427 and 354 tumor cores, respectively. CD45+/CD68+ are referred to as macrophages; CD45+/CD68- cells are referred to as other immune cells. (B-D) Representative cores are shown demonstrating the different staining patterns in human tumors. (B) Bacterial LPS and 16S rRNA are demonstrated in breast cancer cells. (C) Bacterial LPS and 16S rRNA are demonstrated in CD45+/CD68- cells of a highly inflamed breast tumor. (D) A melanoma tumor demonstrating typical staining of macrophage-associated bacteria (M), with positive LPS and LTA staining, but no 16S rRNA staining. Nearby tumor cells (T) show the typical LPS and 16S rRNA staining, with negative LTA staining. Each insert demonstrates a low magnification of the entire core. Asterisks mark the region that was selected for higher magnification. Scale bar in high magnification images represent 20 µm. (E) Combined light and electron microscopy (CLEM) demonstrates intra-cellular bacteria in human breast cancer. IF image shows DAPI in blue and LPS in red. Two bacteria are marked with arrows. TEM images of the same cell are shown in grayscale. High magnification image of the boxed area is shown on the right. ‘N’ marks the cell nucleus.

FIGS. 3A-G. The microbiome of breast tumors is richer and more diverse than that of other tumor types. (A) Graphic representation of the bacterial ribosomal 16S rRNA gene with its conserved (blue) and variable (yellow) regions. The sequence from E. coli K-12 substrain MG1655 was used as a reference sequence. The five amplicons of the multiplexed 5R PCR method are depicted in gray. (B) Schematic representation of the analysis pipeline applied to 16S rDNA sequencing data. (C) Rarefaction plots showing the number of bacterial genera that passed all filters in the different tumor types per number of samples. Light color sleeves represent confidence intervals based on 100 random sub-sampling for each sample size. (D) Box blot of Shannon diversity indexes of all samples, segregated by tumor type. (E) Box blot of the numbers of bacterial species present in each tumor. For (D) and (E) values were calculated on rarefied data of 40 samples per tumor type, with 10 iterations. For each iteration only bacteria that passed all filters in any of the tumor types were included in the analysis. (F) Rarefaction plots for the number of bacterial genera that passed all filters in breast tumor, breast NAT, and breast normal samples. Light color sleeves represent confidence intervals based on 100 random sub-sampling for each sample size. (G) Fluorescent images from four human breast tumors that were cultured ex-vivo with fluorescently labeled D-alanine for 2 hours (blue). Nuclei were stained with DRAQ5 (orange). Scale bars represent 10 µm.

FIGS. 4A-G. Different tumor types have distinct microbial compositions. (A) Jaccard similarity indexes were computed based on profiles of bacterial species that passed all filters in tumors (n=528) between all possible pairs of samples. The heatmap presents the average of all indexes between sample pairs from any two cancer types. (B) Distribution of order-level phylotypes across different tumor types. Relative abundances were calculated by summing up the reads of species that passed all filters in the different tumor types and belong to the same order. Orders are colored according to their associated phylum. (C) Unsupervised hierarchical clustering of all bacteria species that were hits in one of the tumor types and are present in 10% or more of the samples in at least one of the tumor types (n=137). (D) The prevalence of 19 bacteria from panel C, displayed across the different tumor types. Only bacteria that at a hit in a given tumor type are represented with colored circles.. Circle size indicates the prevalence level in samples. US=unknown species. (E) Bacterial taxa with a significant differential prevalence between different breast tumor subtypes are presented in a bar plot. A two sample proportion z-test was applied on the prevalences of taxa comparing between HER2+ (n=61) and HER2- (n=247), ER+ (n=270) and ER- (n=49) or triple negative (TNG, n=36) and non-TNG (n=284) breast tumors to calculate p-values. The direction of the bars indicates the enrichment direction. All bacteria presented had an FDR corrected q-value <0.25. US=unknown species, UG=unknown genus, UF=unknown family. (F) Principal Coordinate Analysis (PCoA) biplot on the Jaccard similarity indexes between bacterial species profiles of the different tissue types. Only bacteria that passed all filters for the specific tissue type were considered. Tumor types and their normal tissue are grouped in circles. (G) Volcano plot demonstrating the differential prevalence of bacteria between tumors and their NAT in breast, lung and ovary. A two sample proportion z-test was used to calculate the p-values. Sizes of dots reflect phylotype levels, gradually increasing from species to phylum. Bacteria are color-filled according to the tumor type (breast-pink, lung-green, ovary-purple) if they passed significance thresholds (effect size > 5%, p-value < 0.05 and FDR-corrected q-value<0.25).

FIGS. 5A-G. Predicted bacterial metabolic functions are associated with clinical metadata. (A) A heat map demonstrating unsupervised hierarchical clustering of the frequencies of 287 MetaCyc pathways across the different tumor types. Only pathways that are abundant (frequency > 10% in at least one tumor type) and variable (standard deviation/average of frequencies > 0.4) were included. (B,C) Volcano plots demonstrating bacterial MetaCyc pathways (B) and taxa (C) that are enriched in lung tumors from smokers (n=100) vs. never-smokers (n=43). Effect size represents the difference in the proportion between the groups. A two sample proportion z-test was used to calculate the p-values. Green filled circles indicate pathways with FDR-corrected q-values<0.25. Degrading pathways of smoke chemicals are indicated by blue circles in (B); plant related metabolic pathways are indicated by red circles in (B). (D) Bacterial species that contributed to cigarette smoke metabolites degradation functions (blue ring) and to the biosynthesis of plant metabolites functions (red ring) are indicated on the phylogenetic tree, together with all bacteria that are hits in lung tumors (green ring). (E) Volcano plot demonstrating enriched bacterial MetaCyc functions in ER+ vs. ER- breast tumors. A two sample proportion z-test was used to calculate the p-values. Colored circles indicate pathways with FDR-corrected q-values<0.25. (F) Volcano plot demonstrating the bacterial taxa enriched in melanoma patients who responded to immune checkpoint inhibitors (ICI) vs. non-responders. A binomial test was used to calculate the p-values for the enrichment or depletion of bacterial taxa in the responder cohort vs the non-responder cohort. The size of dots reflects phylotype levels gradually increasing from species to phylum. Circles with color indicate taxa with FDR-corrected q-values<0.25. (G) Differentially prevalent bacterial taxa from panel F (n=46) were used to stratify the melanoma patients cohort according to the presence or absence of a favorable bacterial signature (supplementary methods). Progression-free survival is demonstrated for both groups of patients. P-value was calculated using a log rank test.

FIG. 6. Distribution of family-level phylotypes across the different tumor types. Relative abundances were calculated by summing up all reads of tumor (including colon tumors) hits species belonging to the same family. Families are colored according to their associated phylum: (Proteobacteria, blue; Firmicutes, green; Actinobacteria, yellow; Bacteroidetes, purple; Fusobacteria, red; and Cyanobacteria, pink).

FIG. 7. Reads frequency and prevalence of specific bacteria across different tumor types. The reads frequencies of five bacteria in all tumor samples and negative controls are plotted according to tumor type. The prevalence of the bacteria (% positive samples in each tumor type) is depicted at the top of the graph for each tumor type. Dots are colored if the bacteria were a hit for a given tumor type or kept grey if they weren’t a hit for a given tumor. For graphs 1, 3, and 4, data were clipped at 1%, for graph 2 at 4% and for graph 5 at 10%.

### DESCRIPTION OF SPECIFIC EMBODIMENTS OF THE INVENTION

The present invention, in some embodiments thereof, relates to methods of diagnosing cancer and predicting responsiveness to therapy based on tumor microbiome profiles.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Despite substantial advances in cancer treatment, resistance to therapy remains a foremost challenge. Several laboratories have reported that nonmalignant cells in the tumor microenvironment contribute to anticancer drug resistance. In addition, the present inventors have previously shown that pancreatic ductal adenocarcinoma PDACs contain bacteria that can potentially modulate tumor sensitivity to gemcitabine (Geller et al., Science, Vol 357, Issue 6356, 15 Sep. 2017).

The present inventors have now characterized the microbiome of 1,526 samples from seven human tumor types (including controls), whilst using multiple measures to minimize and control for contaminations. The exploration of multiple tumor types with a single platform allowed them to accurately compare different tumor types and uncover cancer type-specific microbial signatures. Extending their analysis to the functional level demonstrated that despite a very large variation in taxa levels, certain tumor environments are enriched for common, relevant bacterial functional traits.

Whilst further reducing the present invention to practice, the present inventors used multiple visualization methods and culturomics in order to validate the presence of bacteria in the tumors and demonstrate their intra-cellular localization in both cancer and immune cells.

As is illustrated hereinunder and in the examples section which follows, the present inventors show that it is possible to predict the response to immune checkpoint inhibitors on the basis of a bacterial signature in the tumor microbiome.

Consequently, the present teachings suggest both diagnosis and treatment selection based on the turmor microbiome.

Thus, according to a first aspect of the present invention, there is provided a method of treating cancer in a subject in need thereof the method comprising:
(a) analyzing for the abundance of at least one bacteria in the tumor microbiome of the subject;
(b) administering to the subject a therapeutically effective amount of an immune checkpoiont inhibitor on the basis of the abundance of the at least one bacteria.

As used herein, the term “treating” includes abrogating, substantially inhibiting, slowing or reversing the progression of a condition, substantially ameliorating clinical or aesthetical symptoms of a condition. According to a particular embodiment, the term treating also refers to substantially preventing the appearance of clinical or aesthetical symptoms of a condition.

Particular subjects which are treated are mammalian subjects - e.g. humans.

According to a particular embodiment, the subject has been diagnosed as having cancer.

### Cancer

The term “cancer” as used herein refers to an uncontrolled, abnormal growth of a host’s own cells which may lead to invasion of surrounding tissue and potentially tissue distal to the initial site of abnormal cell growth in the host. Major classes include carcinomas which are cancers of the epithelial tissue (e.g., skin, squamous cells); sarcomas which are cancers of the connective tissue (e.g., bone, cartilage, fat, muscle, blood vessels, etc.); leukemias which are cancers of blood forming tissue (e.g., bone marrow tissue); lymphomas and myelomas which are cancers of immune cells; and central nervous system cancers which include cancers from brain and spinal tissue. “Cancer(s),” “neoplasm(s),” and “tumor(s)” are used herein interchangeably. As used herein, “cancer” refers to all types of cancer or neoplasm or malignant tumors including leukemias, carcinomas and sarcomas, whether new or recurring.

Specific examples of cancers that may be treated using the bacteria described herein include, but are not limited to adrenocortical carcinoma, hereditary; bladder cancer; breast cancer; breast cancer, ductal; breast cancer, invasive intraductal; breast cancer, sporadic; breast cancer, susceptibility to; breast cancer, type 4; breast cancer, type 4; breast cancer-1; breast cancer-3; breast-ovarian cancer; triple negative breast cancer, Burkitt’s lymphoma; cervical carcinoma; colorectal adenoma; colorectal cancer; colorectal cancer, hereditary nonpolyposis, type 1; colorectal cancer, hereditary nonpolyposis, type 2; colorectal cancer, hereditary nonpolyposis, type 3; colorectal cancer, hereditary nonpolyposis, type 6; colorectal cancer, hereditary nonpolyposis, type 7; dermatofibrosarcoma protuberans; endometrial carcinoma; esophageal cancer; gastric cancer, fibrosarcoma, glioblastoma multiforme; glomus tumors, multiple; hepatoblastoma; hepatocellular cancer; hepatocellular carcinoma; leukemia, acute lymphoblastic; leukemia, acute myeloid; leukemia, acute myeloid, with eosinophilia; leukemia, acute nonlymphocytic; leukemia, chronic myeloid; Li-Fraumeni syndrome; liposarcoma, lung cancer; lung cancer, small cell; lymphoma, non-Hodgkin’s; lynch cancer family syndrome II; male germ cell tumor; mast cell leukemia; medullary thyroid; medulloblastoma; melanoma, malignant melanoma, meningioma; multiple endocrine neoplasia; multiple myeloma, myeloid malignancy, predisposition to; myxosarcoma, neuroblastoma; osteosarcoma; osteocarcinoma, ovarian cancer; ovarian cancer, serous; ovarian carcinoma; ovarian sex cord tumors; pancreatic cancer; pancreatic endocrine tumors; paraganglioma, familial nonchromaffin; pilomatricoma; pituitary tumor, invasive; prostate adenocarcinoma; prostate cancer; renal cell carcinoma, papillary, familial and sporadic; retinoblastoma; rhabdoid predisposition syndrome, familial; rhabdoid tumors; rhabdomyosarcoma; small-cell cancer of lung; soft tissue sarcoma, squamous cell carcinoma, basal cell carcinoma, head and neck; T-cell acute lymphoblastic leukemia; Turcot syndrome with glioblastoma; tylosis with esophageal cancer; uterine cervix carcinoma, Wilms’ tumor, type 2; and Wilms’ tumor, type 1, and the like.

According to a particular embodiment, the cancer is cancer is selected from the group consisting of breast, melanoma, pancreatic cancer, ovarian cancer, bone cancer and brain cancer (e.g. glioblastoma).

According to another embodiment, the cancer is melanoma.

Malignant melanomas are clinically recognized based on the ABCD(E) system, where A stands for asymmetry, B for border irregularity, C for color variation, D for diameter >5 mm, and E for evolving. Further, an excision biopsy can be performed in order to corroborate a diagnosis using microscopic evaluation. Infiltrative malignant melanoma is traditionally divided into four principal histopathological subgroups: superficial spreading melanoma (SSM), nodular malignant melanoma (NMM), lentigo maligna melanoma (LMM), and acral lentiginous melanoma (ALM). Other rare types also exists, such as desmoplastic malignant melanoma. A substantial subset of malignant melanomas appear to arise from melanocytic nevi and features of dysplastic nevi are often found in the vicinity of infiltrative melanomas. Melanoma is thought to arise through stages of progression from normal melanocytes or nevus cells through a dysplastic nevus stage and further to an in situ stage before becoming invasive. Some of the subtypes evolve through different phases of tumor progression, which are called radial growth phase (RGP) and vertical growth phase (VGP).

In a particualar embodiment, the melanoma resistant to treatment with inhibitors of BRAF and/or MEK.

The term “tumor microbiome” refers to the totality of microbes (bacteria, fungae, protists), their genetic elements (genomes) in a defined environment, e.g. within the tumor of a host. In a particular embodiment, the microbiome refers only to the totality of bacteria in a defined environment, e.g. within the tumor of a host.

The tumor may be a primary tumor or a secondary tumor (i.e. metastasized tumor).

Methods of quantifying levels (i.e. abundance) of bacteria are described herein below and in the Example section below. Care should be taken to take a sufficient number of measurements to minimize and control for contaminations.

In some embodiments, determining a level of one or more bacteria or components or products thereof comprises determining a level or set of levels of one or more DNA sequences. In some embodiments, one or more DNA sequences comprises any DNA sequence that can be used to differentiate between different bacterial types. In certain embodiments, one or more DNA sequences comprises 16S rRNA gene sequences. In certain embodiments, one or more DNA sequences comprises 18S rRNA gene sequences. In some embodiments, 1, 2, 3, 4, 5, 10, 15, 20, 25, 50, 100, 1,000, 5,000 or more sequences are amplified.

In some embodiments, a microbiota sample (e.g. tumor sample) is directly assayed for a level or set of levels of one or more DNA sequences. In some embodiments, DNA is isolated from a microbiota sample and isolated DNA is assayed for a level or set of levels of one or more DNA sequences. Methods of isolating microbial DNA are well known in the art. Examples include but are not limited to phenol-chloroform extraction and a wide variety of commercially available kits, including QIAamp DNA Stool Mini Kit (Qiagen, Valencia, Calif.).

In some embodiments, a level or set of levels of one or more DNA sequences is determined by amplifying DNA sequences using PCR (e.g., standard PCR, semi-quantitative, or quantitative PCR). In some embodiments, a level or set of levels of one or more DNA sequences is determined by amplifying DNA sequences using quantitative PCR. These and other basic DNA amplification procedures are well known to practitioners in the art and are described in Ausebel et al. (Ausubel F M, Brent R, Kingston R E, Moore D D, Seidman J G, Smith J A, Struhl K (eds). 1998. Current Protocols in Molecular Biology. Wiley: New York).

In some embodiments, DNA sequences are amplified using primers specific for one or more sequence that differentiate(s) individual microbial types from other, different microbial types. In some embodiments, 16S rRNA gene sequences or fragments thereof are amplified using primers specific for 16S rRNA gene sequences. In some embodiments, 18S DNA sequences are amplified using primers specific for 18S DNA sequences.

In some embodiments, a level or set of levels of one or more 16S rRNA gene sequences is determined using phylochip technology. Use of phylochips is well known in the art and is described in Hazen et al. (“Deep-sea oil plume enriches indigenous oil-degrading bacteria.” Science, 330, 204-208, 2010), the entirety of which is incorporated by reference. Briefly, 16S rRNA genes sequences are amplified and labeled from DNA extracted from a microbiota sample. Amplified DNA is then hybridized to an array containing probes for microbial 16S rRNA genes. Level of binding to each probe is then quantified providing a sample level of microbial type corresponding to 16S rRNA gene sequence probed. In some embodiments, phylochip analysis is performed by a commercial vendor. Examples include but are not limited to Second Genome Inc. (San Francisco, Calif.).

In some embodiments, determining a level or set of levels of one or more types of microbes or components or products thereof comprises determining a level or set of levels of one or more microbial RNA molecules (e.g., transcripts). Methods of quantifying levels of RNA transcripts are well known in the art and include but are not limited to northern analysis, semi-quantitative reverse transcriptase PCR, quantitative reverse transcriptase PCR, and microarray analysis.

In some embodiments, determining a level or set of levels of one or more types of microbes or components or products thereof comprises determining a level or set of levels of one or more microbial polypeptides. Methods of quantifying polypeptide levels are well known in the art and include but are not limited to Western analysis and mass spectrometry. These and all other basic polypeptide detection procedures are described in Ausebel et al.

In some embodiments, determining a level or set of levels of one or more types of microbes or components or products thereof comprises determining a level or set of levels of one or more microbial metabolites. In some embodiments, levels of metabolites are determined by mass spectrometry. In some embodiments, levels of metabolites are determined by nuclear magnetic resonance spectroscopy. In some embodiments, levels of metabolites are determined by enzyme-linked immunosorbent assay (ELISA). In some embodiments, levels of metabolites are determined by colorimetry. In some embodiments, levels of metabolites are determined by spectrophotometry.

According to a specific embodiment, the bacteria which is used to analyze whether a subject should be treated with an immunotherapy is one which is set forth in Table 4, herein below.

According to another embodiment, the bacteria which is used to analyze whether a subject should be treated with an immunotherapy is one which is set forth in Table 4.1, herein below.

More specifically, wherein when the abundance of a bacteria in the tumor microbiome set forth in Table 5 is increased above a predetermined amount, the subject is deemed a suitable candidate for therapy using the immunotherapy treatment. In a particular embodiment, the presence of a bacteria set forth in Table 5 is indicative that the candidate is suitable for immunotherapy treatment.

The term “increase” means a change, such that the difference is at least 5 %, 10%, 20%, 30%, 40%, 50%, 60%, 70%, 80%, 90%, 2-fold, 4-fold, 10-fold, 100-fold, 10³fold, 10⁴ fold, 10⁵ fold, 10⁶ fold, and/or 10⁷ fold greater than the amount of bacteria retrieved from the same organ of a subject who does not have cancer (e.g. a healthy subject).

When the abundance of a bacteria in the tumor microbiome set forth in Table 6 is decreased below a predetermined amount, the subject is deemed a suitable candidate for therapy using the immunotherapy treatment. In a particular embodiment, the absence of a bacteria set forth in Table 6 is indicative that the candidate is suitable for immunotherapy treatment.

The term “decrease” means a change, such that the difference is at least 5 %, 10%, 20%, 30%, 40%, 50%, 60%, 70%, 80%, 90%, 2-fold, 4-fold, 10-fold, 100-fold, 10³fold, 10⁴ fold, 10⁵ fold, 10⁶ fold, and/or 10⁷ fold less than the amount of bacteria retrieved from the same organ of a subject who does not have cancer (e.g. a healthy subject).

According to a particular embodiment, at least two of the bacteria from Table 4 are analyzed and when the level of at least two of the bacteria change (in the direction specified in Tables 5 and 6, the subject is deemed a candidate for immunotherapy. In one embodiment, the at least two bacteria are the top two bacteria listed in Table 4.

According to a particular embodiment, at least five of the bacteria from Table 4 are analyzed and when the level of at least two of the bacteria change (in the direction specified in Tables 5 and 6, the subject is deemed a candidate for immunotherapy. In one embodiment, the at least five bacteria are the top five bacteria listed in Table 4.

According to a particular embodiment, at least ten of the bacteria from Table 4 are analyzed and when the level of at least two of the bacteria change (in the direction specified in Tables 5 and 6, the subject is deemed a candidate for immunotherapy. In one embodiment, the at least ten bacteria are the top ten bacteria listed in Table 4.

According to a particular embodiment, at least fifteen of the bacteria from Table 4 are analyzed and when the level of at least two of the bacteria change (in the direction specified in Tables 5 and 6, the subject is deemed a candidate for immunotherapy. In one embodiment, the at least 15 bacteria are the top 15 bacteria listed in Table 4.

According to a particular embodiment, at least twenty of the bacteria from Table 4 are analyzed and when the level of at least two of the bacteria change (in the direction specified in Tables 5 and 6, the subject is deemed a candidate for immunotherapy. In one embodiment, the at least 20 bacteria are the top 20 bacteria listed in Table 4.

According to a particular embodiment, at least twenty five of the bacteria from Table 4 are analyzed and when the level of at least two of the bacteria change (in the direction specified in Tables 5 and 6, the subject is deemed a candidate for immunotherapy. In one embodiment, the at least 25 bacteria are the top 25 bacteria listed in Table 4.

According to a particular embodiment, at least thirty of the bacteria from Table 4 are analyzed and when the level of at least two of the bacteria change (in the direction specified in Tables 5 and 6, the subject is deemed a candidate for immunotherapy. In one embodiment, the at least 30 bacteria are the top 30 bacteria listed in Table 4.

According to a particular embodiment, at least thirty five of the bacteria from Table 4 are analyzed and when the level of at least two of the bacteria change (in the direction specified in Tables 5 and 6, the subject is deemed a candidate for immunotherapy. In one embodiment, the at least 35 bacteria are the top 35 bacteria listed in Table 4.

According to a particular embodiment, at least forty of the bacteria from Table 4 are analyzed and when the level of at least two of the bacteria change (in the direction specified in Tables 5 and 6, the subject is deemed a candidate for immunotherapy. In one embodiment, the at least 40 bacteria are the top 40 bacteria listed in Table 4.

According to a particular embodiment, at least forty five of the bacteria from Table 4 are analyzed and when the level of at least two of the bacteria change (in the direction specified in Tables 5 and 6, the subject is deemed a candidate for immunotherapy. In one embodiment, the at least two bacteria are the top 45 bacteria listed in Table 4.

According to a particular embodiment, all of the bacteria from Table 4 are analyzed and when the level of at least two of the bacteria change (in the direction specified in Tables 5 and 6), the subject is deemed a candidate for immunotherapy.

When referring to Tables 4, 5 or 6, it will be appreciated that the bacteria which is analysed is defined by its highest (most specific) taxonomic assignment.

Thus, for example in the case of the first row of Table 4, bacteria of the genus Mycobacterium are analysed. An enrichment of this genus signifies that the subject may be a candidate for immunotherapy. In the case of the second row of Table 4, bacteria of the species Veillonella dispar, having the 16 s rRNA sequence as set forth in SEQ ID NO: 311 are analysed. A depletion of this species signifies that the subject may be a candidate for immunotherapy. In the case of the third row of Table 4, bacteria of the family Veillonellaceae are analysed. A depletion of this family signifies that the subject may be a candidate for immunotherapy.

The term “immunotherapy” refers to a treatment that uses a subject’s immune system to treat cancer and includes, for example, checkpoint inhibitors, cancer vaccines, cytokines, cell therapy, CAR-T cells, and dendritic cell therapy.

According to a particular embodiment, the immunotherapy treatment includes an immune checkpoint inhibitor.

As used herein, the phrase “immune checkpoint inhibitor” refers to a compound capable of inhibiting the function of an immune checkpoint protein. Inhibition includes reduction of function and full blockade. In particular the immune checkpoint protein is a human immune checkpoint protein. Thus the immune checkpoint protein inhibitor preferably is an inhibitor of a human immune checkpoint protein. Immune checkpoint proteins are described in the art (see for instance Pardoll, 2012. Nature Rev. Cancer 12: 252-264). The designation immune checkpoint includes the experimental demonstration of stimulation of an antigen-receptor triggered T lymphocyte response by inhibition of the immune checkpoint protein in vitro or in vivo, e.g. mice deficient in expression of the immune checkpoint protein demonstrate enhanced antigen-specific T lymphocyte responses or signs of autoimmunity (such as disclosed in Waterhouse et al., 1995. Science 270:985-988; Nishimura et al., 1999. Immunity 11:141-151). It may also include demonstration of inhibition of antigen-receptor triggered CD4+ or CD8+ T cell responses due to deliberate stimulation of the immune checkpoint protein in vitro or in vivo (e.g. Zhu et al., 2005. Nature Immunol. 6:1245-1252).

Preferred immune checkpoint protein inhibitors are antibodies that specifically recognize immune checkpoint proteins. A number of CTLA-4, PD1, PDL-1, PD-L2, LAG-3, BTLA, B7H3, B7H4, TIM3 and KIR inhibitors are known and in analogy of these known immune checkpoint protein inhibitors, alternative immune checkpoint inhibitors may be developed in the (near) future. For example ipilimumab is a fully human CTLA-4 blocking antibody presently marketed under the name Yervoy (Bristol-Myers Squibb). A second CTLA-4 inhibitor is tremelimumab (referenced in Ribas et al, 2013, J. Clin. Oncol. 31:616-22).

Examples of PD-1 inhibitors include without limitation humanized antibodies blocking human PD-1 such as lambrolizumab (e.g. disclosed as hPD109A and its humanized derivatives h409A11, h409A16 and h409A17 in WO2008/156712; Hamid et al., N. Engl. J. Med. 369: 134-144 2013,), or pidilizumab (disclosed in Rosenblatt et al., 2011. J. Immunother. 34:409-18), as well as fully human antibodies such as nivolumab (previously known as MDX-1106 or BMS-936558, Topalian et al., 2012. N. Eng. J. Med. 366:2443-2454, disclosed in U.S. Pat. No. 8,008,449 B2). Other PD-1 inhibitors may include presentations of soluble PD-1 ligand including without limitation PD-L2 Fc fusion protein also known as B7-DC-Ig or AMP-244 (disclosed in Mkrtichyan M, et al. J Immunol. 189:2338-47 2012) and other PD-1 inhibitors presently under investigation and/or development for use in therapy.

In addition, immune checkpoint inhibitors may include without limitation humanized or fully human antibodies blocking PD-L such as MEDI-4736 (disclosed in WO2011066389 A1), MPDL3280A (disclosed in U.S. Pat. No. 8,217,149 B2) and MIH1 (Affymetrix obtainable via eBioscience (16.5983.82)) and other PD-L1 inhibitors presently under investigation. According to this invention an immune checkpoint inhibitor is preferably selected from a CTLA-4, PD-1 or PD-L1 inhibitor, such as selected from the known CTLA-4, PD-1 or PD-L1 inhibitors mentioned above (ipilimumab, tremelimumab, labrolizumab, nivolumab, pidilizumab, AMP-244, MEDI-4736, MPDL3280A and MIH1). Known inhibitors of these immune checkpoint proteins may be used as such or analogues may be used, in particular chimerized, humanized or human forms of antibodies.

Other agents used in the arsenal of cancer immunotherapy agents include T cell populations that are capable of binding to the peptide epitopes of the tumor for adoptive cell therapy (ACT).

ACT refers to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73).In one embodiment TCRs are selected for administering to a subject based on binding to neoantigens. In one embodiment T cells are expanded using methods known in the art. Expanded T cells that express tumor specific TCRs may be administered back to a subject. In another embodiment PBMCs are transduced or transfected with polynucleotides for expression of TCRs and administered to a subject. T cells expressing TCRs specific to neoantigens are expanded and administered back to a subject.

Other immunotherapy treatments include T cell populations expressing chimeric antibodies (CAR-T cells) on the surface thereof that can bind to at least one peptide epitope of the tumor.

If the subject is found not to be a candidate for immunotherapy, alternative drugs and treatments may be offered. Such treatments include radiotherapy, chemotherapeutic agents etc. The other drugs/treatments may be the typical gold standard for that cancer.

According to another aspect of the present invention, there is provided a method of diagnosing a cancer in a subject, comprising analyzing the abundance of a bacteria of at least one family, order, genus or species set forth in any of Tables 1-3 in a tumor sample of the subject, wherein an abundance of said bacteria above a predetermined level is indicative of the cancer.

As used herein, the term “diagnosing” refers to determining presence or absence of the cancer, classifying the cancer, determining a severity of the cancer, monitoring cancer progression, forecasting an outcome of a pathology and/or prospects of recovery and/or screening of a subject for the cancer.

According to some embodiments of the invention, diagnosing of the subject for cancer is followed by substantiation of the screen results using gold standard methods.

According to some embodiments of the invention, the method further comprising informing the subject of the diagnosis.

As used herein the phrase “informing the subject” refers to advising the subject that based on the diagnosis the subject should seek a suitable treatment regimen.

Once the diagnosis is determined, the results can be recorded in the subject’s medical file, which may assist in selecting a treatment regimen and/or determining prognosis of the subject.

Cancers which may be diagnosed have been disclosed herein above.

The abundance of bacterial taxa set forth in Tables 1-3 are analyzed.

It will be appreciated that the level of a particular bacteria correlates with a particular cancer. Thus, particular taxa of bacteria for diagnosing breast cancer are recorded in Table 1.

Particular taxa of bacteria for diagnosing breast, lung and ovarian cancer are disclosed in Table 2.

Particular taxa of bacteria for diagnosing breast, melanoma, pancreatic cancer, ovarian cancer, bone cancer and brain cancer are disclosed in Table 3.

Tumor samples are described herein above.

Quantifying levels of bacteria are described herein above.

More specifically, wherein when the abundance of a bacteria in the tumor microbiome set forth in any of Tables 1-3 is increased/decreased above/below a predetermined amount, the subject is diagnosed as having cancer. It will be appreciated that the direction of change in Tables 1 and 3 is an increase. The direction of change in Table 2 is marked in the column with the heading E/D (enriched or depleted).

The term “increase” throughout the application, refers to a change, such that the difference is at least 10%, 20%, 30%, 40%, 50%, 60%, 70%, 80%, 90%, 2-fold, 4-fold, 10-fold, 100-fold, 10³fold, 10⁴ fold, 10⁵ fold, 10⁶ fold, and/or 10⁷ fold greater than the amount of bacteria retrieved from the same organ of a subject who does not have cancer (e.g. a healthy subject).

According to a particular embodiment, at least three or at least five of the bacterial taxa from any of Tables 1-3 are analyzed and when the level of each of the bacterial taxa is altered above a predetermined amount, the subject is diagnosed as having cancer.

In one embodiment, at least the top 5 bacterial taxa from tables 2 and/or 3 are analysed. In another embodiment, at least the top 10 bacterial taxa from tables 2 and/or 3 are analyzed. In another embodiment, at least the top 20 bacterial taxa from tables 2 and/or 3 are analyzed. In another embodiment, at least the top 30 bacterial taxa from tables 2 and/or 3 are analyzed. In another embodiment, at least the top 40 bacterial taxa from tables 2 and/or 3 are analyzed. In another embodiment, at least the top 50 bacterial taxa from tables 2 and/or 3 are analyzed. In another embodiment, at least the top 60 bacterial taxa from tables 2 and/or 3 are analyzed. In another embodiment, at least the top 70 bacteria from tables 2 and/or 3 are analyzed.

According to another aspect of the present invention there is provided a method of delivering an agent to a tumor of a subject, the method comprising administering to the subject bacteria which comprise or are linked to the agent, wherein the bacteria is of a family, order, genus or species set forth in Tables 1-3.

The particular bacteria for the tumor type is specified in Tables 1-3

In some embodiments, the bacteria constitutively express the agent. In some embodiments, the bacteria conditionally express the agent (e.g., in response to a quorum sensing switch and/or an environmental change, such as a change in pH, a change in bacterial population density, a change in the environmental oxygen levels and a change in available sugar sources).

In one embodiment, the agent is an RNA or a protein and is “overexpressed” in the bacteria. The term “overexpressed in a bacteria” refers to the expression at a higher level in an engineered bacteria under at least some conditions than it is expressed by a wild-type bacteria of the same species under the same conditions. Similarly, a gene is “underexpressed” in a bacteria if it is expressed at a lower level in an engineered bacteria under at least some conditions than it is expressed by a wild-type bacteria of the same species under the same conditions.

In some embodiments, the conditionally expressed gene is operably linked to a low-pH induced promoter, such as STM1787. In some embodiments, the conditionally expressed gene is operably linked to a hypoxia-induced promoter, such as pepT, pflE, ansB, vhb or FF+20*. In some embodiments, the gene encodes a cancer therapeutic described herein. In some embodiments, the gene encodes a prodrug enzyme described herein. In some embodiments, the gene encodes a protein that causes the lysis of the bacteria. In some embodiments, such bacteria comprise a cancer therapeutic and/or a prodrug enzyme described herein that is released upon lysis of the bacteria.

In some embodiments, the bacteria are quorum-sensing bacteria. Quorum-sensing allows bacteria to measure the density of their local population and adjust gene expression depending on the cell density. Thus, in some embodiments, the quorum-sensing bacteria comprise a gene that is conditionally expressed when by the bacteria described herein when the bacteria are present at a certain density (e.g., at a tumor). In some embodiments, the conditionally expressed gene is under the control of the p(1uxI) promoter, as described in, for example, Swofford C. A., et al., Proc. Natl. Acad. Sci. USA, 2015, 112(11):3457-62, which is hereby incorporated by reference in its entirety. In some embodiments, the gene is expressed when the bacteria reach a cell density of 1 CFU/ml, 10 CFU/ml, 100 CFU/ml, 1x10⁴ CFU/ml, 1x10⁵ CFU/ml, 1x10⁶ CFU/ml, 1x10⁷ CFU/ml, 1x10⁸ CFU/ml, 1x10⁹ CFU/ml, or 1x10¹⁰ CFU/ml.

In some embodiments, the bacteria described herein are modified such that they release cancer therapeutic agents after a time delay. In some embodiments, the time delay is the result of an inhibition of a bacterial efflux pump in the bacteria. In some embodiments, the bacteria comprise a small molecule cancer therapeutic that is capable of being extruded by a bacterial efflux pump. In some embodiments, the function of the bacterial efflux pumps of the bacteria are inhibited (e.g., using a covalent inhibitor) such that the bacteria are not able to extrude the cancer therapeutic until new efflux pumps are generated by the bacteria.

In some embodiments, the bacteria described herein are modified such that the release of a cancer therapeutic by the bacteria is facilitated by the presence of a second modified bacteria. For example, in some embodiments, a cancer therapeutic (e.g., the anti-cancer drug doxorubicin) is attached to the bacteria by a double-stranded nucleic acid that comprises a cleavage site (e.g., a restriction site and/or a zinc finger nuclease target site. The modified bacteria can then be administered to a subject in conjunction with a second bacteria that localizes to a tumor and that expresses and/or is bound by a restriction enzyme or zinc finger nuclease that is able to cleave the nucleic acid linker. When the bacteria described herein and the second bacteria co-localize to the tumor, the nucleic acid linker is cleaved and the cancer therapeutic is released.

In some embodiments, the bacteria provided herein are bound to a cancer therapeutic by a cross-linker. As used herein, the term “cross-linker” broadly refers to compositions that can be used to join various molecules, including proteins, together. Examples of cross-linkers include, but are not limited to, 1,5-difluoro-2,4-dinitrobenzene, 3,3′-dithiobis(succinimidyl propionate), bis(2-succinimidooxycarbonyloxy)ethyl)sulfone, bis(sulfosuccinimidyl)suberate, dimethyl 3,3′-dithiobispropionimidate, dimethyl adipimidate, dimethyl pimelimidate, dimethyl suberimidate, disuccinimidyl glutarate, disuccinimidyl suberate, disuccinimidyl tartrate, dithiobis(succinimidyl propionate), ethylene glycosl bis(succinimidyl succinate), ethylene glycosl bis(sulfosuccinimidyl succinate), PEGylated bis(sulfosuccinimidyl)suberate (with PEGS), PEGylated bis(sulfosuccinimidyl)suberate (with PEGS) and tris-(succinimidyl)aminotriacetate.

In some embodiments, the bacteria described herein is linked to a cancer therapeutic through a nucleic acid linker. For example, in some embodiments, the bacteria described herein display a first single-stranded nucleic acid oligonucleotide (e.g., an oligonucleotide of at least 10, 11, 12, 13, 14, 15, 16, 17, 18, 19, 20, 21, 22, 23, 24, 25, 26, 27, 28, 29 or 30 nucleotides in length and/or no more than 30, 35, 40, 45, 50, 55, 60, 65, 70, 75, 80, 85, 90, 95 or 100 nucleotides in length) on their surface that can serve binding site for an agent that comprises and/or is linked to second nucleic acid oligonucleotide (e.g., an oligonucleotide of at least 10, 11, 12, 13, 14, 15, 16, 17, 18, 19, 20, 21, 22, 23, 24, 25, 26, 27, 28, 29 or 30 nucleotides in length and/or no more than 30, 35, 40, 45, 50, 55, 60, 65, 70, 75, 80, 85, 90, 95 or 100 nucleotides in length) that specifically hybridizes to the first nucleic acid oligonucleotide. Methods for attaching oligonucleotides to the surface of bacterial cells are known in the art and described in, for example, Twite A. A., et al., Adv. Mater., 2012, 24(18):2380-5, which is hereby incorporated by reference. In some embodiments, the first oligonucleotide has a sequence that is at least 80%, 85%, 90%, 91%, 92%, 93%, 94%, 95%, 96%, 97%, 98%, 99% or 100% identical to a sequence of the second oligonucleotide. Exemplary methods for linking agents to oligonucleotides are provided, for example, in David A. Rusling & Keith R. Fox, Small Molecule-Oligonucleotide Conjugates, DNA Conjugates and Sensors, 2012, Ch3, 75-102, which is hereby incorporated by reference. In some embodiments, a cancer therapeutic is covalently linked to a single-stranded nucleic acid oligonucleotide that specifically hybridizes to a single-stranded nucleic acid oligonucleotide displayed on the cell surface of a bacteria described herein. The hybridized oligonucleotides hybridize and the resulting double-stranded nucleic acid duplex is stable for days. In some embodiments, the stability of the duplex is improved by incorporating phosphorothioate bonds (e.g., 1, 2, 3, 4, 5, 6, 7 or more phosphorothioate bonds) on the 5′ and/or 3′ ends of one or both oligonucleotides.

In some embodiments, the bacteria described herein are linked to a cancer therapeutic through a biotin/streptavidin interaction. In some embodiments, the bacteria described herein are linked to biotin or to a cancer therapeutic using amine-reactive N-hydroxysuccinimide (NHS) esters or N-hydroxysulfosuccinimide (Sulfo-NHS) esters. NHS esters or Sulfo-NHS esters (Life Technologies can be made of virtually any carboxyl-containing molecule of interest by mixing the NHS or Sulfo-NHS with the carboxyl-containing molecule of interest and a dehydrating agent such as the carbodimide EDC using methods available in the art. Exemplary methods of labeling bacteria using NHS esters are provided in Bradburne J. A., et al., AppL Environ. Microbiol, 1993, 59(3):663-8, which is hereby incorporated by reference.

In some embodiments, the bacteria described herein are linked to a cancer therapeutic through a sequence-specific DNA hybridization interaction. For example, a molecule of interest is covalently linked to a single-stranded DNA oligonucleotide and then attached to a bacterial cell that displays the complementary single-stranded DNA oligonucleotide on its cell surface. The two complementary oligonucleotides hybridize and the resulting double-stranded DNA duplex is stable for days. The stability of the DNA duplex and resistance to nucleases is further improved by incorporating 4 phosphorothioate bonds on the 5′ and 3′ ends of both oligonucleotides.

In some embodiments, unnatural amino acids containing ketones, azides, alkynes or other functional groups that are incorporated into surface-expressed proteins of the bacteria described herein are used to link the bacteria to a cancer therapeutic. Unnatural amino acids containing ketones, azides, alkynes or other functional groups known to one skilled in the art can be incorporated into target proteins in a residue-specific manner using, for example, an auxotrophic bacterial strain as described in Marquis H., et al, Infect. Immun., 1993, 61(9):3756-60, which is hereby incorporated by reference. For example, labeling of the bacterial cell surface can be accomplished by growing a methionine auxotrophic bacterial strain in the presence of the unnatural amino acid azidohomoalanine, which acts as a methionine surrogate and is incorporated during protein biosynthesis in place of methionine. Wild-type proteins on the bacterial surface that normally contain a surface-exposed methionine are now functionalized with a surface-exposed azide group, which can then modified with a molecule of interest that contains an alkyne group (e.g., an alkyne-derivatized small-molecule drug or an alkyne-derivatized protein) using Click Chemistry as described in Link A. J. & Tirrell D. A., Cell surface labeling of Escherichia coli via copper(I)-catalyzed [3+2] cycloaddition, J. Am. Chem. Soc., 2003, 125(37):11164-5, which is hereby incorporated by reference. After incorporation into the surface-expressed protein, these functional groups can serve as attachment points for a small-molecule of interest using, for example, the methods described in Prescher J. A. & Bertozzi C. R., Nat. Chem. Biol, 2005, 1(1):13-21, which is hereby incorporated by reference.

In some embodiments, the bacteria described herein is a gram-negative bacteria and the cancer therapeutic is linked to a surface-associated glycan. Linking a cancer therapeutic to a surface-associated glycan can be accomplished, for example, using a two-step metabolic/chemical labeling protocol. First, the surface-associated polymeric sugar is modified by metabolic labeling of the gram-negative bacterium with a chemically modified monosaccharide, which contains an azide functional group that is incorporated into the polymeric structure on the bacterial surface. Second, the cancer therapeutic is selectively ligated to the modified polymer on the bacterial cell surface using Click chemistry, for example, as described in Dumont A., et al., Angew. Chem. Int. Ed. Engl., 2012, 51(13):3143-6), which is hereby incorporated by reference.

In some embodiments, the bacteria described herein is a gram-positive bacteria and the cancer therapeutic is linked to the bacterial cell wall. The cell wall of gram-positive bacteria comprises of many interconnected layers of peptidoglycan (PG). A two-step metabolic/chemical labeling approach can be used for attaching an exogenously added molecule of interest to the PG. The gram-positive bacterial cells are first metabolically labeled by growing the cells in the presence of an alkyne-functionalized D alanine analog, which is incorporated into nascent PG layers during cell wall biosynthesis. Incorporation of the alkyne group then allows labeling of the PG with an azide-functionalized molecule of interest using the copper-catalyzed Click reaction as described in, for example, Siegrist M. S., et al., ACS Chem. Biol., 2013, 8(3):500-5, which is hereby incorporated by reference. In some embodiments, the gram-positive bacterial cells are grown in medium that contains a cyclooctyne-functionalized D alanine analog (e.g., exobcnDala or endobcnDala), which is then incorporated into the PG of the growing cells. The cells are washed with fresh medium and incubated with a cancer therapeutic that is derivatized with an azido-PEG3 group to attach the molecule of interest to the PG in a copper-free reaction as described in, for example, Shieh P., et al., Proc. Natl. Acad. Sci. USA, 2014, 111(15):5456-61, which is hereby incorporated by reference. In some embodiments, the gram-positive bacterial cells are grown in medium that contains an unnatural D-amino acid with a norbornene (NB) group (e.g., D-Lys-NB--OH, D-Dap-NB--OH, D-Dap-NB--NH.sub.2). The unnatural amino acid is metabolically incorporated into the PG of the growing bacterial cells and equips the bacterial cell surface with alkene functional groups with increased reactivity because of the strained alkene within the ring of the norbornene. The cells are then incubated with a tetrazine derivative of the cancer therapeutic to allow ligation of the cancer therapeutic to the PG, as described in Pidgeon S. E. & Pires M. M., Chem. Commun. (Camb)., 2015, 51(51):10330-3, which is hereby incorporated by reference.

In some embodiments, a cancer therapeutic is incorporated into the PG layer of a gram-negative bacterium described herein. Methods for incorporation molecules into the PG layer of a gram-negative bacterium are provided, for example, in Liechti G. W., et al., Nature, 2014, 506(7489):507-10, which is hereby incorporated by reference. In some embodiments, the gram-negative bacterium is grown in the presence of the D amino acid dipeptide EDA-DA (ethynyl-D alanine-D alanine) or DA-EDA (D alanine-ethynyl-D alanine). The EDA-DA, or DA-EDA, is incorporated into the PG layer of the actively growing bacteria and equips the PG with surface-exposed alkyne groups. Copper-catalyzed Click chemistry is used to attach a cancer therapeutic that contains a terminal azide group to the newly introduced alkyne groups of the PG layer. In some embodiments, a D amino acid derivative of a cancer therapeutic is be incorporated directly into the PG layer of a growing bacterium using, for example, the method described in Kuru E., et al., Nat. Protoc., 2015, 10(1):33-52, which is hereby incorporated by reference.

In some embodiments, the bacteria described herein are modified such that an immune modulatory protein, such as a cytokine is attached to the outside of a bacterium of interest using an attachment method described herein. Examples of immune modulating proteins include, but are not limited to, B lymphocyte chemoattractant (“BLC”), C—C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon gamma (“IFN-gamma”), Interlukin-1 alpha (“IL-1 alpha”), Interlukin-1 beta (“IL-1 beta”), Interleukin 1 receptor antagonist (“IL-1ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin- 12 (“IL-12 p40” or “IL-12 p70”), Interleukin-13 (“IL-13”), Interleukin-15 (“IL-15”), Interleukin-16 (“IL-16”), Interleukin-17 (“IL-17”), Chemokine (C—C motif) Ligand 2 (“MCP-1”), Macrophage colony-stimulating factor (“M-CSF”), Monokine induced by gamma interferon (“MIG”), Chemokine (C—C motif) ligand 2 (“MIP-1 alpha”), Chemokine (C--C motif) ligand 4 (“MIP-1 beta”), Macrophage inflammatory protein- 1 -delta (“MIP-1 delta”), Platelet-derived growth factor subunit B (“PDGF-BB”), Chemokine (C—C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (‘BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing comples (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3 ”), VEGF-D 6Ckine, Tyrosine-protein kinase receptor UFO (“Axl”) , Betacellulin (“BTC”), Mucosae-associated epithelial chemokine (“CCL28”), Chemokine (C—C motif) ligand 27 (“CTACK”), Chemokine (C—X—C motif) ligand 16 (“CXCL16”), C—X—C motif chemokine 5 (“ENA-78”), Chemokine (C—C motif) ligand 26 (“Eotaxin-3”), Granulocyte chemotactic protein 2 (“GCP-2”), GRO, Chemokine (C—C motif) ligand 14 (“HCC-1”), Chemokine (C—C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C—X—C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C—C motif) ligand 20 (“MIP-3 alpha”), C—C motif chemokine 19 (“MIP-3 beta”), Chemokine (C—C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C—C motif) ligand 17 (“TARC”), Thymus-expressed chemokine (“TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin“), Lymphatic endothelium (“LYVE-1”), MEC class I polypeptide-related sequence A (“MICA”), MEC class I polypeptide-related sequence B (“MICB”), NRG1-beta1, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDARActivin A, Agouti-related protein (“AgRP”), Ribonuclease 5 (“Angiogenin”), Angiopoietin 1, Angiostatin, Catheprin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 R1, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgp130, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C—X—C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (”TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/ Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-gammalpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Tolllike receptor 2 (“TLR2”), Tumor necrosis factor receptor superfamily member 10A (“TRAIL R1”), Transferrin (“TRF”), WIF-1ACE-2, Albumin, AMICA, Angiopoietin 4, B-cell activating factor (“BAFF”), Carbohydrate antigen 19-9 (“CA19-9”), CD 163 , Clusterin, CRT AM, Chemokine (C—X—C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNT1-inducible-signaling pathway protein 1 (“WISP-1”), and Receptor Activator of Nuclear Factor .kappa. B (“RANK”). The immune modulatory protein can be made recombinantly using methods known to one skilled in the art. The immune modulatory protein can be presented on the surface of a bacterium using bacterial surface display, where the bacterium expresses a genetically engineered protein-protein fusion of e.g., a membrane protein and the immune modulatory protein.

In some embodiments, the bacteria described herein are engineered to express a peptide (e.g., an antigen) and/or a protein (e.g., a protein cancer therapeutic), intracellularly and/or on the bacterial surface (i.e., genetic surface display). For example, in some embodiments, the bacteria comprises a nucleic acid encoding a peptide or protein cancer therapeutic operably linked to transcriptional regulatory elements, such as a promotor. In some embodiments, the peptide or protein is constitutively expressed by the bacteria. In some embodiments, the peptide or protein is inducibly expressed by the bacteria (e.g., it is expressed upon exposure to a sugar or an environmental stimulus like low pH or an anaerobic environment). In some embodiments, the bacteria comprises a plurality of nucleic acid sequences that encode for multiple different recombinant peptides and/or proteins that can be expressed by the same bacterial cell.

In some embodiments, the bacteria displays a recombinantly produced peptide or protein (e.g., a peptide or protein cancer therapeutic) on its surface using a bacterial surface display system. Examples of bacterial surface display systems include outer membrane protein systems (e.g., LamB, FhuA, Ompl, OmpA, OmpC, OmpT, eCPX derived from OmpX, OprF, and PgsA), surface appendage systems (e.g., F pillin, FimH, FimA, FliC, and FliD), lipoprotein systems (e.g., INP, Lpp-OmpA, PAL, Tat-dependent, and TraT), and virulence factor-based systems (e.g., AIDA-1, EaeA, EstA, EspP, MSP1 a, and invasin). Exemplary surface display systems are described, for example, in van Bloois, E., et al., Trends in Biotechnology, 2011, 29:79-86, which is hereby incorporated by reference.

In some embodiments, the bacteria display on their surface a peptide or protein of interest that alters the invasion or adhesion capability of the bacteria. In some embodiments, the protein that alters the invasion or adhesion capability of the bacteria is a bacterial adhesion, such as FadA (e.g., as described in Xu M., et al., J. Biol. Chem., 2007, 282(34):25000-9, which is hereby incorporated by reference) and or a synthetic adhesion (e.g., as described in Pinero-Lambea C., et al., ACS Synth. Biol., 2015, 4(4):463-73, which is hereby incorporated by reference). In some embodiments, the peptide or protein that alters the invasion or adhesion capability of the bacteria is an antibody or antigen binding fragment thereof having binding specificity for a cancer-specific antigen.

In some embodiments, the bacteria described herein comprise a cancer therapeutic (e.g., the cancer therapeutic is loaded into the bacteria prior to administration to a subject). In some embodiments, the cancer therapeutic is loaded into the bacteria by growing the bacteria in a medium that contains a high concentration (e.g., at least 1 mM) of the cancer therapeutic, which leads to either uptake of the cancer therapeutic during cell growth or binding of the cancer therapeutic to the outside of the bacteria. The cancer therapeutic can be taken up passively (e.g. by diffusion and/or partitioning into the lipophilic cell membrane) or actively through membrane channels or transporters. In some embodiments, drug loading is improved by adding additional substances to the growth medium that either increase uptake of the molecule of interest (e.g., Pluronic F-127) or prevent extrusion of the molecules after uptake by the bacterium (e.g., efflux pump inhibitors like Verapamil, Reserpine, Carsonic acid, or Piperine). In some embodiments, the bacteria is loaded with the cancer therapeutic by mixing the bacteria with the cancer therapeutic and then subjecting the mixture to electroporation, for example, as described in Sustarsic M., et al., Cell Biol., 2014, 142(1):113-24, which is hereby incorporated by reference. In some embodiments, the cells can also be treated with an efflux pump inhibitor (see above) after the electroporation to prevent extrusion of the loaded molecules.

Isolated bacteria which have been modified to comprise agents for targeting to specific organs may be provided per se or as part of a pharmaceutical composition.

The term “isolated” or “enriched” encompasses bacteria that has been (1) separated from at least some of the components with which it was associated when initially produced (whether in nature or in an experimental setting), and/or (2) produced, prepared, purified, and/or manufactured by the hand of man. Isolated microbes may be separated from at least about 10%, about 20%, about 30%, about 40%, about 50%, about 60%, about 70%, about 80%, about 90%, or more of the other components with which they were initially associated. In some embodiments, isolated microbes are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The terms “purify,” “purifying” and “purified” refer to a microbe or other material that has been separated from at least some of the components with which it was associated either when initially produced or generated (e.g., whether in nature or in an experimental setting), or during any time after its initial production. A microbe or a microbial population may be considered purified if it is isolated at or after production, such as from a material or environment containing the microbe or microbial population, and a purified microbe or microbial population may contain other materials up to about 10%, about 20%, about 30%, about 40%, about 50%, about 60%, about 70%, about 80%, about 90%, or above about 90% and still be considered “isolated.” In some embodiments, purified microbes or microbial population are more than about 80%, about 85%, about 90%, about 91%, about 92%, about 93%, about 94%, about 95%, about 96%, about 97%, about 98%, about 99%, or more than about 99% pure. In the instance of microbial compositions provided herein, the one or more microbial types present in the composition can be independently purified from one or more other microbes produced and/or present in the material or environment containing the microbial type. Microbial compositions and the microbial components thereof are generally purified from residual habitat products.

In certain embodiments, at least 50%, 55%, 60%, 65%, 70%, 75%, 80%, 85%, 90%, 95%, 96%, 97%, 98%, 99% of the bacteria in the bacterial composition are of a genus, species or strain listed in Tables 1-3.

In certain embodiments, the bacterial composition comprises at least 1x10³ colony forming units (CFUs), 1x10⁴ colony forming units (CFUs), 1x10⁵ colony forming units (CFUs), 1x10⁶ colony forming units (CFUs), 1x10⁷ colony forming units (CFUs), 1x10⁸ colony forming units (CFUs), 1x109 colony forming units (CFUs), 1x10¹⁰ colony forming units (CFUs) of bacteria of a family/genus/species/strain listed in Tables 1-3.

Methods for producing bacteria may include three main processing steps. The steps are: organism banking, organism production, and preservation.

For banking, the strains included in the bacteria may be (1) isolated directly from a specimen or taken from a banked stock, (2) optionally cultured on a nutrient agar or broth that supports growth to generate viable biomass, and (3) the biomass optionally preserved in multiple aliquots in long-term storage.

In embodiments using a culturing step, the agar or broth may contain nutrients that provide essential elements and specific factors that enable growth. An example would be a medium composed of 20 g/L glucose, 10 g/L yeast extract, 10 g/L soy peptone, 2 g/L citric acid, 1.5 g/L sodium phosphate monobasic, 100 mg/L ferric ammonium citrate, 80 mg/L magnesium sulfate, 10 mg/L hemin chloride, 2 mg/L calcium chloride, 1 mg/L menadione. Another examples would be a medium composed of 10 g/L beef extract, 10 g/L peptone, 5 g/L sodium chloride, 5 g/L dextrose, 3 g/L yeast extract, 3 g/L sodium acetate, 1 g/L soluble starch, and 0.5 g/L L-cysteine HCl, at pH 6.8. A variety of microbiological media and variations are well known in the art (e.g., R. M. Atlas, Handbook of Microbiological Media (2010) CRC Press). Culture media can be added to the culture at the start, may be added during the culture, or may be intermittently/continuously flowed through the culture. The strains in the bacterial composition may be cultivated alone, as a subset of the microbial composition, or as an entire collection comprising the microbial composition. As an example, a first strain may be cultivated together with a second strain in a mixed continuous culture, at a dilution rate lower than the maximum growth rate of either cell to prevent the culture from washing out of the cultivation.

The inoculated culture is incubated under favorable conditions for a time sufficient to build biomass. For microbial compositions for human use this is often at 37 degree C temperature, pH, and other parameter with values similar to the normal human niche. The environment may be actively controlled, passively controlled (e.g., via buffers), or allowed to drift. For example, for anaerobic bacterial compositions, an anoxic/reducing environment may be employed. This can be accomplished by addition of reducing agents such as cysteine to the broth, and/or stripping it of oxygen. As an example, a culture of a bacterial composition may be grown at 37° C., pH 7, in the medium above, pre-reduced with 1 g/L cysteine-HC 1.

When the culture has generated sufficient biomass, it may be preserved for banking. The organisms may be placed into a chemical milieu that protects from freezing (adding ‘cryoprotectants’), drying (‘lyoprotectants’), and/or osmotic shock (‘osmoprotectants’), dispensing into multiple (optionally identical) containers to create a uniform bank, and then treating the culture for preservation. Containers are generally impermeable and have closures that assure isolation from the environment. Cryopreservation treatment is accomplished by freezing a liquid at ultra-low temperatures (e.g., at or below -80° C.). Dried preservation removes water from the culture by evaporation (in the case of spray drying or ‘cool drying’) or by sublimation (e.g., for freeze drying, spray freeze drying). Removal of water improves long-term microbial composition storage stability at temperatures elevated above cryogenic. If the microbial composition comprises, for example, spore forming species and results in the production of spores, the final composition may be purified by additional means such as density gradient centrifugation preserved using the techniques described above. Microbial composition banking may be done by culturing and preserving the strains individually, or by mixing the strains together to create a combined bank. As an example of cryopreservation, a microbial composition culture may be harvested by centrifugation to pellet the cells from the culture medium, the supernatant decanted and replaced with fresh culture broth containing 15% glycerol. The culture can then be aliquoted into 1 mL cryotubes, sealed, and placed at -80° C. for long-term viability retention. This procedure achieves acceptable viability upon recovery from frozen storage.

Microbial production may be conducted using similar culture steps to banking, including medium composition and culture conditions. It may be conducted at larger scales of operation, especially for clinical development or commercial production. At larger scales, there may be several subcultivations of the microbial composition prior to the final cultivation. At the end of cultivation, the culture is harvested to enable further formulation into a dosage form for administration. This can involve concentration, removal of undesirable medium components, and/or introduction into a chemical milieu that preserves the microbial composition and renders it acceptable for administration via the chosen route. After drying, the powder may be blended to an appropriate potency, and mixed with other cultures and/or a filler such as microcrystalline cellulose for consistency and ease of handling, and the bacterial composition formulated as provided herein.

In certain aspects, provided are bacterial compositions for administration to subjects. In some embodiments, the bacterial compositions are combined with additional active and/or inactive materials in order to produce a final product, which may be in single dosage unit or in a multi-dose format.

The compositions may be administered using any route such as for example oral administration, rectal administration, topical administration, inhalation (nasal) or injection. Administration by injection includes intravenous (IV), intramuscular (IM), intratumoral (IT), subtumoral (ST), peritumoral (PT), and subcutaneous (SC) administration. The pharmaceutical compositions described herein can be administered in any form by any effective route, including but not limited to intratumoral, oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), intravesical, intrapulmonary, intraduodenal, intragastrical, and intrabronchial. In preferred embodiments, the pharmaceutical compositions described herein are administered orally, rectally, intratumorally, topically, intravesically, by injection into or adjacent to a draining lymph node, intravenously, by inhalation or aerosol, or subcutaneously.

In some embodiments the composition comprises at least one carbohydrate. A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula C.sub.nH.sub.2nO.sub.n. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

In some embodiments the composition comprises at least one lipid. As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In some embodiments the composition comprises at least one modified lipid, for example a lipid that has been modified by cooking.

In some embodiments the composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments the composition comprises at least one supplemental vitamin. The at least one vitamin can be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments the composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments the excipient is a buffering agent. Non-limiting examples of suitable buffering agents include sodium citrate, magnesium carbonate, magnesium bicarbonate, calcium carbonate, and calcium bicarbonate.

In some embodiments the excipient comprises a preservative. Non-limiting examples of suitable preservatives include antioxidants, such as alpha-tocopherol and ascorbate, and antimicrobials, such as parabens, chlorobutanol, and phenol.

In some embodiments the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C.sub.12-C.sub.18 fatty acid alcohol, polyethylene glycol, polyols, saccharides, oligosaccharides, and combinations thereof.

In some embodiments the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments the composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments the composition comprises a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments, the composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In certain embodiments, the bacteria disclosed herein are administered in conjunction with a prebiotic to the subject. Prebiotics are carbohydrates which are generally indigestible by a host animal and are selectively fermented or metabolized by bacteria. Prebiotics may be short-chain carbohydrates (e.g., oligosaccharides) and/or simple sugars (e.g., mono- and di-saccharides) and/or mucins (heavily glycosylated proteins) that alter the composition or metabolism of a microbiome in the host. The short chain carbohydrates are also referred to as oligosaccharides, and usually contain from 2 or 3 and up to 8, 9, 10, 15 or more sugar moieties. When prebiotics are introduced to a host, the prebiotics affect the bacteria within the host and do not directly affect the host. In certain aspects, a prebiotic composition can selectively stimulate the growth and/or activity of one of a limited number of bacteria in a host. Prebiotics include oligosaccharides such as fructooligosaccharides (FOS) (including inulin), galactooligosaccharides (GOS), trans-galactooligosaccharides, xylooligosaccharides (XOS), chitooligosaccharides (COS), soy oligosaccharides (e.g., stachyose and raffinose) gentiooligosaccharides, isomaltooligosaccharides, mannooligosaccharides, maltooligosaccharides and mannanoligosaccharides. Oligosaccharides are not necessarily single components, and can be mixtures containing oligosaccharides with different degrees of oligomerization, sometimes including the parent disaccharide and the monomeric sugars. Various types of oligosaccharides are found as natural components in many common foods, including fruits, vegetables, milk, and honey. Specific examples of oligosaccharides are lactulose, lactosucrose, palatinose, glycosyl sucrose, guar gum, gum Arabic, tagalose, amylose, amylopectin, pectin, xylan, and cyclodextrins. Prebiotics may also be purified or chemically or enzymatically synthesized.

As used herein the term “about” refers to ± 10 %

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization - A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Dealing With Contaminations in Bacterial 16S RDNA Profiling of Low Microbial Biomass Samples

The increased sensitivity of next-generation sequencing of the 16S rDNA marker-gene for microbial discovery, has led to a remarkable bloom of microbiome research. Yet, this same advantage also yields a higher rate of detection of contaminating microbes in samples. This phenomena is compounded when exploring samples containing low microbial biomass, where contaminant species tend to outcompete and dominate the biological signal from the true positive microbial species in the samples(20-23, 59-62).

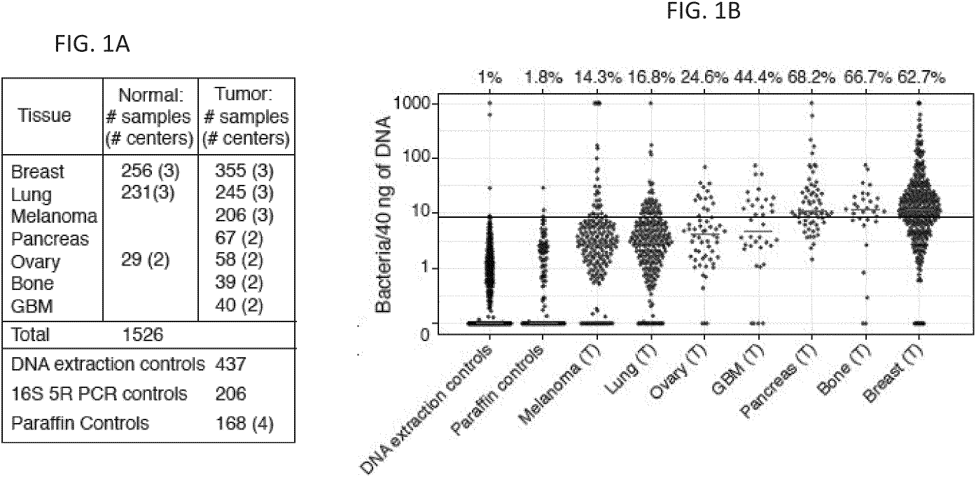

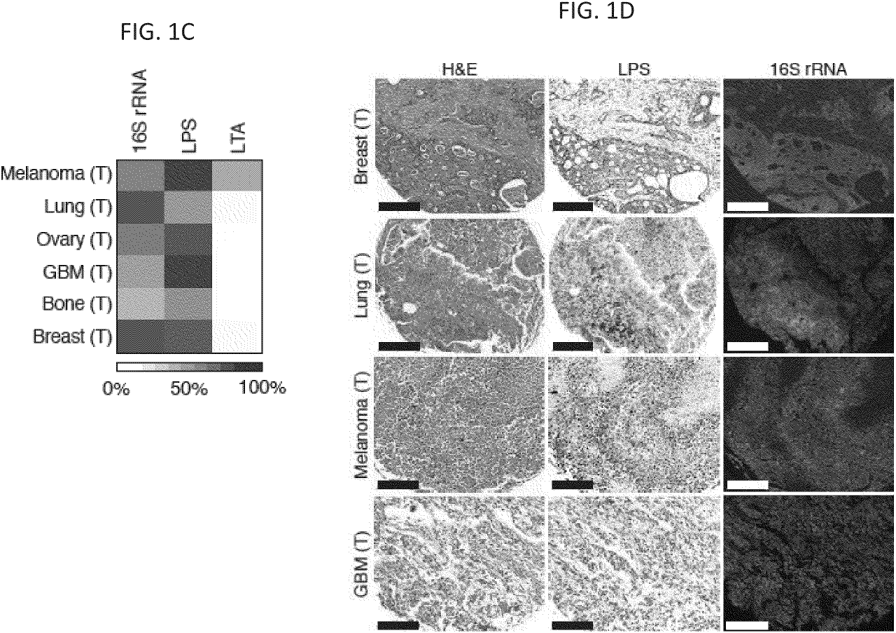

Our samples originate from solid tumor resections and are akin to tissue biopsies, predicted to be of low microbial biomass. Therefore, we have included DNA extraction controls, no-template PCR amplification controls, sequencing run controls and empty paraffin controls to account for the various sources of contamination from the hospital and laboratory environments (dust, air, human commensals), and the different stages of handling and processing of the samples (plastics, kits, reagents, paraffin, etc...). By comparing the 16S qPCR results of different types of tumor tissue samples alongside the negative controls in FIGS. 1A-D, this low biomass quality can be detected, albeit, qPCR levels of samples are distinguishable from controls indicating a true biological signal and reinforcing the multiple findings of microbes in solid tumors recently amassing in the cancer field.

1) Report the experimental design to reduce all types of contamination: DNA from samples was extracted in a clean room environment, akin to the methodologies of an ancient DNA lab as this was shown to greatly reduce certain types of contaminations (62). Ultraclean kits and reagents were used and pre and post PCR experiments were performed in separate designated areas.

2) Include controls to assess contaminant DNA: As samples of different solid tumor types were collected from multiple medical centers across the world across several years, a strict and detailed documentation was carried out of batch sources, such as center origin, DNA extraction batch and date, PCR amplification batch and sequencing library number. Concomitantly, empty negative controls were incorporated into each batch type enabling us to detect different types of batch effects. A total of 811 controls were performed, 801 of them passing our 1000 minimum reads filter and employed in our statistical approach to in-silico decontamination described in the next section. This represents an unprecedented -50% control-to-sample ratio in one of the largest cohorts of low-microbial-biomass samples (1526 samples).

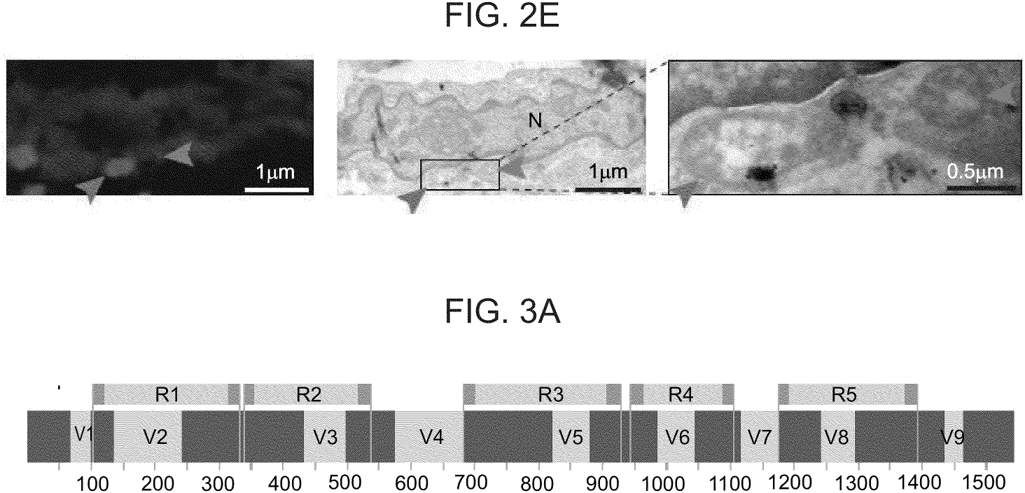

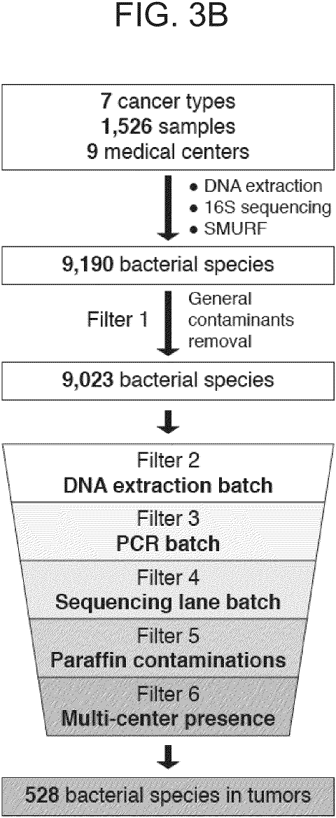

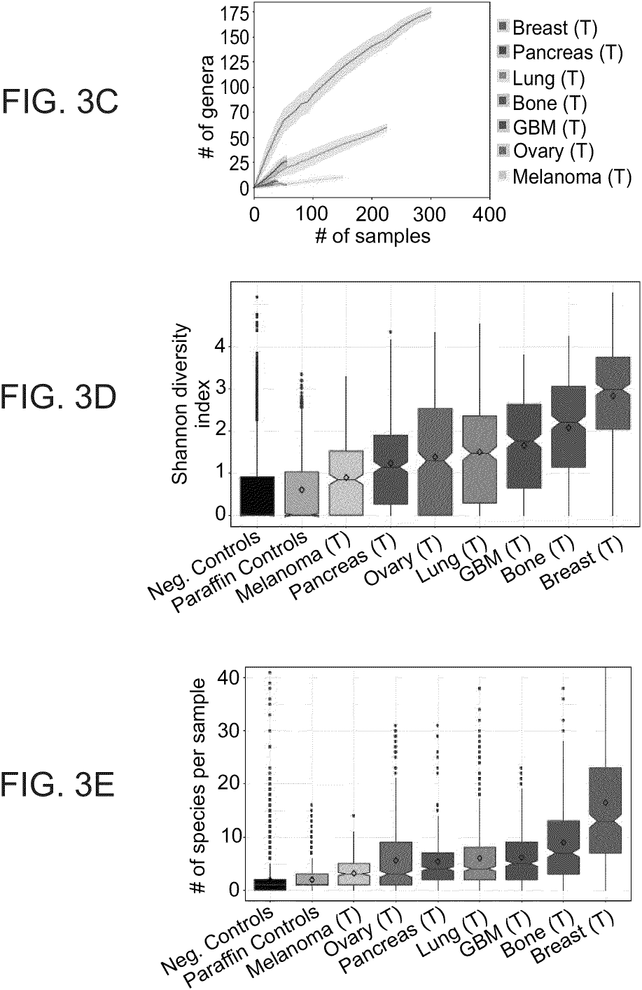

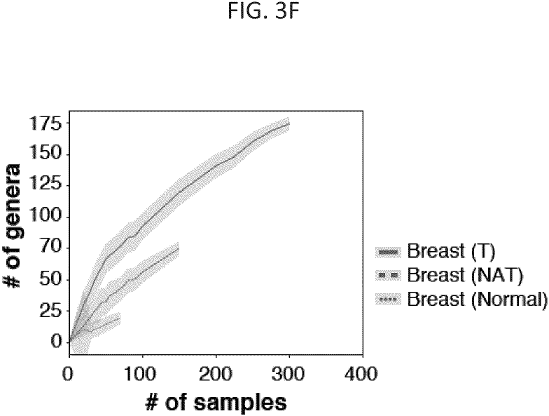

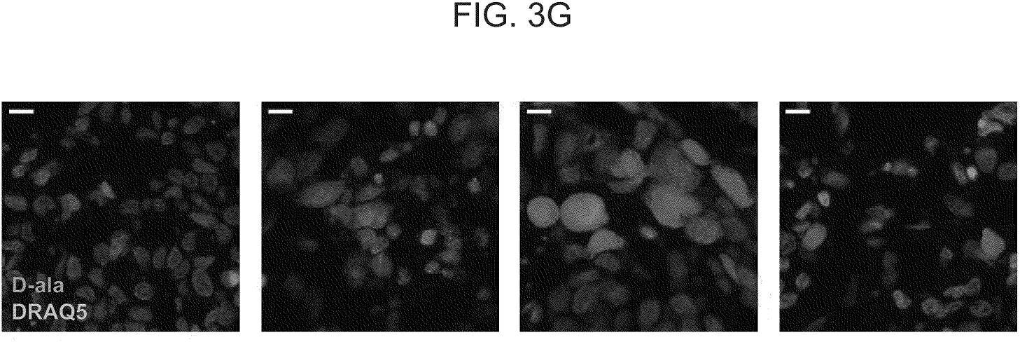

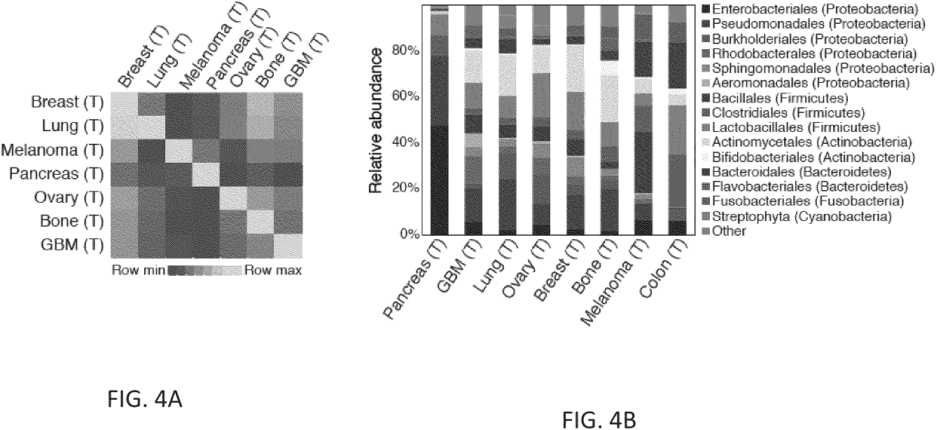

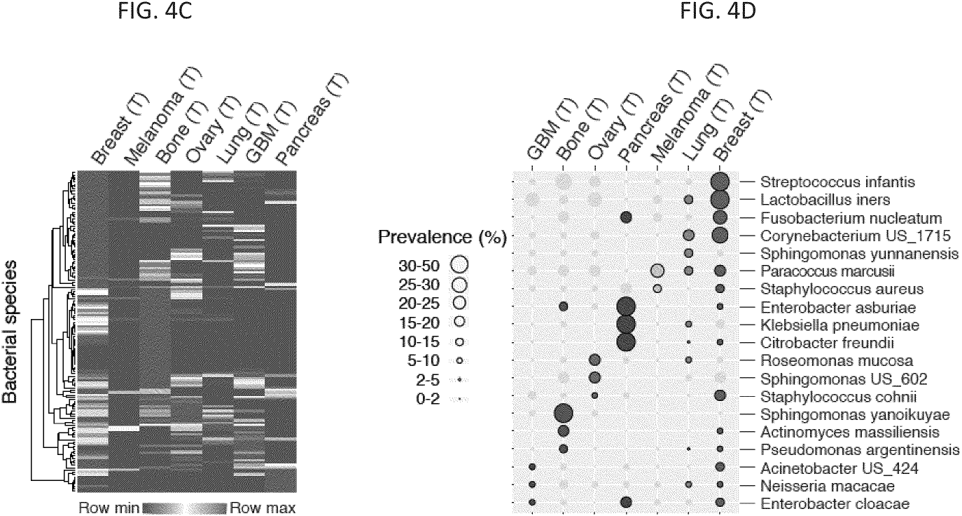

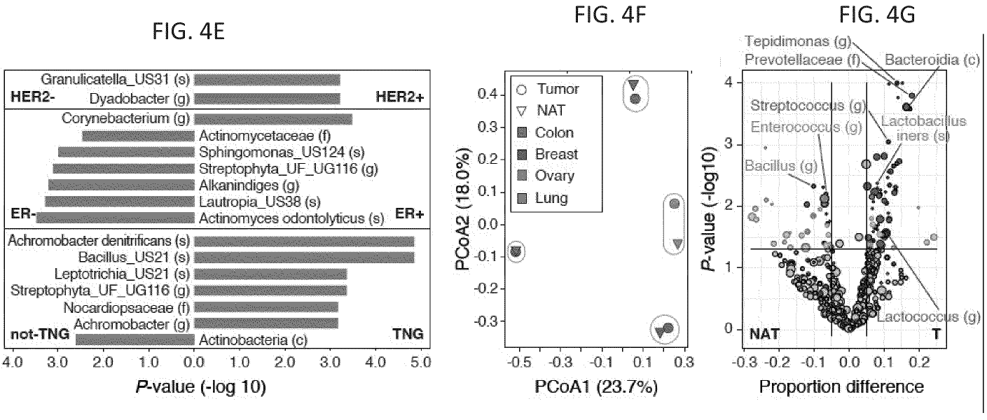

3) Determine and mitigate the effect of contaminant DNA: There are several approaches to remove false-positive contaminating species from further analysis in order to prevent a misleading characterization of samples’ microbiomes or discovery of false associations between microbes and clinical phenotypes (61). “Contaminant subtraction”, namely a straightforward removal of all species detected in control samples, prevents the false-positive rate running rampant in the microbiome field. However, this approach also runs the risk of losing biological signal resulting from rare contamination of control samples during their handling and processing with bacteria that are also genuinely present in the samples. Technical cross-contamination from tumor samples to control samples can also drive such a contamination of control samples. Our inclusion of over 800 controls enabled us to be highly sensitive to contaminations on the one hand, but to mitigate/reduce false-negatives errors by recognizing that there is a spectrum of bacterial prevalence across controls. While the obvious highly prevalent species in control samples could be discounted altogether, the lower prevalent species could be more appropriately classified as contaminants by incorporating a statistical comparison of bacterial prevalence in samples to their background distributions in negative controls. Another possibility for telling apart contaminations from true signal is by selecting species that are detected concordantly among sample replicates. Technical replicates of the same sample performed with different DNA extraction kits can tease out kit-related contaminants, but also run the risk of discounting discordant bacteria due to kit specific extraction efficiencies. Indeed, by testing several DNA extraction kits and protocols using multiple mock bacterial mixtures we demonstrated that they harbor different bacterial extraction efficiencies (data not shown). Therefore, we chose to apply a more biologically-oriented version of concordance and integrate it with the previous methodologies, implemented as a series of filters we named the ‘hit calling pipeline’:
Conversion of relative abundance to read counts and normalization of these read counts per library to the average read count of all libraries. This reduces the variation introduced by the type of sequencing platform (e.g. Illumina Hiseq/NextSeq/MiSeq) and the varying read depths of each library. Note that since many biases can affect the read counts (e.g. the number of 16S rDNA copies are different between different bacteria) we have mainly used absent/present calls in the analysis rather then normalized read counts.
Initial QC: samples that didn’t reach the minimum of 1000 normalized reads were removed. Very few control samples were removed at this stage (10 out of 811), as most negative controls contain contaminants which are successfully sequenced.
Flooring the relative abundance at 10⁻⁴ to reduce the noise variation at the very low abundance levels. This is a prerequisite to absence/presence calling of species since contaminating species have a large impact on the lowly abundant species, either by relatively reducing their abundance or eliminating their detection. This may reduce some rare bacterial species, but enable within-sample diversity calculations such as sample richness (FIG. 3D) and also enable more robust between-sample diversity calculations (FIGS. 4A, 4F).
Removal of global environmental and paraffin contaminants (filter 1). This stage deals with the highly prevalent contaminations. Here, any species that was prevalent across more than 7.5% of negative DNA/PCR/Sequencing controls or more than 7.5% of empty paraffin controls was completely removed. The first set of controls represents our lab environment and the latter represents the accumulating contamination from the hospital environment, including sample preparation, storage and block processing. We set the threshold of 7.5% based on the inflection point in the bimodal distribution of the species abundance percentage in control samples. Several of the bacterial species exhibiting prevalences higher than this threshold had a clear time and batch dependant profile.

It is worth mentioning here, that our novel 5-plex bacterial 16S rDNA amplification sequencing methodology, has greatly increased classification power of 16S sequences at species resolution. Therefore this global contaminant filtering step was employed on species, enabling a distinction between contaminating and non-contaminating species even from the same genus. We, therefore, didn’t have to rule out entire genera of bacteria because of one representative contaminating species.

After removing the highly prevalent contaminations, we could aggregate the species data to higher taxonomic levels in order to report findings at different taxonomic levels.
In order to remove the less prevalent contaminations, we compared taxa prevalences in samples to their prevalences in controls based on the assumption that “even in a low-biomass regime, the non-contaminants will appear in a larger fraction of true samples that in negative controls” (22). This type of approach (also implemented in the decontam package in R) has been demonstrated to be able to discriminate between true positives and contaminations both in high biomass situations such as the oral cavity and even concurs with the latest literature that extremely low biomass placental tissue does not contain an indigenous microbiome (22, 23, 63). This approach veers away from skewed relative abundance data which occur when contaminations are high and relies more heavily on absent/present calls, which along with flooring by relative abundance (see above), reduces the noise variation of presence/absence calling at the very low abundance levels.

As the RIDE guidelines and additional research indicate, different contamination profiles can vary across processing batches and time (21, 23, 62). We also took into account that different tissue types were retrieved from different medical centers and processed at different times using different batches of kits. We thus shifted to a ‘per-condition’ (a combined tissue+tumor/NAT/normal status) contamination analysis. Therefore, our comparisons between taxon prevalence in samples and controls were done on a per-condition, per-batch basis, comparing samples from the same condition to the controls that were processed alongside them. This was done for the three different processing batch types; DNA extraction batch (filter 2), PCR amplification batch (filter 3), and sequencing library batch (filter 4). We employed the non-parametric, exact, binomial test on each taxa’s prevalence, x=number of successes in the sample & n=sample size to the background p= taxa’s prevalence in the relevant batch controls. Only taxa that passed a p-value cutoff of 0.05 in all batch comparisons (filters 2-4) were considered in the next filtering stage.
To clear out center-specific contaminations, we compared the prevalence of taxa in all samples per-condition to their prevalence in a set of empty paraffin controls originating from the same centers as the samples, requiring a p-value cutoff of 0.05 to pass (filter 5).
Lastly, in another effort to account for center-specific contamination we applied another filter demanding that a bacteria in a specific condition be present in samples from at least two medical centers (filter 6). To this end we compared the prevalence of taxa in samples, again per-condition, this time per-medical-center to the same-center specific controls (DNA extraction, PCR, library controls that ran with these specific center samples). This was performed via binomial test (as described above) on each center per-condition. Only taxa that passed a p-value cutoff of 0.05 in at least two centers, and a Fisher’s combined p-value across all centers, FDR corrected < 0.2, passed this filter. It is worthy to note, that the two-center requirement is a very harsh one because it requires “biological concordance” of taxa significantly appearing beyond controls in more than one medical center. This eliminates hospital-specific contaminants, but also focuses on taxa that are shared among patients with the same cancer condition, regardless of their country of origin’s environment and bacterial commensals. We were aiming to find a common tumor microbiome signature for each tumor type.

As in all in-silico analyses, it is always advisable to support and validate findings with orthogonal experimental methodologies. We included a plethora of visualizing techniques on hundreds of tumor samples, including IHC, IF, FISH, electron microscopy and D-alanine incorporation. We have also used culturomics to provide further evidence for the presence of live bacteria in human tumors.

### MATERIALS AND METHODS

Sample collection. Fresh frozen samples and Formalin-Fixed Paraffin-Embedded (FFPE) samples were collected from nine medical centers in four countries. All samples were collected and analyzed according to IRB-approved protocols. In addition, we introduced 643 negative controls including 437 DNA extraction controls which are empty tubes that were processed together with the samples, and 206 no-template controls (NTCs) for the 16S amplification for sequencing (FIG. 1A). For 168 FFPE blocks we sliced paraffin from the margins of the block, sampling paraffin only without tissue. These paraffin negative control samples were generated from blocks representing all 4 medical centers from which we acquired tissue from FFPE blocks. Out of the 355 breast tumors, only three were retrieved from patients with breast implants. For 32 of the tumors we have no medical records for the presence or absence of implants.

Bacterial DNA extraction. Frozen tissue (40-70 mg) was extracted with the UltraClean Tissue & Cells DNA Isolation kit (MoBio - #12334) according to the manufacturer’s instructions. A 30 minute proteinase K (15 µl) digestion was done, and bead-beating was performed before (10 minutes) and after (5 minutes) digestion (vortex adapter - Qiagen #13000-V1-24 on Vortex-Genie 2 at maximal speed). Bacterial DNA from paraffin blocks was extracted using the same kit with some adaptations. Paraffin slices (3-5, 10 µm thick) were mixed with TD1 buffer (200 µl) and a paraffin pastille (Histosec pastilles, Merck #111609) and heated at 90° C. for 10 min in 1.5 ml microcentrifuge tubes. Tubes were immediately spun in a pre-cooled centrifuge (4° C.) for 5 min (14,000 RPM) followed by 15 min incubation on ice to enable easier wax removal. Wax was then removed with a steel needle followed by the addition of kit TD1 buffer (200µl) and proteinase K (40µl). Protein digestion was done at 56° C. overnight with constant shaking (400 RPM). Tubes were immediately transferred to 90° C. for 45 min and then cooled at room temperature for 10 min. Lysates underwent bead beating with 200µl 0.1 mm zirconialsilica beads (Biospec - #11079101z) for 10 minutes at full speed. Ethanol was added (100%, 200 µl) to the tubes followed by a short vortex mix and the whole mixture (together with beads) was loaded onto the columns in two steps. Wash and elution steps were done according to manufacturer’s instructions. Pre-heated (70° C.) elution buffer (100ul) was added to the columns. Columns were incubated at 70° C. for 4 min before DNA was eluted. All negative controls were processed according to the exact same protocols.

16S real-time quantitative PCR. The following bacterial primers(24) for the V6 region of the 16S ribosomal RNA (rRNA) region were used in combination: 5′-CNACGCGAAGAACCTTANC-3′ (SEQ ID NO: 1), 5′- ATACGCGARGAACCTTACC-3′ (SEQ ID NO: 2), 5′-CTAACCGANGAACCTYACC-3′ (SEQ ID NO: 3), 5′-CAACGCGMARAACCTTACC-3′ (SEQ ID NO: 4), 5′-CGACRRCCATGCANCACCT-3′ (SEQ ID NO: 5).

Forty ng of DNA was used per reaction. For 1% of the samples in which DNA concentrations were too low only 10-40 ng of DNA was added per reaction. For the 168 paraffin negative controls we added to the reaction the same volume of eluate that was added for each matched block from the tissue-containing sample. We also included 209 non-template controls (NTC) for the qPCR reactions. Note that these 209 negative controls are in addition to the controls reported in FIG. 1A. DNA was combined with 500 nM primers, and 1X Kapa SYBR FAST qPCR Master Mix (2X) (Kapa Biosystems, #KK4605) into a 25 µl reaction. The qPCR reaction was performed on the Applied Biosystems StepOnePlus™ Real-Time PCR System at 95° C. for 3 min, followed by 40 cycles of 95° C. for 3 sec and 64° C. for 30 sec and completed with a dissociation curve. Amount of bacteria was estimated by comparing threshold cycles (Ct) values to a bacteria standard curve produced with Brevibacterium frigoritolerans DNA. Bacterial load was normalized by subtracting from it the averaged batch-related NTC’s bacterial load.

Immunostaining assays. Human tumor tissue microarrays (TMAs) were purchased from US Biomax (except for lung tumor TMAs that were a gift from Dr. Dan Raz at City of Hope, California, USA) and included over 400 cores representing six tumor types. TMAs were stained for bacterial LPS (Lipopolysaccharide Core, mAb WN1 222-5, HycultBiotech #HM6011, 1:1000 dilution) and LTA (Lipoteichoic acid, mAb 55, HycultBiotech #HM2048, 1:400 dilution) or no primary antibody (negative control) with the automated slide stainer BOND RX^{m} (Leica Biosystems) using the Bond polymer refine detection kit (Leica Biosystems #DS9800), according to manufacturer’s instructions. Acidic antigen retrieval was done by a 20 min heating step with the epitope retrieval solution 1 (Leica Biosystems #AR9961). Slides were scanned using the Pannoramic SCAN II automated slide scanner (3D HISTECH) at 40X.

Analysis was done using two software packages. First, the TMA cores location was detected and manually corrected using the TMA dearrayer function in QuPath (64). The individual cores of each slide were then exported to tiff files. Analysis of all the cores was done automatically using ImageJ/Fiji (65) macro to produce statistics and quality control images with overlay of the whole tissue and bacteria detected areas. Segmentation of whole tissue was done by first converting the RGB image to grayscale, smoothing it with Gaussian blur, applying a fixed threshold and discarding small segments (< 100 µm²). Finally, small holes in the tissue were closed (< 1000 µm²), while big holes were discarded. Bacteria were segmented by first applying color deconvolution (66) (www(dot)blog(dot)bham.ac(dot)uk/intellimic/g-landini-software/) with built-in H-DAB vectors. Candidate bacteria areas were detected by applying fixed threshold to the DAB “channel”. Soot areas appear darker, thus objects for which the mean intensity in each of the three color-deconvolved channels is lower than a channel-dependent fixed threshold were discarded. Elongated and thin objects were also discarded based on their axial ratio and area.

Tumor cores were classified as positive for LPS or LTA according to the proportion of the total area that was calculated to be positive for DAB (area >0.2% - positive). This threshold was determined by measuring background signals as detected in control stains of the TMAs (no primary Ab stain) so that more than 95% of the negative control cores are classified as negative. The macro correctly distinguishes between bacteria and soot areas in most cases. However, in some cases they look very similar, so we manually verified all the cores and corrected the classification as needed. For melanoma, cores were manually corrected as well for melanin signals.

Immunofluorescence assays. Immunofluorescence staining was performed according to standard staining methods, including a deparaffinization and rehydration step, endogenous peroxidase quenching (1% H₂O₂, 0.185% HCl), an acidic antigen retrieval step (10 min at 95° C. in Citric acid pH6) and blocking with 1%BSA and 0.2% Triton. Primary antibodies (anti-CD45 -eBioscience #14-0459-82, anti-CD68 - Invitrogen #MA5-12407) were applied on slides overnight at 4° C. and secondary antibodies (Goat anti-Mouse IgG1 Cross-Adsorbed Secondary Antibody, Alexa Fluor 555 - Invitrogen #A-21127, Goat anti-Mouse IgG3 Cross-Adsorbed Secondary Antibody, Alexa Fluor 488 - Invitrogen #A-21151) were added for 30 min at room temperature. Slides were mounted with ProLong Gold Antifade Mountant (Life technologies #P36930). Slides were scanned using the Pannoramic SCAN II automated slide scanner (3D HISTECH) at 20X.

16S RNA FISH. FFPE tissue slides were deparaffinized, rehydrated and incubated in 70% ethanol at 4° C. for 2 hours. Slides were washed in 2X SSC (Ambion #AM9765) and incubated with proteinase K (10 µg/ml in 2x SSC, Ambion #AM2546) for 10 minutes. Slides then underwent two washes with 2XSSC followed by two washes in 2XSSC with 15% formamide (Ambion #AM9342). Cy5 labelled Probes (EUB338(27)- GCTGCCTCCCGTAGGAGT (SEQ ID NO: 6) and non-specific complement probe - CGACGGAGGGCATCCTCA (SEQ ID NO: 7), at 1680 nM) were hybridized overnight at 30° C. in 2XSSC, 10% Dextran sulfate (Sigma #D8906), 1 mg/ml E.coli tRNA (Sigma #R4251), 0.02% BSA (Ambion #AM2616), 2 mM Vanadyl-ribonucleoside (New England Biolabs #S1402S) and 15% formamide. Sections were washed for 30 minutes at 30° C. in 2XSSC with 15% formamide followed by a 30 minutes incubation step with 2XSSC, 15% formamide and DAPI at 30° C. Slides were washed in 2XSSC, 10 mM TRIS pH8 and 0.4% glucose and mounted with ProLong Gold Antifade Mountant (Life technologies #P36930). Staining was visualized with the Pannoramic SCAN II automated slide scanner (3D HISTECH) at 40X. Analysis was done using the same tools and steps as for the IHC slides with some modifications. Segmentation of whole tissue was done using the DAPI image, smoothing it with Gaussian blur, applying a fixed threshold and discarding small segments (< 500 µm2). Finally, small holes in the tissue were closed (< 5000 µm2), while big holes were discarded. Bacteria were segmented by applying median filter on the Cy5 image with an intensity threshold set at 40. Small Cy5 positive segments (<5 µm²) were discarded. Tumor cores were classified as positive for bacterial 16S rRNA according to the proportion of the total area that was calculated to be positive for Cy5 (area >0.799% - positive). This threshold was determined by measuring background signals as detected in control stains of the TMAs using the non-specific complement probe.

Correlative Light and Electron Microscopy (CLEM). The CLEM imaging was done at the Moskowitz Center for Nano and Nano-Bio Imaging, Weizmann Institute of Science. Samples were fixed in 4% paraformaldehyde with 0.1% glutaraldehyde in 0.1 M cacodylate buffer (pH=7.4) for 1 hour at room temperature, and kept overnight at 4° C. Samples were then soaked overnight in 2.3 M sucrose and rapidly frozen in liquid nitrogen. Frozen ultrathin (70-90 nm) sections were cut with a diamond knife at -120° C. on a Leica EM UC6 ultramicrotome. The sections were collected on 200-mesh Formvar coated nickel grids. Sections were labeled with DAPI (1 µg/ml for 20 minutes at RT), primary antibody for LPS (Lipopolysaccharide Core, mAb WN1 222-5, HycultBiotech #HM6011, 1:300 dilution) and secondary antibody conjugated to AlexaFluor555 (Goat anti-Mouse IgG2a Cross-Adsorbed Secondary Antibody, Alexa Fluor 555, Invitrogen #A-21137, 1:200 dilution). Wide-field fluorescence images were taken in order to identify bacteria-AlexaFluore555 using VUTARA SR352 system (Bruker). Contrast staining and embedding of the sections were performed as previously described (29). The same grids were viewed with an FEI Tecnai SPIRIT (FEI, Eindhoven, Netherlands) transmission electron microscope operated at 120 kV, and equipped with an EAGLE CCD Camera. LPS channel was used to identify bacteria in the section and provided a desired region for TEM investigation. The TEM grid mesh was used to identify the general region of a selected cell, while the nucleus marked with DAPI and easily identified in the TEM was used as fiducial markers in order to overlay the fluorescence image and the TEM image in high accuracy.

Improving Greengenes (GG) taxonomy. While each sequence in the GG database has an assigned taxonomy, species-level taxonomies are missing for about 80% of the sequences. Moreover, the taxonomy is sometimes inconsistent or contains typos. We took a few steps to curate and augment the GG taxonomy.

1. Detecting general inconsistencies. A taxonomy that was specified at a certain level while its higher levels were unknown was replaced by the term ‘unknown’.

2. Detecting inconsistencies at the species level. The GG taxonomy file (May 2013 version) was validated using the DSMZ nomenclature (www(dot)dsmz(dot)de). GG species that did not appear in DSMZ although their genus was present in DSMZ were changed to ‘unknown species’ in the GG database.

3. Using RDP-based taxonomy to augment GG taxonomy. All GG sequences were classified via the Ribosomal Database Project (RDP) Sequence Match (SeqMatch) engine recoding 250 matches per query sequence. Each such match provided a tentative taxonomy and a similarity score, S_ab, between the GG query sequence and the retrieved match. The lowest taxonomic level for each of these 250 matches was set using a threshold on S_ab: species - 0.99, genus - 0.97, family - 0.93, order - 0.85, class - 0.75, phylum - 0.65, domain - 0.5. Each GG sequence was assigned a taxonomy according to the maximal S_ab among the 250 matches. The GG taxonomy was considered as baseline to be augmented by RDP-based taxonomy only when the latter was consistent with GG and provided more information.

4. Assigning missing genus and species names. Novel assignments were first sought at the genus level. All sequences having an ‘unknown’ genus were divided into groups based on their known taxonomy (e.g., all sequences belonging to a specific phylum, class, order and family but lacking a genus, were grouped together). All sequences that share the same taxonomy but also have a known genus were added to each group (e.g., in case the group’s family is ‘unknown’ all sequences whose genus is known were added irrespective of their family). A similarity matrix between all pairs of sequences in a group was calculated using Mothur [16]. Taxonomy of each sequence having an unknown genus was assigned by a majority vote over sequences whose genus is known and whose similarity to the query sequence was higher than 97% (in case of ties, the lexicographically first taxonomy was selected). The Mothur similarity matrix over sequences whose genus remained unknown was clustered using the complete linkage agglomerative clustering algorithm and then split into clusters using a 97% cutoff. Sequences in each cluster were assigned an arbitrary genus name (e.g., “unknown genus #5”). Subsequent to processing the genus level, the same procedure was repeated for species.

5. 16S amplification and deep sequencing. Five regions of the 16S rRNA gene were amplified using 100 ng DNA as an input and a set of 10 multiplexed primers (0.2 µM each primer, F1-TGGCGAACGGGTGAGTAA (SEQ ID NO: 8), F2-ACTCCTACGGGAGGCAGC (SEQ ID NO: 9), F3-GTGTAGCGGTGRAATGCG (SEQ ID NO: 10), F4-GGAGCATGTGGWTTAATTCGA (SEQ ID NO: 11), F5-GGAGGAAGGTGGGGATGAC (SEQ ID NO: 12), R1-AGACGTGTGCTCTTCCGATCTCCGTGTCTCAGTCCCARTG (SEQ ID NO: 13), R2-AGACGTGTGCTCTTCCGATCTGTATTACCGCGGCTGCTG (SEQ ID NO: 14), R3-AGACGTGTGCTCTTCCGATCTCCCGTCAATTCMTTTGAGTT (SEQ ID NO: 15), R4-AGACGTGTGCTCTTCCGATCTCGTTGCGGGACTTAACCC (SEQ ID NO: 16), R5-AGACGTGTGCTCTTCCGATCTAAGGCCCGGGAACGTATT (SEQ ID NO: 17)), 0.2 mM dNTPs (Larova GmbH) and 0.02U/ul of Phusion Hot Start II DNA Polymerase (Thermo Scientific #F549). Amplification was done with an initial heating step of 98° C. for 2 min, 30 cycles of 10 seconds at 98° C., 15 seconds at 62° C., 35 seconds at 72° C. followed by a final elongation step of 5 min at 72° C. Barcodes and Illumina adaptors were added to the amplicon with a second PCR reaction with 5 forward primers (0.2 µM each primer, FF1-AATGATACGGCGACCACCGAGATCTACACTCTTTCCCTACACGACGCTCTTCC GATCTTGGCGAACGGGTGAGTAA (SEQ ID NO: 18), FF2-AATGATACGGCGACCACCGAGATCTACACTCTTTCCCTACACGACGCTCTTCC GATCTACTCCTACGGGAGGCAGC (SEQ ID NO: 19), FF3-AATGATACGGCGACCACCGAGATCTACACTCTTTCCCTACACGACGCTCTTCC GATCTGTGTAGCGGTGRAATGCG (SEQ ID NO: 20), FF4-AATGATACGGCGACCACCGAGATCTACACTCTTTCCCTACACGACGCTCTTCC GATCTGGAGCATGTGGWTTAATTCGA (SEQ ID NO: 21), FF5-AATGATACGGCGACCACCGAGATCTACACTCTTTCCCTACACGACGCTCTTCC GATCTGGAGGAAGGTGGGGATGAC (SEQ ID NO: 22)) and one 8 nucleotide barcode-specific reverse primer (0.4 µM, RR5-CAAGCAGAAGACGGCATACGAGAT-NNNNNNNN-GTGACTGGAGTTCAGACGTGTGCTCTTCCGATCT (SEQ ID NO: 23)). The amplicon was diluted into the reaction (10-fold) and amplified with 6 cycles of 10 seconds at 98° C., 15 seconds at 64° C., 25 seconds at 72° C.’. Amplicons were then combined into sub-libraries (40-50 amplicons) and each library was purified using Qiaquick PCR purification kit (Qiagen #28104) according to the manufacturer’s instructions. Multiple sub-libraries were then combined into the final library (100-430 amplicons) and further purified from primer dimers using Agencourt AMPure XP (Beckman Coulter #A63881) at a volume ratio of 1:0.85 (library : beads). The library (7pM) was supplemented with 15% PhiX (8pM) and sequenced on Illumina Hi-seq 2500 v4 (paired end 2x125),Mi-seq v2 (paired-end 2x150) or NextSeq 500 mid output (paired end 2x150) sequencers.

Reconstruction was performed using the Short MUltiple Regions Framework (SMURF)(34). SMURF is a computational method for high-resolution 16S rRNA profiling, that combines sequencing results from any number of short amplified regions into a coherent solution. In brief, an ad hoc database is prepared by extracting the k-mers from each 16S rRNA sequence in each region, for a given set of primer pairs and desired read length (the database used in the current application of SMURF was Greengenes(35) containing ~1.2 M sequences). As preprocessing steps, low quality reads from the fastq files were filtered out in three cases: (i) A Phred score of less than 30 in more than 25% of the nucleotides; (ii) More than three nucleotides having a Phred score of less than 10; or (iii) When containing one or more ambiguous base calling (e.g., ‘N’). Remaining reads in each region are matched to the ad hoc database of k-mers in each region, and reconstruction is performed by applying the expectation-maximization algorithm for maximizing the multinomial likelihood of the observed reads. SMURF’s output comprises a list of ‘groups’ and their relative abundances, where a group is a set of full-length GG 16S rRNA sequences that share the same sequence over the de facto long amplicon (hence are indistinguishable). In almost all cases a group contains the same species, and in many cases contains a single 16S rRNA sequence. Each group is assigned a species-level taxonomy to allow taxonomy-based analysis (for groups comprising more than one 16S rRNA sequence, taxonomy is assigned by a majority vote). For pancreatic tumors only and additonal sequencing analysis method was applied as previously described(16).

16S data analysis. Relative abundances were converted to read counts by multiplying by the total number of reads. Sample reads were normalized within each sequencing library, by a factor representing the ratio of the average number of reads/sample in the specific library to the overall average across libraries. Samples with fewer than 1000 normalized reads (including negative controls) and species with relative abundances of less than 10⁻⁴ were discounted from further analysis.

We then apply a series of six filters to detect and remove contamination:

Filter 1: We removed species that had a high prevalence in negative controls; 113 species that appeared in over 7.5% (this number represents the inflection point of the bimodal distribution between most species that appear in very few controls, to species that exhibit a higher range of prevalences of control samples (n=636) were assumed to come from lab handling procedures, biological reagents and kits and were removed from further analysis. 139 species that appeared in over 7.5% of empty paraffin control samples that had more than 1,000 normalized reads (n=165) were assumed to come from global paraffin processing or represent global hospital environment contamination. 84 of them overlapped with the previous list so only an additional 55 and were removed from further analysis. Note that overall 168 species were removed as general contaminants (113+55) while in FIG. 3B it looks like only 167 species were removed as general contaminants (9,190 - 9,023). This difference stems from the fact that one general contaminant that was found in paraffin did not appear at all in our tumor/normal samples and is thus not included in the 9,190 species presented on FIG. 3B. Many of these general contaminant species corroborate with previously reported lists of contaminating species (21, 23). Next, the relative abundances and normalized reads values of higher level taxonomies such as genera and families were calculated based on the sums of those measures in their lower taxonomic members.

Filter 2-4: In order to remove the less prevalent contaminations (that could originate from rarer contamination events that occur during processing or cross-contaminations between samples) we compared taxa prevalences in samples to their prevalences in controls. We took into account that different contamination spectrums can vary across processing batches and time and that different tissue types may have different microbial biomass levels, efficiencies of DNA extraction and PCR amplification so we shifted to a ‘per-condition’ (a combined tissue+tumor/NAT/normal status) contamination analysis. Therefore, our comparisons between taxon prevalence in samples and controls were done on a per-condition, per-batch basis, comparing samples from the same condition to the controls that were processed alongside them. This was done for the three different processing batch types; DNA extraction batch (filter 2), PCR amplification batch (filter 3), and sequencing library batch (filter 4). We employed the non-parametric, exact, one-tailed binomial test on each taxon’s prevalence, x=number of successes in the sample and n=sample size to the background p= taxa’s prevalence in the relevant batch controls. Only taxa that passed a p-value cutoff of 0.05 in all batch comparisons were considered for the center filter (filter 6). Specifically for the colon tumor and NAT conditions, only one center was sampled so an FDR<0.2 was applied to the p-values of the sequencing library run filter (filter 4) and the center filter was not applied.

Filter 5: We compared prevalence of taxa in all samples per-condition to their prevalence in a set of empty paraffin controls originating from the same centers as the samples, requiring a one-tailed binomial p-value<=0.05 to pass.

Filter 6: To account for a center-specific batch effect, we compared prevalence of taxa in samples, again per-condition, this time per-medical-center to the same-center specific controls (DNA extraction, PCR, and library controls that ran with these specific center samples). This was performed via binomial test (as described above) on each center per-condition. Only taxa with p-value<=0.05 in at least two centers, and a Fisher’s combined p-value across all centers, FDR corrected < 0.20, passed this filter.

In order to eliminate sample size biases among comparisons between tissue types, this method was also applied on rarefied data; a range of sub-samplings from each tumor/NAT tissue across a spectrum of sample sizes. We performed 100 random sub-samplings per sample size. Averages and confidence intervals of the number of genera that passed all filters per tumor type per sample size calculated across the 100 randomizations are plotted as lines and sleeves, respectively (FIGS. 3C, 3F).

Richness and alpha-diversity measures across tissue types (FIGS. 3D, 3E) were calculated on rarefied data of 40 samples per tumor type, with 10 iterations. For each iteration the full hit calling pipeline was applied per tumor type and only bacterial species that passed all filters in any of the tumor types (‘hits’) were included in the analysis for all tumor types. For the control box plots (negative controls and paraffin controls) the presence of any of the ‘hits’ was counted in each of 636 negative controls and 165 paraffin controls.

The relative abundances in FIG. 4B were calculated taking into consideration all species (n=907) that passed all filters in any of the tumor types, including colon tumors.

Binary vectors representing the absence or presence of bacterial species that passed all filters in any of the tumor/NAT tissue types on the entire dataset were calculated for each tumor/NAT tissue type. The Jaccard similarity index was used to calculate an unweighted beta-diversity index amongst the tumor/NAT types, which was then used as the metric of a principal coordinate analysis. The resulting PCoA1 vs PCoA2 biplot is depicted in FIG. 4F.

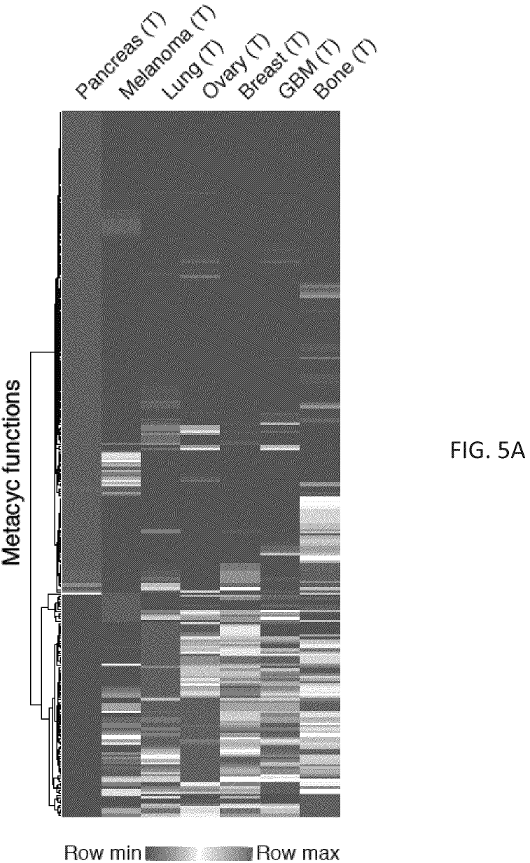

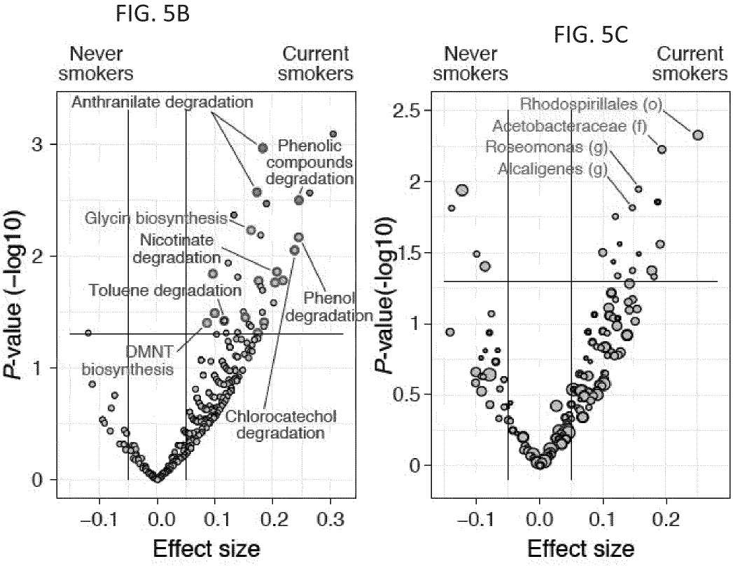

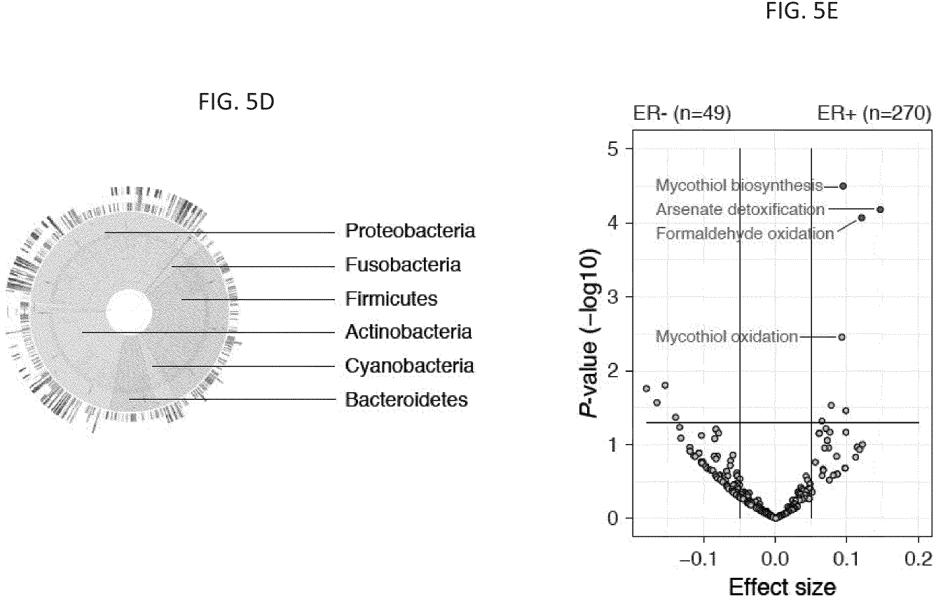

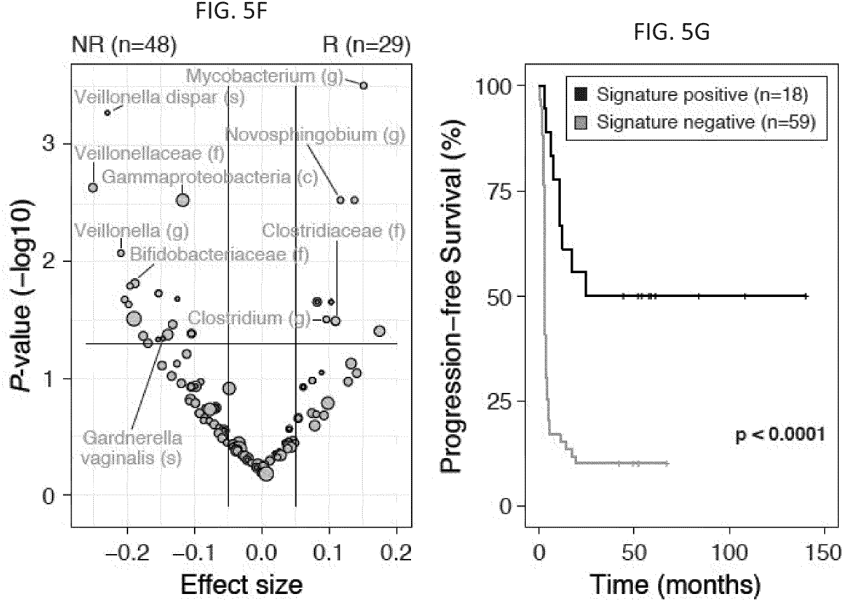

Clustering in FIGS. 4C and 5A was done with Morpheus tool (www(dot)software(dot)broadinstitute(dot)org/morpheus/, one minus Pearson correlation metric and average linkage method was used).

Comparisons of taxa or functions between clinical cohorts (e.g. smokers and never-smokers in lung tumors, breast tumor subtypes, tumor versus NAT tissue status) were carried out using the two-proportion z-test, comparing the proportion of samples positive for a given bacterial taxa (or function) in one clinical cohort to its proportion in a second cohort. Taxa or functions were tested if their prevalence was => 10% in at least one of the clinical cohorts tested. For the comparison of MetaCyc pathways in smokers vs non-smokers, functions were tested if their prevalence was => 10% in at least one of the clinical cohorts and not higher than 90% in both cohorts. The two-tailed p-value is reported for each taxon (or function) and it’s inverse log version is used as the Y axis of the volcano plot figures. The effect size on the X axis of the volcano plots is the difference in proportions of a taxon between the two compared cohorts. In addition, the one-tailed p-values of a non-parametric, hypergeometric test for enrichment of a taxa/function in one clinical condition (chosen as the condition in which the taxa was more prevalent) versus both and the one tailed p-values of an exact binomial test comparing the number of samples in which a taxa/function appear in the first clinical condition to the background of its proportion of positive samples (p) in the second condition are also included in the supplementary data tables. The latter test is used in the event of comparison between smaller sample sizes such as response status to immune-checkpoint inhibitors in melanoma. For this comparison, the relevant binomial test p-values are used to generate the Y axis of the volcano plot (FIG. 5F).

The comparison of bacteria between melanoma patients who responded to ICI treatment and patients who didn’t respond was carried out per patient. In patients for which multiple biopsies were analyzed, we have only used the sample that was retrieved at the earliest time point. If more than one biopsy was available from the same patient at the same earliest time point, these samples were united to one sample by combining their normalized reads. The cohort represents 79 samples from 49 non-responding patients and 38 samples from 29 responding patients. One patient was excluded for these analyses because no PFS data was available for that patient (48 NR and 29 R). All 46 bacterial taxa that were significantly differentially present in patients who responded to ICI treatment versus patients who didn’t respond to treatment (FIG. 5F) were used to further stratify the patients into two groups (18 taxa in responders and 28 in non-responders). For each patient, the proportion of response associated bacteria that are present out of 18 and the proportion of non-response associated bacteria that are present out of 28 was calculated (Table 4). Patients that had a higher (or equal) proportion of response associated bacteria compared to the proportion of non-response associated bacteria were assigned to the group of response signature positive patients. Progression-free survival plots for these two cohorts are shown in FIG. 5G.

Culturomics. Tissues were collected from five patients undergoing a scheduled surgery for the removal of a breast tumor with appropriate written informed consent and used under approval from the Institutional Review Boards of Tel-Aviv Sourasky Medical Center and Weizmann Institute of Science. Tumor tissues were collected in a sterile manner during surgery and were transferred on ice to our lab. Each tissue was dissected into 10 pieces under sterile conditions. One piece of the tissue was fixed in formalin and then embedded in paraffin, while another piece was fresh-frozen in liquid nitrogen. For isolation of anaerobic or aerobic bacteria, tissue pieces were placed in tubes containing one steel bead (3 mm) and 0.5 ml BHI media with or without 0.05% L-cystein, respectively. Four tubes were collected for each bacteria isolation condition (aerobic and anaerobic) and kept on ice until tissue dissociation. In parallel, control tubes for each condition were prepared in the same working area to account for workspace contaminants. Tubes were vigorously vortexed for 10 minutes to dissociate the tissue. All steps following tissue dissociation for anaerobic bacteria isolation were carried out inside an anaerobic chamber. Samples of the same condition were combined and diluted 1:1 with fresh BHI supplemented with 0.05% L-cystein for the anaerobic condition. Each dissociated tissue was plated on 35 different growth medium agar plates (100 µl per plate) and incubated at 37° C. for either 3 days in aerobic conditions or 5 days in anaerobic conditions. Control samples were plated and incubated for the same duration. Single colonies were picked and grown in appropriate liquid growth media and condition for 1-3 days. When multiple colonies with similar morphology grew on a specific plate, only two were picked for further growth. Plates were then scraped into BHI medium to bulk collect all cultured bacteria from all 35 plates grown in the same condition.

Bacterial genomic DNA from specific colonies or bulk bacteria populations were isolated according to the above-mentioned protocol for snap frozen tissues. DNA of each colony was diluted to 1.5 ng, and Illumina libraries were prepared using Nextera DNA library preparation kit, Ref# 15028211; by Tecan Freedom Evo 200 robot platform. IDT for Illumina Nextera DNA Unique Dual Indexes Sets A-D were used for library preparation. Library concentration was measured using iQuantTM dsDNA HS Assay Kit, ABP biosciences (Cat# AP-N011) and library size quantified by automated electrophoresis nucleic acid QC -Tape-Station system. Libraries were sequenced to a minimum depth of 1.3 million reads by NextSeq 500 machine with IlluminaNS 500/550 High Output V2 75 cycle kit, Cat# FC-404-2005.

Culturomics genome assembly and species Identification. First, reads that mapped to the hg19 human genome assembly were filtered out (GEM mapper(67) version 1.7.1; maximum of 5 mismatches allowed). Next, genome assembly of the filtered reads for each sample was performed with the de novo assembler MEGAHIT(68) (version 1.1.2). 16S rRNA sequences were detected by applying the Barrnap tool (www(dot)github(dot)com/tseemann/barrnap) to assembled contigs. Detections of length smaller than 1000nt were discarded and BLAST against the Greengenes database was applied to the remaining sequences. A match was declared whenever similarity was higher than 99.5%. In case no such match was found, a threshold of 97.0% similarity was set and the most prominent genus of matching Greengenes sequences was provided.

Live in-situ bacteria labeling with fluorescent D-alanine. Tumor tissues were collected from patients undergoing a scheduled surgery for the removal of a breast tumor with appropriate written informed consent and used under approval from the Institutional Review Boards of Tel-Aviv Sourasky Medical Center and Weizmann Institute of Science. Freshly-removed tumors were sliced into 250 µm thick slices using the Compresstome VF-300 microtome (Precissionary instruments), and were incubated ex-vivo for 2 hours with DMEM/F12 growth medium (without antibiotics) containing either blue fluorescently labeled D-alanine (HADA #6647, R&D Systems, at a final concentration of 1 µM) or DMSO control at 37oC and 80% oxygen. Slices were then washed twice with PBS, fixed in 4% PFA for 4 hours at room temperature and paraffin embedded. 5 µm histology cuts were deparaffinized, stained with 5 µM DRAQ5 (#62251, Thermo Scientific) for 30 minutes and mounted. Slides were scanned with the Pannoramic SCAN II automated slide scanner (3D HISTECH) at 40X.

PICRUSt2 analysis. We used PICRUSt2 (version alpha.2) to generate a mapping between the bacterial 16S sequences found in our samples to a list of functions predicted for them. Two separate mappings were generated for MetaCyc metabolic pathways and KEGG orthologs. To facilitate the interpretation of functional results, for the MetaCyc pathways we used the supporting data from classes.dat and pathways.dat (provided to subscribers of BioCyc) to generate the hierarchies of each pathway and for KEGG orthologs, we used the KEGG BRITE hierarchy.

Binary tables representing the absence/presence of a function (row) in a bacterium (column) were matrix multiplied against the tables representing the species normalized reads per sample. This resulted in a function per sample table, where every cell [i,j] is the sum of normalized reads of function ‘i’s contributing species per sample ‘j’. Each cell [i,j] was then divided by the total number of normalized reads per sample ‘j’ from the original species table to generate average read frequencies of functions per sample. For all functional comparison analyses, function tables were generated from all present bacteria excluding global contaminants.

These read frequency values were used in the unsupervised clustering of 287 MetaCyc pathways that were most variable between the different tumor types in FIG. 5A (Morpheus — www(dot)software(dot)broadinstitute(dot)org/morpheus/, one minus Pearson correlation metric and average linkage method was used).

Binarized versions of these tables were used to calculate percent prevalence of a given function in a set of samples. FIGS. 5B and 5E represent comparisons between function prevalences from two different sets of samples. A two-tailed proportion ztest, which takes the sample sizes into account, was applied on all functions, that had a minimal prevalence of 10% in at least one group of patients, to assess the significance of the comparisons.

The MetaCyc pathways that were found to be significantly enriched in lung tumors of smokers when compared to never-smokers, were manually categorized into two classes of functions: cigarette smoke metabolites degradation functions and the biosynthesis of plant-related metabolites. The contributing species for each class were determined based on the original PICRUSt2 mappings between species and function and the presence of the species in the relevant samples. The normalized reads of each class of species were plotted as color-gradiented spokes in rings on a modified GraPhlAn(70) plot depicting taxonomic distribution of the bacterial species hits across the seven tumor types (FIG. 5D).

### EXAMPLE 1 Bacterial DNA, RNA and Lipopolysaccharide are Frequently Present in Human Solid Tumors

We focused on seven human solid tumor types that represent either common cancer types or cancer types for which the tumor microbiome has never been reported before like melanoma, bone, and brain tumors (FIG. 1A). To address lab-bome contaminants, we introduced 643 negative controls that were processed together with the samples, including 437 DNA extraction controls and 206 PCR no-template controls (NTC). To address contamination that might have occurred before the samples reached our lab, we also included 168 paraffin only samples taken from the margins of the paraffin blocks (without tissue) that were used in the study (FIG. 1A).

Overall, we profiled 1,010 tumor samples and 516 normal samples, including normal adjacent tissues (NAT) from the same patients (FIG. 1A). In the case of ovarian cancer, our normal samples came from the ovaries or uterus of the patients, or from normal fallopian tube fimbria of normal unmatched patients. To quantify bacterial DNA, we used a real-time quantitative PCR (qPCR) assay with universal primers 967F-1064R against the bacterial ribosomal 16S gene (16S rDNA) (24). Levels of bacterial DNA in all tumor types were significantly higher than in both DNA extraction and paraffin controls (FIG. 1B, p-value<10⁻¹⁰ for each tumor type, Wilcoxon rank-sum test). We found that different cancer types vary in the proportion of tumors that are positive for bacterial DNA, ranging from only 14.3% in melanoma to over 60% in breast, pancreas, and bone tumors. Bacterial DNA was also detected in solid tumors that have no direct connection with the external environment, such as ovarian cancer, glioblastoma multiforme (GBM), and bone cancer.

To further validate the presence of bacteria in human tumors, we stained more than 400 additional tumors (not related to the 1,526 samples described above), representing six of our seven profiled tumor types, for the presence of bacteria. We conducted immunohistochemistry (IHC) using antibodies against bacterial lipopolysaccharide (LPS) and lipoteichoic acid (LTA) to detect Gram-negative and Gram-positive bacteria, respectively (25, 26). We also used RNA fluorescent in situ hybridization (FISH), with a universal probe against bacterial 16S rRNA, to detect bacterial RNA in these tumors (27). To control for non-specific staining, IHC negative controls (no primary antibody) and FISH negative controls (non-specific complement probe) were also applied to the samples. Overall, bacterial LPS and 16S rRNA were frequently detected in all tumor types (FIG. 1C), and demonstrated a similar spatial distribution (FIG. 1D). LTA was detected mostly in melanoma tumors, and remained largely absent in other tumor types. In general, more tumors were found to be positive for bacteria using visualization methods than qPCR. This disparity may be due to some limitation in the sensitivity of our qPCR assay, as well as our strict cutoff for confirming a sample as positive.

### EXAMPLE 2 Intra-Tumor Bacteria Are Mostly Intracellular, and Can Be Found in Both Cancer and Immune Cells

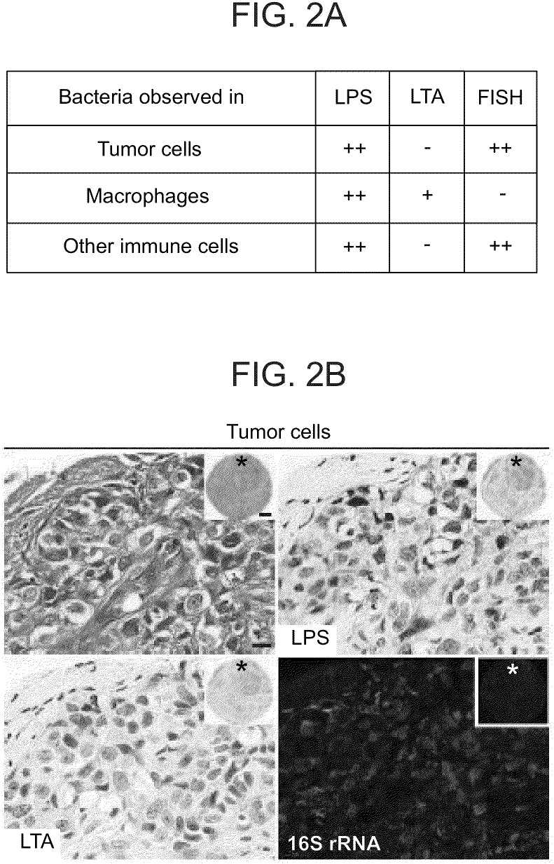

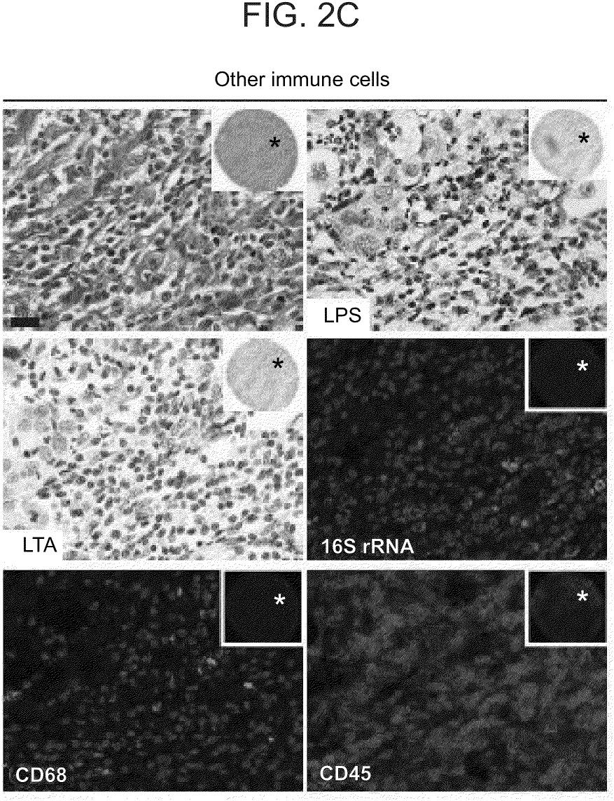

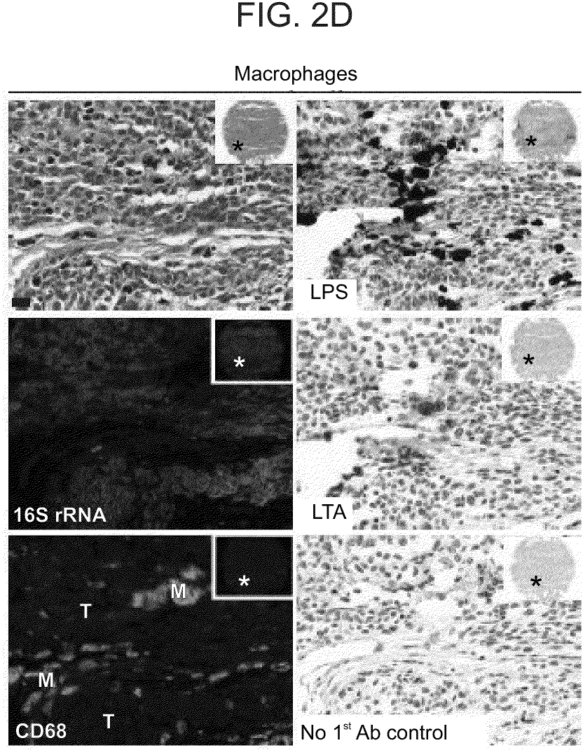

Pathological examination of tumor cores indicated that LPS and bacterial 16S rRNA were detected mostly in cancer and immune cells (FIG. 2A). In cancer cells, bacterial 16S rRNA was detected mostly in the cytoplasm, whereas LPS staining was associated with both the cytoplasm and the nucleus (FIG. 2B). CD45-positive leukocytes generally exhibited a stronger cytoplasmic bacterial staining by 16S rRNA staining than did cancer cells (FIG. 2C). LTA-positive bacteria were almost exclusively found in macrophages, as detected by H&E staining and verified by immunofluorescence (IF) for CD68 (FIG. 2D). LTA was rarely detected in cancer cells or in CD45+/CD68- immune cells (FIGS. 2A-E). While the intensity of bacterial LPS and LTA staining was very strong in CD45+/CD68+ cells, bacterial 16S rRNA was only rarely found in macrophages by FISH (FIGS. 2A and 2D). This discrepancy may reflect macrophage ingestion of bacterial components rather than live bacteria or may result from the accumulation of LPS and LTA in macrophages long after the bacteria have been phagocytized and processed by the macrophages. Indeed, it has been previously demonstrated that processing of bacterial LPS by macrophages is very slow, therefore LPS can be found in these cells months after the bacteria were ingested and processed (28).

To further verify the presence of bacteria inside cancer cells, we performed correlative light and electron microscopy (CLEM) (29, 30) on four human breast tumors that were positive for bacterial LPS and 16S rRNA. Combined LPS fluorescence staining and transmission electron microscopy (TEM) imaging of the same cells clearly demonstrated the intra-cellular localization of bacteria in all four tumors (FIG. 2E). In many cases, the bacteria were found in close proximity to the nuclear membrane. Since we did not detect intra-nuclear bacteria by TEM, we suspect that the appearance of LPS nuclear localization in some tumors represents staining of cytoplasmic perinuclear bacteria.

Whereas bacterial 16S rRNA FISH signals were found to be mostly diffused inside cells, typical bacterial rods or cocci were only rarely detected (in 3 out of 426 cores). This observation, together with the fact that no cell wall polymer LTA was detected in cancer cells, despite the detection of many Gram-positive bacteria in human tumors by 16S rDNA sequencing, suggests that it is possible that bacteria in tumor cells may have altered their envelope, perhaps leading to a cell-wall deficient state, akin to L-forms (31). Indeed, cell wall-deficient bacteria are known to be found exclusively inside cells, where their morphology transforms into less defined structures of highly variable sizes and shapes (32, 33). Our TEM images also suggest that many of the intra-cellular bacteria lack a cell wall (FIG. 2E).

### EXAMPLE 3 The Microbiome of Breast Tumors is Richer and More Diverse Than That of Other Tumor Types

To characterize the intra-tumor microbiome, we developed a novel multiplexed 16S rDNA sequencing protocol that amplifies five short regions along the 16S rRNA gene: the “5R” 16S rDNA sequencing method (FIG. 3A). By amplifying 68% of the bacterial 16S rRNA gene using short amplicons, this method increases the coverage and resolution of bacterial species detection compared to the widely used V4 or V3-V4 amplification. Moreover, it can be applied to relatively degraded DNA, originating from formalin-fixed paraffin-embedded (FFPE) tumors. Reads from 1,526 samples and 811 negative controls (DNA extraction controls, 16S 5R PCR controls and paraffin controls), were computationally combined into long amplicons, using Short MUltiple Regions Framework (SMURF) (13), and the Greengenes database as a reference. To improve taxonomic assignment, we used the Ribosomal Database Project (RDP) classifier to augment the Greengenes database by assigning a species-level taxonomy to 380,000 bacterial 16S rRNA sequences that originally lacked such taxonomy (35). Thirty nine samples and 10 controls that had less than 1,000 normalized reads were discarded from further analysis.

Overall, we detected 9,190 bacterial species across the different tumor or normal tissue types (FIG. 3B). Because some of these species may represent contamination of the samples, we applied a strict set of six filters to control for potential sources of contamination. To account for the most frequent general contaminants, filter 1 removed 167 bacterial species that were detected in more than 7.5% of our DNA extraction/NTC negative control samples or in the paraffin controls. This threshold demarcates the transition between the great majority of species that are mostly absent or very rarely present in controls, and species that appear much more commonly in controls. We then applied three filters to control for batch effects originating from DNA extraction, PCR amplification or sequencing lane using hundreds of negative controls as background for lab-borne contamination (filters 2-4). Filters 5 and 6 were added to control for contamination that might have been introduced to the samples prior to their processing in the lab. Filter 5 uses paraffin only samples (without tissue) from the margins of the same paraffin blocks that were used in the study, to control for contamination in the process of paraffin blocks preparation and storage. Lastly, to account for other potential sources of medical-center-specific contamination, filter 6 excluded bacteria that were not significantly enriched in a specific tumor type across multiple medical centers. Only bacteria that passed all six filters in a specific cancer type or its NAT were considered as ‘hits’ that are present in this cancer or NAT condition (FIG. 3B).

We found that breast tumors had a richer and more diverse microbiome than all other tumor types tested (p-value<10⁻¹⁵ for each tumor type, Wilcoxon rank-sum test, FIGS. 3C, 3D). An average of 16.4 bacterial species were detected in any single breast tumor sample, whereas the average was lower than 9 in all other tumor types (p-value<10⁻¹⁷ for each tumor type, Wilcoxon rank-sum test, FIG. 3E). We also found that bacterial load and richness were higher in breast tumor samples than in normal breast samples from healthy subjects. Tumor-adjacent normal breast tissue had an intermediate bacterial load and richness, between those of the breast tumor and normal samples (FIG. 3F). In contrast, we did not find a higher bacterial load in lung and ovarian cancer as compared to their tumor-adjacent normal tissue.

To determine whether live bacteria are present in human tumors, we collected fresh breast tumor samples from five women undergoing breast surgery. All tissues were gently dissociated in sterile conditions, plated on 35 types of agar growth media, and incubated in both aerobic and anaerobic conditions, representing a broad span of growth conditions to accommodate a high diversity of bacteria (36). Bacteria and fungi that were identified are presented in Table 1 below.

TABLE 1

| king dom | phylum | class | order | family | genus | species | SEQ ID |
|---|---|---|---|---|---|---|---|
| Bacteria | Actinobacteria | Actinobacteria | Actinomycetales | Actinomycetaceae | Trueperella | | 24 |
| Bacteria | Actinobacteria | Actinobacteria | Actinomycetales | Bogoriellaceae | Georgenia | | 25 |
| Bacteria | Actinobacteria | Actinobacteria | Actinomycetales | Cellulomonadaceae | Cellulomonas | | 26 |
| Bacteria | Actinobacteria | Actinobacteria | Actinomycetales | Cellulomonadaceae | Oerskovia | | 27 |
| Bacteria | Actinobacteria | Actinobacteria | Actinomycetales | Corynebacteriaceae | Corynebacterium | Corynebacterium tuberculos tearicum | 29 |
| Bacteria | Actinobacteria | Actinobacteria | Actinomycetales | Corynebacteriaceae | Corynebacterium | Corynebacterium tuberculos tearicum | 31 |
| Bacteria | Actinobacteria | Actinobacteria | Actinomycetales | Corynebacteriaceae | Corynebacterium | Corynebacterium variabile | 32 |
| Bacteria | Actinobacteria | Actinobacteria | Actinomycetales | Corynebacteriaceae | Corynebacterium | | 33 |
| Bacteria | Actinobacteria | Actinobacteria | Actinomycetales | Corynebacteriaceae | Corynebacterium | | 35 |
| Bacteria | Actinobacteria | Actinobacteria | Actinomycetales | Corynebacteriaceae | Corynebacterium | | 36 |
| Bacteria | Actinobacteria | Actinobacteria | Actinomycetales | Corynebacteriaceae | Corynebacterium | | 37 |
| Bacteria | Actinobacteria | Actinobacteria | Actinomycetales | Dermabacteraceae | Dermabacter | | 38 |
| Bacteria | Actinobacteria | Actinobacteria | Actinomycetales | Dermacoccaceae | Dermacoccus | | 39 |
| Bacteria | Actinobacteria | Actinobacteria | Actinomycetales | Dermacoccaceae | Dermacoccus | | 40 |
| Bacteria | Actinobacteria | Actinobacteria | Actinomycetales | Dietziaceae | Dietzia | | 41 |
| Bacteria | Actinobacteria | Actinobacteria | Actinomycetales | Geodermato philaceae | Blastococcus | | 42 |
| Bacteria | Actinobacteria | Actinobacteria | Actinomycetales | Intrasporangiaceae | Janibacter | | 43 |
| Bacteria | Actinobacteria | Actinobacteria | Actinomycetales | Intrasporangiaceae | Ornithinimicrobium | | 44 |
| Bacteria | Actinobacteria | Actinobacteria | Actinomycetales | Microbacteriaceae | Agrococcus | | 45 |
| Bacteria | Actinobacteria | Actinobacteria | Actinomycetales | Microbacteriaceae | Agrococcus | | 46 |
| Bacteria | Actinobacteria | Actinobacteria | Actinomycetales | Microbacteriaceae | Microbacterium | | 47 |
| Bacteria | Actinobacteria | Actinobacteria | Actinomycetales | Microbacteriaceae | Microbacterium | | 48 |
| Bacteria | Actinobacteria | Actinobacteria | Actinomycetales | Microbacteriaceae | Microbacterium | | 49 |
| Bacteria | Actinobacteria | Actinobacteria | Actinomycetales | Microbacteriaceae | Microbacterium | | 50 |
| Bacteria | Actinobacteria | Actinobacteria | Actinomycetales | Microbacteriaceae | Microbacterium | | 51 |
| Bacteria | Actinobacteria | Actinobacteria | Actinomycetales | Micrococcaceae | Arthrobacter | Arthrobacter aurescens | 52 |
| Bacteria | Actinobacteria | Actinobacteria | Actinomycetales | Micrococcaceae | Micrococcus | Micrococcus luteus | 53 |
| Bacteria | Actinobacteria | Actinobacteria | Actinomycetales | Micrococcaceae | Micrococcus | Micrococcus luteus | 54 |
| Bacteria | Actinobacteria | Actinobacteria | Actinomycetales | Micrococcaceae | Micrococcus | Micrococcus luteus | 55 |
| Bacteria | Actinobacteria | Actinobacteria | Actinomycetales | Micrococcaceae | Micrococcus | Micrococcus luteus | 56 |
| Bacteria | Actinobacteria | Actinobacteria | Actinomycetales | Micrococcaceae | Micrococcus | Micrococcus luteus | 57 |
| Bacteria | Actinobacteria | Actinobacteria | Actinomycetales | Micrococcaceae | Micrococcus | Micrococcus luteus | 58 |
| Bacteria | Actinobacteria | Actinobacteria | Actinomycetales | Micrococcaceae | Micrococcus | Micrococcus luteus | 59 |
| Bacteria | Actinobacteria | Actinobacteria | Actinomycetales | Micrococcaceae | Micrococcus | Micrococcus luteus | 60 |
| Bacteria | Actinobacteria | Actinobacteria | Actinomycetales | Micrococcaceae | Micrococcus | Micrococcus luteus | 61 |
| Bacteria | Actinobacteria | Actinobacteria | Actinomycetales | Micrococcaceae | Micrococcus | Micrococcus luteus | 62 |
| Bacteria | Actinobacteria | Actinobacteria | Actinomycetales | Micrococcaceae | Micrococcus | Micrococcus luteus | 63 |
| Bacteria | Actinobacteria | Actinobacteria | Actinomycetales | Micrococcaceae | Micrococcus | Micrococcus luteus | 64 |
| Bacteria | Actinobacteria | Actinobacteria | Actinomycetales | Micrococcaceae | Micrococcus | Micrococcus luteus | 65 |
| Bacteria | Actinobacteria | Actinobacteria | Actinomycetales | Micrococcaceae | Micrococcus | Micrococcus luteus | 66 |
| Bacteria | Actinobacteria | Actinobacteria | Actinomycetales | Micrococcaceae | Micrococcus | Micrococcus luteus | 67 |
| Bacteria | Actinobacteria | Actinobacteria | Actinomycetales | Micrococcaceae | Micrococcus | Micrococcus luteus | 68 |
| Bacteria | Actinobacteria | Actinobacteria | Actinomycetales | Micrococcaceae | Micrococcus | Micrococcus luteus | 69 |
| Bacteria | Actinobacteria | Actinobacteria | Actinomycetales | Micrococcaceae | Micrococcus | Micrococcus luteus | 70 |
| Bacteria | Actinobacteria | Actinobacteria | Actinomycetales | Micrococcaceae | Micrococcus | Micrococcus luteus | 71 |
| Bacteria | Actinobacteria | Actinobacteria | Actinomycetales | Micrococcaceae | Arthrobacter | | 72 |
| Bacteria | Actinobacteria | Actinobacteria | Actinomycetales | Micrococcaceae | Kocuria | | 73 |
| Bacteria | Actinobacteria | Actinobacteria | Actinomycetales | Micrococcaceae | Microbispora | | 74 |
| Bacteria | Actinobacteria | Actinobacteria | Actinomycetales | Micrococcaceae | Microbispora | | 75 |
| Bacteria | Actinobacteria | Actinobacteria | Actinomycetales | Micrococcaceae | Micrococcus | | 76 |
| Bacteria | Actinobacteria | Actinobacteria | Actinomycetales | Micrococcaceae | Micrococcus | | 77 |
| Bacteria | Actinobacteria | Actinobacteria | Actinomycetales | Micrococcaceae | Micrococcus | | 78 |
| Bacteria | Actinobacteria | Actinobacteria | Actinomycetales | Micrococcaceae | Micrococcus | | 79 |
| Bacteria | Actinobacteria | Actinobacteria | Actinomycetales | Mycobacteriaceae | Mycobacterium | | 80 |
| Bacteria | Actinobacteria | Actinobacteria | Actinomycetales | Nocardiaceae | Rhodococcus | Rhodococcus erythropolis | 81 |
| Bacteria | Actinobacteria | Actinobacteria | Actinomycetales | Propionibacteriaceae | Propionibacterium | Propionibacterium acnes | 82 |
| Bacteria | Actinobacteria | Actinobacteria | Actinomycetales | Propionibacteriaceae | Propionibacterium | Propionibacterium acnes | 83 |
| Bacteria | Actinobacteria | Actinobacteria | Actinomycetales | Propionibacteriaceae | Propionibacterium | Propionibacterium acnes | 84 |
| Bacteria | Actinobacteria | Actinobacteria | Actinomycetales | Propionibacteriaceae | Propionibacterium | Propionibacterium avidum | 85 |
| Bacteria | Actinobacteria | Actinobacteria | Actinomycetales | Propionibacteriaceae | Propionibacterium | Propionibacterium avidum | 86 |
| Bacteria | Firmicutes | Bacilli | Bacillales | Bacillaceae | Bacillus | Bacillus flexus | 87 |
| Bacteria | Firmicutes | Bacilli | Bacillales | Bacillaceae | Bacillus | Bacillus flexus | 88 |
| Bacteria | Firmicutes | Bacilli | Bacillales | Bacillaceae | Bacillus | Bacillus muralis | 89 |
| Bacteria | Firmicutes | Bacilli | Bacillales | Bacillaceae | Bacillus | | 90 |
| Bacteria | Firmicutes | Bacilli | Bacillales | Bacillaceae 1 | Bacillus | Bacillus subtilis | 91 |
| Bacteria | Firmicutes | Bacilli | Bacillales | Bacillaceae 1 | Bacillus | Bacillus subtilis | 92 |
| Bacteria | Firmicutes | Bacilli | Bacillales | Bacillaceae 1 | Bacillus | Bacillus subtilis | 93 |
| Bacteria | Firmicutes | Bacilli | Bacillales | Bacillaceae 1 | Bacillus | Bacillus foraminis | 94 |
| Bacteria | Firmicutes | Bacilli | Bacillales | Bacillaceae 1 | Bacillus | Bacillus nealsonii | 95 |
| Bacteria | Firmicutes | Bacilli | Bacillales | Bacillaceae 2 | Terribacillus | | 96 |
| Bacteria | Firmicutes | Bacilli | Bacillales | Planococcaceae | Chryseomicrobium | Chryseomicrobium imtechense | 97 |
| Bacteria | Firmicutes | Bacilli | Bacillales | Planococcaceae | Chryseomicrobium | | 98 |
| Bacteria | Firmicutes | Bacilli | Bacillales | Planococcaceae | Sporosarcina | | 99 |
| Bacteria | Firmicutes | Bacilli | Bacillales | Planococcaceae | Sporosarcina | | 100 |
| Bacteria | Firmicutes | Bacilli | Bacillales | Staphylococcaceae | Staphylococcus | Staphylococcus epidermidis | 101 |
| Bacteria | Firmicutes | Bacilli | Bacillales | Staphylococcaceae | Staphylococcus | Staphylococcus epidermidis | 102 |
| Bacteria | Firmicutes | Bacilli | Bacillales | Staphylococcaceae | Staphylococcus | Staphylococcus epidermidis | 103 |
| Bacteria | Firmicutes | Bacilli | Bacillales | Staphylococcaceae | Staphylococcus | Staphylococcus epidermidis | 104 |
| Bacteria | Firmicutes | Bacilli | Bacillales | Staphylococcaceae | Staphylococcus | Staphylococcus epidermidis | 105 |
| Bacteria | Firmicutes | Bacilli | Bacillales | Staphylococcaceae | Staphylococcus | Staphylococcus epidermidis | 106 |
| Bacteria | Firmicutes | Bacilli | Bacillales | Staphylococcaceae | Staphylococcus | Staphylococcus epidermidis | 107 |
| Bacteria | Firmicutes | Bacilli | Bacillales | Staphylococcaceae | Staphylococcus | Staphylococcus epidermidis | 108 |
| Bacteria | Firmicutes | Bacilli | Bacillales | Staphylococcaceae | Staphylococcus | Staphylococcus epidermidis | 109 |
| Bacteria | Firmicutes | Bacilli | Bacillales | Staphylococcaceae | Staphylococcus | Staphylococcus epidermidis | 110 |
| Bacteria | Firmicutes | Bacilli | Bacillales | Staphylococcaceae | Staphylococcus | Staphylococcus epidermidis | 111 |
| Bacteria | Firmicutes | Bacilli | Bacillales | Staphylococcaceae | Staphylococcus | Staphylococcus haemolyticus | 112 |
| Bacteria | Firmicutes | Bacilli | Bacillales | Staphylococcaceae | Staphylococcus | Staphylococcus hominis | 113 |
| Bacteria | Firmicutes | Bacilli | Bacillales | Staphylococcaceae | Staphylococcus | Staphylococcus hominis | 114 |
| Bacteria | Firmicutes | Bacilli | Bacillales | Staphylococcaceae | Staphylococcus | Staphylococcus hominis | 115 |
| Bacteria | Firmicutes | Bacilli | Bacillales | Staphylococcaceae | Staphylococcus | Staphylococcus lugdunensis | 116 |
| Bacteria | Firmicutes | Bacilli | Bacillales | Staphylococcaceae | Staphylococcus | Staphylococcus lugdunensis | 117 |
| Bacteria | Firmicutes | Bacilli | Bacillales | Staphylococcaceae | Staphylococcus | Staphylococcus lugdunensis | 118 |
| Bacteria | Firmicutes | Bacilli | Bacillales | Staphylococcaceae | Staphylococcus | Staphylococcus lugdunensis | 119 |
| Bacteria | Firmicutes | Bacilli | Bacillales | Staphylococcaceae | Staphylococcus | Staphylococcus lugdunensis | 120 |
| Bacteria | Firmicutes | Bacilli | Bacillales | Staphylococcaceae | Staphylococcus | Stahylococcus lugdunensis | 121 |
| Bacteria | Firmicutes | Bacilli | Bacillales | Staphylococcaceae | Staphylococcus | Staphylococcus lugdunensis | 122 |
| Bacteria | Firmicutes | Bacilli | Bacillales | Staphylococcaceae | Staphylococcus | Staphylococcus succinus | 123 |
| Bacteria | Firmicutes | Bacilli | Bacillales | Staphylococcaceae | Staphylococcus | Staphylococcus succinus | 124 |
| Bacteria | Firmicutes | Bacilli | Bacillales | Staphylococcaceae | Staphylococcus | Staphylococcus succinus | 125 |
| Bacteria | Firmicutes | Bacilli | Bacillales | Staphylococcaceae | Staphylococcus | | 126 |
| Bacteria | Firmicutes | Bacilli | Bacillales | Unknown species | Exiguobacterium | Exiguobacterium mexicanum | 127 |
| Bacteria | Firmicutes | Bacilli | Bacillales | Unknown species | Exiguobacterium | Exiguobacterium profundum | 128 |
| Bacteria | Firmicutes | Bacilli | Bacillales | Unknown species | Exiguobacterium | | 129 |
| Bacteria | Firmicutes | Bacilli | Lactobacillales | Aerococcaceae | Aerococcus | Aerococcus viridans | 130 |
| Bacteria | Firmicutes | Bacilli | Lactobacillales | Enterococcaceae | Enterococcus | Enterococcus faecalis | 131 |
| Bacteria | Firmicutes | Bacilli | Lactobacillales | Streptococcaceae | Streptococcus | Streptococcus infantis | 132 |
| Bacteria | Firmicutes | Bacilli | Lactobacillales | Streptococcaceae | Streptococcus | Streptococcus infantis | 133 |
| Bacteria | Firmicutes | Bacilli | Lactobacillales | Streptococcaceae | Streptococcus | Streptococcus infantis | 134 |
| Bacteria | Firmicutes | Bacilli | Lactobacillales | Streptococcaceae | Streptococcus | Streptococcus infantis | 135 |
| Bacteria | Firmicutes | Bacilli | Lactobacillales | Streptococcaceae | Streptococcus | Streptococcus oralis | 136 |
| Bacteria | Firmicutes | Bacilli | Lactobacillales | Streptococcaceae | Streptococcus | Streptococcus pneumoniae | 137 |
| Bacteria | Firmicutes | Bacilli | Lactobacillales | Streptococcaceae | Streptococcus | Streptococcus pneumoniae | 138 |
| Bacteria | Firmicutes | Bacilli | Lactobacillales | Streptococcaceae | Streptococcus | Streptococcus sanguinis | 139 |
| Bacteria | Firmicutes | Bacilli | Lactobacillales | Streptococcaceae | Streptococcus | Streptococcus vestibularis | 140 |
| Bacteria | Firmicutes | Bacilli | Lactobacillales | Streptococcaceae | Streptococcus | Streptococcus vestibularis | 141 |
| Bacteria | Firmicutes | Bacilli | Lactobacillales | Streptococcaceae | Streptococcus | Assigned species 1791 | 142 |
| Bacteria | Proteobacteria | Alphaproteobacteria | Rhodobacterales | Rhodobacteraceae | Paracoccus | Paracoccus aminovorans | 143 |
| Bacteria | Proteobacteria | Alphaproteobacteria | Rhodobacterales | Rhodobacteraceae | Paracoccus | | 144 |
| Bacteria | Proteobacteria | Alphaproteobacteria | Rhodospirillales | Acetobacteraceae | Roseomonas | Roseomonas mucosa | 145 |
| Bacteria | Proteobacteria | Alphaproteobacteria | Rhodospirillales | Acetobacteraceae | Roseomonas | | 146 |
| Bacteria | Proteobacteria | Alphaproteobacteria | Sphingomonadales | Sphingomonadaceae | Sphingomonas | Sphingomonas desiccabilis | 147 |
| Bacteria | Proteobacteria | Betaproteobacteria | Burkholderiales | Oxalobacteraceae | Massilia | | 148 |
| Bacteria | Proteobacteria | Betaproteobacteria | Neisseriales | Neisseriaceae | Neisseria | Neisseria macacae | 149 |
| Bacteria | Proteobacteria | Betaproteobacteria | Neisseriales | Neisseriaceae | Neisseria | Neisseria subflava | 150 |
| Bacteria | Proteobacteria | Betaproteobacteria | Neisseriales | Neisseriaceae | Neisseria | Neisseria subflava | 151 |
| Bacteria | Proteobacteria | Gammaproteobacteria | Enterobacteriales | Enterobacteriaceae | Enterobacter | Enterobacter cloacae | 152 |
| Bacteria | Proteobacteria | Gammaproteobacteria | Enterobacteriales | Enterobacteriaceae | Proteus | Proteus mirabilis | 153 |
| Bacteria | Proteobacteria | Gammaproteobacteria | Enterobacteriales | Enterobacteriaceae | Proteus | Proteus mirabilis | 154 |
| Bacteria | Proteobacteria | Gammaproteobacteria | Enterobacteriales | Enterobacteriaceae | Proteus | Proteus mirabilis | 155 |
| Bacteria | Proteobacteria | Gammaproteobacteria | Enterobacteriales | Enterobacteriaceae | Erwinia | | 156 |
| Bacteria | Proteobacteria | Gammaproteobacteria | Enterobacteriales | Enterobacteriaceae | Erwinia | | 157 |
| Bacteria | Proteobacteria | Gammaproteobacteria | Enterobacteriales | Enterobacteriaceae | Erwinia | | 158 |
| Bacteria | Proteobacteria | Gammaproteobacteria | Enterobacteriales | Enterobacteriaceae | Erwinia | | 159 |
| Bacteria | Proteobacteria | Gammaproteobacteria | Pseudomonadales | Moraxellaceae | Acinetobacter | Acinetobacter radioresistens | 160 |
| Bacteria | Proteobacteria | Gammaproteobacteria | Pseudomonadales | Moraxellaceae | Enhydrobacter | Enhydrobacter aerosaccus | 161 |
| Bacteria | Proteobacteria | Gammaproteobacteria | Pseudomonadales | Moraxellaceae | Enhydrobacter | Enhydrobacter aerosaccus | 162 |
| Bacteria | Proteobacteria | Gammaproteobacteria | Pseudomonadales | Moraxellaceae | Enhydrobacter | Enhydrobacter aerosaccus | 163 |
| Bacteria | Proteobacteria | Gammaproteobacteria | Pseudomonadales | Moraxellaceae | Enhydrobacter | | 164 |
| Bacteria | Proteobacteria | Gammaproteobacteria | Pseudomonadales | Pseudomonadaceae | Pseudomonas | | 165 |
| Bacteria | Proteobacteria | Gammaproteobacteria | Pseudomonadales | Pseudomonadaceae | Pseudomonas | | 166 |
| Fungi | Ascomycota | Eurotiomycetes | Eurotiales | Trichocomaceae | Aspergillus | Aspergillus kawachii | 167 |
| Fungi | Ascomycota | Eurotiomycetes | Eurotiales | Trichocomaceae | Aspergillus | Aspergillus niger | 168 |
| Fungi | Ascomycota | Eurotiomycetes | Eurotiales | Trichocomaceae | Aspergillus | Aspergillus pseudoglaucus | 169 |
| Fungi | Ascomycota | Saccharomycetes | Saccharomycetales | Saccharomycetaceae | Saccharomyces | Saccharo mycescerevisiae | 170 |

In agreement with the positive staining of these tumors for LPS and 16S rRNA FISH, more than 1,000 colonies were grown per tumor from four of the tumors, and 37 colonies were grown from one tumor. In contrast, applying the same steps of tissue dissociation and culturing protocol to five full sets of negative control plates (350 plates) using PBS only, yielded only five colonies in total. Whole-genome sequencing of 474 representative colonies from all 5 tumors demonstrated that they represented 37 different bacterial species. For 105 of the colonies we could not identify the bacteria at the species level. Overall, these results support the presence of live bacteria from the three most predominant phyla of breast tumors - Proteobacteria, Firmicutes and Actinobacteria.

To further validate the presence of live, metabolically active, bacteria in human tumors, we cultured slices from four freshly resected human breast tumors ex-vivo in the presence of fluorescently-labeled D-alanine or DMSO control. While D-alanine is used by bacteria to generate peptidoglycan, an essential component of the bacterial cell wall, it is not used by mammalian cells. Reassuringly we detected intra-cellular labeling in all four tumors, supporting the presence of live intra-cellular bacteria in them (FIG. 3G).

### EXAMPLE 4 Different Tumor Types Have Distinct Microbial Compositions

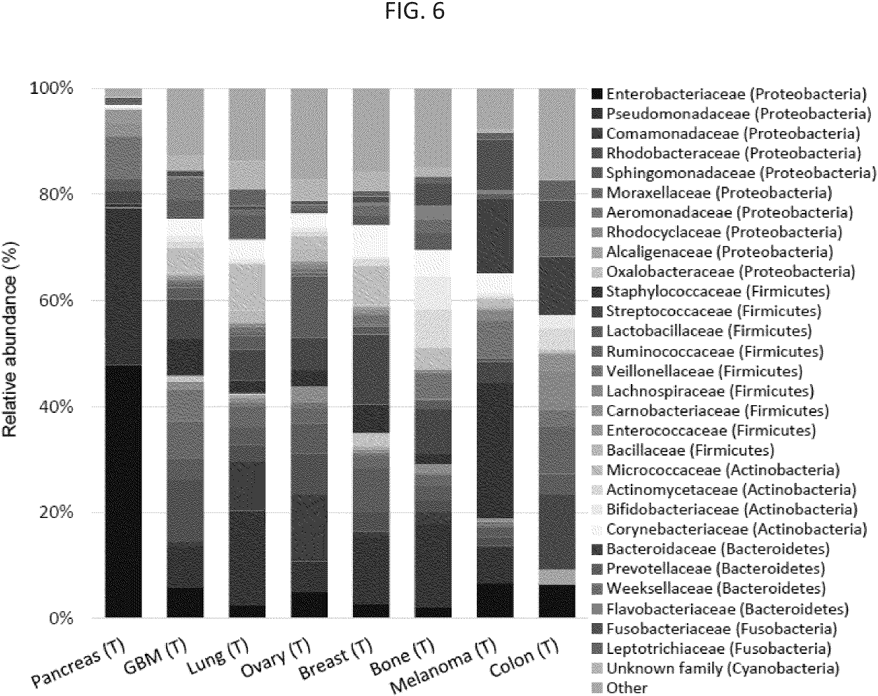

Using a single sequencing methodology and platform for the characterization of the microbiome in multiple tumor types enabled us to pioneer a direct comparison of the microbiomes of these tumors. Comparing the beta-diversity between all pairs of samples within a cancer type and across different cancer types revealed that microbiomes of tumors belonging to the same cancer type tend to be more similar than those of different cancer types (FIG. 4A). The distribution of order-level phylotypes revealed marked changes between the bacterial composition of the different tumor types (FIG. 4B, FIG. 6). We added 22 colorectal tumors from one medical center to our cohort to help relate some of our findings to the known colorectal cancer microbiome (11, 12). Consistent with previous reports, bacteria belonging to the Firmicutes and Bacteroidetes phyla were the most abundant species in colorectal cancer tumors (FIG. 4B) (10). In contrast, Proteobacteria dominated the microbiome of pancreatic cancer, in a manner similar to the normal duodenal microbiome makeup (16, 17, 38, 39). This may reflect a retrograde bacterial migration from the duodenum, to which the pancreatic duct opens, as we have previously reported (16). Although species belonging to the Proteobacteria and Firmicutes phyla accounted for the majority of the reads in all cancer types, the Proteobacteria to Firmicutes ratio appears to vary between tumor types (FIG. 4B). We also detected a larger representation of the Actinobacteria phylum, including the Corynebacteriaceae and Micrococcaceae families, in non-gastrointestinal tumors (FIG. 4B and FIG. 6). This is in agreement with previous reports describing the microbiome of breast, lung, and ovarian cancer (2, 4, 6, 9, 14, 15, 18).

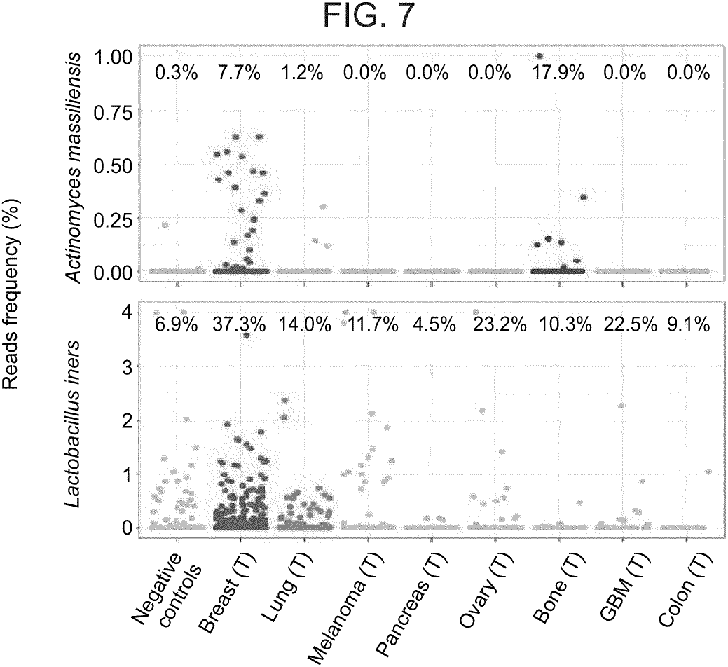

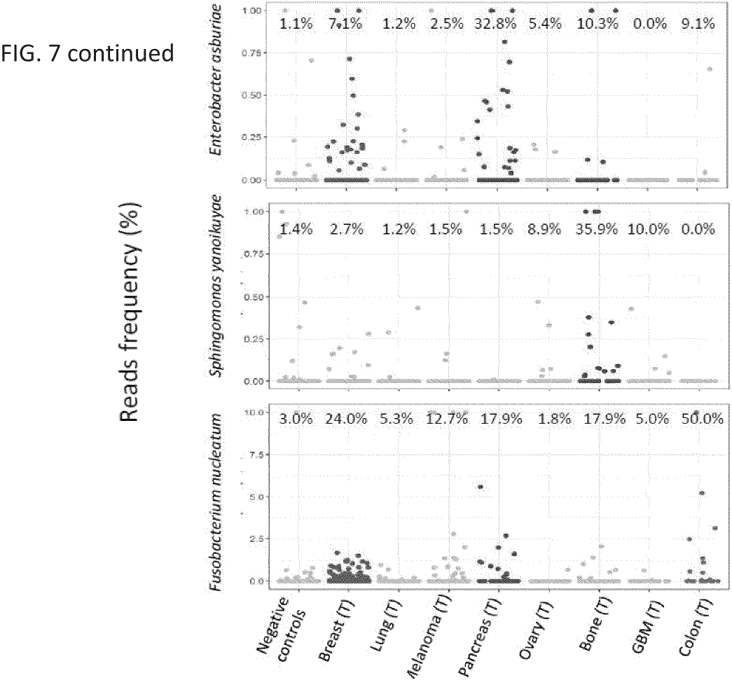

A tumor-type distinctive microbiome composition was also apparent at the species level. Unsupervised clustering of the most prevalent intra-tumor bacterial species (n=137) demonstrated that many of these species are enriched in certain tumor types (FIGS. 4C and 4D and FIG. 7). Fusobacterium nucleatum, previously reported to be enriched in colorectal tumors, was also a hit in our breast and pancreas tumor cohorts (FIG. 7). We also observed a distinct microbiome across subtypes of the same tumor type. For example, when comparing different subtypes of breast cancer according to their estrogen, progesterone and HER2 receptors’ status, we found multiple bacterial taxa whose prevalence was different between the subtypes (FIG. 4E). Lastly, although the overall microbial composition of the different tumor types was relatively similar to their normal adjacent tissue microbiome (FIG. 4F), we detected bacteria with a different prevalence in tumors and in their normal adjacent tissue (FIG. 4G). Consistent with our observation that bacterial load and richness of breast tumors is higher than in breast NAT (FIG. 3F), we found many bacteria that are significantly enriched in breast tumors compared to their NAT (FIG. 4G).

Table 2 includes bacterial taxa that were differentially prevalent in breast, lung or ovarian cancers as compared to their normal adjacent to tumor (NAT) tissue. Bacteria are sorted according to their p-values (lowest to highest) for enrichment/depletion per tumor type.

TABLE 2

| bact_ID | phylum | class | order | family | genus | species | Tumor type | E/D | SEQ ID NO: |
|---|---|---|---|---|---|---|---|---|---|
| 12873 | Proteobacteria | Alphaproteobacteria | Sphingomonadales | Sphingomonadaceae | Sphingomonas | Unknown species 602 | Breast | E | 171 |
| 13620 | Proteobacteria | Betaproteobacteria | Burkholderiales | Comamon adaceae | Tepidimonas | Unknown species 11 | Breast | E | 172 |
| 32080 | Proteobacteria | Betaproteobacteria | Burkholderiales | Comamon adaceae | Tepidimonas | | Breast | E | |
| 11657 | Proteobacteria | Alphaproteobacteria | Rhizobiales | Methylobacteriaceae | Methylobacterium | Methylobacterium organophilum | Breast | E | 173 |
| 11656 | Proteobacteria | Alphaproteobacteria | Rhizobiales | Methylobacteriaceae | Methylobacterium | Methylobacterium mesophilicum | Breast | E | 174 |
| 30362 | Bacteroidetes | Bacteroidia | Bacteroidales | Prevotellaceae | Prevotella | | Breast | E | |
| 50030 | Bacteroidetes | Bacteroidia | Bacteroidales | | | | Breast | E | |
| 30867 | Firmicutes | Bacilli | Lactobacillales | Streptococcaceae | Streptococcus | | Breast | E | |
| 50075 | Firmicutes | Bacilli | Bacillales | | | | Breast | E | |
| 50148 | Proteobacteria | Gammaproteobacteria | Pseudomonadales | | | | Breast | E | |
| 31477 | Firmicutes | Clostridia | Clostridiales | Tissierellaceae | Finegoldia | | Breast | E | |
| 40195 | Firmicutes | Clostridia | Clostridiales | Tissierellaceae | | | Breast | E | |
| 70016 | Firmicutes | | | | | | Breast | E | |
| 30663 | Cyanobacteria | Chloroplast | Streptophyta | Unknown family | Unknown genus 116 | | Breast | E | |
| 4523 | Cyanobacteria | Chloroplast | Streptophyta | Unknown family | Unknown genus 116 | Unknown species 19 | Breast | E | 175 |
| 40168 | Firmicutes | Bacilli | Bacillales | Staphylococcaceae | | | Breast | E | |
| 9900 | Firmicutes | Clostridia | Clostridiales | Tissierellaceae | Finegoldia | Unknown species 11 | Breast | E | 176 |
| 10778 | Fusobacteria | Fusobacteriia | Fusobacteriales | Leptotrichiaceae | Leptotrichia | Leptotrichia shahii | Breast | D | 177 |
| 5302 | Firmicutes | Bacilli | Bacillales | Staphylococcaceae | Staphylococcus | Unknown species 8 | Breast | E | 178 |
| 5324 | Firmicutes | Bacilli | Bacillales | Staphylococcaceae | Staphylococcus | Staphylococcus haemolyticus | Breast | E | 179 |
| 15324 | Proteobacteria | Gammaproteobacteria | Pseudomonadales | Moraxellaceae | Acinetobacter | Acinetobacter ursingii | Breast | E | 180 |
| 30817 | Firmicutes | Bacilli | Bacillales | Staphylococcaceae | Staphylococcus | | Breast | E | |
| 30858 | Firmicutes | Bacilli | Lactobacillales | Lactobacillaceae | Lactobacillus | | Breast | E | |
| 31799 | Proteobacteria | Alphaproteobacteria | Rhizobiales | Methylobacteriaceae | Methylobacterium | | Breast | E | |
| 40245 | Proteobacteria | Alphaproteobacteria | Rhizobiales | Methylobacteriaceae | | | Breast | E | |
| 60052 | Firmicutes | Bacilli | | | | | Breast | E | |
| 60078 | Proteobacteria | Betaproteobacteria | | | | | Breast | E | |
| 13182 | Proteobacteria | Betaproteobacteria | Burkholderiales | Burkholderiaceae | Ralstonia | Ralstonia mannitolilytica | Breast | E | 181 |
| 31786 | Proteobacteria | Alphaproteobacteria | Rhizobiales | Hyphomicrobiaceae | Devosia | | Breast | E | |
| 40181 | Firmicutes | Bacilli | Lactobacillales | Streptococcaceae | | | Breast | E | |
| 40330 | Proteobacteria | Gammaproteobacteria | Pseudomonadales | Pseudomonadaceae | | | Breast | E | |
| 969 | Actinobacteria | Actinobacteria | Actinomycetales | Corynebacteriaceae | Corynebacterium | Corynebacterium stationis | Breast | E | 182 |
| 32410 | Proteobacteria | Gammaproteobacteria | Pseudomonadales | Pseudomonadaceae | Pseudomonas | | Breast | E | |
| 40242 | Proteobacteria | Alphaproteobacteria | Rhizobiales | Bradyrhizobiaceae | | | Breast | D | |
| 230 | Actinobacteria | Actinobacteria | Actinomycetales | Actinomycetaceae | Actinomyces | Actinomycesoris | Breast | E | 183 |
| 5687 | Firmicutes | Bacilli | Lactobacillales | Lactobacillaceae | Lactobacillus | Lactobacillus iners | Breast | E | 184 |
| 5466 | Firmicutes | Bacilli | Lactobacillales | Aerococcaceae | Abiotrophia | Abiotrophia defectiva | Breast | D | 185 |
| 40176 | Firmicutes | Bacilli | Lactobacillales | Aerococcaceae | | | Breast | E | |
| 50079 | Firmicutes | Clostridia | Clostridiales | | | | Breast | E | |
| 40179 | Firmicutes | Bacilli | Lactobacillales | Lactobacillaceae | | | Breast | E | |
| 5915 | Firmicutes | Bacilli | Lactobacillales | Streptococcaceae | Streptococcus | Unknown species 1871 | Breast | D | 186 |
| 3941 | Bacteroidetes | Flavobacteriia | Flavobacteriales | Weeksellaceae | Wautersiella | Unknown species 18 | Breast | E | 187 |
| 30113 | Actinobacteria | Actinobacteria | Actinomycetales | Cellulomonadaceae | Cellulomonas | | Breast | E | |
| 6166 | Firmicutes | Bacilli | Lactobacillales | Streptococcaceae | Streptococcus | Streptococcus cristatus | Breast | E | 188 |
| 15055 | Proteobacteria | Gammaproteobacteria | Enterobacteriales | Enterobacteriaceae | Klebsiella | Klebsiella pneumoniae | Breast | E | 189 |
| 30866 | Firmicutes | Bacilli | Lactobacillales | Streptococcaceae | Lactococcus | | Breast | E | |
| 40144 | Cyanobacteria | Chloroplast | Streptophyta | Unknown family | | | Breast | E | |
| 40276 | Proteobacteria | Betaproteobacteria | Neisseriales | Neisseriaceae | | | Breast | E | |
| 50085 | Fusobacteria | Fusobacteriia | Fusobacteriales | | | | Breast | E | |
| 40046 | Actinobacteria | Actinobacteria | Actinomycetales | Propionibacteriaceae | | | Breast | E | |
| 50077 | Firmicutes | Bacilli | Lactobacillales | | | | Breast | E | |
| 60081 | Proteobacteria | Gammaproteobacteria | | | | | Breast | E | |
| 1021 | Actinobacteria | Actinobacteria | Actinomycetales | Geodermatophilaceae | Blastococcus | Unknown species 13 | Breast | E | 190 |
| 4719 | Firmicutes | Bacilli | Bacillales | Bacillaceae | Anoxybacillus | Anoxybacillus kestanbolensis | Breast | E | 191 |
| 40042 | Actinobacteria | Actinobacteria | Actinomycetales | Nocardiaceae | | | Breast | E | |
| 32393 | Proteobacteria | Gammaproteobacteria | Pasteurellales | Pasteurellaceae | Haemophilus | | Breast | D | |
| 2556 | Bacteroidetes | Bacteroidia | Bacteroidales | Paraprevotellaceae | Prevotella | Prevotellatannerae | Breast | E | 192 |
| 14172 | Proteobacteria | Betaproteobacteria | Neisseriales | Neisseriaceae | Neisseria | Neisseria oralis | Breast | D | 193 |
| 30099 | Actinobacteria | Actinobacteria | Actinomycetales | Actinomy cetaceae | Actinomyces | | Breast | E | |
| 15408 | Proteobacteria | Gammaproteobacteria | Pseudomonadales | Moraxellaceae | Acinetobacter | Unknown species 422 | Breast | D | 194 |
| 9261 | Firmicutes | Clostridia | Clostridiales | Ruminococcaceae | Faecalibacterium | Faecalibacterium prausnitzii | Breast | E | 195 |
| 1082 | Actinobacteria | Actinobacteria | Actinomycetales | Intrasporangiaceae | Janibacter | Unknown species 29 | Breast | D | 196 |
| 32068 | Proteobacteria | Betaproteobacteria | Burkholderiales | Comamon adaceae | Limnohabitans | | Breast | D | |
| 4521 | Cyanobacteria | Chloroplast | Streptophyta | Unknown family | Unknown genus 116 | Unknown species 17 | Breast | E | 197 |
| 30190 | Actinobacteria | Actinobacteria | Actinomycetales | Mycobacteriaceae | Mycobacterium | | Breast | E | |
| 30225 | Actinobacteria | Actinobacteria | Actinomycetales | Propionibacteriaceae | Propionibacterium | | Breast | E | |
| 32336 | Proteobacteria | Gammaproteobacteria | Enterobacteriales | Enterobacteriaceae | Enterobacter | | Breast | E | |
| 40021 | Actinobacteria | Actinobacteria | Actinomycetales | Actinomycetaceae | | | Breast | E | |
| 40038 | Actinobacteria | Actinobacteria | Actinomycetales | Micrococcaceae | | | Breast | E | |
| 40193 | Firmicutes | Clostridia | Clostridiales | Ruminococcaceae | | | Breast | E | |
| 40198 | Firmicutes | Clostridia | Clostridiales | Veillonellaceae | | | Breast | E | |
| 40278 | Proteobacteria | Betaprote obacteria | Rhodocyclales | Rhodocyclaceae | | | Breast | E | |
| 30845 | Firmicutes | Bacilli | Lactobacillales | Aerococcaceae | Alloiococcus | | Breast | E | |
| 9771 | Firmicutes | Clostridia | Clostridiales | Tissierellaceae | 1-68 | 1-68 Unknown | Breast | E | 198 |
| 5687 | Firmicutes | Bacilli | Lactobacillales | Lactobacillaceae | Lactobacillus | Lactobacillus iners | Lung | E | 199 |
| 30736 | Firmicutes | Bacilli | Bacillales | Bacillaceae | Bacillus | | Lung | D | |
| 30855 | Firmicutes | Bacilli | Lactobacillales | Enterococcaceae | Enterococcus | | Lung | D | |
| 40260 | Proteobacteria | Alphaproteobacteria | Sphingomonadales | Erythrobacteraceae | | | Lung | E | |
| 70002 | Actinobacteria | | | | | | Lung | D | |
| 40178 | Firmicutes | Bacilli | Lactobacillales | Enterococcaceae | | | Lung | D | |
| 50011 | Actinobacteria | Actinobacteria | Actinomycetales | | | | Lung | D | |
| 15657 | Proteobacteria | Gammaproteobacteria | Pseudomonadales | Pseudomonadaceae | Pseudomonas | Pseudomonas aeruginosa | Ovary | D | 200 |

Table 3 summarizes the different bacterial species that are prevalent in specific tumor types.

TABLE 3

| Tumor type | bactID | phylum | class | order | family | genus | species | Prevalence in specific tumor type | SEQ ID NO: |
|---|---|---|---|---|---|---|---|---|---|
| Breast | 1824 | Actinobacteria | Actinobacteria | Actinomycetales | Propionibacteriaceae | Propionibacterium | Propionibacterium granulosum | 38% | 201 |
| Breast | 1346 | Actinobacteria | Actinobacteria | Actinomycetales | Micrococcaceae | Rothia | Rothia mucilaginosa | 37% | 202 |
| Breast | 5687 | Firmicutes | Bacilli | Lactobacillales | Lactobacillaceae | Lactobacillus | Lactobacillus iners | 37% | 203 |
| Breast | 6175 | Firmicutes | Bacilli | Lactobacillales | Streptococcaceae | Streptococcus | Streptococcus infantis | 36% | 204 |
| Breast | 10545 | Firmicutes | Clostridia | Clostridiales | Veillonellaceae | Veillonella | Veillonella dispar | 36% | 205 |
| Breast | 1344 | Actinobacteria | Actinobacteria | Actinomycetales | Micrococcaceae | Rothia | Rothiadentocariosa | 32% | 206 |
| Breast | 587 | Actinobacteria | Actinobacteria | Actinomycetales | Corynebacteriaceae | Corynebacterium | Unknown species 1715 | 28% | 207 |
| Breast | 5330 | Firmicutes | Bacilli | Bacillales | Staphylococcaceae | Staphylococcus | Staphylococcus pasteuri | 28% | 208 |
| Breast | 3046 | Bacteroidetes | Bacteroidia | Bacteroidales | Prevotellaceae | Prevotella | Prevotella melaninogenica | 27% | 209 |
| Breast | 10726 | Fusobacteria | Fusobacteriia | Fusobacteriales | Fusobacteriaceae | Fusobacterium | Fusobacterium nucleatum | 24% | 210 |
| Breast | 4523 | Cyanobacteria | Chloroplast | Streptophyta | Unknown family | Unknown genus 116 | Unknown species 19 | 23% | 211 |
| Breast | 9900 | Firmicutes | Clostridia | Clostridiales | Tissierellaceae | Finegoldia | Unknown species 11 | 23% | 212 |
| Breast | 15324 | Proteobacteria | Gammaproteobacteria | Pseudomonadales | Moraxellaceae | Acinetobacter | Acinetobacterursingii | 23% | 213 |
| Breast | 6184 | Firmicutes | Bacilli | Lactobacillales | Streptococcaceae | Streptococcus | Streptococcus pneumoniae | 22% | 214 |
| Breast | 2555 | Bacteroidetes | Bacteroidia | Bacteroidales | Paraprevotellaceae | Prevotella | Prevotella Unknown | 22% | 215 |
| Breast | 5286 | Firmicutes | Bacilli | Bacillales | Staphylococcaceae | Staphylococcus | Unknown species 691 | 22% | 216 |
| Breast | 10546 | Firmicutes | Clostridia | Clostridiales | Veillonellaceae | Veillonella | Veillonella parvula | 22% | 217 |
| Breast | 12106 | Proteobacteria | Alphaproteobacteria | Rhodobacterales | Rhodobacteraceae | Paracoccus | Paracoccus chinensis | 21% | 218 |
| Breast | 13904 | Proteobacteria | Betaproteobacteria | Burkholderiales | Oxalobacteraceae | Massilia | Massiliatimonae | 21% | 219 |
| Breast | 12109 | Proteobacteria | Alphaproteobacteria | Rhodobacterales | Rhodobacteraceae | Paracoccus | Paracoccusmarcusii | 20% | 220 |
| Lung | 1824 | Actinobacteria | Actinobacteria | Actinomycetales | Propionibacteriaceae | Propionibacterium | Propionibacterium granulosum | 19% | 221 |
| Lung | 12551 | Proteobacteria | Alphaproteobacteria | Sphingomonadales | Sphingomonadaceae | Kaistobacter | Kaistobacter Unknown | 16% | 222 |
| Lung | 10545 | Firmicutes | Clostridia | Clostridiales | Veillonellaceae | Veillonella | Veillonella dispar | 16% | 223 |
| Lung | 587 | Actinobacteria | Actinobacteria | Actinomycetales | Corynebacteriaceae | Corynebacterium | Unknown species 1715 | 16% | 224 |
| Lung | 1346 | Actinobacteria | Actinobacteria | Actinomycetales | Micrococcaceae | Rothia | Rothia mucilaginosa | 16% | 225 |
| Lung | 2555 | Bacteroidetes | Bacteroidia | Bacteroidales | Paraprevotellaceae | Prevotella | Prevotella Unknown | 14% | 226 |
| Lung | 5687 | Firmicutes | Bacilli | Lactobacillales | Lactobacillaceae | Lactobacillus | Lactobacillus iners | 14% | 227 |
| Lung | 12937 | Proteobacteria | Alphaproteobacteria | Sphingomonadales | Sphingomonadaceae | Sphingomonas | Sphingomonas yunnanensis | 13% | 228 |
| Lung | 1766 | Actinobacteria | Actinobacteria | Actinomycetales | Propionibacteriaceae | Unknown genus 24 | Unknown species 1 | 12% | 229 |
| Lung | 12109 | Proteobacteria | Alphaproteobacteria | Rhodobacterales | Rhodobacteraceae | Paracoccus | Paracoccus marcusii | 11% | 230 |
| Lung | 12289 | Proteobacteria | Alphaproteobacteria | Rhodospirillales | Acetobacteraceae | Roseomonas | Roseomonasmucosa | 10% | 231 |
| Lung | 15666 | Proteobacteria | Gammaproteobacteria | Pseudomonadales | Pseudomonadaceae | Pseudomonas | Pseudomonasbaetica | 9% | 232 |
| Lung | 1815 | Actinobacteria | Actinobacteria | Actinomycetales | Propionibacteriaceae | Propionibacterium | Unknown species 18 | 8% | 233 |
| Lung | 12808 | Proteobacteria | Alphaproteobacteria | Sphingomonadales | Sphingomonadaceae | Sphingomonas | Unknown species 45 | 8% | 234 |
| Lung | 5338 | Firmicutes | Bacilli | Bacillales | Staphylococcaceae | Staphylococcus | Staphylococcus warneri | 7% | 235 |
| Lung | 13018 | Proteobacteria | Betaproteobacteria | Burkholderiales | Alcaligenaceae | Alcaligenes | Alcaligenes faecalis | 7% | 236 |
| Lung | 13194 | Proteobacteria | Betaproteobacteria | Burkholderiales | Comamonadaceae | Acidovorax | Acidovoraxtemperans | 7% | 237 |
| Lung | 545 | Actinobacteria | Actinobacteria | Actinomycetales | Corynebacteriaceae | Corynebacterium | Unknown species 1626 | 7% | 238 |
| Lung | 6066 | Firmicutes | Bacilli | Lactobacillales | Streptococcaceae | Streptococcus | Unknown species 346 | 7% | 239 |
| Lung | 9900 | Firmicutes | Clostridia | Clostridiales | Tissierellaceae | Finegoldia | Unknown species 11 | 7% | 240 |
| Melanoma | 12109 | Proteobacteria | Alphaproteobacteria | Rhodobacterales | Rhodobacteraceae | Paracoccus | Paracoccus marcusii | 20% | 241 |
| Melanoma | 5315 | Firmicutes | Bacilli | Bacillales | Staphylococcaceae | Staphylococcus | Staphylococcus aureus | 14% | 242 |
| Melanoma | 2495 | Bacteroidetes | Bacteroidia | Bacteroidales | Bacteroidaceae | Bacteroides | Bacteroidesdorei | 10% | 243 |
| Melanoma | 15607 | Proteobacteria | Gammaproteobacteria | Pseudomonadales | Pseudomonadaceae | Pseudomonas | Unknown species 632 | 5% | 244 |
| Melanoma | 10444 | Firmicutes | Clostridia | Clostridiales | Veillonellaceae | Selenomonas | Unknown species 18 | 5% | 245 |
| Melanoma | 15733 | Proteobacteria | Gammaproteobacteria | Pseudomonadales | Pseudomonadaceae | Pseudomonas | Pseudomonas viridiflava | 4% | 246 |
| Melanoma | 4886 | Firmicutes | Bacilli | Bacillales | Bacillaceae | Geobacillus | Unknown species 208 | 4% | 247 |
| Melanoma | 15043 | Proteobacteria | Gammaproteobacteria | Enterobacteriales | Enterobacteriaceae | Klebsiella | Unknown species 25 | 4% | 248 |
| Melanoma | 15608 | Proteobacteria | Gammaproteobacteria | Pseudomonadales | Pseudomonadaceae | Pseudomonas | Unknown species 643 | 4% | 249 |
| Melanoma | 4619 | Cyanobacteria | Chloroplast | Streptophyta | Unknown family | Unknown genus 39 | Unknown species 8 | 3% | 250 |
| Melanoma | 15956 | Proteobacteria | Gammaproteobacteria | Xanthomonadales | Xanthomonadaceae | Xanthomonas | Xanthomonasarboricola | 3% | 251 |
| Melanoma | 436 | Actinobacteria | Actinobacteria | Actinomycetales | Corynebacteriaceae | Corynebacterium | Unknown species 1061 | 2% | 252 |
| Melanoma | 6291 | Firmicutes | Clostridia | Clostridiales | Clostridiaceae | Clostridium | Unknown species 19 | 2% | 253 |
| Melanoma | 15368 | Proteobacteria | Gammaproteobacteria | Pseudomonadales | Moraxellaceae | Acinetobacter | Unknown species 31 | 2% | 254 |
| Melanoma | 13810 | Proteobacteria | Betaproteobacteria | Burkholderiales | Oxalobacteraceae | Massilia | Unknown species 177 | 2% | 255 |
| Melanoma | 6703 | Firmicutes | Clostridia | Clostridiales | Lachnospiraceae | Unknown genus 3008 | Unknown species 1 | 2% | 256 |
| Melanoma | 14087 | Proteobacteria | Betaproteobacteria | Neisseriales | Neisseriaceae | Eikenella | Eikenellacorrodens | 2% | 257 |
| Melanoma | 2437 | Bacteroidetes | Bacteroidia | Bacteroidales | Bacteroidaceae | Bacteroides | Unknown species 388 | 2% | 258 |
| Melanoma | 10458 | Firmicutes | Clostridia | Clostridiales | Veillonellaceae | Selenomonas | Unknown species 208 | 2% | 259 |
| Melanoma | 8343 | Firmicutes | Clostridia | Clostridiales | Lachnospiraceae | Lachnoanaerobaculum | Eubacterium saburreum | 1% | 260 |
| Melanoma | 10504 | Firmicutes | Clostridia | Clostridiales | Veillonellaceae | Selenomonas | Unknown species 59 | 1% | 261 |
| Pancreas | 14775 | Proteobacteria | Gammaproteobacteria | Enterobacteriales | Enterobacteriaceae | Citrobacter | Citrobacter freundii | 45% | 262 |
| Pancreas | 15055 | Proteobacteria | Gammaproteobacteria | Enterobacteriales | Enterobacteriaceae | Klebsiella | Klebsiella pneumoniae | 42% | 263 |
| Pancreas | 14847 | Proteobacteria | Gammaproteobacteria | Enterobacteriales | Enterobacteriaceae | Enterobacter | Enterobacterasburiae | 33% | 264 |
| Pancreas | 10545 | Firmicutes | Clostridia | Clostridiales | Veillonellaceae | Veillonella | Veillonelladispar | 19% | 265 |
| Pancreas | 10726 | Fusobacteria | Fusobacteriia | Fusobacteriales | Fusobacteriaceae | Fusobacterium | Fusobacterium nucleatum | 18% | 266 |
| Pancreas | 14849 | Proteobacteria | Gammaproteobacteria | Enterobacteriales | Enterobacteriaceae | Enterobacter | Enterobactercloacae | 18% | 267 |
| Pancreas | 15054 | Proteobacteria | Gammaproteobacteria | Enterobacteriales | Enterobacteriaceae | Klebsiella | Klebsiella oxytoca | 15% | 268 |
| Pancreas | 14846 | Proteobacteria | Gammaproteobacteria | Enterobacteriales | Enterobacteriaceae | Enterobacter | Enterobacter aerogenes | 13% | 269 |
| Pancreas | 6161 | Firmicutes | Bacilli | Lactobacillales | Streptococcaceae | Streptococcus | Streptococcus anginosus | 13% | 270 |
| Pancreas | 15695 | Proteobacteria | Gammaproteobacteria | Pseudomonadales | Pseudomonadaceae | Pseudomonas | Pseudomonas mendocina | 12% | 271 |
| Pancreas | 1346 | Actinobacteria | Actinobacteria | Actinomycetales | Micrococcaceae | Rothia | Rothia mucilaginosa | 10% | 272 |
| Pancreas | 14680 | Proteobacteria | Gammaproteobacteria | Alteromonadales | Shewanellaceae | Shewanella | Shewanella decolorationis | 10% | 273 |
| Pancreas | 5583 | Firmicutes | Bacilli | Lactobacillales | Enterococcaceae | Enterococcus | Enterococcus gallinarum | 10% | 274 |
| Pancreas | 5552 | Firmicutes | Bacilli | Lactobacillales | Carnobacteriaceae | Granulicatella | Granulicatella adiacens | 9% | 275 |
| Pancreas | 986 | Actinobacteria | Actinobacteria | Actinomycetales | Dermabacteraceae | Brachybacterium | Brachybacterium conglomeratum | 7% | 276 |
| Pancreas | 14174 | Proteobacteria | Betaproteobacteria | Neisseriales | Neisseriaceae | Neisseria | Neisseriasubflava | 7% | 277 |
| Pancreas | 2555 | Bacteroidetes | Bacteroidia | Bacteroidales | Paraprevotellaceae | Prevotella | Prevotella Unknown | 6% | 278 |
| Pancreas | 5582 | Firmicutes | Bacilli | Lactobacillales | Enterococcaceae | Enterococcus | Enterococcus faecium | 6% | 279 |
| Pancreas | 1344 | Actinobacteria | Actinobacteria | Actinomycetales | Micrococcaceae | Rothia | Rothia dentocariosa | 4% | 280 |
| Pancreas | 10757 | Fusobacteria | Fusobacteriia | Fusobacteriales | Leptotrichiaceae | Leptotrichia | Unknown species 235 | 4% | 281 |
| Ovary | 12289 | Proteobacteria | Alphaproteobacteria | Rhodospirillales | Acetobacteraceae | Roseomonas | Roseomonas mucosa | 20% | 282 |
| Ovary | 12873 | Proteobacteria | Alphaproteobacteria | Sphingomonadales | Sphingomonadaceae | Sphingomonas | Unknown species 602 | 20% | 283 |
| Ovary | 5319 | Firmicutes | Bacilli | Bacillales | Staphylococcaceae | Staphylococcus | Staphylococcus cohnii | 9% | 284 |
| Ovary | 16227 | Thermi | Deinococci | Deinococcales | Deinococcaceae | Deinococcus | Unknown species 124 | 7% | 285 |
| Ovary | 5627 | Firmicutes | Bacilli | Lactobacillales | Lactobacillaceae | Lactobacillus | Unknown species 479 | 5% | 286 |
| Bone | 12674 | Proteobacteria | Alphaproteobacteria | Sphingomonadales | Sphingomonadaceae | Sphingobium | Sphingomonas yanoikuyae | 36% | 287 |
| Bone | 1824 | Actinobacteria | Actinobacteria | Actinomycetales | Propionibacteriaceae | Propionibacterium | Propionibacterium granulosum | 28% | 288 |
| Bone | 225 | Actinobacteria | Actinobacteria | Actinomycetales | Actinomycetaceae | Actinomyces | Actinomyces massiliensis | 18% | 289 |
| Bone | 15662 | Proteobacteria | Gammaproteobacteria | Pseudomonadales | Pseudomonadaceae | Pseudomonas | Pseudomonas argentinensis | 13% | 290 |
| Bone | 14847 | Proteobacteria | Gammaproteobacteria | Enterobacteriales | Enterobacteriaceae | Enterobacter | Enterobacterasburiae | 10% | 291 |
| Bone | 15568 | Proteobacteria | Gammaproteobacteria | Pseudomonadales | Pseudomonadaceae | Pseudomonas | Unknown species 39 | 10% | 292 |
| Bone | 5968 | Firmicutes | Bacilli | Lactobacillales | Streptococcaceae | Streptococcus | Unknown species 2029 | 8% | 293 |
| Bone | 16119 | TM7 | TM7-3 | CW040 | Unknown family | Unknown genus 3 | Unknown species 7 | 5% | 294 |
| Bone | 16025 | Spirochaetes | Spirochaetes | Spirochaetales | Spirochaetaceae | Treponema | Treponemasocranskii | 5% | 295 |
| Bone | 4868 | Firmicutes | Bacilli | Bacillales | Bacillaceae | Bacillus | Bacillus clausii | 5% | 296 |
| Bone | 527 | Actinobacteria | Actinobacteria | Actinomycetales | Corynebacteriaceae | Corynebacterium | Unknown species 1534 | 5% | 297 |
| GBM | 14849 | Proteobacteria | Gammaproteobacteria | Enterobacteriales | Enterobacteriaceae | Enterobacter | Enterobacter cloacae | 10% | 298 |
| GBM | 14169 | Proteobacteria | Betaproteobacteria | Neisseriales | Neisseriaceae | Neisseria | Neisseria macacae | 8% | 299 |
| GBM | 1311 | Actinobacteria | Actinobacteria | Actinomycetales | Micrococcaceae | Kocuria | Kocuria atrinae | 8% | 300 |
| GBM | 15409 | Proteobacteria | Gammaproteobacteria | Pseudomonadales | Moraxellaceae | Acinetobacter | Unknown species 424 | 8% | 301 |
| GBM | 14934 | Proteobacteria | Gammaproteobacteria | Enterobacteriales | Enterobacteriaceae | Escherichia/Shigella | Unknown species 231 | 8% | 302 |
| GBM | 14795 | Proteobacteria | Gammaproteobacteria | Enterobacteriales | Enterobacteriaceae | Enterobacter | Unknown species 196 | 8% | 303 |
| GBM | 1129 | Actinobacteria | Actinobacteria | Actinomycetales | Microbacteriaceae | Agromyces | Agromyces mediolanus | 5% | 304 |
| GBM | 11842 | Proteobacteria | Alphaproteobacteria | Rhizobiales | Rhizobiaceae | Agrobacterium | Unknown species 298 | 5% | 305 |
| GBM | 15853 | Proteobacteria | Gammaproteobacteria | Xanthomonadales | Xanthomonadaceae | Luteimonas | Unknown species 76 | 5% | 306 |
| GBM | 5106 | Firmicutes | Bacilli | Bacillales | Planococcaceae | Lysinibacillus | Lysinibacillus boronitolerans | 5% | 307 |
| GBM | 5009 | Firmicutes | Bacilli | Bacillales | Exiguobacteraceae | Exiguobacterium | Unknown species 29 | 5% | 308 |
| GBM | 15333 | Proteobacteria | Gammaproteobacteria | Pseudomonadales | Moraxellaceae | Acinetobacter | Unknown species 127 | 5% | 309 |
| GBM | 15494 | Proteobacteria | Gammaproteobacteria | Pseudomonadales | Moraxellaceae | Psychrobacter | Unknown species 28 | 5% | 310 |

### EXAMPLE 5 Bacterial Metabolic Functions Have Strong Associations With Clinical Metadata and Are Attributed to Large Communities of Intra-Tumor Bacteria

Our results demonstrate that intra-tumor bacteria span a wide spectrum of the bacterial kingdom. To uncover the functional activities of intra-tumor bacteria, we used the PICRUSt2 tool (40-42) to map the 16S sequences to the genes and pathways that these bacterial species may harbor via full-genome content data.

Unsupervised clustering of 287 predicted metabolic MetaCyc pathways that showed the greatest variability between the tumor types, demonstrated a tumor type-distinctive signature of these microbiome metabolic pathways (FIG. 5A). We found tumor type-specific enrichment of bacterial pathways that can degrade metabolites known to be enriched in these same tumor types. For example, degradation of hydroxyprolines by bacteria (MetaCyc #PWY-5159) was enriched in bone tumors (effect size 14.6%, p-value < 0.01, proportion test). Bone collagen is a main source of hydroxyproline, and many bone pathologies, like bone tumors, have been shown to result in elevated hydroxyproline levels (44). In the case of lung cancer, MetaCyc pathways responsible for the degradation of cigarette smoke chemicals, like toluene, acrylonitrile, and aminobenzoates (#TOLUENE-DEG-2-OH-PWY, #P344-PWY, #PWY-6077), were significantly enriched in bacteria found in lung tumors compared to other tumor types (effect size 8.4%, 8%, 7.2%, p-value<0.001 for all, proportion test).

The enrichment for bacteria with the predicted capability to degrade cigarette smoke metabolites in lung tumors may suggest that high levels of these metabolites create a preferred niche for bacteria that can utilize these metabolites. To confirm this hypothesis, we compared the bacterial functions found in non-small cell lung cancer (NSCLC) tumors of 100 current smokers to 43 people who had never smoked (“never-smokers”). We found that 17 of the 49 MetaCyc pathways that were significantly enriched in tumors of current smokers, were pathways that degrade chemicals found in cigarette smoke like nicotine, anthranilate, toluene, and phenol (FIG. 5B, blue circles). We also found eight MetaCyc pathways related to the biosynthesis of metabolites that can be used by plants like the biosynthesis of glycine, a key intermediate in plant photorespiration (FIG. 5B, red circles). We suspect that some plant-associated bacteria, or their DNA, are present in cigarette tobacco, and are thus enriched in the lung tumors of smokers.

To determine which bacteria contribute to the MetaCyc pathways that are enriched in the lung tumors of current smokers, we compared the proportion of all bacterial taxa found in lung tumors of current smokers (n=100) to those in the tumors of never-smokers (n=43). We found that most of the enriched taxa in lung tumors of smokers belong to the Proteobacteria phylum. However, none of these bacteria reached significance after correction for multiple-hypothesis testing (FIG. 5C) indicating that there was no homogenous population of species conferring this functionality across samples. We reasoned that although bacterial ecology differs between tumors, there is a shared functional signal related to the unique environment within the lungs of smokers. Indeed, we were able to demonstrate that a very large number of heterogeneous bacteria contribute to the degradation functions of cigarette smoke metabolites and the biosynthesis of plant metabolites (FIG. 5D). Bacteria expressing these functions are found mainly in the Proteobacteria, Actinobacteria, and Cyanobacteria phyla, and are depleted from the Firmicutes phylum (FIG. 5D).

We also found enrichment of bacterial functions when comparing different tumor subtypes. For example, multiple MetaCyc pathways were enriched in bacteria from 270 ER+ compared to 49 ER- human breast tumors (FIG. 5E). The most significantly enriched pathways in bacteria of ER+ breast tumors were arsenate detoxification and mycothiol biosynthesis. Arsenic is a Group 1 carcinogen that can increase the risk of breast cancer (45), and has been shown to induce expression of the estrogen receptor in human breast cancer (46). Mycothiol is used by bacteria to detoxify reactive oxygen species (47). Because ER+ breast tumors are known to have increased oxidative stress compared to ER- tumors (48), we hypothesize that bacteria with the ability to synthesize mycothiol can better survive in the ER+ tumor microenvironment. We also found enrichment of bacterial functions when comparing tumor to NAT samples. For example, enzymes related to anaerobic respiration were enriched in bacteria from breast cancer vs NAT. Overall, analysis of MetaCyc pathways suggests a connection between the functions of bacteria present in the tumor and their tumor microenvironment.

Lastly, as our immunofluorescent staining suggests (FIGS. 2A-E), bacteria can be found inside CD45+ immune cells, indicating that they may influence or reflect the immune state of the tumor microenvironment. To look for an intra-tumor microbial signature that is correlated with response to immunotherapy, we compared metastatic melanoma tumors that responded to immune checkpoint inhibitors (ICI) (n=29) to those that did not respond (n=48). While we did not find significant changes in the load of bacteria between responders and non-responders to ICI, we did find multiple taxa that were differentially more (n=18) or less (n=28) abundant in melanoma tumors of responders compared to non-responders (FIG. 5F). Taxa that were more abundant in tumors of responders included Clostridium, whereas Gardnerella vaginalis was more abundant in tumors of non-responders. Importantly, this is in line with differential abundances of taxa in the gut microbiome of melanoma patients responding to ICI (49-51). Lastly, we stratified the 77 patients on ICI according to the presence or absence of a favorable tumor-microbiome signature that was generated using the 46 differentially prevalent bacteria. We found that patients with a favorable response-associated tumor-microbiome signature had prolonged progression free survival compared to those without this signature (FIG. 5G and Table 4).

Table 4 includes 46 bacterial taxa that were differentially present in melanoma tumors from responders or non-responders (FDR corrected p-values <0.2).

Taxa are sorted according their p-value (lowest to highest).

TABLE 4

| BactID | phylum | class | order | family | genus | species | SEQ ID NO: | E / D |
|---|---|---|---|---|---|---|---|---|
| 30190 | Actinobacteria | Actinobacteria | Actinomy cetales | Mycobacteriaceae | Mycobacterium | | | E |
| 10545 | Firmicutes | Clostridia | Clostridiales | Veillonellaceae | Veillonella | Veillonella dispar | 311 | D |
| 40198 | Firmicutes | Clostridia | Clostridiales | Veillonellaceae | | | | D |
| 30663 | Cyanobacteria | Chloroplast | Streptophyta | Unknown family | Unknown genus116 | | | E |
| 32003 | Proteobacteria | Alphaproteobacteria | Sphingomonadales | Sphingomonadaceae | Novosphingobium | | | E |
| 60081 | Proteobacteria | Gammaproteobacteria | | | | | | D |
| 31606 | Firmicutes | Clostridia | Clostridiales | Veillonellaceae | Veillonella | | | D |
| 40057 | Actinobacteria | Actinobacteria | Bifidobacteriales | Bifidobacteriaceae | | | | D |
| 30858 | Firmicutes | Bacilli | Lactobacillales | Lactobacillaceae | Lactobacillus | | | D |
| 30683 | Cyanobacteria | Chloroplast | Streptophyta | Unknown family | Unknown genus39 | | | D |
| 31635 | Fusobacteria | Fusobacteriia | Fusobacteriales | Leptotrichiaceae | Leptotrichia | | | D |
| 15367 | Proteobacteria | Gammaproteobacteria | Pseudomonadales | Moraxellaceae | Acinetobacter | Unknown species 300 | 312 | D |
| 32394 | Proteobacteria | Gammaproteobacteria | Pseudomonadales | Moraxellaceae | Acinetobacter | | | D |
| 5216 | Firmicutes | Bacilli | Bacillales | Staphylococcaceae | Staphylococcus | Unknown species 270 | 313 | E |
| 12937 | Proteobacteria | Alphaproteobacteria | Sphingomonadales | Sphingomonadaceae | Sphingomonas | Sphingomonas yunnanensis | 314 | E |
| 13952 | Proteobacteria | Betaproteobacteria | Burkholderiales | Oxalobacteraceae | Ralstonia | Unknown species 139 | 315 | E |
| 14724 | Proteobacteria | Gammaprotobacteria | Enterobacteriales | Enterobacteriaceae | Unknown genus 122 | Unknown species 1 | 316 | E |
| 15528 | Proteobacteria | Gammaproteobacteria | Pseudomonadales | Pseudomonadaceae | Unknown genus 86 | Unknown species 1 | 317 | E |
| 15691 | Proteobacteria | Gammaproteobacteria | Pseudomonadales | Pseudomonadaceae | Pseudomonas | Pseudomonas libanensis | 318 | E |
| 40094 | Bacteroidetes | Cytophagia | Cytophagales | Cytophagaceae | | | | E |
| 30115 | Actinobacteria | Actinobacteria | Actinomycetales | Corynebacteriaceae | Corynebacterium | | | D |
| 70010 | Cyanobacteria | | | | | | | D |
| 13608 | Proteobacteria | Betaproteobacteria | Burkholderiales | Comamonadaceae | Rubriviva x | Unknown species 95 | 319 | E |
| 30283 | Actinobacteria | Actinobacteria | Coriobacteriales | Coriobacteriaceae | Atopobium | | | E |
| 30890 | Firmicutes | Clostridia | Clostridiales | Clostridiaceae | Clostridium | | | E |
| 31476 | Firmicutes | Clostridia | Clostridiales | Tissierellaceae | Anaerococcus | | | E |
| 32077 | Proteobacteria | Betaproteobacteria | Burkholderiales | Comamonadaceae | Rubriviva x | | | E |
| 40058 | Actinobacteria | Actinobacteria | Coriobacteriales | Coriobacteriaceae | | | | E |
| 40185 | Firmicutes | Clostridia | Clostridiales | Clostridiaceae | | | | E |
| 40302 | Proteobacteria | Gammaproteobacteria | Aeromonadales | Aeromonadaceae | | | | D |
| 50119 | Proteobacteria | Betaproteobacteria | Burkholderiales | | | | | E |
| 4619 | Cyanobacteria | Chloroplast | Streptophyta | Unknown family | Unknown genus 39 | Unknown species 8 | 320 | D |
| 5318 | Firmicutes | Bacilli | Bacillales | Staphylococcaceae | Staphylococcus | Staphylococcus caprae | 321 | D |
| 6182 | Firmicutes | Bacilli | Lactobacillales | Streptococcaceae | Streptococcus | Streptococcus parasanguinis | 322 | D |
| 14727 | Proteobacteria | Gammaproteobacteria | Enterobacteriales | Enterobacteriaceae | Unknown genus 153 | Unknown species 1 | 323 | D |
| 30843 | Firmicutes | Bacilli | Lactobacillales | Aerococcaceae | Aerococcus | | | D |
| 30939 | Firmicutes | Clostridia | Clostridiales | Lachnospiraceae | Ruminococcus | | | D |
| 32056 | Proteobacteria | Betaproteobacteria | Burkholderiales | Comamonadaceae | Aquabacterium | | | D |
| 32358 | Proteobacteria | Gammaproteobacteria | Enterobacteriales | Enterobacteriaceae | Unknown genus153 | | | D |
| 40260 | Proteobacteria | Alphaproteobacteria | Sphingomonadales | Erythrobacteraceae | | | | D |
| 31733 | Proteobacteria | Alphaproteobacteria | Caulobacterales | Caulobacteraceae | Caulobacter | | | D |
| 50139 | Proteobacteria | Gammaproteobacteria | Alteromonadales | | | | | D |
| 40329 | Proteobacteria | Gammaproteobacteria | Pseudomonadales | Moraxellaceae | | | | D |
| 2061 | Actinobacteria | Actinobacteria | Bifidobacteriales | Bifidobacteriaceae | Gardnerella | Gardnerella vaginalis | 324 | D |
| 12109 | Proteobacteria | Alphaproteobacteria | Rhodobaterales | Rhodobacteraceae | Paracoccus | Paracoccus marcusii | 325 | D |
| 40144 | Cyanobacteria | Chloroplast | Streptophyta | Unknown family | | | | D |

Table 4.1 includes a subset of bacteria listed in Table 4 that are differentially present in melanoma tumors from responders or non-responders

TABLE 4.1

| BactID | phylum | class | order | family | genus | species | SEQ ID NO: | E / D |
|---|---|---|---|---|---|---|---|---|
| 30663 | Cyanobacteria | Chloroplast | Streptophyta | Unknown family | Unknown genus 116 | | | E |
| 30858 | Firmicutes | Bacilli | Lactobacillales | Lactobacillaceae | Lactobacillus | | | D |
| 30683 | Cyanobacteria | Chloroplast | Streptophyta | Unknown family | Unknown genus39 | | | D |
| 31635 | Fusobacteria | Fusobacteriia | Fusobacteriales | Leptotrichiaceae | Leptotrichia | | | D |
| 15367 | Proteobacteria | Gammaproteobacteria | Pseudomonadales | Moraxellaceae | Acinetobacter | Unknown species 300 | 312 | D |
| 32394 | Proteobacteria | Gammaproteobacteria | Pseudomonadales | Moraxellaceae | Acinetobacter | | | D |
| 5216 | Firmicutes | Bacilli | Bacillales | Staphylococcaceae | Staphylococcus | Unknown species 270 | 313 | E |
| 12937 | Proteobacteria | Alphaproteobacteria | Sphingomonadales | Sphingomonadaceae | Sphingomonas | Sphingomonasyunnanensis | 314 | E |
| 13952 | Proteobacteria | Betaproteobacteria | Burkholderiales | Oxalobacteraceae | Ralstonia | Unknown species 139 | 315 | E |
| 14724 | Proteobacteria | Gammaproteobacteria | Enterobacteriales | Enterobacteriaceae | Unknown genus 122 | Unknown species 1 | 316 | E |
| 15528 | Proteobacteria | Gammaproteobacteria | Pseudomonadales | Pseudomonadaceae | Unknown genus 86 | Unknown species 1 | 317 | E |
| 15691 | Proteobacteria | Gammaproteobacteria | Pseudomonadales | Pseudomonadaceae | Pseudomonas | Pseudomonaslibanensis | 318 | E |
| 40094 | Bacteroidetes | Cytophagia | Cytophagales | Cytophagaceae | | | | E |
| 30115 | Actinobacteria | Actinobacteria | Actinomycetales | Corynebacteriaceae | Corynebacterium | | | D |
| 70010 | Cyanobacteria | | | | | | | D |
| 13608 | Proteobacteria | Betaproteobacteria | Burkholderiales | Comamonadaceae | Rubrivivax | Unknown species 95 | 319 | E |
| 30283 | Actinobacteria | Actinobacteria | Coriobacteriales | Coriobacteriaceae | Atopobium | | | E |
| 31476 | Firmicutes | Clostridia | Clostridiales | Tissierellaceae | Anaerococcus | | | E |
| 32077 | Proteobacteria | Betaproteobacteria | Burkholderiales | Comamonadaceae | Rubrivivax | | | E |
| 40058 | Actinobacteria | Actinobacteria | Coriobacteriales | Coriobacteriaceae | | | | E |
| 40302 | Proteobacteria | Gammaproteobacteria | Aeromonadales | Aeromonadaceae | | | | D |
| 50119 | Proteobacteria | Betaproteobacteria | Burkholderiales | | | | | E |
| 4619 | Cyanobacteria | Chloroplast | Streptophyta | Unknown family | Unknown genus 39 | Unknown species 8 | 320 | D |
| 5318 | Firmicutes | Bacilli | Bacillales | Staphylococcaceae | Staphylococcus | Staphylococcuscaprae | 321 | D |
| 6182 | Firmicutes | Bacilli | Lactobacillales | Streptococcaceae | Streptococcus | Streptococcusparasanguinis | 322 | D |
| 14727 | Proteobacteria | Gammaproteobacteria | Enterobacteriales | Enterobacteriaceae | Unknown genus153 | Unknown species 1 | 323 | D |
| 30843 | Firmicutes | Bacilli | Lactobacillales | Aerococcaceae | Aerococcus | | | D |
| 30939 | Firmicutes | Clostridia | Clostridiales | Lachnospiraceae | Ruminococcus | | | D |
| 32056 | Proteobacteria | Betaproteobacteria | Burkholderiales | Comamonadaceae | Aquabacterium | | | D |
| 32358 | Proteobacteria | Gammaproteobacteria | Enterobacteriales | Enterobacteriaceae | Unknown genus153 | | | D |
| 40260 | Proteobacteria | Alphaproteobacteria | Sphingomonadales | Erythrobacteraceae | | | | D |
| 31733 | Proteobacteria | Alphaproteobacteria | Caulobacterales | Caulobacte raceae | Caulobacter | | | D |
| 50139 | Proteobacteria | Gammaproteobacteria | Alteromonadales | | | | | D |
| 40329 | Proteobacteria | Gammaproteobacteria | Pseudomonadales | Moraxellaceae | | | | D |
| 12109 | Proteobacteria | Alphaproteobacteria | Rhodobacterales | Rhodobacteraceae | Paracoccus | Paracoccus marcusii | 325 | D |
| 40144 | Cyanobacteria | Chloroplast | Streptophyta | Unknown family | | | | D |

D/E = depleted/enriched in responders to immune checkpoint inhibitors

TABLE 5

bacteria that are enriched in responders

| BactID | phylu m | class | order | family | genus | species | SEQ ID NO: |
|---|---|---|---|---|---|---|---|
| 30190 | Actinobacteria | Actinobacteria | Actinomycetales | Mycobacteriaceae | Mycobacterium | | |
| 30663 | Cyanobacteria | Chloroplast | Streptophyta | Unknown family | Unknown | genus116 | |
| 32003 | Proteobacteria | Alphaproteobacteria | Sphingomonadales | Sphingomonadaceae | Novosphingobium | | |
| 5216 | Firmicutes | Bacilli | Bacillales | Staphylococcaceae | Staphylococcus | Unknown species270 | 313 |
| 12937 | Proteobacteria | Alphaproteobacteria | Sphingomonadales | Sphingomonadaceae | Sphingomonas | Sphingomonas yunnanensis | 314 |
| 13952 | Proteobacteria | Betaproteobacteria | Burkholderiales | Oxalobacteraceae | Ralstonia | Unknown species 139 | 315 |
| 14724 | Proteobacteria | Gammaproteobacteria | Enterobacteriales | Enterobacteriaceae | Unknown genus122 | Unknown species 1 | 316 |
| 15528 | Proteobacteria | Gammaproteobacteria | Pseudomonadales | Pseudomonadaceae | Unknown genus86 | Unknown species 1 | 317 |
| 15691 | Proteobacteria | Gammaproteobacteria | Pseudomonadales | Pseudomonadaceae | Pseudomonas | Pseudomonas libanensis | 318 |
| 40094 | B acteroidetes | Cytophagia | Cytophagales | Cytophagaceae | | | |
| 13608 | Proteobacteria | Betaproteobacteria | Burkholderiales | Comamon adaceae | Rubrivivax | Unknown species95 | 319 |
| 30283 | Actinobacteria | Actinobacteria | Coriobacteriales | Coriobacteriaceae | Atopobium | | |
| 30890 | Firmicutes | Clostridia | Clostridiales | Clostridiaceae | Clostridium | | |
| 31476 | Firmicutes | Clostridia | Clostridiales | Tissierellaceae | Anaerococcus | | |
| 32077 | Proteobacteria | Betaproteobacteria | Burkholderiales | Comamon adaceae | Rubrivivax | | |
| 40058 | Actinobacteria | Actinobacteria | Coriobacteriales | Coriobacteriaceae | | | |
| 40185 | Firmicutes | Clostridia | Clostridiales | Clostridiaceae | | | |
| 50119 | Proteobacteria | Betaproteobacteria | Burkholderiales | | | | |

TABLE 6

bacteria that are depleted in responders

| BactID | phylum | class | order | family | genus | species | SEQ ID NO: |
|---|---|---|---|---|---|---|---|
| 10545 | Firmicutes | Clostridia | Clostridiales | Veillonellaceae | Veillonella | Veillonelladispar | 311 |
| 40198 | Firmicutes | Clostridia | Clostridiales | Veillonellaceae | | | |
| 60081 | Proteobacteria | Gammaproteobacteria | | | | | |
| 31606 | Firmicutes | Clostridia | Clostridiales | Veillonellaceae | Veillonella | | |
| 40057 | Actinobacteria | Actinobacteria | Bifidobacteriales | Bifidobacteriaceae | | | |
| 30858 | Firmicutes | Bacilli | Lactobacillales | Lactobacillaceae | Lactobacillus | | |
| 30683 | Cyanobacteria | Chloroplast | Streptophyta | Unknown family | Unknown genus39 | | |
| 31635 | Fusobacteria | Fusobacteriia | Fusobacteriales | Leptotrichiaceae | Leptotrichia | | |
| 15367 | Proteobacteria | Gammaproteobacteria | Pseudomonadales | Moraxellaceae | Acinetobacter | Unknown species300 | 312 |
| 32394 | Proteobacteria | Gammaproteobacteria | Pseudomonadales | Moraxellaceae | Acinetobacter | | |
| 30115 | Actinobacteria | Actinobacteria | Actinomycetales | Corynebacteriaceae | Corynebacterium | | |
| 70010 | Cyanobacteria | | | | | | |
| 40302 | Proteobacteria | Gammaproteobacteria | Aeromonadales | Aeromonadaceae | | | |
| 4619 | Cyanobacteria | Chloroplast | Streptophyta | Unknown family | Unknown genus39 | Unknown species8 | 320 |
| 5318 | Firmicutes | Bacilli | Bacillales | Staphylococcaceae | Staphylococcus | Staphylococcus caprae | 321 |
| 6182 | Firmicutes | Bacilli | Lactobacillales | Streptococcaceae | Streptococcus | Streptococcus parasanguinis | 322 |
| 14727 | Proteobacteria | Gammaproteobacteria | Enterobacteriales | Enterobacteriaceae | Unknown genus153 | Unknown species 1 | 323 |
| 30843 | Firmicutes | Bacilli | Lactobacillales | Aerococcaceae | Aerococcus | | |
| 30939 | Firmicutes | Clostridia | Clostridiales | Lachnospiraceae | Ruminococcus | | |
| 32056 | Proteobacteria | Betaproteobacteria | Burkholderiales | Comamonadaceae | Aquabacterium | | |
| 32358 | Proteobacteria | Gammaproteobacteria | Enterobacteriales | Enterobacteriaceae | Unknown genus153 | | |
| 40260 | Proteobacteria | Alphaproteobacteria | Sphingomonadales | Erythrobacteraceae | | | |
| 31733 | Proteobacteria | Alphaproteobacteria | Caulobacterales | Caulobacteraceae | Caulobacter | | |
| 50139 | Proteobacteria | Gammaproteobacteria | Alteromonadales | | | | |
| 40329 | Proteobacteria | Gammaproteobacteria | Pseudomonadales | Moraxellaceae | | | |
| 2061 | Actinobacteria | Actinobacteria | Bifidobacteriales | Bifidobacteriaceae | Gardnerella | Gardnerella vaginalis | 324 |
| 12109 | Proteobacteria | Alphaproteobacteria | Rhodobacterales | Rhodobacteraceae | Paracoccus | Paracoccus marcusii | 325 |
| 40144 | Cyanobacteria | Chloroplast | Streptophyta | Unknown family | | | |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1. C. de Martel, J. Ferlay, S. Franceschi, J. Vignat, F. Bray, D. Forman, M. Plummer, Global burden of cancers attributable to infections in 2008: a review and synthetic analysis. Lancet Oncol. 13, 607-615 (2012).

2. C. Xuan, J. M. Shamonki, A. Chung, M. L. Dinome, M. Chung, P. A. Sieling, D. J. Lee, Microbial dysbiosis is associated with human breast cancer.PloS One. 9, e83744 (2014).

3. K. J. Thompson, J. N. Ingle, X. Tang, N. Chia, P. R. Jeraldo, M. R. Walther-Antonio, K. K. Kandimalla, S. Johnson, J. Z. Yao, S. C. Harrington, V. J. Suman, L. Wang, R. L. Weinshilboum, J. C. Boughey, J.-P. Kocher, H. Nelson, M. P. Goetz, K. R. Kalari, A comprehensive analysis of breast cancer microbiota and host gene expression. PloS One. 12, e0188873 (2017).

4. S. Banerjee, T. Tian, Z. Wei, N. Shih, M. D. Feldman, K. N. Peck, A. M. DeMichele, J. C. Alwine, E. S. Robertson, Distinct Microbial Signatures Associated With Different Breast Cancer Types. Front. Microbiol. 9 (2018), doi:10.3389/fmicb.2018.00951.

5. L. Costantini, S. Magno, D. Albanese, C. Donati, R. Molinari, A. Filippone, R. Masetti, N. Merendino, Characterization of human breast tissue microbiota from core needle biopsies through the analysis of multi hypervariable 16S-rRNA gene regions.Sci. Rep. 8, 16893 (2018).

6. K. L. Greathouse, J. R. White, A. J. Vargas, V. V. Bliskovsky, J. A. Beck, N. von Muhlinen, E. C. Polley, E. D. Bowman, M. A. Khan, A. I. Robles, T. Cooks, B. M. Ryan, N. Padgett, A. H. Dzutsev, G. Trinchieri, M. A. Pineda, S. Bilke, P. S. Meltzer, A. N. Hokenstad, T. M. Stickrod, M. R. Walther-Antonio, J. P. Earl, J. C. Mell, J. E. Krol, S. V. Balashov, A. S. Bhat, G. D. Ehrlich, A. Valm, C. Deming, S. Conlan, J. Oh, J. A. Segre, C. C. Harris, Interaction between the microbiome and TP53 in human lung cancer. Genome Biol. 19, 123 (2018).

7. B. A. Peters, R. B. Hayes, C. Goparaju, C. Reid, H. I. Pass, J. Ahn, The Microbiome in Lung Cancer Tissue and Recurrence-Free Survival. Cancer Epidemiol. Biomark. Prev. Publ. Am. Assoc. Cancer Res. Cosponsored Am. Soc. Prev. Oncol. 28, 731-740 (2019).

8. P. Apostolou, A. Tsantsaridou, I. Papasotiriou, M. Toloudi, M. Chatziioannou, G. Giamouzis, Bacterial and fungal microflora in surgically removed lung cancer samples. J. Cardiothorac. Surg. 6, 137 (2011).

9. S. Banerjee, T. Tian, Z. Wei, N. Shih, M. D. Feldman, J. C. Alwine, G. Coukos, E. S. Robertson, The ovarian cancer oncobiome. Oncotarget. 8, 36225-36245 (2017).

10. Z. Gao, B. Guo, R. Gao, Q. Zhu, H. Qin, Microbiota disbiosis is associated with colorectal cancer. Front. Microbiol. 6 (2015), doi:10.3389/fmicb.2015.00020.

11. M. Castellarin, R. L. Warren, J. D. Freeman, L. Dreolini, M. Krzywinski, J. Strauss, R. Barnes, P. Watson, E. Allen-Vercoe, R. A. Moore, R. A. Holt, Fusobacterium nucleatum infection is prevalent in human colorectal carcinoma. Genome Res. 22, 299-306 (2012).

12. A. D. Kostic, D. Gevers, C. S. Pedamallu, M. Michaud, F. Duke, A. M. Earl, A. I. Ojesina, J. Jung, A. J. Bass, J. Tabernero, J. Baselga, C. Liu, R. A. Shivdasani, S. Ogino, B. W. Birren, C. Huttenhower, W. S. Garrett, M. Meyerson, Genomic analysis identifies association of Fusobacterium with colorectal carcinoma. Genome Res. 22, 292-298 (2012).

13. K. S. Sfanos, J. Sauvageot, H. L. Fedor, J. D. Dick, A. M. De Marzo, W. B. Isaacs, A molecular analysis of prokaryotic and viral DNA sequences in prostate tissue from patients with prostate cancer indicates the presence of multiple and diverse microorganisms. The Prostate. 68, 306-320 (2008).

14. C. Urbaniak, G. B. Gloor, M. Brackstone, L. Scott, M. Tangney, G. Reid, The Microbiota of Breast Tissue and Its Association with Breast Cancer. Appl. Environ. Microbiol. 82, 5039-5048 (2016).

15. C. Urbaniak, J. Cummins, M. Brackstone, J. M. Macklaim, G. B. Gloor, C. K. Baban, L. Scott, D. M. O′Hanlon, J. P. Burton, K. P. Francis, M. Tangney, G. Reid, Microbiota of human breast tissue. Appl. Environ. Microbiol. 80, 3007-3014 (2014).

16. L. T. Geller, M. Barzily-Rokni, T. Danino, O. H. Jonas, N. Shental, D. Nejman, N. Gavert, Y. Zwang, Z. A. Cooper, K. Shee, C. A. Thaiss, A. Reuben, J. Livny, R. Avraham, D. T. Frederick, M. Ligorio, K. Chatman, S. E. Johnston, C. M. Mosher, A. Brandis, G. Fuks, C. Gurbatri, V. Gopalakrishnan, M. Kim, M. W. Hurd, M. Katz, J. Fleming, A. Maitra, D. A. Smith, M. Skalak, J. Bu, M. Michaud, S. A. Trauger, I. Barshack, T. Golan, J. Sandbank, K. T. Flaherty, A. Mandinova, W. S. Garrett, S. P. Thayer, C. R. Ferrone, C. Huttenhower, S. N. Bhatia, D. Gevers, J. A. Wargo, T. R. Golub, R. Straussman, Potential role of intratumor bacteria in mediating tumor resistance to the chemotherapeutic drug gemcitabine. Science. 357, 1156-1160 (2017).

17. S. Pushalkar, M. Hundeyin, D. Daley, C. P. Zambirinis, E. Kurz, A. Mishra, N. Mohan, B. Aykut, M. Usyk, L. E. Torres, G. Werba, K. Zhang, Y. Guo, Q. Li, N. Akkad, S. Lall, B. Wadowski, J. Gutierrez, J. A. Kochen Rossi, J. W. Herzog, B. Diskin, A. Torres-Hernandez, J. Leinwand, W. Wang, P. S. Taunk, S. Savadkar, M. Janal, A. Saxena, X. Li, D. Cohen, R. B. Sartor, D. Saxena, G. Miller, The Pancreatic Cancer Microbiome Promotes Oncogenesis by Induction of Innate and Adaptive Immune Suppression. Cancer Discov. (2018), doi:10.1158/2159-8290.CD-17-1134.

18. G. Yu, M. H. Gail, D. Consonni, M. Carugno, M. Humphrys, A. C. Pesatori, N. E. Caporaso, J. J. Goedert, J. Ravel, M. T. Landi, Characterizing human lung tissue microbiota and its relationship to epidemiological and clinical features. Genome Biol. 17, 163 (2016).

19. B. Goodman, H. Gardner, The microbiome and cancer. J. Pathol. (2018), doi:10.1002/path.5047.

20. S. J. Salter, M. J. Cox, E. M. Turek, S. T. Calus, W. O. Cookson, M. F. Moffatt, P. Turner, J. Parkhill, N. J. Loman, A. W. Walker, Reagent and laboratory contamination can critically impact sequence-based microbiome analyses. BMC Biol. 12, 87 (2014).

21. R. Eisenhofer, J. J. Minich, C. Marotz, A. Cooper, R. Knight, L. S. Weyrich, Contamination in Low Microbial Biomass Microbiome Studies: Issues and Recommendations. Trends Microbiol. 27, 105-117 (2019).

22. N. M. Davis, D. M. Proctor, S. P. Holmes, D. A. Relman, B. J. Callahan, Simple statistical identification and removal of contaminant sequences in marker-gene and metagenomics data. Microbiome. 6, 226 (2018).

23. M. C. de Goffau, S. Lager, U. Sovio, F. Gaccioli, E. Cook, S. J. Peacock, J. Parkhill, D. S. Chamock-Jones, G. C. S. Smith, Human placenta has no microbiome but can contain potential pathogens. Nature. 572, 329-334 (2019).

24. M. L. Sogin, H. G. Morrison, J. A. Huber, D. Mark Welch, S. M. Huse, P. R. Neal, J. M. Arrieta, G. J. Herndl, Microbial diversity in the deep sea and the underexplored “rare biosphere.” Proc. Natl. Acad. Sci. U. S. A. 103, 12115-12120 (2006).

25. C. R. H. Raetz, C. Whitfield, Lipopolysaccharide endotoxins. Annu. Rev. Biochem. 71, 635-700 (2002).

26. W. Fischer, in New Comprehensive Biochemistry (Elsevier Science, Amsterdam, 1994), vol. 27, pp. 199-215.

27. R. I. Amann, B. J. Binder, R. J. Olson, S. W. Chisholm, R. Devereux, D. A. Stahl, Combination of 16S rRNA-targeted oligonucleotide probes with flow cytometry for analyzing mixed microbial populations. Appl. Environ. Microbiol. 56, 1919-1925 (1990).

28. C. Forestier, E. Moreno, J. Pizarro-Cerda, J. P. Gorvel, Lysosomal accumulation and recycling of lipopolysaccharide to the cell surface of murine macrophages, an in vitro and in vivo study. J. Immunol. Baltim. Md 1950. 162, 6784-6791 (1999).

29. K. T. Tokuyasu, Application of cryoultramicrotomy to immunocytochemistry. J. Microsc. 143, 139-149 (1986).

30. A. Abada, S. Levin-Zaidman, Z. Porat, T. Dadosh, Z. Elazar, SNARE priming is essential for maturation of autophagosomes but not for their formation. Proc. Natl. Acad. Sci. U. S. A. 114, 12749-12754 (2017).

31. E. Klieneberger-Nobel, Origin, development and significance of L-forms in bacterial cultures. J. Gen. Microbiol. 3, 434-443 (1949).

32. J. Errington, L-form bacteria, cell walls and the origins of life. Open Biol. 3, 120143 (2013).

33. J. Errington, Cell wall-deficient, L-form bacteria in the 21st century: a personal perspective. Biochem. Soc. Trans. 45, 287-295 (2017).

34. G. Fuks, M. Elgart, A. Amir, A. Zeisel, P. J. Turnbaugh, Y. Soen, N. Shental, Combining 16S rRNA gene variable regions enables high-resolution microbial community profiling. Microbiome. 6, 17 (2018).

35. T. Z. DeSantis, P. Hugenholtz, N. Larsen, M. Rojas, E. L. Brodie, K. Keller, T. Huber, D. Dalevi, P. Hu, G. L. Andersen, Greengenes, a Chimera-Checked 16S rRNA Gene Database and Workbench Compatible with ARB. Appl. Environ. Microbiol. 72, 5069-5072 (2006).

36. J. T. Lau, F. J. Whelan, I. Herath, C. H. Lee, S. M. Collins, P. Bercik, M. G. Surette, Capturing the diversity of the human gut microbiota through culture-enriched molecular profiling. Genome Med. 8, 72 (2016).

37. M. S. Siegrist, S. Whiteside, J. C. Jewett, A. Aditham, F. Cava, C. R. Bertozzi, (D)-Amino acid chemical reporters reveal peptidoglycan dynamics of an intracellular pathogen. ACS Chem. Biol. 8, 500-505 (2013).

38. G. Ou, M. Hedberg, P. Hörstedt, V. Baranov, G. Forsberg, M. Drobni, O. Sandström, S. N. Wai, I. Johansson, M.-L. Hammarström, O. Hernell, S. Hammarström, Proximal small intestinal microbiota and identification of rod-shaped bacteria associated with childhood celiac disease. Am. J. Gastroenterol. 104, 3058-3067 (2009).

39. E. Nistal, A. Caminero, A. R. Herrán, L. Arias, S. Vivas, J. M. R. de Morales, S. Calleja, L. E. S. de Miera, P. Arroyo, J. Casqueiro, Differences of small intestinal bacteria populations in adults and children with/without celiac disease: effect of age, gluten diet, and disease. Inflamm. Bowel Dis. 18, 649-656 (2012).

40. G. M. Douglas, V. J. Maffei, J. Zaneveld, S. N. Yurgel, J. R. Brown, C. M. Taylor, C. Huttenhower, M. G. I. Langille, PICRUSt2: An improved and extensible approach for metagenome inference. bioRxiv, 672295 (2019).

41. Y. Ye, T. G. Doak, A Parsimony Approach to Biological Pathway Reconstruction/Inference for Genomes and Metagenomes. PLOS Comput. Biol. 5, e1000465 (2009).

42. S. Louca, M. Doebeli, Efficient comparative phylogenetics on large trees. Bioinformatics. 34, 1053-1055 (2018).

43. K. Palanichamy, A. Chakravarti, Diagnostic and Prognostic Significance of Methionine Uptake and Methionine Positron Emission Tomography Imaging in Gliomas. Front. Oncol. 7 (2017), doi:10.3389/fonc.2017.00257.

44. M. Nakagawa, Y. Sugiura, T. Oshima, G. Kajino, H. Hirako, Urinary hydroxyproline excretion in orthopedic disease, with special reference to systemic bone disease and bone tumor (secondary report). Nagoya J. Med. Sci. 29, 345-367 (1967).

45. N. Khanjani, A.-B. Jafarnejad, L. Tavakkoli, Arsenic and breast cancer: a systematic review of epidemiologic studies. Rev. Environ. Health. 32, 267-277 (2017).

46. J. Du, N. Zhou, H. Liu, F. Jiang, Y. Wang, C. Hu, H. Qi, C. Zhong, X. Wang, Z. Li, Arsenic induces functional re-expression of estrogen receptor α by demethylation of DNA in estrogen receptor-negative human breast cancer. PloS One. 7, e35957 (2012).

47. A. M. Reyes, B. Pedre, M. I. De Armas, M.-A. Tossounian, R. Radi, J. Messens, M. Trujillo, Chemistry and Redox Biology of Mycothiol. Antioxid. Redox Signal. 28, 487-504 (2018).

48. P. Karihtala, S. Kauppila, Y. Soini, Arja-Jukkola-Vuorinen, Oxidative stress and counteracting mechanisms in hormone receptor positive, triple-negative and basal-like breast carcinomas. BMC Cancer. 11, 262 (2011).

49. V. Gopalakrishnan, C. N. Spencer, L. Nezi, A. Reuben, M. C. Andrews, T. V. Karpinets, P. A. Prieto, D. Vicente, K. Hoffman, S. C. Wei, A. P. Cogdill, L. Zhao, C. W. Hudgens, D. S. Hutchinson, T. Manzo, M. Petaccia de Macedo, T. Cotechini, T. Kumar, W. S. Chen, S. M. Reddy, R. Szczepaniak Sloane, J. Galloway-Pena, H. Jiang, P. L. Chen, E. J. Shpall, K. Rezvani, A. M. Alousi, R. F. Chemaly, S. Shelburne, L. M. Vence, P. C. Okhuysen, V. B. Jensen, A. G. Swennes, F. McAllister, E. Marcelo Riquelme Sanchez, Y. Zhang, E. Le Chatelier, L. Zitvogel, N. Pons, J. L. Austin-Breneman, L. E. Haydu, E. M. Burton, J. M. Gardner, E. Sirmans, J. Hu, A. J. Lazar, T. Tsujikawa, A. Diab, H. Tawbi, I. C. Glitza, W. J. Hwu, S. P. Patel, S. E. Woodman, R. N. Amaria, M. A. Davies, J. E. Gershenwald, P. Hwu, J. E. Lee, J. Zhang, L. M. Coussens, Z. A. Cooper, P. A. Futreal, C. R. Daniel, N. J. Ajami, J. F. Petrosino, M. T. Tetzlaff, P. Sharma, J. P. Allison, R. R. Jenq, J. A. Wargo, Gut microbiome modulates response to anti-PD-1 immunotherapy in melanoma patients. Science. 359, 97-103 (2018).

50. V. Matson, J. Fessler, R. Bao, T. Chongsuwat, Y. Zha, M.-L. Alegre, J. J. Luke, T. F. Gajewski, The commensal microbiome is associated with anti-PD-1 efficacy in metastatic melanoma patients. Science. 359, 104-108 (2018).

51. B. Routy, E. Le Chatelier, L. Derosa, C. P. M. Duong, M. T. Alou, R. Daillère, A. Fluckiger, M. Messaoudene, C. Rauber, M. P. Roberti, M. Fidelle, C. Flament, V. Poirier-Colame, P. Opolon, C. Klein, K. Iribarren, L. Mondragón, N. Jacquelot, B. Qu, G. Ferrere, C. Clémenson, L. Mezquita, J. R. Masip, C. Naltet, S. Brosseau, C. Kaderbhai, C. Richard, H. Rizvi, F. Levenez, N. Galleron, B. Quinquis, N. Pons, B. Ryffel, V. Minard-Colin, P. Gonin, J.-C. Soria, E. Deutsch, Y. Loriot, F. Ghiringhelli, G. Zalcman, F. Goldwasser, B. Escudier, M. D. Hellmann, A. Eggermont, D. Raoult, L. Albiges, G. Kroemer, L. Zitvogel, Gut microbiome influences efficacy of PD-1-based immunotherapy against epithelial tumors. Science. 359, 91-97 (2018).

52. R. Z. Gharaibeh, C. Jobin, Microbiota and cancer immunotherapy: in search of microbial signals. Gut (2018), doi:10.1136/gutjnl-2018-317220.

53. D. Schroter, A. Höhn, Role of Advanced Glycation End Products in Carcinogenesis and their Therapeutic Implications. Curr. Pharm. Des. 24, 5245-5251 (2018).

54. Human Microbiome Project Consortium, Structure, function and diversity of the healthy human microbiome. Nature. 486, 207-214 (2012).

55. A. Eng, E. Borenstein, Taxa-function robustness in microbial communities. Microbiome. 6, 45 (2018).

56. J. L. Pope, S. Tomkovich, Y. Yang, C. Jobin, Microbiota as a mediator of cancer progression and therapy. Transl. Res. J. Lab. Clin. Med. (2016), doi:10.1016/j.trs1.2016.07.021.

57. J. Cummins, M. Tangney, Bacteria and tumours: causative agents or opportunistic inhabitants? Infect. Agent. Cancer. 8, 11 (2013).

58. C. K. Baban, M. Cronin, D. O′Hanlon, G. C. O′Sullivan, M. Tangney, Bacteria as vectors for gene therapy of cancer. Bioeng. Bugs. 1, 385-394 (2010).

59. H. A. Barton, N. M. Taylor, B. R. Lubbers, A. C. Pemberton, DNA extraction from low-biomass carbonate rock: An improved method with reduced contamination and the low-biomass contaminant database. J. Microbiol. Methods. 66, 21-31 (2006).

60. A. Glassing, S. E. Dowd, S. Galandiuk, B. Davis, R. J. Chiodini, Inherent bacterial DNA contamination of extraction and sequencing reagents may affect interpretation of microbiota in low bacterial biomass samples. Gut Pathog. (2016), (available at www(dot)link(dot)galegroup(dot)com/apps/doc/A453529475/AONE?sid=lms).

61. A. P. Lauder, A. M. Roche, S. Sherrill-Mix, A. Bailey, A. L. Laughlin, K. Bittinger, R. Leite, M. A. Elovitz, S. Parry, F. D. Bushman, Comparison of placenta samples with contamination controls does not provide evidence for a distinct placenta microbiota. Microbiome. 4, 29 (2016).

62. L. S. Weyrich, A. G. Farrer, R. Eisenhofer, L. A. Arriola, J. Young, C. A. Selway, M. Handsley-Davis, C. J. Adler, J. Breen, A. Cooper, Laboratory contamination over time during low-biomass sample analysis. Mol. Ecol. Resour. 19, 982-996 (2019).

63. A. A. Kuperman, A. Zimmerman, S. Hamadia, O. Ziv, V. Gurevich, B. Fichtman, N. Gavert, R. Straussman, H. Rechnitzer, M. Barzilay, S. Shvalb, J. Bornstein, I. Ben-Shachar, S. Yagel, I. Haviv, O. Koren, Deep microbial analysis of multiple placentas shows no evidence for a placental microbiome. BJOG Int. J. Obstet. Gynaecol. 127, 159-169 (2020).

64. P. Bankhead, M. B. Loughrey, J. A. Fernández, Y. Dombrowski, D. G. McArt, P. D. Dunne, S. McQuaid, R. T. Gray, L. J. Murray, H. G. Coleman, J. A. James, M. Salto-Tellez, P. W. Hamilton, QuPath: Open source software for digital pathology image analysis. Sci. Rep. 7, 16878 (2017).

65. J. Schindelin, I. Arganda-Carreras, E. Frise, V. Kaynig, M. Longair, T. Pietzsch, S. Preibisch, C. Rueden, S. Saalfeld, B. Schmid, J.-Y. Tinevez, D. J. White, V. Hartenstein, K. Eliceiri, P. Tomancak, A. Cardona, Fiji: an open-source platform for biological-image analysis. Nat. Methods. 9, 676-682 (2012).

66. A. C. Ruifrok, D. A. Johnston, Quantification of histochemical staining by color deconvolution. Anal. Quant. Cytol. Histol. 23, 291-299 (2001).

67. S. Marco-Sola, M. Sammeth, R. Guigó, P. Ribeca, The GEM mapper: fast, accurate and versatile alignment by filtration. Nat. Methods. 9, 1185-1188 (2012).

68. D. Li, C.-M. Liu, R. Luo, K. Sadakane, T.-W. Lam, MEGAHIT: an ultra-fast single-node solution for large and complex metagenomics assembly via succinct de Bruijn graph. 31, 1674-1676 (2015).

69. B. Langmead, S. L. Salzberg, Fast gapped-read alignment with Bowtie 2. Nat. Methods. 9, 357-359 (2012).

70. F. Asnicar, G. Weingart, T. L. Tickle, C. Huttenhower, N. Segata, Compact graphical representation of phylogenetic data and metadata with GraPhlAn. PeerJ. 3 (2015), doi: 10.7717/peerj. 1029.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating cancer in a subject in need thereof the method comprising:
(a) analyzing for the abundance of at least one bacteria in the tumor microbiome of the subject; and
(b) administering to the subject a therapeutically effective amount of an immunotherapy treatment on the basis of the abundance of said at least one bacteria, thereby treating the cancer in the subject.

2. The method of claim 1, wherein when the abundance of a bacteria in the tumor microbiome set forth in Table 5 is above a predetermined amount, the subject is deemed a suitable candidate for therapy using said immunotherapy treatment.

3. The method of claim 1, wherein when the abundance of a bacteria in the tumor microbiome set forth in Table 6 is below a predetermined amount, the subject is deemed a suitable candidate for therapy using said immunotherapy treatment.

4. The method of claim 1, wherein said immunotherapy treatment comprises an immune checkpoint inhibitor.

5. The method of claim 1, wherein said cancer is melanoma.

6. The method of claim 1, wherein said at least one bacteria is set forth in Table 4.

7. The method of claim 1, wherein said at least one bacteria comprises each of the bacteria set forth in Table 4.

8. The method of claim 1, wherein said tumor is a metastasized tumor.

9. The method of claim 1, wherein said tumor is a non-metastasized tumor.

10. A method of treating a cancer in a subject, comprising:
(a)analyzing the abundance of a bacteria of at least one family, order, genus or species set forth in any of Tables 1-3 in a tumor sample of the subject, wherein an abundance of said bacteria above a predetermined level is indicative of the cancer; and
(b) treating the cancer with an anti-cancer agent, thereby treating the cancer.

11. The method of claim 10, wherein the cancer is selected from the group consisting of breast, melanoma, pancreatic cancer, ovarian cancer, bone cancer and brain cancer.

12. The method of claim 11, wherein said brain cancer comprises glioblastoma.

13. The method of claim 10, wherein said tumor sample is a non-metastasized tumor sample.

14. The method of claim 10, wherein said tumor sample is a metastasized tumor sample.

15. A composition of matter comprising a bacteria of a family, order, genus or species set forth in Tables 1-3, wherein said bacteria comprise or are linked to a therapeutic or diagnostic agent.

16. A method of delivering an agent to a tumor of a subject, the method comprising administering to the subject the composition of claim 15, thereby delivering the agent to a tumor of the subject.

17. The composition of matter of claim 15, wherein said therapeutic agent is a cytotoxic agent.

18. The composition of matter of claim 15, wherein said bacteria are genetically modified to express said agent.

19. The method of claim 16, wherein said tumor is selected from the group consisting of a breast tumor, a lung tumor, a skin tumor, a pancreas tumor, an ovarian tumor, a bone tumor and a brain tumor.


SEQUENCE LISTING (325 sequences)

SEQ ID NO: 1 - DNA, 19 bp, synthetic construct
  misc_feature 1..19: note = Single strand DNA oligonucleotide
  misc_feature 2: note = n is a, c, g, or t
  misc_feature 18: note = n is a, c, g, or t
  source 1..19: mol_type = other DNA
cnacgcgaagaaccttanc

SEQ ID NO: 2 - DNA, 19 bp, synthetic construct
  misc_feature 1..19: note = Single strand DNA oligonucleotide
  source 1..19: mol_type = other DNA
atacgcgargaaccttacc

SEQ ID NO: 3 - DNA, 19 bp, synthetic construct
  misc_feature 1..19: note = Single strand DNA oligonucleotide
  misc_feature 9: note = n is a, c, g, or t
  source 1..19: mol_type = other DNA
ctaaccgangaacctyacc

SEQ ID NO: 4 - DNA, 19 bp, synthetic construct
  misc_feature 1..19: note = Single strand DNA oligonucleotide
  source 1..19: mol_type = other DNA
caacgcgmaraaccttacc

SEQ ID NO: 5 - DNA, 19 bp, synthetic construct
  misc_feature 1..19: note = Single strand DNA oligonucleotide
  misc_feature 14: note = n is a, c, g, or t
  source 1..19: mol_type = other DNA
cgacrrccatgcancacct

SEQ ID NO: 6 - DNA, 18 bp, synthetic construct
  misc_feature 1..18: note = Single strand DNA oligonucleotide
  source 1..18: mol_type = other DNA
gctgcctcccgtaggagt

SEQ ID NO: 7 - DNA, 18 bp, synthetic construct
  misc_feature 1..18: note = Single strand DNA oligonucleotide
  source 1..18: mol_type = other DNA
cgacggagggcatcctca

SEQ ID NO: 8 - DNA, 18 bp, synthetic construct
  misc_feature 1..18: note = Single strand DNA oligonucleotide
  source 1..18: mol_type = other DNA
tggcgaacgggtgagtaa

SEQ ID NO: 9 - DNA, 18 bp, synthetic construct
  misc_feature 1..18: note = Single strand DNA oligonucleotide
  source 1..18: mol_type = other DNA
actcctacgggaggcagc

SEQ ID NO: 10 - DNA, 18 bp, synthetic construct
  misc_feature 1..18: note = Single strand DNA oligonucleotide
  source 1..18: mol_type = other DNA
gtgtagcggtgraatgcg

SEQ ID NO: 11 - DNA, 21 bp, synthetic construct
  misc_feature 1..21: note = Single strand DNA oligonucleotide
  source 1..21: mol_type = other DNA
ggagcatgtggwttaattcga

SEQ ID NO: 12 - DNA, 19 bp, synthetic construct
  misc_feature 1..19: note = Single strand DNA oligonucleotide
  source 1..19: mol_type = other DNA
ggaggaaggtggggatgac

SEQ ID NO: 13 - DNA, 40 bp, synthetic construct
  misc_feature 1..40: note = Single strand DNA oligonucleotide
  source 1..40: mol_type = other DNA
agacgtgtgctcttccgatctccgtgtctcagtcccartg

SEQ ID NO: 14 - DNA, 39 bp, synthetic construct
  misc_feature 1..39: note = Single strand DNA oligonucleotide
  source 1..39: mol_type = other DNA
agacgtgtgctcttccgatctgtattaccgcggctgctg

SEQ ID NO: 15 - DNA, 41 bp, synthetic construct
  misc_feature 1..41: note = Single strand DNA oligonucleotide
  source 1..41: mol_type = other DNA
agacgtgtgctcttccgatctcccgtcaattcmtttgagtt

SEQ ID NO: 16 - DNA, 39 bp, synthetic construct
  misc_feature 1..39: note = Single strand DNA oligonucleotide
  source 1..39: mol_type = other DNA
agacgtgtgctcttccgatctcgttgcgggacttaaccc

SEQ ID NO: 17 - DNA, 39 bp, synthetic construct
  misc_feature 1..39: note = Single strand DNA oligonucleotide
  source 1..39: mol_type = other DNA
agacgtgtgctcttccgatctaaggcccgggaacgtatt

SEQ ID NO: 18 - DNA, 76 bp, synthetic construct
  misc_feature 1..76: note = Single strand DNA oligonucleotide
  source 1..76: mol_type = other DNA
aatgatacggcgaccaccgagatctacactctttccctacacgacgctcttccgatcttggcgaacgggtgagtaa

SEQ ID NO: 19 - DNA, 76 bp, synthetic construct
  misc_feature 1..76: note = Single strand DNA oligonucleotide
  source 1..76: mol_type = other DNA
aatgatacggcgaccaccgagatctacactctttccctacacgacgctcttccgatctactcctacgggaggcagc

SEQ ID NO: 20 - DNA, 76 bp, synthetic construct
  misc_feature 1..76: note = Single strand DNA oligonucleotide
  source 1..76: mol_type = other DNA
aatgatacggcgaccaccgagatctacactctttccctacacgacgctcttccgatctgtgtagcggtgraatgcg

SEQ ID NO: 21 - DNA, 79 bp, synthetic construct
  misc_feature 1..79: note = Single strand DNA oligonucleotide
  source 1..79: mol_type = other DNA
aatgatacggcgaccaccgagatctacactctttccctacacgacgctcttccgatctggagcatgtggwttaattcga

SEQ ID NO: 22 - DNA, 77 bp, synthetic construct
  misc_feature 1..77: note = Single strand DNA oligonucleotide
  source 1..77: mol_type = other DNA
aatgatacggcgaccaccgagatctacactctttccctacacgacgctcttccgatctggaggaaggtggggatgac

SEQ ID NO: 23 - DNA, 66 bp, synthetic construct
  misc_feature 1..66: note = Single strand DNA oligonucleotide
  misc_feature 25..32: note = n is a, c, g, or t
  source 1..66: mol_type = other DNA
caagcagaagacggcatacgagatnnnnnnnngtgactggagttcagacgtgtgctcttccgatct

SEQ ID NO: 24 - DNA, 1522 bp, Trueperella
  source 1..1522: mol_type = genomic DNA
gtggagagtttgatcctggctcaggacgaacgctggcggcgtgcttaacacatgcaagtcgaacgatgaagctacagcttgctgtggtggattagtggcgaacgggtgagtaatacgtgagtaacctgcccttgtctttgggataagcctgggaaactgggtctaataccggatattctgctttcatcgcatggtgggggttggaaagggtttctggatagggatgggctcacggcctatcagcttgttggtggggtgatggcctaccaaggcgtcgacgggtagccggcctgagagggtgaccggccacattgggactgagatacggcccagactcctacgggaggcagcagtggggaatattgcacaatggacgcaagtctgatgcagcgacgccgcgtgggggatgaaggctttcgggttgtaaactcctttcagtacagaacaaggcctttttggttgagggtatgtgcagaagaagcgccggctaactacgtgccagcagccgcggtaatacgtagggcgcgagcgttgtccggaattattgggcgtaaagagctcgtaggcggtttgttgcgcctgctgtgaaagaccggggcttaacttcggggttgcagtgggtacgggcagactagagtgtggtaggggtaattggaattcctggtgtagcggtggaatgcgcagatatcaggaggaacaccgatggcgaaggcaggttactgggccaccactgacgctgaggagcgaaagcgtgggtagcgaacaggattagataccctggtagtccacgccgtaaacgttgggcactaggtgtggggccttttccacgggttctgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatacactgcgatgtgccagagatggtgcagccttcggggtggtgtacaggtggtgcatggttgtcgtcagctcgtgtcgtgagatgttgggttaagtcccgcaacgagcgcaacccttgtcctgtgttgccagcaattcggttggggactcacgggagactgccggggttaactcggaggaaggtggggatgacgtcaaatcatcatgccccttatgtcttgggcttcacgcatgctacaatggccggtacagagggttgcgagcctgtgagggtgagctaatctcttaaagctggtctcagttcggattggggtctgcaactcgaccccatgaagtcggagtcgctagtaatcgcagatcagcaacgctgcggtgaatacgttctcgggccttgtacacaccgcccgtcacgtcacgaaagttggtaacacccgaagcccgtggcctaaccttttggggggagcggtcgaaggtgggattggcgattgggacgaagtcgtaacaaggtagccgtaccggaaggtgcggctggatcacctcctt

SEQ ID NO: 25 - DNA, 1527 bp, Georgenia
  source 1..1527: mol_type = genomic DNA
aacggagagtttgatcctggctcaggacgaacgctggcggcgtgcttaacacatgcaagtcgaacgatgatggcccgggcttgcccgggtctgattagtggcgaacgggtgagtaacacgtgagtaacctgcccctgacttcgggataaccgcgagaaatcgtggctaataccggatatgacaccccggcgcatggtggggtgtggaaagactgtgtcggttggggatgggctcgcggcctatcagcttgttggtggggtgatggcctaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagctcttcggggtgacggtacctgcagaagaagcgccggctaactacgtgccagcagccgcggtaatacgtagggcgcaagcgttgtccggaattattgggcgtaaagagctcgtaggcggtctgtcgcgtctgctgtgaaaacgcggggctcaaccccgcgcctgcagtgggtacgggcagactagagtgcggtaggggagactggaattcctggtgtagcggtggaatgcgcagatatcaggaggaacaccggtggcgaaggcgggtctctgggccgttactgacgctgaggagcgaaagcatggggagcgaacaggattagataccctggtagtccatgccgtaaacgttgggcactaggtgtgggacccattccacgggttccgtgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatacaccggaaacgtccagagatgggcgccccgcaaggtcggtgtacaggtggtgcatggttgtcgtcagctcgtgtcgtgagatgttgggttaagtcccgcaacgagcgcaacccttgtcctgtgttgccagcgggttatgccggggactcatgggagactgccggggtcaactcggaggaaggtggggatgacgtcaaatcatcatgccccttatgtcttgggcttcacgcatgctacaatggccggtacaaagggctgcgatgccgtaaggtggagcgaatcccaaaaagccggtctcagttcggattggggtctgcaactcgaccccatgaagtcggagtcgctagtaatcgcagatcagcaacgctgcggtgaatacgttctcgggccttgtacacaccgcccgtcacgtcacgaaagtcggtaacacccgaagccggtggcccaacccttgtggggggagccgtcgaaggtgggactggcgattgggacgaagtcgtaacaaggtagccgtaccggaaggtgcggctggatcacctccttt

SEQ ID NO: 26 - DNA, 1520 bp, Cellulomonas
  source 1..1520: mol_type = genomic DNA
acggagagtttgattctggctcaggacgaacgctggcggcgtgcttaacacatgcaagtcgaacggtgatctccagcttgctggggtgatcagtggcgaacgggtgagtaacacgtgagtaacctgcccctgactctgggataactgctggaaacggtggctaataccggatatgacgtgtgcctgcatgggtgtgcgtggaaagtttttcggtcagggatgggctcgcggcctatcagcttgttggtggggtgatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgagagtgacggtacctgcagaagaagcgccggctaactacgtgccagcagccgcggtaatacgtagggcgcaagcgttgtccggaattattgggcgtaaagagctcgtaggcggtgtgtcgcgtctgctgtgaaatcctcaggctcaacctggggcttgcagtgggtacgggcacactagagtgcggtaggggtgactggaattcctggtgtagcggtggaatgcgcagatatcaggaggaacaccgatggcgaaggcaggtcactgggccgcaactgacgctgaggagcgaaagcatggggagcgaacaggattagataccctggtagtccatgccgtaaacgttgggcactaggtgtggggtccattccacggattccgtgccgcagcaaacgcattaagtgccccgcctggggagtacggccgcaaggctaaaactcaaagaaattgacgggggcccgcacaagcggcggagcatgcggattaattcgatgcaacgcgaagaaccttaccaaggcttgacatgcaccggtaacactcagagatgggtgccccgcaaggtcggtgcacaggtggtgcatggttgtcgtcagctcgtgtcgtgagatgttgggttaagtcccgcaacgagcgcaaccctcgttccatgttgccagcgggtcgtgccggggactcatgggagactgccggggtcaactcggaggaaggtggggatgacgtcaaatcatcatgccccttatgtcttgggcttcacgcatgctacaatggccggtacaaagggctgcgataccgcgaggtggagcgaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcccaacctccttgtgaggggggagctgtcgaaggtgggactggcgattgggactaagtcgtaacaaggtagccgtaccggaaggtgcggctggatcacctccttt

SEQ ID NO: 27 - DNA, 1520 bp, Oerskovia
  source 1..1520: mol_type = genomic DNA
tacggagagtttgattctggctcaggacgaacgctggcggcgtgcttaacacatgcaagtcgaacggtgaaggtcagcttgctgaccggatcagtggcgaacgggtgagtaacacgtgagcaacctgccccctactctgggataagccttggaaacgaggtctaatactggatacgaggcgcctgggcatctgggtgtctggaaagatttatcggtgggggatgggctcgcggcctatcagcttgttggtggggtgatggcctaccaaggcgacgacgggtagccggcctgagagggcgaccggccacactgggactgagacacggcccagactcctacgggaggcagcagtggggaatattgcacaatgggcgcaagcctgatgcagcgacgccgcgtgagggatgacggccttcgggttgtaaacctctttcagcagggaagaagcgcaagtgacggtacctgcagaagaagcgccggctaactacgtgccagcagccgcggtaatacgtagggcgcaagcgttgtccggaattattgggcgtaaagagctcgtaggcggtctgtcgcgtctgctgtgaaaacctcaggctcaacctggggcttgcagtgggtacgggcagactagagtgcggtaggggtgactggaattcctggtgtagcggtggaatgcgcagatatcaggaggaacaccgatggcgaaggcaggtcactgggccgcaactgacgctgaggagcgaaagcatggggagcgaacaggattagataccctggtagtccatgccgtaaacgttgggcactaggtgtggggtccattccacggattccgtgccgcagcaaacgcattaagtgccccgcctggggagtacggccgcaaggctaaaactcaaagaaattgacgggggcccgcacaagcggcggagcatgcggattaattcgatgcaacgcgaagaaccttaccaaggcttgacatgcaccggtaacactcagagatgggtgccccgcaaggtcggtgcacaggtggtgcatggttgtcgtcagctcgtgtcgtgagatgttgggttaagtcccgcaacgagcgcaaccctcgtcccatgttgccagcgggttatgccggggactcatgggagactgccggggtcaactcggaggaaggtggggatgacgtcaaatcatcatgccccttatgtcttgggcttcacgcatgctacaatggccggtacaaagggctgcgataccgcgaggtggagcgaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcatgaaagtcggtaacacccgaagccggtggcccaaccccttgtgggggggagccgtcgaaggtgggactggcgattaggactaagtcgtaacaaggtagccgtaccggaaggtgcggctggatcacctccttt

SEQ ID NO: 28 - skipped

SEQ ID NO: 29 - DNA, 1514 bp, Corynebacterium tuberculostearicum
  source 1..1514: mol_type = genomic DNA
gtggagagtttgatcctggctcaggacgaacgctggcggcgtgcttaacacatgcaagtcgaacggaaaggccctgcttgcagggtactcgagtggcgaacgggtgagtaacacgtgggtgatctgccctgcacttcgggataagcctgggaaactgggtctaataccggataggagccatttttagtgtgatggttggaaagttttttcggtgtaggatgagctcgcggcctatcagcttgttggtggggtaatggcctaccaaggcggcgacgggtagccggcctgagagggtggacggccacattgggactgagatacggcccagactcctacgggaggcagcagtggggaatattgcacaatgggcgcaagcctgatgcagcgacgccgcgtgggggatgacggccttcgggttgtaaactcctttcgctagggacgaagctttttgtgacggtacctagataagaagcaccggctaactacgtgccagcagccgcggtaatacgtagggtgcgagcgttgtccggaattactgggcgtaaagggctcgtaggtggtttgtcgcgtcgtctgtgaaattccggggcttaactccgggcgtgcaggcgatacgggcataacttgagtactgtaggggtaactggaattcctggtgtagcggtgaaatgcgcagatatcaggaggaacaccgatggcgaaggcaggttactgggcagttactgacgctgaggagcgaaagcatgggtagcgaacaggattagataccctggtagtccatgccgtaaacggtgggcgctaggtgtgagggtcttttcacgactttcgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatacactggatcgctgcagagatgtagtttccctttgtggctggtgtacaggtggtgcatggttgtcgtcagctcgtgtcgtgagatgttgggttaagtcccgcaacgagcgcaacccttgtcttatgttgccagcatttggttggggactcatgagagactgccggggtcaactcggaggaaggtggggatgacgtcaaatcatcatgccccttatgtccagggcttcacacatgctacaatggtcggtacaacgcgcagcgacactgtgaggtggagcgaatcgctgaaagccggccttagttcggattggggtctgcaactcgaccccatgaagtcggagtcgctagtaatcgcagatcagcaatgctgcggtgaatacgttcccgggccttgtacacaccgcccgtcacgtcatgaaagttggtaacacccgaagccggtggcccaaacttgttagggagccgtcgaaggtgggatcggcgattgggacgaagtcgtaacaaggtacccgtaccggaaggtgcgggtggatcacctcctt

SEQ ID NO: 30 - skipped

SEQ ID NO: 31 - DNA, 1514 bp, Corynebacterium tuberculostearicum
  source 1..1514: mol_type = genomic DNA
gtggagagtttgatcctggctcaggacgaacgctggcggcgtgcttaacacatgcaagtcgaacggaaaggccctgcttgcagggtactcgagtggcgaacgggtgagtaacacgtgggtgatctgccctgcacttcgggataagcctgggaaactgggtctaataccggataggagccatttttagtgtgatggttggaaagttttttcggtgtaggatgagctcgcggcctatcagcttgttggtggggtaatggcctaccaaggcggcgacgggtagccggcctgagagggtggacggccacattgggactgagatacggcccagactcctacgggaggcagcagtggggaatattgcacaatgggcgcaagcctgatgcagcgacgccgcgtgggggatgacggccttcgggttgtaaactcctttcgctagggacgaagctttttgtgacggtacctagataagaagcaccggctaactacgtgccagcagccgcggtaatacgtagggtgcgagcgttgtccggaattactgggcgtaaagggctcgtaggtggtttgtcgcgtcgtctgtgaaattccggggcttaactccgggcgtgcaggcgatacgggcataacttgagtactgtaggggtaactggaattcctggtgtagcggtgaaatgcgcagatatcaggaggaacaccgatggcgaaggcaggttactgggcagttactgacgctgaggagcgaaagcatgggtagcgaacaggattagataccctggtagtccatgccgtaaacggtgggcgctaggtgtgagggtcttttcacgactttcgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatacactggatcgggctagagatagtctttccctttgtggctggtgtacaggtggtgcatggttgtcgtcagctcgtgtcgtgagatgttgggttaagtcccgcaacgagcgcaacccttgtcttatgttgccagcatttggttggggactcatgagagactgccggggtcaactcggaggaaggtggggatgacgtcaaatcatcatgccccttatgtccagggcttcacacatgctacaatggtcggtacaacgcgcagcgacactgtgaggtggagcgaatcgctgaaagccggccttagttcggattggggtctgcaactcgaccccatgaagtcggagtcgctagtaatcgcagatcagcaacgctgcggtgaatacgttcccgggccttgtacacaccgcccgtcacgtcatgaaagttggtaacacccgaagccggtggcccaaacttgttagggagccgtcgaaggtgggatcggcgattgggacgaagtcgtaacaaggtacccgtaccggaaggtgcgggtggatcacctcctt

SEQ ID NO: 32 - DNA, 1515 bp, Corynebacterium variabile
  source 1..1515: mol_type = genomic DNA
tatggagagtttgatcctggctcaggacgaacgctggcggcgtgcttaacacatgcaagtcgaacggaaaggccctgcttgcagggtactcgagtggcgaacgggtgagtaacacgtgggtgatctgccctgcacttcgggataagcctgggaaactgggtctaataccggataggaccatcgtttagtgtcggtggtggaaagtttttcggtgcaggatgagctcgcggcctatcagcttgttggtggggtaatggcctaccaaggcgtcgacgggtagccggcctgagagggtggacggccacattgggactgagacacggcccagactcctacgggaggcagcagtggggaatattgcacaatgggcgcaagcctgatgcagcgacgccgcgtgggggatgaaggccttcgggttgtaaactcctttcaaccatgacgaagcattatgtgacggtagtggtagaagaagcaccggctaactacgtgccagcagccgcggtaatacgtagggtgcgagcgttgtccggaattactgggcgtaaagagctcgtaggtggtttgtcgcgtcgtctgtgaaattccggggcttaactccgggcgtgcaggcgatacgggcataacttgagtgctgtaggggagactggaattcctggtgtagcggtgaaatgcgcagatatcaggaggaacaccgatggcgaaggcaggtctctgggcagtaactgacgctgaggagcgaaagcatgggtagcgaacaggattagataccctggtagtccatgccgtaaacggtgggcgctaggtgtgggggtcttccacgacttctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatatgccggatcggcgcagagatgcgttttcccttgtggtcggtatacaggtggtgcatggttgtcgtcagctcgtgtcgtgagatgttgggttaagtcccgcaacgagcgcaacccttgtcttgtgttgccagcacgttatggtggggactcgcgagagactgccggggttaactcggaggaaggtggggatgacgtcaaatcatcatgccccttatgtccagggcttcacacatgctacaatggtcggtacagtgggttgctacaccgtgaggtggtgctaatcccttaaagccggtctcagttcggattggagtctgcaactcgactccatgaagtcggagtcgctagtaatcgcagatcagcaacgctgcggtgaatacgttcccgggccttgtacacaccgcccgtcacgtcatgaaagttggtaacacccgaagccagtggcccaaactcgttagggagctgtcgaaggtgggatcggcgattgggacgaagtcgtaacaaggtagccgtaccggaaggtgcggctggatcacctccttt

SEQ ID NO: 33 - DNA, 1317 bp, Corynebacterium
  source 1..1317: mol_type = genomic DNA
gaaagttttttcggtgtgggatgggcccgcggcctatcagcttgttggtggggtaatggcctaccaaggcggcgacgggtagccggcctgagagggtggacggccacattgggactgagacacggcccagactcctacgggaggcagcagtggggaatattgcacaatgggcggaagcctgatgcagcgacgccgcgtgggggatgacggccttcgggttgtaaactcctttcaccatcgacgaagggtttctgacggtagatggagaagaagcaccggctaactacgtgccagcagccgcggtaatacgtagggtgcgagcgttgtccggaattactgggcgtaaagagctcgtaggtggtttgtcgcgtcgtctgtgaaattccggggcttaactccgggcgtgcaggcgatacgggcataacttgagtactgtaggggagactggaattcctggtgtagcggtgaaatgcgcagatatcaggaggaacaccggtggcgaaggcgggtctctgggcagtaactgacgctgaggagcgaaagcatggggagcgaacaggattagataccctggtagtccatgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatatacaggatcgcgccagagatggtgtttcccttgtggcttgtatacaggtggtgcatggttgtcgtcagctcgtgtcgtgagatgttgggttaagtcccgcaacgagcgcaacccttgtcttatgttgccagcacgttgtggtggggactcgtaagaaactgccggggttaactcggaggaaggtggggatgacgtcaaatcatcatgccccttatgtccagggcttcacacatgctacaatggtcggtacagtgggttgccagtccgtgagggcgagctaatcccgcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaaactcgttagggagctgtcgaaggtgggattggcgattgggacgaagtcgtaacaaggtagccgtaccggaaggtgcggctggatcacctccttt

SEQ ID NO: 34 - skipped

SEQ ID NO: 35 - DNA, 1524 bp, Corynebacterium
  source 1..1524: mol_type = genomic DNA
gtggagagtttgatcctggctcaggatgaacgctggcggcgtgcttaacacatgcaagtcgaacggaaaggccagtgcttgcactggtactcgagtggcgaacgggtgagtaacacgtgggtgatctgccctcaactccgggataagcctgggaaactgggtctaatactggataggaccacatcgtaggtggtgtggtggaaagcgtttgtagcggttggggatgagctcgcggcctatcagcttgttggtggggtaatggcctaccaaggcggcgacgggtagccggcctgagagggtgtacggtcacattgggactgagatacggcccagactcctacgggaggcagcagtggggaatattgcacaatgggcgcaagcctgatgcagcgacgccgcgtgggggatgacggccttcgggttgtaaacctctttcgaccgtgacgaagcctttggtgacggtagcggtagaagaagcaccggctaactacgtgccagcagccgcggtaatacgtagggtgcgagcgttgtccggatttactgggcgtaaagggctcgtaggtggtttgtcgcgtcgtctgtgaaattccggggctcaactccgggcgtgcaggcgatacgggcataacttgagtactgcaggggtaactggaattcctggtgtagcggtggaatgcgcagatatcaggaggaacaccgatggcgaaggcaggttactgggcagttactgacgctgaggagcgaaagcatgggtagcgaacaggattagataccctggtagtccatgctgtcaacggtgggcgctaggtgtgagacccttccacgggttttgtgccgtagctaacgttttaagcgccccgcctggggagtacggccgcaaggctaaaactcaaaggaattgacgggggcccgcacaagcggcggagcatgtggattaattcgatgcaacgcgaagaaccttacctgggtttgacatacactggatcgcctcggagacggggtttccctttgtggtcggtgtacaggtggtgcatggttgtcgtcagctcgtgtcgtgagatgttgggttaagtcccgcaacgagcgcaacccttgtcctgtgttgccagcacgttgtggtggggactcacgggagactgccggggtgaactcggaggaaggtggggatgacgtcaaatcatcatgccccttatgtccagggcttcacacatgctacaatggtcggtacaacaggttgcgataccgtgaggtggagctaatccttttaaagccggcctcagttcggattggggtctgcaactcgaccccatgaagtcggagtcgctagtaatcgcagatcagcagtgctgcggtgaatacgttcccgggccttgtacacaccgcccgtcacgtcatgaaagtcggtaacacccgaagcccacggcccaacccttgtggggggagtggtcgaaggtgggatcggcgattgggacgaagtcgtaacaaggtagccgtaccggaaggtgcggctggatcacctccttt

SEQ ID NO: 36 - DNA, 1511 bp, Corynebacterium
  source 1..1511: mol_type = genomic DNA
gtggagagtttgatcctggctcaggatgaacgctggcggcgtgcttaacacatgcaagtcgaacggaaaggccagtgcttgcactggtactcgagtggcgaacgggtgagtaacacgtgggtgatctgccctgtacttcgggataagcttgggaaactgggtctaataccggataggacaactttttggatattgttgtggaaagcttttgcggtatgggatgagctcgcggcctatcagcttgttggtggggtaatggcctaccaaggcgtcgacgggtagccggcctgagagggtgtacggccacattgggactgagatacggcccagactcctacgggaggcagcagtggggaatattgcacaatgggcgcaagcctgatgcagcgacgccgcgtgggggatgaaggccttcgggttgtaaactcctttcgctagggacgaagcgcaagtgacggtacctggagaagaagcaccggctaactacgtgccagcagccgcggtaatacgtagggtgcgagcgttgtccggaattactgggcgtaaagagctcgtaggtggtttgtcgcgtcgtttgtgtaagtccacagcttaactgtgggactgcaggcgatacgggcataacttgagtgctgtaggggagactggaattcctggtgtagcggtgaaatgcgcagatatcaggaggaacaccgatggcgaaggcaggtctctgggcagtaactgacgctgaggagcgaaagcatgggtagcgaacaggattagataccctggtagtccatgccgtaaacggtgggcgctaggtgtgagtcccttccacgggatttgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatgtagcggatcgccgcagagatgtggtttcccttgtggtcgctatacaggtggtgcatggttgtcgtcagctcgtgtcgtgagatgttgggttaagtcccgcaacgagcgcaacccttgtcttatgttgccagcacgtaatggtggggactcatgagagactgccggggttaactcggaggaaggtggggatgacgtcaaatcatcatgccccttatgtccagggcttcacacatgctacaatggtcggtacagcgcgtttgctaccttgtgaggggatgctaatcgcacaaagccggccttagttcggattggggtctgcaactcgaccccatgaagtcggagtcgctagtaatcgcagatcagcaacgctgcggtgaatacgttcccgggccttgtacacaccgcccgtcacgtcatgaaagttggtaacacccgaagccagtggcctgtcatgggagctgtcgaaggtgggatcggcgattgggacgaagtcgtaacaaggtagccgtaccggaaggtgcggctggatcacctcctt

SEQ ID NO: 37 - DNA, 1518 bp, Corynebacterium
  source 1..1518: mol_type = genomic DNA
gtggagagtttgatcctggctcaggatgaacgctggcggcgtgcttaacacatgcaagtcgaacggaaaggccacagcttgctgtggtactcgagtggcgaacgggtgagtaacacgtgggtgatctgccctgtacttcgggacaagcttgggaaactgggtctaataccggataggaatcacatgtgggtgtgtggttggaaagcttttgcggtatgggatgagctcgcggcctatcagcttgttggtggggtaatggcctaccaaggcgtcgacgggtagccggcctgagagggtgtacggccacattgggactgagatacggcccagactcctacgggaggcagcagtggggaatattgcacaatgggcgcaagcctgatgcagcgacgccgcgtgggggatgacggccttcgggttgtaaactcctttcgtcagggacgaagcgaaagtgacggtacctggataagaagcaccggctaactacgtgccagcagccgcggtaatacgtagggtgcgagcgttgtccggaattactgggcgtaaagagctcgtaggtggtttgtcgcgtcgtttgtgtaagtccacggcttaactgtgggatggcaggcgatacgggcataacttgagtgctgtaggggagactggaattcctggtgtagcggtgaaatgcgcagatatcaggaggaacaccgatggcgaaggcaggtctctgggcagtaactgacgctgaggagcgaaagcatgggtagcgaacaggattagataccctggtagtccatgccgtaaacggtgggcgctaggtgtgagtcccttccacggggttcgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatacaccagatcggcgcagagatgcgttttcccttgtggttggtgtacaggtggtgcatggttgtcgtcagctcgtgtcgtgagatgttgggttaagtcccgcaacgagcgcaacccttgtcttatgttgccagcacgtgatggtggggactcatgagagactgccggggttaactcggaggaaggtggggatgacgtcaaatcatcatgccccttatgtccagggcttcacacatgctacaatggtcggtacaacgcgtgtgcgacactgtgaggtgaagctaatcgctctaaagccggtcgtagttcggattggggtctgcaactcgaccccatgaagtcggagtcgctagtaatcgcagatcagcaacgctgcggtgaatacgttcccgggccttgtacacaccgcccgtcacgtcatgaaagttggtaacacccgaagccagtggcctaaacctgttagggagctgtcgaaggtgggatcggcgattgggacgaagtcgtaacaaggtagccgtaccggaaggtgcggctggatcacctccttt

SEQ ID NO: 38 - DNA, 1516 bp, Dermabacter
  source 1..1516: mol_type = genomic DNA
aatggagagtttgatcctggctcaggacgaacgctggcggcgtgcttaacacatgcaagtcgaacgatgaagctgtgcttgcacagtggattagtggcgaacgggtgagtaacacgtgagtaacctgccctccactttgggataactccgagaaatcggggctaatactggatatgactatggcccgcatgggttgtggtggaaagtttttcggtgggggatgggctcgcggcctatcagtttgttggtgaggtagtggctcaccaaggcgatgacgggtagccggcctgagagggcgaccggccacactgggactgagacacggcccagactcctacgggaggcagcagtggggaatattgcacaatgggcgaaagcctgatgcagcgacgccgcgtgggggatgacggccttcgggttgtaaacctctttcagcaaggaagaagcgaaagtgacggtacttgcagaagaagcgccggctaactacgtgccagcagccgcggtaatacgtagggcgcaagcgttgtccggaattattgggcgtaaagggcttgtaggtggcttgtcgcgtctgccgtgaaaacccagggcttaactctgggcgtgcggtgggtacgggcaggctagagtgcggtaggggagactggaattcctggtgtagcggtgaaatgcgcagatatcaggaggaacaccgatggcgaaggcaggtctctgggccgttactgacactgagaagcgaaagcatggggagcgaacaggattagataccctggtagtccatgccgtaaacgttgggcactaggtgtggggagcattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgcactggatcgctgcagagatgtggttttctttggactggtgcacaggtggtgcatggttgtcgtcagctcgtgtcgtgagatgttgggttaagtcccgcaacgagcgcaaccctcgttccatgttgccagcacttcgggtggggactcatgggagactgccggggtcaactcggaggaaggtggggacgacgtcaaatcatcatgccccttatgtcttgggcttcacgcgtgctacaatggtcggtacaatgggttgcgatactgtgaggtggagctaatcccaaaaagccggtctcagttcggattggggtctgcaactcgaccctatgaagtcggagtcgctagtaatcgcagatcagcaatgctgcggtgaatacgttcccgggccttgtacacaccgcccgtcaagtcacgaaagtcggtaacacccgaagccgatggcccaagctttatgcggggagtcgtcgaaggtgggatcggtgattgggactaagtcgtaacaaggtagccgtaccggaaggtgcggctggatcacctccttt

SEQ ID NO: 39 - DNA, 1521 bp, Dermacoccus
  source 1..1521: mol_type = genomic DNA
aacggagagtttgatcctggctcaggacgaacgctggcggcgtgcttaacacatgcaagtcgaacgatgaagcgccagcttgctggtgtggattagtggcgaacgggtgagtaacacgtgagtaacctgccccctactctgggataagcctgggaaactgggtctaatactggatatgaccaatcactgcatggtgtgttggtggaaagattttttggtgggggatggactcgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcaccagggacgaagctaacgtgacggtacctggagaagaagcaccggctaactacgtgccagcagccgcggtaatacgtagggtgcgagcgttgtccggaattattgggcgtaaagagcttgtaggcggtttgtcgcgtctgctgtgaaagaccggggcttaactccggttctgcagtgggtacgggcagactagagtatggtaggggagactggaattcctggtgtagcggtgaaatgcgcagatatcaggaggaacaccgatggcgaaggcaggtctctgggccattactgacgctgagaagcgaaagcatggggagcgaacaggattagataccctggtagtccatgccgtaaacgttgggcgctaggtgtggggctcattccacgagttccgtgccgcagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaatcatgcagagatgtgtgcgtcttcggactggtgtacaggtggtgcatggttgtcgtcagctcgtgtcgtgagatgttgggttaagtcccgcaacgagcgcaaccctcgttccatgttgccagcacgtgatggtggggactcatgggagactgccggggtcaactcggaggaaggtggggatgacgtcaaatcatcatgccccttatgtcttgggcttcacgcatgctacaatggccggtacagagggttgcgaaactgtgaggtggagctaatcccaaaaaaccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggactggcgattgggactaagtcgtaacaaggtagccgtaccggaaggtgcggctggatcacctccttt

SEQ ID NO: 40 - DNA, 1521 bp, Dermacoccus
  source 1..1521: mol_type = genomic DNA
aacggagagtttgatcctggctcaggacgaacgctggcggcgtgcttaacacatgcaagtcgaacgatgaagcgccagcttgctggtgtggattagtggcgaacgggtgagtaacacgtgagtaacctgccccctactctgggataagcctgggaaactgggtctaatactggatatgaccaatcactgcatggtgtgttggtggaaagattttttggtgggggatggactcgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcaccagggacgaagctaacgtgacggtacctggagaagaagcaccggctaactacgtgccagcagccgcggtaatacgtagggtgcgagcgttgtccggaattattgggcgtaaagagcttgtaggcggtttgtcgcgtctgctgtgaaagaccggggcttaactccggttctgcagtgggtacgggcagactagagtatggtaggggagactggaattcctggtgtagcggtgaaatgcgcagatatcaggaggaacaccgatggcgaaggcaggtctctgggccattactgacgctgagaagcgaaagcatggggagcgaacaggattagataccctggtagtccatgccgtaaacgttgggcgctaggtgtggggctcattccacgagttccgtgccgcagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaatcatgcagagatgtgtgcgtcttcggactggtgtacaggtggtgcatggttgtcgtcagctcgtgtcgtgagatgttgggttaagtcccgcaacgagcgcaaccctcgttccatgttgccagcacgtcatggtggggactcatgggagactgccggggtcaactcggaggaaggtggggatgacgtcaaatcatcatgccccttatgtcttgggcttcacgcatgctacaatggccggtacagagggttgcgaaactgtgaggtggagctaatcccaaaaaaccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggactggcgattgggactaagtcgtaacaaggtagccgtaccggaaggtgcggctggatcacctccttt

SEQ ID NO: 41 - DNA, 1515 bp, Dietzia
  source 1..1515: mol_type = genomic DNA
tatggagagtttgatcctggctcaggacgaacgctggcggcgtgcttaacacatgcaagtcgaacggtaaggccccttcgggggtacacgagtggcgaacgggtgagtaacacgtgggtaatctgccctgcacttcgggataagcctgggaaaccgggtctaataccggatatgaactcctgccgcatggtgggggttggaaagtttttcggtgcaggatgagcccgcggcctatcagcttgttggtggggtaatggcctaccaaggcgacgacgggtagccggcctgagagggtgatcggccacactgggactgagacacggcccagactcctacgggaggcagcagtggggaatattgcacaatgggcgaaagcctgatgcagcgacgccgcgtgggggatgacggtcttcggattgtaaacccctttcagtagggacgaagcgcaagtgacggtacctgcagaagaagcaccggctaactacgtgccagcagccgcggtaatacgtagggtgcgagcgttgtccggaattactgggcgtaaagagctcgtaggcggtttgtcacgtcgtctgtgaaatccctcggcttaaccgggggcgtgcaggcgatacgggcagacttgagtactacaggggagactggaattcctggtgtagcggtgaaatgcgcagatatcaggaggaacaccggtggcgaaggcgggtctctgggtagtaactgacgctgaggagcgaaagcatgggtagcgaacaggattagataccctggtagtccatgccgtaaacggtgggcgctaggtgtggggtccttccacggactccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctaggcttgacatatacaggacgacggcagagatgtcgtttcccttgtggcttgtatacaggtggtgcatggttgtcgtcagctcgtgtcgtgagatgttgggttaagtcccgcaacgagcgcaacccctgtctcatgttgccagcacgtaatggtggggactcgtgagagactgccggggtcaactcggaggaaggtggggatgacgtcaaatcatcatgccccttatgtctagggcttcacacatgctacaatggctagtacagagggctgcgataccgcgaggtggagcgaatcccttaaagctggtctcagttcggattggggtctgcaactcgaccccatgaagtcggagtcgctagtaatcgcagatcagcaacgctgcggtgaatacgttcccgggccttgtacacaccgcccgtcacgtcatgaaagtcggtaacacccgaagccggtggcctaacccttgtggagggagccgtcgaaggtgggatcggcgattgggacgaagtcgtaacaaggtagccgtaccggaaggtgcggctggatcacctccttt

SEQ ID NO: 42 - DNA, 1516 bp, Blastococcus
  source 1..1516: mol_type = genomic DNA
acggagagtttgatcctggctcaggacgaacgctggcggcgtgcttaacacatgcaagtcgaacggtgaacctcgcttgcggggggatcagtggcgaacgggtgagtaacacgtgggcaacctgccctcagctctgggataactccaagaaattggggctaataccggatatgactgctgaccgcatggtctggtggtggaaagatttatcggctggggatgggcccgcggcctatcagcttgttggtggggtagtggcctaccaaggcgacgacgggtagccggcctgagagggtgaccggccacactgggactgagacacggcccagactcctacgggaggcagcagtggggaatattgcgcaatgggcgaaagcctgacgcagcgacgccgcgtgggggatgacggccttcgggttgtaaacctctttcagcagggacgaagcggaagtgacggtacctgcagaagaagcaccggccaactacgtgccagcagccgcggtaatacgtagggtgcaagcgttgtccggaattattgggcgtaaagagctcgtaggcggtttgttgcgtcggctgtgaaaacctgaggctcaacctcaggcctgcagtcgatacgagcagactagagtgttgcaggggagactggaattcctggtgtagcggtgaaatgcgcagatatcaggaggaacaccggtggcgaaggcgggtctctgggcaacaactgacgctgaggagcgaaagcgtggggagcgaacaggattagataccctggtagtccacgccgtaaacgttgggcgctaggtgtggggctcattccacgagttccgtgccgcagctaacgcattaagcgccccgcctggggagtacggccgcaaggctaaaactcaaaggaattgacgggggcccgcacaagcggcggagcatgttgcttaattcgatgcaacgcgaagaaccttacctaggcttgacatgcacggaaatcctccagagatggtgggtccgtaagggccgtgcacaggtggtgcatggttgtcgtcagctcgtgtcgtgagatgttgggttaagtcccgcaacgagcgcaaccctcgttccatgttgccagcacgtgatggtggggactcatgggagactgccggggtcaactcggaggaaggtggggatgacgtcaaatcatcatgccccttatgtctagggctgcaaacatgctacaatggccggtacaaagggctgcgataccgcgaggtggagcgaatcccaaaaagccggtctcagttcggattggggtctgcaactcgaccccatgaagttggagtcgctagtaatcgcagatcagcaacgctgcggtgaatacgttcccgggccttgtacacaccgcccgtcacgtcacgaaagtcggtaacgcccgaagccggtggcccaacccttgtggagggagccgtcgaaggcgggatcggcgattgggacgaagtcgtaacaaggtagccgtaccggaaggtgcggctggatcacctccttt

SEQ ID NO: 43 - DNA, 1520 bp, Janibacter
  source 1..1520: mol_type = genomic DNA
acggagagtttgatcctggctcaggacgaacgctggcggcgtgcttaacacatgcaagtcgaacggtgatgctccagcttgctggagtggatcagtggcgaacgggtgagtaacacgtgagcaacctgccccagactctggaataagcgctggaaacggcgtctaatactggatacgaaactggcccgcatgggcatggtttggaaagtttttcggtttgggatgggctcgcggcctatcagcttgttggtgaggtaatggctcaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcaccggctaactacgtgccagcagccgcggtaatacgtagggtgcgagcgttgtccggaattattgggcgtaaagagcttgtaggcggtttgtcgcgtctgctgtgaaaatccggggctcaaccccggacttgcagtgggtacgggcagactagagtgtggtaggggagactggaattcctggtgtagcggtgaaatgcgcagatatcaggaggaacaccgatggcgaaggcaggtctctgggccactactgacgctgagaagcgaaagcatggggagcgaacaggattagataccctggtagtccatgccgtaaacgttgggcgctaggtgtgggactcattccacgagttccgtgccgcagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatataccggaaacatccagagatgggtgccccttttaggtcggtatacaggtggtgcatggttgtcgtcagctcgtgtcgtgagatgttgggttaagtcccgcaacgagcgcaaccctcgttctatgttgccagcacgttatggtggggactcatggaagactgccggggtcaactcggaggaaggtggggatgacgtcaaatcatcatgccccttatgtcttgggcttcacgcatgctacaatggccggtacaaagggctgcgaaaccgtaaggtggagcgaatcccaaaaaaccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcccaacccttgcggagggagccgtcgaaggtgggactggcgattgggactaagtcgtaacaaggtagccgtaccggaaggtgcggctggatcacctccttt

SEQ ID NO: 44 - DNA, 1518 bp, Ornithinimicrobium
  source 1..1518: mol_type = genomic DNA
atcggagagtttgatcctggctcaggacgaacgctggcggcgtgcttaacacatgcaagtcgaacgatgaagcccagcttgctgggtggattagtggcgaacgggtgagtaacacgtgagtaacctgccctccactctgggataactccgggaaaccggtgctaatactggatatgaccttggcccgcatgggttggggtggaaagatttatcggtggtggatggactcgcggcctatcagcttgttggtggggtaatggcctaccaaggcgacgacgggtagccggcctgagagggtgaccggccacactgggactgagacacggcccagactcctacgggaggcagcagtggggaatattgcacaatgggcgaaagcctgatgcagcgacgccgcgtgcgggatgacggccttcgggttgtaaaccgctttcagctctgacgaagcttttgtgacggtaggagcagaagaagcaccggctaactacgtgccagcagccgcggtaatacgtagggtgcgagcgttgtccggaattattgggcgtaaagagcttgtaggcggcttgtcgcgtctgctgtgaaatcccggggcttaaccccgggcttgcagtgggtacgggcaggctagagtgtggtaggggagactggaattcctggtgtagcggtggaatgcgcagatatcaggaggaacaccgatggcgaaggcaggtctctgggccattactgacgctgagaagcgaaagcgtggggagcgaacaggattagataccctggtagtccacgccgtaaacgttgggcgctaggtgtgggtcccattccacggggtccgtgccgcagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatataccggaaactcccagagatgggggccccgcaaggtcggtatacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggatgacgtcaaatcatcatgccccttacgtcttgggcttcacgcatgctacaatggccggtacagagggctgcgaaaccgtgaggtggagcgaatcccttaaagctggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcccaacccttgtggggggagccgtcgaaggtgggactggtgattgggactaagtcgtaacaaggtagccgtaccggaaggtgcggctggatcacctccttt

SEQ ID NO: 45 - DNA, 1519 bp, Agrococcus
  source 1..1519: mol_type = genomic DNA
tacggagagtttgatcctggctcaggacgaacgctggcggcgtgcctaacacatgcaagtcgaacgatgaaggaggagcttgctcctctggattagtggcgaacgggtgagtaatacgtgagcaatgtgcccttgactctgggataagcgctggaaacggcgtctaataccggatacgaccctcggaggcatctcctgatggtggaaagaacttcggtcaaggatcagctcacggcctatcagctagttggtgaggtaacggctcaccaaggcgacgacgggtagccggcctgagagggtgaccggccacactgggactgagacacggcccagactcctacgggaggcagcagtggggaatattgcacaatgggcgcaagcctgatgcagcaacgccgcgtgagggacgaaggccttcgggttgtaaacctcttttagcagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaaacccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcatggggagcaaacaggcttagataccctggtagtccatgccgtaaacgttgggaactagatgtggggaccattccacggtctccgtgtcgtagctaacgcattaagttccccgcctggggagtacggccgcaaggctaaaactcaaaggaattgacgggggcccgcacaagcggcggagcatgcggattaattcgatgcaacgcgaagaaccttaccaaggcttgacatataccggaaacggccagagatggtcgccccgcaaggtcggtatacaggtggtgcatggttgtcgtcagctcgtgtcgtgagatgttgggttaagtcccgcaacgagcgcaaccctcgtcctatgttgccagcacgtaatggtgggaactcatgggatactgccggggtcaactcggaggaaggtggggatgaggtcaaatcatcatgccccttatgtcttgggcttcacgcatgctacaatggccggtacaatgggctgcgataccgcaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccagtggcctaaccttttggagggagctgtcgaaggtgggatcggtgattaggactaagtcgtaacaaggtagccgtaccggaaggtgcggctggatcacctccttt

SEQ ID NO: 46 - DNA, 1521 bp, Agrococcus
  source 1..1521: mol_type = genomic DNA
tacggagagtttgatcctggctcaggacgaacgctggcggcgtgcttaacacatgcaagtcgaacgatgaaggaggagcttgctcctctggattagtggcgaacgggtgagtaacacgtgagcaatctgcccttgactctgggataagcgctggaaacggcgtctaataccggatacgaccctcggaggcatctcctgtgggtggaaagaacttcggtcaaggatgagctcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcaacgccgcgtgagggacgacggccttcgggttgtaaacctcttttagcagggaagaagcgagagtgacggtacctgcagaaaaagcaccggctaactacgtgccagcagccgcggtaatacgtagggtgcaagcgttgtccggaattattgggcgtaaagagctcgtaggcggtctgtcgcgtctgctgtgaaaacccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcgtggggagcaaacaggcttagataccctggtagtccacgccgtaaacgttgggaactagatgtggggaccattccacggtctccgtgtcgtagctaacgcattaagttccccgcctggggagtacggccgcaaggctaaaactcaaaggaattgacgggggcccgcacaagcggcggagcatgcggattaattcgatgcaacgcgaagaaccttaccaaggcttgacatatacgagaacgcgctggagacagcgaactctttggacactcgtatacaggtggtgcatggttgtcgtcagctcgtgtcgtgagatgttgggttaagtcccgcaacgagcgcaaccctcgtcctatgttgccagcacgtcatggtgggaactcatgggatactgccggggtcaactcggaggaaggtggggatgaggtcaaatcatcatgccccttatgtcttgggcttcacgcatgctacaatggccggtacaatgggctgcgataccgcgaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaaccttttggagggagccgtcgaaggtgggatcggtgattaggactaagtcgtaacaaggtagccgtaccggaaggtgcggctggatcacctccttt

SEQ ID NO: 47 - DNA, 1521 bp, Microbacterium
  source 1..1521: mol_type = genomic DNA
tacggagagtttgatcctggctcaggatgaacgctggcggcgtgcttaacacatgcaagtcgaacggtgaagcccagcttgctgggtggatcagtggcgaacgggtgagtaacacgtgagcaacctgcccctgactctgggataagcgctggaaacggcgtctaatactggatatgtgatgtgaccgcatggtctgcgtctggaaagatttttcggttggggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatcccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggtccattccacggattccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgcgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcctaacccttgtggagggagccgtcgaaggtgggatcggtaattaggactaagtcgtaacaaggtagccgtaccggaaggtgcggctggatcacctccttt

SEQ ID NO: 48 - DNA, 1520 bp, Microbacterium
  source 1..1520: mol_type = genomic DNA
tacggagagtttgatcctggctcaggatgaacgctggcggcgtgcttaacacatgcaagtcgaacggtgaaagagagcttgctctctggatcagtggcgaacgggtgagtaacacgtgagcaacctgccccggactctgggataacagctggaaacagctgctaataccggatacgagctgcgaaggcatcttcagcagctggaaagaatttcggtccgggatgggctcgcggcctatcagctagttggtgaggtaatggctcaccaaggcgtcgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaaacccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggtccattccacggattccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgcaaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagtcgtaacaaggtagccgtaccggaaggtgcggctggatcacctccttt

SEQ ID NO: 49 - DNA, 1521 bp, Microbacterium
  source 1..1521: mol_type = genomic DNA
tatggagagtttgatcctggctcaggatgaacgctggcggcgtgcttaacacatgcaagtcgaacggtgaagcccagcttgctgggtggatcagtggcgaacgggtgagtaacacgtgagcaacctgccctggactctgggataagcgctggaaacggcgtctaatactggatatgaaccacgaaggcatcttcagtggttggaaagatttttcggtctgggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcggaagcctgatgcagcaacgccgcgtgagggatgacggccttcgggttgtaaacctcttttagcaaggaagaagcttttgtgacggtacttgcagaaaaagcgccggctaactacgtgccagcagccgcggtaatacgtagggcgcaagcgttatccggaattattgggcgtaaagagctcgtaggcggtttgtcgcgtctgctgtgaaatcccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggtcctttccacggattccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatacacgagaacaccctagaaataggggactctttggacactcgtgaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggggggagccgtcgaaggtgggatcggtaattaggactaagtcgtaacaaggtagccgtaccggaaggtgcggctggatcacctccttt

SEQ ID NO: 50 - DNA, 1521 bp, Microbacterium
  source 1..1521: mol_type = genomic DNA
tacggagagtttgatcctggctcaggatgaacgctggcggcgtgcttaacacatgcaagtcgaacggtgaagcagagcttgctctgtggatcagtggcgaacgggtgagtaacacgtgagcaacctgccccggactctgggataagcgctggaaacggcgtctaatactggatacgagctgcgaccgcatggtcagcagttggaaagatttttcggtctgggatgggctcacggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagcagggaagaagcgagagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatcccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggtccattccacggattccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgcgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagtcgtaacaaggtagccgtaccggaaggtgcggctggatcacctccttt

SEQ ID NO: 51 - DNA, 1520 bp, Microbacterium
  source 1..1520: mol_type = genomic DNA
tatggagagtttgatcctggctcaggatgaacgctggcggcgtgcttaacacatgcaagtcgaacggtgaagccccgcttgcggggtggatcagtggcgaacgggtgagtaacacgtgagcaacctgccctggactctgggataagcgctggaaacggcgtctaatactggatatgagctctcatcgcatggtgggggttggaaagattttttggtctgggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcggaagcctgatgcagcaacgccgcgtgagggatgacggccttcgggttgtaaacctcttttagcaaggaagaagcttttgtgacggtacttgcagaaaaagcgccggctaactacgtgccagcagccgcggtaatacgtagggcgcaagcgttatccggaattattgggcgtaaagagctcgtaggcggtttgtcgcgtctgctgtgaaatcccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggtcctttccacggattccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatacacgagaacaccccagaaatgggggactctttggacactcgtgaacaggtggtgcatggttgtcgtcagctcgtgtcgtgagatgttgggttaagtcccgcaacgagcgcaaccctcgttctatgttgccagcacgttatggtgggaactcatgggatactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagtcgtaacaaggtagccgtaccggaaggtgcggctggatcacctccttt

SEQ ID NO: 52 - DNA, 1517 bp, Arthrobacter aurescens
  source 1..1517: mol_type = genomic DNA
acggagagtttgatcctggctcaggatgaacgctggcggcgtgcttaacacatgcaagtcgaacgatgatcccagcttgctgggggattagtggcgaacgggtgagtaacacgtgagtaacctgcccttgactctgggataagcctgggaaactgggtctaataccggatatgactgtctgacgcatgtcaggtggtggaaagcttttgtggttttggatggactcgcggcctatcagcttgttggtggggtaatggcctaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcgccggctaactacgtgccagcagccgcggtaatacgtagggcgcaagcgttatccggaattattgggcgtaaagagctcgtaggcggtttgtcgcgtctgctgtgaaagaccggggctcaactccggttctgcagtgggtacgggcagactagagtgcagtaggggagactggaattcctggtgtagcggtgaaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggtaatacctggaaacaggtgccccgcttgcggtcggtttacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggaccggcgattgggactaagtcgtaacaaggtagccgtaccggaaggtgcggctggatcacctccttt

SEQ ID NO: 53 - DNA, 1091 bp, Micrococcus luteus
  source 1..1091: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccct

SEQ ID NO: 54 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtaatggtggggactcatggaagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 55 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtcgtggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 56 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactaggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 57 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttccccttgggggcgggttcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 58 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctccccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtcgtggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 59 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 60 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 61 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgcctaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 62 - DNA, 1397 bp, Micrococcus luteus
  source 1..1397: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactaggtctaataccggataggagcgcctaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaag

SEQ ID NO: 63 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggtgggttcacaggtggtgcatggttgtcgtcagctcgtgtcgtgagatgttgggttaagtcccgcaacgagcgcaaccctcgttccatgttgccagcacgtcgtggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagctgtcgaaggtgggaccagcgattgggactaagtcgtaacaaggtagccgtaccggaaggtgcggctggatcacctccttt

SEQ ID NO: 64 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactaggtctaataccggataggagcgcctaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 65 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 66 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgcctaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 67 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggtgggttcacaggtggtgcatggttgtcgtcagctcgtgtcgtgagatgttgggttaagtcccgcaacgagcgcaaccctcgttccatgttgccagcacgtcgtggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 68 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactaggtctaataccggataggagcgcctaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtcgtggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 69 - DNA, 1058 bp, Micrococcus luteus
  source 1..1058: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgcctaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggcgcaagcgttgtccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgaga

SEQ ID NO: 70 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 71 - DNA, 1521 bp, Micrococcus luteus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtcgtggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 72 - DNA, 1521 bp, Arthrobacter
  source 1..1521: mol_type = genomic DNA
aacggagagtttgatcctggctcaggacgaacgctggcggcgtgcttaacacatgcaagtcgaacgatgatccggtgcttgcaccggggattagtggcgaacgggtgagtaacacgtgagtaacctgcccttgactctgggataagcctgggaaaccgggtctaataccggatatgactcttcaccgcatggtggggggtggaaagattttttggttttggatggactcgcggcctatcagcttgttggtggggtaatggcctaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagcagggaagaagcggaagtgacggtacctgcagaagaagcgccggctaactacgtgccagcagccgcggtaatacgtagggcgcaagcgttgtccggaattattgggcgtaaagagctcgtaggcggtttgtcgcgtctgctgtgaaagcccggggctcaaccccgggtctgcagtgggtacgggcagactggagtgcagtaggggagactggaattcctggtgtagcggtgaaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgggccggatcgccgcagaaatgcggtttcccttcggggccggttcacaggtggtgcatggttgtcgtcagctcgtgtcgtgagatgttgggttaagtcccgcaacgagcgcaaccctcgttctatgttgccagcgcgtgatggcggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcacgaaagttggtaacacccgaagccggtggcctaaccccttggggagggagccgtcgaaggtgggaccggcgattgggactaagtcgtaacaaggtagccgtaccggaaggtgcggctggatcacctccttt

SEQ ID NO: 73 - DNA, 1529 bp, Kocuria
  source 1..1529: mol_type = genomic DNA
acggagagtttgatcctggctcaggacgaacgctggcggcgtgcttaacacatgcaagtcgaacgctgaagcttggtgcttgcactgggtggatgagtggcgaacgggtgagtaatacgtgagtaacctgcccttgactctgggataagcctgggaaactgggtctaatactggatatgacctcctgtcgcatggtggggggtggaaagggtttgtactggttgtggatgggctcacggcctatcagcttgttggtggggtaatggcctaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagcacggaagaagcgaaagtgacggtacgtgcagaagaagcgccggctaactacgtgccagcagccgcggtaatacgtagggcgcaagcgttgtccggaattattgggcgtaaagagctcgtaggcggtttgtcgcgtctgctgtgaaagcccggggcttaaccccgggtgtgcagtgggtacgggcagacttgagtgcagtaggggagactggaactcctggtgtagcggtgaaatgcgcagatatcaggaagaacaccgatggcgaaggcaggtctctgggctgttactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatacaccggatcggctcagagatgggttttcctccttgtggggctggtgtacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggatgacgtcaaatcatcatgccccttatgtcttgggcttcacgcatgctacaatggccagtacaatgggttgcgataccgtgaggtggagctaatcccaaaaagctggtctcagttcggatcgtggtctgcaactcgaccacgtgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggactggcgattgggactaagtcgtaacaaggtagccgtaccggaaggtgcggctggatcacctccttt

SEQ ID NO: 74 - DNA, 1522 bp, Microbispora
  source 1..1522: mol_type = genomic DNA
acggagagtttgatcctggctcaggacgaacgctggcggcgtgcttaacacatgcaagtcgaacgatgatgcccagcttgctgggcggattagtggcgaacgggtgagtaatacgtgagtaacctgcccctgactctgggataagcctgggaaaccgggtctaatactggatacgacctcctgccgcatggtggggggtggaaagggttcactggttttggatgggctcacggcctatcagcttgttggtggggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgcaagtgacggtacctgcagaagaagcgccggctaactacgtgccagcagccgcggtaatacgtagggcgcaagcgttgtccggaattattgggcgtaaagagctcgtaggcggtttgtcgcgtccgctgtgaaagcccggggctcaaccccgggtcggcagtgggtacgggcagactagagtgcagtaggggagactggaattcctggtgtagcggtgaaatgcgcagatatcaggaggaacaccgatggcgaaggcaggtctctgggctgttactgacgctgaggagcgaaagcatggggagcgaacaggattagataccctggtagtccatgccgtaaacgttgggcactaggtgtgggggacattccacgttctccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatccaccggaccgccccagagatggggtttcccttcggggctggtggacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggatgacgtcaaatcatcatgccccttatgtcttgggcttcacgcatgctacaatggccggtacaaagggttgcgatactgtgaggtggagctaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcacgaaagttggtaacacccgaagccggtggcctaaccccttgtgggagggagccgtcgaaggtgggactggcgattgggactaagtcgtaacaaggtagccgtaccggaaggtgcggctggatcacctccttt

SEQ ID NO: 75 - DNA, 1523 bp, Microbispora
  source 1..1523: mol_type = genomic DNA
acggagagtttgatcctggctcaggacgaacgctggcggcgtgcttaacacatgcaagtcgaacgatgatgtcccgcttgcgggacggattagtggcgaacgggtgagtaatacgtgagtaacctgcccctgactctgggataagcctgggaaaccgggtctaatactggatactacttctcaccgcatggtgggtggtggaaagggttttactggttttggatgggctcacggcctatcagcttgttggtggggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcgccggctaactacgtgccagcagccgcggtaatacgtagggcgcaagcgttgtccggaattattgggcgtaaagagctcgtaggcggtttgtcgcgtctgctgtgaaagcccggggctcaaccccgggtctgcagtgggtacgggcagactagagtgcagtaggggagactggaattcctggtgtagcggtgaaatgcgcagatatcaggaggaacaccgatggcgaaggcaggtctctgggctgttactgacgctgaggagcgaaagcatggggagcgaacaggattagataccctggtagtccatgccgtaaacgttgggcactaggtgtgggggacattccacgttctccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatccgccggaccgccccagagatggggtttcccttcggggccggtggacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggatgacgtcaaatcatcatgccccttatgtcttgggcttcacgcatgctacaatggccggtacaaagggttgcgatactgtgaggtggagctaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcacgaaagttggtaacacccgaagccggtggcctaaccccttgtgggagggagccgtcgaaggtgggactggcgattgggactaagtcgtaacaaggtagccgtaccggaaggtgcggctggatcacctccttt

SEQ ID NO: 76 - DNA, 1521 bp, Micrococcus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctacccttgactctgggataagcctgggaaactgggtctaataccggataggaacgtccaccgcatggtggttgttggaaagatttatcggtcatggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggatcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtggggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcctaacccttgtggggggagccgtcgaaggtgggaccagcgattgggactaagtcgtaacaaggtagccgtaccggaaggtgcggctggatcacctccttt

SEQ ID NO: 77 - DNA, 1521 bp, Micrococcus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctacagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggtgggttcacaggtggtgcatggttgtcgtcagctcgtgtcgtgagatgttgggttaagtcccgcaacgagcgcaaccctcgttccatgttgccagcacgtcgtggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagctgtcgaaggtgggaccagcgattgggactaagtcgtaacaaggtagccgtaccggaaggtgcggctggatcacctccttt

SEQ ID NO: 78 - DNA, 1523 bp, Micrococcus
  source 1..1523: mol_type = genomic DNA
tacggagagtttgatcctggctcaggatgaacgctggcggcgtgcttaacacatgcaagtcgaacgctgaagccccagcttgctggggtggatgagtggcgaacgggtgagtaacacgtgagtaaccttcccttgactctgggataagcctgggaaaccgggtctaataccggatgcgaccgtccaccgcatggtggggggtggaaagatttatcggtcttggatggactcgcggcctatcagcttgttggtggggtaatggcccaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcgccggctaactacgtgccagcagccgcggtaatacgtagggcgcgagcgttatccggaattattgggcgtaaagagctcgtaggcggtttgtcacgtctgccgtgaaagtccggggcttaaccccggatctgcggtgggtacgggcagactggagtgcagtaggggagactggaattcctggtgtagcggtgaaatgcgcagatatcaggaggaacaccgatggcgaaggcaggtctctgggctgttactgacgctgaggagcgaaagcatggggagcgaacaggattagataccctggtagtccatgccgtaaacgttgggcactaggtgtgggggccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttcccgaccgccacagagatgtggtttcccctttggggcgggttcacaggtggtgcatggttgtcgtcagctcgtgtcgtgagatgttgggttaagtcccgcaacgagcgcaaccctcgttccatgttgccagcgggttatgccggggactcatgggagactgccggggtcaactcggaggaaggtggggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcccaacccttgtggggggagccgtcgaaggtgggactggcgattgggactaagtcgtaacaaggtagccgtaccggaaggtgcggctggatcacctccttt

SEQ ID NO: 79 - DNA, 1521 bp, Micrococcus
  source 1..1521: mol_type = genomic DNA
tacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgtccaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagcgaaagtgacggtacctacagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggtgggttcacaggtggtgcatggttgtcgtcagctcgtgtcgtgagatgttgggttaagtcccgcaacgagcgcaaccctcgttccatgttgccagcacgtcgtggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagctgtcgaaggtgggaccagcgattgggactaagtcgtaacaaggtagccgtaccggaaggtgcggctggatcacctccttt

SEQ ID NO: 80 - DNA, 1516 bp, Mycobacterium
  source 1..1516: mol_type = genomic DNA
ttggagagtttgatcctggctcaggacgaacgctggcggcgtgcttaacacatgcaagtcgaacggaaaggccccttcgggggtactcgagtggcgaacgggtgagtaacacgtgggtaatctgccctgcacttcgggataagcctgggaaactgggtctaataccggataggaccacttggcgcatgccttgtggtggaaagcttttgcggtgtgggatgggcccgcggcctatcagcttgttggtggggtgacggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgggggatgacggccttcgggttgtaaacctctttcagcagggacgaagcgcaagtgacggtacctgcagaagaagcaccggccaactacgtgccagcagccgcggtaatacgtagggtgcgagcgttgtccggaattactgggcgtaaagagctcgtaggtggtttgtcgcgttgttcgtgaaaaccgggggcttaaccctcggcgtgcgggcgatacgggcagactggagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttggaatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgccggcagagatgtcggttcccttgtggcctgtgtgcaggtggtgcatggctgtcgtcagctcgtgtcgtgagatgttgggttaagtcccgcaacgagcgcaacccttgtctcatgttgccagcgggtaatgccggggactcgtgagagactgccggggtcaactcggaggaaggtggggatgacgtcaagtcatcatgccccttatgtccagggcttcacacatgctacaatggccggtacaaagggctgcgatgccgcaaggttaagcgaatccttttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaaccttttggagggagctgtcgaaggtgggatcggcgattgggacgaagtcgtaacaaggtagccgtaccggaaggtgcggctggatcacctccttt

SEQ ID NO: 81 - DNA, 1515 bp, Rhodococcus erythropolis
  source 1..1515: mol_type = genomic DNA
acggagagtttgatcctggctcaggacgaacgctggcggcgtgcttaacacatgcaagtcgagcggtaaggcctttcggggtacacgagcggcgaacgggtgagtaacacgtgggtgatctgccctgcacttcgggataagcctgggaaactgggtctaataccggatatgacctcctatcgcatggtgggtggtggaaagatttatcggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgcaagtgacggtacctgcagaagaagcaccggctaactacgtgccagcagccgcggtaatacgtagggtgcaagcgttgtccggaattactgggcgtaaagagttcgtaggcggtttgtcgcgtcgtttgtgaaaaccagcagctcaactgctggcttgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcgaacaggattagataccctggtagtccacgccgtaaacggtgggcgctaggtgtgggttccttccacggaatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagctgcagagatgtggccccccttgtggtcggtatacaggtggtgcatggctgtcgtcagctcgtgtcgtgagatgttgggttaagtcccgcaacgagcgcaacccctatcttatgttgccagcacgttatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcttaaccccttgtgggagggagccgtcgaaggtgggatcggcgattgggacgaagtcgtaacaaggtagccgtaccggaaggtgcggctggatcacctccttt

SEQ ID NO: 82 - DNA, 1522 bp, Propionibacterium acnes
  source 1..1522: mol_type = genomic DNA
ttggagagtttgatcctggctcaggacgaacgctggcggcgtgcttaacacatgcaagtcgaacggaaaggccctgcttttgtggggtgctcgagtggcgaacgggtgagtaacacgtgagtaacctgcccttgactttgggataacttcaggaaactggggctaataccggataggagctcctgctgcatggtgggggttggaaagtttcggcggttggggatggactcgcggcttatcagcttgttggtggggtagtggcttaccaaggctttgacgggtagccggcctgagagggtgaccggccacattgggactgagatacggcccagactcctacgggaggcagcagtggggaatattgcacaatgggcggaagcctgatgcagcaacgccgcgtgcgggatgacggccttcgggttgtaaaccgctttcgcctgtgacgaagcgtgagtgacggtaatgggtaaagaagcaccggctaactacgtgccagcagccgcggtgatacgtagggtgcgagcgttgtccggatttattgggcgtaaagggctcgtaggtggttgatcgcgtcggaagtgtaatcttggggcttaaccctgagcgtgctttcgatacgggttgacttgaggaaggtaggggagaatggaattcctggtggagcggtggaatgcgcagatatcaggaggaacaccagtggcgaaggcggttctctgggcctttcctgacgctgaggagcgaaagcgtggggagcgaacaggcttagataccctggtagtccacgctgtaaacggtgggtactaggtgtggggtccattccacgggttccgcgccgtagctaacgctttaagtaccccgcctggggagtacggccgcaaggctaaaactcaaaggaattgacggggccccgcacaagcggcggagcatgcggattaattcgatgcaacgcgtagaaccttacctgggtttgacatggatcgggagtgctcagagatgggtgtgcctcttttggggtcggttcacaggtggtgcatggctgtcgtcagctcgtgtcgtgagatgttgggttaagtcccgcaacgagcgcaacccttgttcactgttgccagcacgttatggtggggactcagtggagaccgccggggtcaactcggaggaaggtggggatgacgtcaagtcatcatgccccttatgtccagggcttcacgcatgctacaatggctggtacagagagtggcgagcctgtgagggtgagcgaatctcggaaagccggtctcagttcggattggggtctgcaactcgacctcatgaagtcggagtcgctagtaatcgcagatcagcaacgctgcggtgaatacgttcccggggcttgtacacaccgcccgtcaagtcatgaaagttggtaacacccgaagccggtggcctaaccgttgtgggggagccgtcgaaggtgggactggtgattaggactaagtcgtaacaaggtagccgtaccggaaggtgcggctggatcacctccttt

SEQ ID NO: 83 - DNA, 1522 bp, Propionibacterium acnes
  source 1..1522: mol_type = genomic DNA
ttggagagtttgatcctggctcaggacgaacgctggcggcgtgcttaacacatgcaagtcgaacggaaaggccctgcttttgtggggtgctcgagtggcgaacgggtgagtaacacgtgagtaacctgcccttgactttgggataacttcaggaaactggggctaataccggataggagctcctgctgcatggtgggggttggaaagtttcggcggttggggatggactcgcggcttatcagcttgttggtggggtagtggcttaccaaggctttgacgggtagccggcctgagagggtgaccggccacattgggactgagatacggcccagactcctacgggaggcagcagtggggaatattgcacaatgggcggaagcctgatgcagcaacgccgcgtgcgggatgacggccttcgggttgtaaaccgctttcgcctgtgacgaagcgtgagtgacggtaatgggtaaagaagcaccggctaactacgtgccagcagccgcggtgatacgtagggtgcgagcgttgtccggatttattgggcgtaaagggctcgtaggtggttgatcgcgtcggaagtgtaatcttggggcttaaccctgagcgtgctttcgatacgggttgacttgaggaaggtaggggagaatggaattcctggtggagcggtggaatgcgcagatatcaggaggaacaccagtggcgaaggcggttctctgggcctttcctgacgctgaggagcgaaagcgtggggagcgaacaggcttagataccctggtagtccacgctgtaaacggtgggtactaggtgtggggtccattccacgggttccgtgccgtagctaacgctttaagtaccccgcctggggagtacggccgcaaggctaaaactcaaaggaattgacggggccccgcacaagcggcggagcatgcggattaattcgatgcaacgcgtagaaccttacctgggtttgacatggatcgggagtgctcagagatgggtgtgcctcttttggggtcggttcacaggtggtgcatggctgtcgtcagctcgtgtcgtgagatgttgggttaagtcccgcaacgagcgcaacccctgttcactgttgccagcacgttatggtggggactcagtggagaccgccggggtcaactcggaggaaggtggggatgacgtcaagtcatcatgccccttatgtccagggcttcacgcatgctacaatggctggtacagagagtggcgagcctgtgagggtgagcgaatctcggaaagccggtctcagttcggattggggtctgcaactcgacctcatgaagtcggagtcgctagtaatcgcagatcagcaacgctgcggtgaatacgttcccggggcttgtacacaccgcccgtcaagtcatgaaagttggtaacacccgaagccggtggcctaaccgttgtgggggagccgtcgaaggtgggactggtgattaggactaagtcgtaacaaggtagccgtaccggaaggtgcggctggatcacctccttt

SEQ ID NO: 84 - DNA, 1522 bp, Propionibacterium acnes
  source 1..1522: mol_type = genomic DNA
ttggagagtttgatcctggctcaggacgaacgctggcggcgtgcttaacacatgcaagtcgaacggaaaggccctgcttttgtggggtgctcgagtggcgaacgggtgagtaacacgtgagtaacctgcccttgactttgggataacttcaggaaactggggctaataccggataggagctcctgctgcatggtgggggttggaaagtttcggcggttggggatggactcgcggcttatcagcttgttggtggggtagtggcttaccaaggctttgacgggtagccggcctgagagggtgaccggccacattgggactgagatacggcccagactcctacgggaggcagcagtggggaatattgcacaatgggcggaagcctgatgcagcaacgccgcgtgcgggatgacggccttcgggttgtaaaccgctttcgcctgtgacgaagcgtgagtgacggtaatgggtaaagaagcaccggctaactacgtgccagcagccgcggtgatacgtagggtgcgagcgttgtccggatttattgggcgtaaagggctcgtaggtggttgatcgcgtcggaagtgtaatcttggggcttaaccctgagcgtgctttcgatacgggttgacttgaggaaggtaggggagaatggaattcctggtggagcggtggaatgcgcagatatcaggaggaacaccagtggcgaaggcggttctctgggcctttcctgacgctgaggagcgaaagcgtggggagcgaacaggcttagataccctggtagtccacgctgtaaacggtgggtactaggtgtggggtccattccacgggttccgtgccgtagctaacgctttaagtaccccgcctggggagtacggccgcaaggctaaaactcaaaggaattgacggggccccgcacaagcggcggagcatgcggattaattcgatgcaacgcgtagaaccttacctgggtttgacatggatcgggagtgctcagagatgggtgtgcctcttttggggtcggttcacaggtggtgcatggctgtcgtcagctcgtgtcgtgagatgttgggttaagtcccgcaacgagcgcaacccttgttcactgttgccagcacgttatggtggggactcagtggagaccgccggggtcaactcggaggaaggtggggatgacgtcaagtcatcatgccccttatgtccagggcttcacgcatgctacaatggctggtacagagagtggcgagcctgtgagggtgagcgaatctcggaaagccggtctcagttcggattggggtctgcaactcgacctcatgaagtcggagtcgctagtaatcgcagatcagcaacgctgcggtgaatacgttcccggggcttgtacacaccgcccgtcaagtcatgaaagttggtaacacccgaagccggtggcctaaccgttgtgggggagccgtcgaaggtgggactggtgattaggactaagtcgtaacaaggtagccgtaccggaaggtgcggctggatcacctccttt

SEQ ID NO: 85 - DNA, 1519 bp, Propionibacterium avidum
  source 1..1519: mol_type = genomic DNA
ttggagagtttgatcctggctcaggacgaacgctggcggcgtgcttaacacatgcaagtcgaacggaaaggcccctttgggggtactcgagtggcgaacgggtgagtaacacgtgagtaacctgcccttgacttcgggataacttcaggaaactggggctaataccggataggaatccttgctgcatggtgggggttggaaagcttcggcggttttggatggactcgcggcttatcagcttgttggtggggtagtggcttaccaaggctttgacgggtagccggcctgagagggcgaccggccacattgggactgagatacggcccagactcctacgggaggcagcagtggggaatattgcacaatgggcggaagcctgatgcagcaacgccgcgtgcgggatgacggccttcgggttgtaaaccgctttcagcaggggcgaagcttttgtgacggtacctgcagaagaagcaccggctaactacgtgccagcagccgcggtgatacgtagggtgcgagcgttgtccggatttattgggcgtaaagagctcgtaggtggttgattgcgtcggaagtgaaaacttggggcttaaccctgagcgtgctttcgatacgggttgacttgaggaaggtaggggagaatggaattcctggtggagcggtggaatgcgcagatatcaggaggaacaccagtggcgaaggcggttctctggacctttcctgacgctgaggagcgaaagcgtggggagcgaacaggcttagataccctggtagtccacgctgtaaacggtgggtactaggtgtggggtccattccacggattccgtgccgtagctaacgcattaagtaccccgcctggggagtacggccgcaaggctaaaactcaaaggaattgacggggccccgcacaagcggcggagcatgcggattaattcgatgcaacgcgaagaaccttacctgggtttgacatggactgggagtgctcagagatgggtacgcctccttgtggggctggttcacaggtggtgcatggctgtcgtcagctcgtgtcgtgagatgttgggttaagtcccgcaacgagcgcaaccctcgttcactgttgccagcacgttatggtggggactcagtggagaccgccggggtcaactcggaggaaggtggggatgacgtcaagtcatcatgccccttatgtccagggcttcacgcatgctacaatggccggtacaaagagttgcgagcctgtgagggtgagcgaatctcggaaagccggtctcagttcggattggggtctgcaactcgaccctatgaagtcggagtcgctagtaatcgcagatcagcaacgctgcggtgaatacgttcccggggcttgtacacaccgcccgtcaagtcatgaaagtcggtaacacccgaagccggtggcctaacctgtgtgggggagccgtcgaaggtgggactggtaattaggactaagtcgtaacaaggtagccgtaccggaaggtgcggctggatcacctccttt

SEQ ID NO: 86 - DNA, 1519 bp, Propionibacterium avidum
  source 1..1519: mol_type = genomic DNA
ttggagagtttgatcctggctcaggacgaacgctggcggcgtgcttaacacatgcaagtcgaacggaaaggcccctttgggggtactcgagtggcgaacgggtgagtaacacgtgagtaacctgcccttgacttcgggataacttcaggaaactggggctaataccggataggaatccttgctgcatggtgggggttggaaagcttcggcggttttggatggactcgcggcttatcagcttgttggtggggtagtggcttaccaaggctttgacgggtagccggcctgagagggcgaccggccacattgggactgagatacggcccagactcctacgggaggcagcagtggggaatattgcacaatgggcggaagcctgatgcagcaacgccgcgtgcgggatgacggccttcgggttgtaaaccgctttcagcaggggcgaagcttttgtgacggtacctgcagaagaagcaccggctaactacgtgccagcagccgcggtgatacgtagggtgcgagcgttgtccggatttattgggcgtaaagagctcgtaggtggttgattgcgtcggaagtgaaaacttggggcttaaccctgagcgtgctttcgatacgggttgacttgaggaaggtaggggagaatggaattcctggtggagcggtggaatgcgcagatatcaggaggaacaccagtggcgaaggcggttctctggacctttcctgacgctgaggagcgaaagcgtggggagcgaacaggcttagataccctggtagtccacgctgtaaacggtgggtactaggtgtggggtccattccacggattccgtgccgtagctaacgcattaagtaccccgcctggggagtacggccgcaaggctaaaactcaaaggaattgacggggccccgcacaagcggcggagcatgcggattaattcgatgcaacgcgaagaaccttacctgggtttgacatggactgggagtgctcagagatgggtatgcctccttgtggggctggttcacaggtggtgcatggctgtcgtcagctcgtgtcgtgagatgttgggttaagtcccgcaacgagcgcaaccctcgttcactgttgccagcacgttatggtggggactcagtggagaccgccggggtcaactcggaggaaggtggggatgacgtcaagtcatcatgccccttatgtccagggcttcacgcatgctacaatggccggtacaaagagttgcgagcctgtgagggtgagcgaatctcggaaagccggtctcagttcggattggggtctgcaactcgaccctatgaagtcggagtcgctagtaatcgcagatcagcaacgctgcggtgaatacgttcccggggcttgtacacaccgcccgtcaagtcatgaaagtcggtaacacccgaagccggtggcctaacctgtgtgggggagccgtcgaaggtgggactggtaattaggactaagtcgtaacaaggtagccgtaccggaaggtgcggctggatcacctccttt

SEQ ID NO: 87 - DNA, 1549 bp, Bacillus flexus
  source 1..1549: mol_type = genomic DNA
tcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacaagagtaactgcttgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtcgtaacaaggtagccgtatcggaaggtgcggctggatcacctccttt

SEQ ID NO: 88 - DNA, 1549 bp, Bacillus flexus
  source 1..1549: mol_type = genomic DNA
tcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacgagagtaactgctcgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtcgtaacaaggtagccgtatcggaaggtgcggctggatcacctccttt

SEQ ID NO: 89 - DNA, 1548 bp, Bacillus muralis
  source 1..1548: mol_type = genomic DNA
tcggagagtttgatcctggctcaggacgaacgctggcggcgtgcctaatacatgcaagtcgagcgaatcgacgggagcttgctccctgagattagcggcggacgggtgagtaacacgtgggcaacctgcctataagactgggataacttcgggaaaccggagctaataccggatacgttcttttctcgcatgagagaagatggaaagacggtttacgctgtcacttatagatgggcccgcggcgcattagctagttggtgaggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgaacgaagaaggccttcgggtcgtaaagttctgttgttagggaagaacaagtaccagagtaactgctggtaccttgacggtacctaaccagaaagccacggctaactacgtgccagcagccgcggtaatacgtaggtggcaagcgttgtccggaattattgggcgtaaagcgcgcgcaggtggttccttaagtctgatgtgaaagcccacggctcaaccgtggagggtcattggaaactggggaacttgagtgcagaagaggaaagtggaattccaagtgtagcggtgaaatgcgtagagatttggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggctttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaaacctgcgaaggtaagcgaatcccataaagccattctcagttcggattgcaggctgcaactcgcctgcatgaagccggaatcgctagtaatcgcggatcagcatgccgcggtgaatacgttcccgggccttgtacacaccgcccgtcacaccacgagagtttgtaacacccgaagtcggtgaggtaaccttcatggagccagccgcctaaggtgggacagatgattggggtgaagtcgtaacaaggtagccgtatcggaaggtgcggctggatcacctccttt

SEQ ID NO: 90 - DNA, 1548 bp, Bacillus
  source 1..1548: mol_type = genomic DNA
cttggagagtttgatcctggctcaggacgaacgctggcggcgtgcctaatacatgcaagtcgagcgaatcttgaggtgcttgcacctcttggttagcggcggacgggtgagtaacacgtgggcaacctgcctgtaagactgggataacttcgggaaaccggagctaataccggataatccttttcctctcatgaggaaaagctgaaagtcggtttcggctgacacttacagatgggcccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagcgatgaaggccttcgggtcgtaaagctctgttgttagggaagaacaagtatcggagtaactgccggtaccttgacggtacctaaccagaaagccacggctaactacgtgccagcagccgcggtaatacgtaggtggcaagcgttgtccggaattattgggcgtaaagcgcgcgcaggcggtcctttaagtctgatgtgaaagcccacggctcaaccgtggagggtcattggaaactgggggacttgagtgcagaagaggaaagcggaattccacgtgtagcggtgaaatgcgtagagatgtggaggaacaccagtggcgaaggcggctttctggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacactcctagagataggacgttccccttcgggggacagagtgacaggtggtgcatggttgtcgtcagctcgtgtcgtgagatgttgggttaagtcccgcaacgagcgcaacccttgttcttagttgccagcattcagttgggcactctaaggagactgccggtgacaaaccggaggaaggtggggatgacgtcaaatcatcatgccccttatgacctgggctacacacgtgctacaatggatggtacaaagggctgcgagaccgcgaggtttagccaatcccataaaaccattctcagttcggattgtaggctgcaactcgcctacatgaagccggaatcgctagtaatcgcggatcagcatgccgcggtgaatacgttcccgggccttgtacacaccgcccgtcacaccacgagagtttgtaacacccgaagtcggtggggtaaccgtaaggagccagccgcctaaggtgggacagatgattggggtgaagtcgtaacaaggtagccgtatcggaaggtgcggctggatcacctccttt

SEQ ID NO: 91 - DNA, 1547 bp, Bacillus subtilis
  source 1..1547: mol_type = genomic DNA
tcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 92 - DNA, 1454 bp, Bacillus subtilis
  source 1..1454: mol_type = genomic DNA
tcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacct

SEQ ID NO: 93 - DNA, 1030 bp, Bacillus subtilis
  source 1..1030: mol_type = genomic DNA
gacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaatttttgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggc

SEQ ID NO: 94 - DNA, 1069 bp, Bacillus foraminis
  source 1..1069: mol_type = genomic DNA
ccttgacggtacctgaccagaaagccacggctaactacgtgccagcagccgcggtaatacgtaggtggcaagcgttgtccggaattattgggcgtaaagcgcgcgcaggcggtcctttaagtctgatgtgaaagcccacggctcaaccgtggagggtcattggaaactgggggacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggcgttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatagaacaaagggcagcgaagccgcgaggtgaagccaatcccataaatctattctcagttcggattgcaggctgcaactcgcctgcatgaagccggaatcgctagtaatcgcggatcagcatgccgcggtgaatacgttcccgggccttgtacacaccgcccgtcacaccacgagagtttgtaacacccgaagtcggtggggtaacctttatggggccagccgcctaaggtgggacagatgattggggtgaagtcgtaacaaggtagccgtatcggaaggtgcggctggatcacctccttt

SEQ ID NO: 95 - DNA, 1546 bp, Bacillus nealsonii
  source 1..1546: mol_type = genomic DNA
tcggagagtttgatcctggctcaggacgaacgctggcggcgtgcctaatacatgcaagtcgagcggacttaaaaagcttgctttttaagttagcggcggacgggtgagtaacacgtgggcaacctgcctgtaagactgggataacttcgggaaaccggagctaataccggataatccttttctactcatgtagaaaagctgaaagacggtttacgctgtcacttacagatgggcccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgttagggaagaacaagtacgagagtaactgctcgtaccttgacggtacctaaccagaaagccacggctaactacgtgccagcagccgcggtaatacgtaggtggcaagcgttgtccggaattattgggcgtaaagcgcgcgcaggcggtcctttaagtctgatgtgaaagcccacggctcaaccgtggagggtcattggaaactgggggacttgagtgcagaagagaagagtggaattccacgtgtagcggtgaaatgcgtagagatgtggaggaacaccagtggcgaaggcgactctttggtctgtaactgacgctgaggcgcgaaagcgtggggagcaaacaggattagataccctggtagtccacgccgtaaacgatgagtgctaagtgttagagggtttccgccctttagtgctgcagcaaacgcattaagcactccgcctggggagtacggccgcaaggctgaaactcaaaggaattgacgggggcccgcacaagcggtggagcatgtggtttaattcgaagcaacgcgaagaaccttaccaggtcttgacatctcctgacaatcctagagataggacgttccccttcgggggacaggatgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggcagcaaaaccgcgaggtcgagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaaccttttaggagccagccgcctaaggtgggatagatgattggggtgaagtcgtaacaaggtagccgtatcggaaggtgcggctggatcacctccttt

SEQ ID NO: 96 - DNA, 1560 bp, Terribacillus
  source 1..1560: mol_type = genomic DNA
catggagagtttgatcttggctcaggacgaacgctggcggcgtgcctaatacatgcaagtcgagcgcaggaagctagatgaccccttcggggtgattctagtggaatgagcggcggacgggtgagtaacacgtgggcaacctgcctgtaagactgggataactccgggaaaccggggctaataccggatagtattttctttctcctgattgaaaatggaaagacggtttcggctgtcacttacagatgggcccgcggtgcattagctagttggtggggtaatggcccaccaaggcgacgatgcatagccgacctgagagggtgatcggccacactgggactgagacacggcccagactcctacgggaggcagcagtagggaatcttccgcaatggacgaaagtctgacggagcaacgccgcgtgagcgatgaaggccttcgggtcgtaaagctctgttgtcagggaagaacaagtacgagagtaactgctcgtaccttgacggtacctgaccagaaagccccggctaactacgtgccagcagccgcggtaatacgtagggggcaagcgttgtccggaattattgggcgtaaagggcgtgtaggcggtttcttaagtctgatgtgaaagcccacagcttaactgtggagggtcattggaaactggggaacttgagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaggtgttagggggtttccgccccttagtgctgaagttaacgcattaagcactccgcctggggagtacggccgcaaggctgaaactcaaaagaattgacgggggcccgcacaagcggtggagcatgtggtttaattcgaagcaacgcgaagaaccttaccaggtcttgacatccgctgacaaccttggagacaaggcattcccttcggggacagcgtgacaggtggtgcatggttgtcgtcagctcgtgtcgtgagatgttgggttaagtcccgcaacgagcgcaacccttgattctagttgccagcattcagttgggcactctagagtgactgccggtgacaaaccggaggaaggtggggatgacgtcaaatcatcatgccccttatgacctgggctacacacgtgctacaatggatggtacaaagggcagcaagaccgcgaggttaagcaaatcccataaaaccattctcagttcggattgcaggctgcaactcgcctgcatgaagccggaatcgctagtaatcgcggatcagcatgccgcggtgaatacgttcccgggccttgtacacaccgcccgtcacaccacgagagttggtaacacccgaagtcggtgaggtaaccttttggagccagccgccgaaggtgggatcaatgattggggtgaagtcgtaacaaggtagccgtatcggaaggtgcggctggatcacctccttt

SEQ ID NO: 97 - DNA, 1044 bp, Chryseomicrobium imtechense
  source 1..1044: mol_type = genomic DNA
tcactacaggatgggcccgcggcgcattagctagttggtgaggtaacggctcaccaaggcgacgatgcgtagccgacctgagagggtgatcggccacactgggactgagacacggcccagactcctacgggaggcagcagtagggaatcttccacaatggacgaaagtctgatggagcaacgccgcgtgagtgaagaaggttctcggatcgtaaaactctgttgtgagggaagaacaagtaccggagtaactgccggtaccttgacggtacctcattagaaagccacggctaactacgtgccagcagccgcggtaatacgtaggtggcaagcgttgtccggaatcattgggcgtaaagcgcgcgcaggcggtcccttaagtctgatgtgaaagcccacggctcaaccgtggagggtcattggaaactgggggacttgagtgcagaagaggaaagcggaattccaagtgtagcggtgaaatgcgtagagatttggaggaacaccagtggcgaaggcggctttctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcccgccgaccgcctaggagactaggccttcccttcggggacggcggtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgc

SEQ ID NO: 98 - DNA, 1328 bp, Chryseomicrobium
  source 1..1328: mol_type = genomic DNA
tcactacaggatgggcccgcggcgcattagctagttggtgaggtaacggctcaccaaggccacgatgcgtagccgacctgagagggtgatcggccacactgggactgagacacggcccagactcctacgggaggcagcagtagggaatcttccacaatggacgaaagtctgatggagcaacgccgcgtgagtgaagaaggttctcggatcgtaaaactctgttgtgagggaagaacaagtaccggagtaactgccggtaccttgacggtacctcattagaaagccacggctaactacgtgccagcagccgcggtaatacgtaggtggcaagcgttgtccggaatcattgggcgtaaagcgcgcgcaggcggtcccttaagtctgatgtgaaagcccacggctcaaccgtggagggtcattggaaactgggggacttgagtgcagaagaggaaagcggaattccaagtgtagcggtgaaatgcgtagagatttggaggaacaccagtggcgaaggcggctttctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcccgccgaccgcctaggagactaggccttcccttcggggacggcggtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcgaacccgcgagggggagccaatcccataaaaccattctcagttcggattgtaggctgcaactcgcctacatgaagctggaatcgctagtaatcgtggatcagcatgccacggtgaatacgttcccgggccttgtacacaccgcccgtcacaccacgagagtttgtaacacccgaagtcggtggggtaacctttatggagccagccgccgaaggtgggacagatgattggggtgaagtcgtaacaaggtagccgtatcggaaggtgcggctggatcacctccttt

SEQ ID NO: 99 - DNA, 1549 bp, Sporosarcina
  source 1..1549: mol_type = genomic DNA
atggagagtttgatcctggctcaggacgaacgctggcggcatgcctaatacatgcaagtcgagcgaatcgataggagcttgctccttgagattagcggcggacgggtgagtaacacgtgggcaacctgccctgcagatggggataactccgggaaaccggggctaataccgaataatcaattccttcgcatgaaggaattctgaaagacggtttcggctgtcactgcaggatgggcccgcggcgcattagctagttggtggggtaatggcctaccaaggcaacgatgcgtagccgacctgagagggtgatcggccacactgggactgagacacggcccagactcctacgggaggcagcagtagggaatcttccacaatggacgaaagtctgatggagcaatgccgcgtgagtgaagaaggttttcggatcgtaaagctctgttgcgagggaagaacacgtacgggagtaactgcccgtaccttgacggtacctcgtcagaaagccacggctaactacgtgccagcagccgcggtaatacgtaggtggcaagcgttgtccggaattattgggcgtaaagcgcgcgcaggcggtcctttaagtctgatgtgaaagcccacggctcaaccgtggagggtcattggaaactgggggacttgagtacagaagaggaaagcggaattccacgtgtagcggtgaaatgcgtagagatgtggaggaacaccagtggcgaaggcggctttctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacggccgcaaggctgaaactcaaaggaattgacggggacccgcacaagcggtggagcatgtggtttaattcgaagcaacgcgaagaaccttaccaggtcttgacatcccactgaccggcatggagacatgtctttcccttcggggacagtggtgacaggtggtgcatggttgtcgtcagctcgtgtcgtgagatgttgggttaagtcccgcaacgagcgcaacccttaatcttagttgccatcatttagttgggcactctaaggtgactgccggtgacaaaccggaggaaggtggggatgacgtcaaatcatcatgccccttatgacctgggctacacacgtgctacaatggacggtacagagggctgcaaacccgcgagggggagccaatcccagaaaaccgttcccagttcggattgtaggctgcaactcgcctgcatgaagccggaatcgctagtaatcgtggatcagcatgccacggtgaatacgttcccgggtcttgtacacaccgcccgtcacaccacgagagtttgtaacacccgaagtcggtggggtaacccttacgggagccagccgccgaaggtgggacagatgattggggtgaagtcgtaacaaggtagccgtatcggaaggtgcggctggatcacctccttt

SEQ ID NO: 100 - DNA, 1550 bp, Sporosarcina
  source 1..1550: mol_type = genomic DNA
tatggagagtttgatcctggctcaggacgaacgctggcggcatgcctaatacatgcaagtcgagcgaatcaataggagcttgctccttgagattagcggcggacgggtgagtaacacgtgggcaacctgccctgcagatggggataactccgggaaaccggggctaataccgaataatcaattccttcgcatgaaggaattctgaaagacggtttcggctgtcactgcaggatgggcccgcggcgcattagctagttggtggggtaatggcctaccaaggcaacgatgcgtagccgacctgagagggtgatcggccacactgggactgagacacggcccagactcctacgggaggcagcagtagggaatcttccacaatggacgaaagtctgatggagcaatgccgcgtgagtgaagaaggttttcggatcgtaaagctctgttgcgagggaagaacacgtacgggagtaactgcccgtaccttgacggtacctcgtcagaaagccacggctaactacgtgccagcagccgcggtaatacgtaggtggcaagcgttgtccggaattattgggcgtaaagcgcgcgcaggcggtcctttaagtctgatgtgaaagcccacggctcaaccgtggagggtcattggaaactgggggacttgagtacagaagaggaaagcggaattccacgtgtagcggtgaaatgcgtagagatgtggaggaacaccagtggcgaaggcggctttctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacggccgcaaggctgaaactcaaaggaattgacggggacccgcacaagcggtggagcatgtggtttaattcgaagcaacgcgaagaaccttaccaggtcttgacatcccactgaccggcatggagacatgtctttcccttcggggacagtggtgacaggtggtgcatggttgtcgtcagctcgtgtcgtgagatgttgggttaagtcccgcaacgagcgcaacccttaatcttagttgccatcatttagttgggcactctaaggtgactgccggtgacaaaccggaggaaggtggggatgacgtcaaatcatcatgccccttatgacctgggctacacacgtgctacaatggacggtacagagggctgcaaacccgcgagggggagccaatcccagaaaaccgttcccagttcggattgtaggctgcaactcgcctgcatgaagccggaatcgctagtaatcgtggatcagcatgccacggtgaatacgttcccgggtcttgtacacaccgcccgtcacaccacgagagtttgtaacacccgaagtcggtggggtaacccttacgggagccagccgccgaaggtgggacagatgattggggtgaagtcgtaacaaggtagccgtatcggaaggtgcggctggatcacctccttt

SEQ ID NO: 101 - DNA, 1250 bp, Staphylococcus epidermidis
  source 1..1250: mol_type = genomic DNA
tatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatgga

SEQ ID NO: 102 - DNA, 1548 bp, Staphylococcus epidermidis
  source 1..1548: mol_type = genomic DNA
tatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggtagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctt

SEQ ID NO: 103 - DNA, 1110 bp, Staphylococcus epidermidis
  source 1..1110: mol_type = genomic DNA
tatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgatccctctagagatagaggtttccccttcgggggacagagtgacaggtggtgcatggttgtcgtcagctcgtgtcgtgagatgttgggttaagtcccgcaa

SEQ ID NO: 104 - DNA, 1548 bp, Staphylococcus epidermidis
  source 1..1548: mol_type = genomic DNA
tatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctt

SEQ ID NO: 105 - DNA, 1548 bp, Staphylococcus epidermidis
  source 1..1548: mol_type = genomic DNA
tatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgatccctctagagatagaggtttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggtagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctt

SEQ ID NO: 106 - DNA, 1138 bp, Staphylococcus epidermidis
  source 1..1138: mol_type = genomic DNA
tatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgcca

SEQ ID NO: 107 - DNA, 1548 bp, Staphylococcus epidermidis
  source 1..1548: mol_type = genomic DNA
tatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgatccctctagagatagaggtttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctt

SEQ ID NO: 108 - DNA, 1549 bp, Staphylococcus epidermidis
  source 1..1549: mol_type = genomic DNA
tatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccagataacatgttgaaccgcatggttcaacagtgaaagacggtcttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggcacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaaccccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccttttggagctagccgtcgaaggtgggacaaatgattggggtgaagtcgtaacaaggtagccgtatcggaaggtgcggctggatcacctccttt

SEQ ID NO: 109 - DNA, 1548 bp, Staphylococcus epidermidis
  source 1..1548: mol_type = genomic DNA
tatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataacatgttgaaccgcatggttcaacagtgaaagacggttttgctgtcacttatagatggatccgcgtcgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgaagaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggtagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttggagctagccgtcgaaggtgggacaaatgattggggtgaagtcgtaacaaggtagccgtatcggaaggtgcggctggatcacctcctt

SEQ ID NO: 110 - DNA, 1548 bp, Staphylococcus epidermidis
  source 1..1548: mol_type = genomic DNA
tatggagagtttgatcctggctcaggatgaacgctggcggcgtgcctaatacatgcaagtcgagcgaacagacgaggagcttgctcctttgacgttagcggcggacgggtgagtaacacgtggataacctacctataagactgggataacttcgggaaaccggagctaataccggataacatgttgaaccgcatggttcaacagtgaaagacggtcttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgaagaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgatccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggtagcaaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttggagctagccgtcgaaggtgggacaaatgattggggtgaagtcgtaacaaggtagccgtatcggaaggtgcggctggatcacctcctt

SEQ ID NO: 111 - DNA, 1265 bp, Staphylococcus epidermidis
  source 1..1265: mol_type = genomic DNA
cgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggtagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctccttt

SEQ ID NO: 112 - DNA, 1548 bp, Staphylococcus haemolyticus
  source 1..1548: mol_type = genomic DNA
tatggagagtttgatcctggctcaggatgaacgctggcggcgtgcctaatacatgcaagtcgagcgaacagacaaggagcttgctcctttgacgttagcggcggacgggtgagtaacacgtgggtaacctacctataagactgggataacttcgggaaaccggagctaataccggataatatttcgaaccgcatggttcgatagtgaaagatggttttgctatcacttatagatggacccgcgccgtattagctagttggtaaggtaacggcttaccaaggcgacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatacgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctt

SEQ ID NO: 113 - DNA, 1110 bp, Staphylococcus hominis
  source 1..1110: mol_type = genomic DNA
tatggagagtttgatcctggctcaggatgaacgctggcggcgtgcctaatacatgcaagtcgagcgaacagacgaggagcttgctcctttgatgttagcggcggacgggtgagtaacacgtaggtaacctacctataagactgggataacttcgggaaaccggagctaataccggataatatttcgaaccgcatggttcgatagtgaaagatggctttgctatcacttatagatggacctgcgccgtattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaacgtgtaagtaactgtgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacccttctagagatagaagtttccccttcgggggacaaagtgacaggtggtgcatggttgtcgtcagctcgtgtcgtgagatgttgggttaagtcccgcaa

SEQ ID NO: 114 - DNA, 1548 bp, Staphylococcus hominis
  source 1..1548: mol_type = genomic DNA
tatggagagtttgatcctggctcaggatgaacgctggcggcgtgcctaatacatgcaagtcgagcgaacagacgaggagcttgctcctttgacgttagcggcggacgggtgagtaacacgtaggtaacctacctataagactgggataacttcgggaaaccggagctaataccggataatatttcgaaccgcatggttcgatagtgaaagatggctttgctatcacttatagatggacctgcgccgtattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaacgtgtaagtaactgtgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacccttctagagatagaagtttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctt

SEQ ID NO: 115 - DNA, 1548 bp, Staphylococcus hominis
  source 1..1548: mol_type = genomic DNA
tatggagagtttgatcctggctcaggatgaacgctggcggcgtgcctaatacatgcaagtcgagcgaacagacgaggagcttgctcctttgacgttagcggcggacgggtgagtaacacgtaggtaacctacctataagactgggataacttcgggaaaccggagctaataccggataatatttcgaaccgcatggttcgatagtgaaagatggctttgctatcacttatagatggacctgcgccgtattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaacgtgtaagtaactgtgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacccttctagagatagaagtttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatgggcaatacaaagggtagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctt

SEQ ID NO: 116 - DNA, 1548 bp, Staphylococcus lugdunensis
  source 1..1548: mol_type = genomic DNA
tatggagagtttgatcctggctcaggatgaacgctggcggcgtgcctaatacatgcaagtcgagcgaacagataaggagcttgctcctttgacgttagcggcggacgggtgagtaacacgtgggtaacctacctataagactgggacaacttcgggaaaccggagctaataccggataatatgttgaaccgcatggttcaatagtgaaagatggttttgctatcacttatagatggacccgcgccgtattagctagttggtgaggtaacggctcaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttaggatcgtaaaactctgttattagggaagaacaaacgtgtaagtaactgtgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgaccgctctagagatagagtcttccccttcgggggacaaagtgacaggtggtgcatggttgtcgtcagctcgtgtcgtgagatgttgggttaagtcccgcaacgagcgcaacccttaagcttagttgccatcatttagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctt

SEQ ID NO: 117 - DNA, 1008 bp, Staphylococcus lugdunensis
  source 1..1008: mol_type = genomic DNA
tatggagagtttgatcctggctcaggatgaacgctggcggcgtgcctaatacatgcaagtcgagcgaacagataaggagcttgctcctttgacgttagcggcggacgggtgagtaacacgtgggtaacctacctataagactgggacaacttcgggaaaccggagctaataccggataatatgttgaaccgcatggttcaatagtgaaagatggttttgctatcacttatagatggacccgcgccgtattagctagttggtgaggtaacggctcaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttaggatcgtaaaactctgttattagggaagaacaaacgtgtaagtaactgtgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatccttt

SEQ ID NO: 118 - DNA, 1548 bp, Staphylococcus lugdunensis
  source 1..1548: mol_type = genomic DNA
tatggagagtttgatcctggctcaggatgaacgctggcggcgtgcctaatacatgcaagtcgagcgaacagataaggagcttgctcctttgacgttagcggcggacgggtgagtaacacgtgggtaacctacctataagactgggacaacttcgggaaaccggagctaataccggataatatgttgaaccgcatggttcaatagtgaaagatggttttgctatcacttatagatggacccgcgccgtattagctagttggtgaggtaacggctcaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttaggatcgtaaaactctgttattagggaagaacaaacgtgtaagtaactgtgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgaccgctctagagatagagtcttccccttcgggggacaaagtgacaggtggtgcatggttgtcgtcagctcgtgtcgtgagatgttgggttaagtcccgcaacgagcgcaacccttaagcttagttgccatcatttagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccattcggagctagccgtcgaaggtgggacaaatgattggggtgaagtcgtaacaaggtagccgtatcggaaggtgcggctggatcacctcctt

SEQ ID NO: 119 - DNA, 1548 bp, Staphylococcus lugdunensis
  source 1..1548: mol_type = genomic DNA
tatggagagtttgatcctggctcaggatgaacgctggcggcgtgcctaatacatgcaagtcgagcgaacagataaggagcttgctcctttgacgttagcggcggacgggtgagtaacacgtgggtaacctacctataagactgggacaacttcgggaaaccggagctaataccggataatatgttgaaccgcatggttcaatagtgaaagatggttttgctatcacttatagatggacccgcgccgtattagctagttggtgaggtaacggctcaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttaggatcgtaaaactctgttattagggaagaacaaacgtgtaagtaactgtgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgaccactctagagatagagttttccccttcgggggacaaagtgacaggtggtgcatggttgtcgtcagctcgtgtcgtgagatgttgggttaagtcccgcaacgagcgcaacccttaagcttagttgccatcatttagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctt

SEQ ID NO: 120 - DNA, 1008 bp, Staphylococcus lugdunensis
  source 1..1008: mol_type = genomic DNA
tatggagagtttgatcctggctcaggatgaacgctggcggcgtgcctaatacatgcaagtcgagcgaacagataaggagcttgttcctttgacgttagcggcggacgggtgagtaacacgtgggtaacctacctataagactgggacaacttcgggaaaccggagctaataccggataatatgttgaaccgcatggttcaatagtgaaagatggttttgctatcacttatagatggacccgcgccgtattagctagttggtgaggtaacggctcaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttaggatcgtaaaactctgttattagggaagaacaaacgtgtaagtaactgtgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatccttt

SEQ ID NO: 121 - DNA, 1275 bp, Staphylococcus lugdunensis
  source 1..1275: mol_type = genomic DNA
accaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttaggatcgtaaaactctgttattagggaagaacaaacgtgtaagtaactgtgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgaccgctctagagatagagtcttccccttcgggggacaaagtgacaggtggtgcatggttgtcgtcagctcgtgtcgtgagatgttgggttaagtcccgcaacgagcgcaacccttaagcttagttgccatcatttagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctccttt

SEQ ID NO: 122 - DNA, 1227 bp, Staphylococcus lugdunensis
  source 1..1227: mol_type = genomic DNA
tggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaacgtgtaagtaactgtgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacccttctagagatagaagtttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctt

SEQ ID NO: 123 - DNA, 1550 bp, Staphylococcus succinus
  source 1..1550: mol_type = genomic DNA
tatggagagtttgatcctggctcaggatgaacgctggcggcgtgcctaatacatgcaagtcgagcgaacggataaggagcttgctcctttgaagttagcggcggacgggtgagtaacacgtgggtaacctacctataagactggaataacttcgggaaaccggagctaatgccggataacatatagaaccgcatggttctatagtgaaagatggttttgctatcacttatagatggacccgcgccgtattagctagttggtaaggtaatggcttaccaaggcgacgatacgtagccgacctgagagggtgatcggccacactggaactgagacacggtccagactcctacgggaggcagcagtagggaatcttccgcaatgggcgaaagcctgacggagcaacgccgcgtgagtgatgaaggttttcggatcgtaaaactctgttattagggaagaacaaatgcgtaagtaactgtgcgcatcttgacggtacctaatcagaaagccacggctaactacgtgccagcagccgcggtaatacgtaggtggcaagcgttatccggaattattgggcgtaaagcgcgcgtaggcggtttcttaagtctgatgtgaaagcccacggctcaaccgtggagggtcattggaaactgggaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgaaaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaggttgactgccggtgacaaaccggaggaaggtggggatgacgtcaaatcatcatgccccttatgatttgggctacacacgtgctacaatggacaatacaaagggcagctaaaccgcgaggtcatgcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccatttatggagctagccgtcgaaggtgggacaaatgattggggtgaagtcgtaacaaggtagccgtatcggaaggtgcggctggatcacctcctt

SEQ ID NO: 124 - DNA, 1261 bp, Staphylococcus succinus
  source 1..1261: mol_type = genomic DNA
gtagccgacctgagagggtgatcggccacactggaactgagacacggtccagactcctacgggaggcagcagtagggaatcttccgcaatgggcgaaagcctgacggagcaacgccgcgtgagtgatgaaggttttcggatcgtaaaactctgttattagggaagaacaaatgcgtaagtaactgtgcgcatcttgacggtacctaatcagaaagccacggctaactacgtgccagcagccgcggtaatacgtaggtggcaagcgttatccggaattattgggcgtaaagcgcgcgtaggcggtttcttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgaaaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccatttatggagctagccgtcgaaggtgggacaaatgattggggtgaagtcgtaacaaggtagccgtatcggaaggtgcggctggatcacctccttt

SEQ ID NO: 125 - DNA, 1551 bp, Staphylococcus succinus
  source 1..1551: mol_type = genomic DNA
tatggagagtttgatcctggctcaggatgaacgctggcggcgtgcctaatacatgcaagtcgagcgaacagataaggagcttgctcctttgaagttagcggcggacgggtgagtaacacgtgggtaacctacctataagactggaataacttcgggaaaccggagctaatgccggataacatatagaaccgcatggttctatagtgaaagatggttttgctatcacttatagatggacccgcgccgtattagctagttggtaaggtaatggcttaccaaggcgacgatacgtagccgacctgagagggtgatcggccacactggaactgagacacggtccagactcctacgggaggcagcagtagggaatcttccgcaatgggcgaaagcctgacggagcaacgccgcgtgagtgatgaaggttttcggatcgtaaaactctgttattagggaagaacaaatgcgtaagtaactgtgcgcatcttgacggtacctaatcagaaagccacggctaactacgtgccagcagccgcggtaatacgtaggtggcaagcgttatccggaattattgggcgtaaagcgcgcgtaggcggtttcttaagtctgatgtgaaagcccacggctcaaccgtggagggtcattggaaactgggaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgaaaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaggttgactgccggtgacaaaccggaggaaggtggggatgacgtcaaatcatcatgccccttatgatttgggctacacacgtgctacaatggacaatacaaagggcagctaaaccgcgaggtcatgcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccattttatggagctagccgtcgaaggtgggacaaatgattggggtgaagtcgtaacaaggtagccgtatcggaaggtgcggctggatcacctcctt

SEQ ID NO: 126 - DNA, 1138 bp, Staphylococcus
  source 1..1138: mol_type = genomic DNA
tatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatgttgaaccgcatggttcaatagtgaaagatggttttgctatcacttatagatggacccgcgccgtattagctagttggtgaggtaacggctcaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgcca

SEQ ID NO: 127 - DNA, 1018 bp, Exiguobacterium mexicanum
  source 1..1018: mol_type = genomic DNA
tttggagagtttgatcctggctcaggacgaacgctggcggcgtgcctaatacatgcaagtcgagcgcaggaagtcatccgaacccttcggggggacgttgacggaatgagcggcggacgggtgagtaacacgtaaagaacctgccctcaggtctgggataaccacgagaaatcggggctaataccggatgggtcatcggaccgcatggtccgaggatgaaaggcgcttcggcgtcgcctggggatggctttgcggtgcattagctagttggtggggtaacggcccaccaaggcgacgatgcatagccgacctgagagggtgatcggccacactgggactgagacacggcccagactcctacgggaggcagcagtagggaatcttccacaatggacgaaagtctgatggagcaacgccgcgtgaacgatgaaggccctcgggtcgtaaagttctgttgtaagggaagaacaagtgccgcaggcaatggcggcaccttgacggtaccttgcgagaaagccacggctaactacgtgccagcagccgcggtaatacgtaggtggcaagcgttgtccggaattattgggcgtaaagcgcgcgcaggcggcctcttaagtctgatgtgaaagcccccggctcaaccggggagggccattggaaactgggaggcttgagtataggagagaagagtggaattccacgtgtagcggtgaaatgcgtagagatgtggaggaacaccagtggcgaaggcgactctttggcctataactgacgctgaggcgcgaaagcgtggggagcaaacaggattagataccctggtagtccacgccgtaaacgatgagtgctaggtgttggagggtttccgcccttcagtgctgaagctaacgcattaagcactccgcctggggagtacggtcgcaaggctgaaactcaaaggaattgacggggacccgcacaagcggtggagcatgtggtttaattcgaagcaacgcgaagaaccttaccaactcttgacatcccc

SEQ ID NO: 128 - DNA, 1321 bp, Exiguobacterium profundum
  source 1..1321: mol_type = genomic DNA
atggatggctttgcggtgcattagctagttggtggggtaacggcccaccaaggcgacgatgcatagccgacctgagagggtgatcggccacactgggactgagacacggcccagactcctacgggaggcagcagtagggaatcttccacaatggacgaaagtctgatggagcaacgccgcgtgaacgatgaaggctttcgggtcgtaaagttctgttgtaagggaagaacaagtgccgcaggcaatggcggcaccttgacggtaccttgcgagaaagccacggctaactacgtgccagcagccgcggtaatacgtaggtggcaagcgttgtccggaattattgggcgtaaagcgcgcgcaggcggcctcttaagtctgatgtgaaagcccccggctcaaccggggagggccattggaaactgggaggcttgagtataggagagaagagtggaattccacgtgtagcggtgaaatgcgtagagatgtggaggaacaccagtggcgaaggcgactctttggcctataactgacgctgaggcgcgaaagcgtggggagcaaacaggattagataccctggtagtccacgccgtaaacgatgagtgctaggtgttggagggtttccgcccttcagtgctgaagctaacgcattaagcactccgcctggggagtacggtcgcaaggctgaaactcaaaggaattgacggggacccgcacaagcggtggagcatgtggtttaattcgaagcaacgcgaagaaccttaccaactcttgacatccccctgaccggtacagagatgtaccttccccttcgggggcaggggtgacaggtggtgcatggttgtcgtcagctcgtgtcgtgagatgttgggttaagtcccgcaacgagcgcaacccttgtccttagttgccagcatttggttgggcactctagggagactgccggtgacaaaccggaggaaggtggggatgacgtcaaatcatcatgccccttatgagttgggctacacacgtgctacaatggacggtacaaagggcagcgaagccgcgaggtggagccaatcccagaaagccgttctcagttcggattgcaggctgcaactcgcctgcatgaagtcggaatcgctagtaatcgcaggtcagcatactgcggtgaatacgttcccgggtcttgtacacaccgcccgtcacaccacgagagtttgcaacacccgaagtcggtgaggtaaccgtaaggagccagccgccgaaggtggggcagatgattggggtgaagtcgtaacaaggtagccgtatcggaaggtgcggctggatcacctccttt

SEQ ID NO: 129 - DNA, 1018 bp, Exiguobacterium
  source 1..1018: mol_type = genomic DNA
tttggagagtttgatcctggctcaggacgaacgctggcggcgtgcctaatacatgcaagtcgagcgcaggaagtcatccgaacccttcggggggacgttgacggaatgagcggcggacgggtgagtaacacgtaaagaacctgccctcaggtctgggataaccacgagaaatcggggctaataccggatgggtcatcggaccgcatggtccgaggatgaaaggcgctccggcgtcgcctggggatggctttgcggtgcattagctagttggtggggtaacggcccaccaaggcgacgatgcatagccgacctgagagggtgatcggccacactgggactgagacacggcccagactcctacgggaggcagcagtagggaatcttccacaatggacgaaagtctgatggagcaacgccgcgtgaacgatgaaggccctcgggtcgtaaagttctgttgtaagggaagaacaagtgccgcaggcaatggcggcaccttgacggtaccttgcgagaaagccacggctaactacgtgccagcagccgcggtaatacgtaggtggcaagcgttgtccggaattattgggcgtaaagcgcgcgcaggcggcctcttaagtctgatgtgaaagcccccggctcaaccggggagggccattggaaactgggaggcttgagtataggagagaagagtggaattccacgtgtagcggtgaaatgcgtagagatgtggaggaacaccagtggcgaaggcgactctttggcctataactgacgctgaggcgcgaaagcgtggggagcaaacaggattagataccctggtagtccacgccgtaaacgatgagtgctaggtgttggagggtttccgcccttcagtgctgaagctaacgcattaagcactccgcctggggagtacggtcgcaaggctgaaactcaaaggaattgacggggacccgcacaagcggtggagcatgtggtttaattcgaagcaacgcgaagaaccttaccaactcttgacatcccc

SEQ ID NO: 130 - DNA, 1107 bp, Aerococcus viridans
  source 1..1107: mol_type = genomic DNA
catgagagtttgatcctggctcaggacgaacgctggcggcatgcctaatacatgcaagtcgagcgaacagatgaagtgcttgcacttctgacgttagcggcgaacgggtgagtaacacgtaaggaatctacctataagcgggggataacattcggaaacgggtgctaataccgcataatattttcttccgcatggaagaaaattgaaagacggctctgctgtcacttatagatgaccttgcggtgcattagttagttggtggggtaatggcctaccaagacgatgatgcatagccgacctgagagggtgatcggccacattgggactgagacacggcccaaactcctacgggaggcagcagtagggaatcttccgcaatgggcgaaagcctgacggagcaatgccgcgtgagtgaagaaggccttcgggtcgtaaaactctgttataagagaagaacaaattgtagagtaactgctacagtcttgacggtatcttatcagaaagccacggctaactacgtgccagcagccgcggtaatacgtaggtggcaagcgttgtccggatttattgggcgtaaagggagcgcaggtggtttcttaagtctgatgtgaaagcccacggcttaaccgtggagggtcattggaaactgggaaacttgagtacagaagaggaatgtggaactccatgtgtagcggtggaatgcgtagatatatggaagaacaccagtggcgaaggcgacattctggtctgttactgacactgaggctcgaaagcgtggggagcaaacaggattagataccctggtagtccacgccgtaaacgatgagtgctaggtgttggagggtttccgcccttcagtgccgcagttaacgcattaagcactccgcctggggagtacgaccgcaaggttgaaactcaaaggaattgacggggacccgcacaagcggtggagcatgtggtttaattcgaagcaacgcgaagaaccttaccaagtcttgacatcctttgaccaccctagagatagggctttcccttcggggacaaagtgacaggtggtgcatggttgtcgtcagctcgtgtcgtgagatgttgggttaagtcccgcaa

SEQ ID NO: 131 - DNA, 1558 bp, Enterococcus faecalis
  source 1..1558: mol_type = genomic DNA
tatgagagtttgatcctggctcaggacgaacgctggcggcgtgcctaatacatgcaagtcgaacgcttctttcctcccgagtgcttgcactcaattggaaagaggagtggcggacgggtgagtaacacgtgggtaacctacccatcagagggggataacacttggaaacaggtgctaataccgcataacagtttatgccgcatggcataagagtgaaaggcgctttcgggtgtcgctgatggatggacccgcggtgcattagctagttggtgaggtaacggctcaccaaggccacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggacgttagtaactgaacgtcccctgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgctagaccgcgaggtcatgcaaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgattggggtgaagtcgtaacaaggtagccgtatcggaaggtgcggctggatcacctccttt

SEQ ID NO: 132 - DNA, 1543 bp, Streptococcus infantis
  source 1..1543: mol_type = genomic DNA
aatgagagtttgatcctggctcaggacgaacgctggcggcgtgcctaatacatgcaagtagaacgctgaaggaggagcttgcttctctggatgagttgcgaacgggtgagtaacgcgtaggtaacctgcctggtagcgggggataactattggaaacgatagctaataccgcataaaattgattattgcatgatagtcaattaaaaggtgcaattgcatcactaccagatggacctgcgttgtattagctagttggtggggtaacggctcaccaaggcaacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgccgtaaacgatgagtgctaggtgttagaccctttccggggtttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttaggagccagccgcctaaggtgggatagatgattggggtgaagtcgtaacaaggtagccgtatcggaaggtgcggctggatcacctcctt

SEQ ID NO: 133 - DNA, 1543 bp, Streptococcus infantis
  source 1..1543: mol_type = genomic DNA
aatgagagtttgatcctggctcaggacgaacgctggcggcgtgcctaatacatgcaagtagaacgctgaaggaggagcttgcttctccggatgagttgcgaacgggtgagtaacgcgtaggtaacctgcctggtagcgggggataactattggaaacgatagctaataccgcataacagtagatatcgcatgataactgcttaaaaggtgcaattgcatcactaccagatggacctgcgttgtattagctagttggtggggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgccgtaaacgatgagtgctaggtgttagaccctttccggggtttagtgccgtagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggttggtacaacgagtcgcaagccggtgacggcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttaggagccagccgcctaaggtgggatagatgattggggtgaagtcgtaacaaggtagccgtatcggaaggtgcggctggatcacctcctt

SEQ ID NO: 134 - DNA, 1542 bp, Streptococcus infantis
  source 1..1542: mol_type = genomic DNA
aatgagagtttgatcctggctcaggacgaacgctggcggcgtgcctaatacatgcaagtagaacgctgaaggaggagcttgcttctctggatgagttgcgaacgggtgagtaacgcgtaggtaacctgcctggtagcgggggataactattggaaacgatagctaataccgcataaaattgattattgcatgatagtcaattaaaaggtgcaattgcatcactaccagatggacctgcgttgtattagctagttggtgaggtaacggctcaccaaggcgacgatacatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgccgtaaacgatgagtgctaggtgttagaccctttccggggtttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccatttggagccagccgcctaaggtgggatagatgattggggtgaagtcgtaacaaggtagccgtatcggaaggtgcggctggatcacctcctt

SEQ ID NO: 135 - DNA, 1543 bp, Streptococcus infantis
  source 1..1543: mol_type = genomic DNA
aatgagagtttgatcctggctcaggacgaacgctggcggcgtgcctaatacatgcaagtagaacgctgaaggaggagcttgcttctctggatgagttgcgaacgggtgagtaacgcgtaggtaacctgcctggtagcgggggataactattggaaacgatagctaataccgcataaaattgattattgcatgatagtcaattaaaaggtgcaattgcatcactaccagatggacctgcgttgtattagctagttggtgaggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgccgtaaacgatgagtgctaggtgttagaccctttccggggtttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcatttagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttaggagccagccgcctaaggtgggatagatgattggggtgaagtcgtaacaaggtagccgtatcggaaggtgcggctggatcacctcctt

SEQ ID NO: 136 - DNA, 1452 bp, Streptococcus oralis
  source 1..1452: mol_type = genomic DNA
tgagttgcgaacgggtgagtaacgcgtaggtaacctgcctggtagcgggggataactattggaaacgatagctaataccgcataagagtagatgttgcatgacatttgcttaaaaggtgcaattgcatcactaccagatggacctgcgttgtattagctagttggtggggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgccgtaaacgatgagtgctaggtgttagaccctttccggggtttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtagccgtatcggaaggtgcggctggatcacctccttt

SEQ ID NO: 137 - DNA, 1225 bp, Streptococcus pneumoniae
  source 1..1225: mol_type = genomic DNA
aatgagagtttgatcctggctcaggacgaacgctggcggcgtgcctaatacatgcaagtagaacgctgaaggaggagcttgctcttctggatgagttgcgaacgggtgagtaacgcgtaggtaacctgcctggtagcgggggataactattggaaacgatagctaataccgcataagagtagatgttgcatgacatttgcttaaaaggtgcaattgcatcactaccagatggacctgcgttgtattagctagttggtggggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgccgtaaacgatgagtgctaggtgttagaccctttccggggtttagtgccgtagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggct

SEQ ID NO: 138 - DNA, 1284 bp, Streptococcus pneumoniae
  source 1..1284: mol_type = genomic DNA
ggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtatgctttggaaactgtttaacttgagtgcagaaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgccgtaaacgatgagtgctaggtgttagaccctttccggggtttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtagccgtatcggaaggtgcggctggatcacctccttt

SEQ ID NO: 139 - DNA, 1544 bp, Streptococcus sanguinis
  source 1..1544: mol_type = genomic DNA
aatgagagtttgatcctggctcaggacgaacgctggcggcgtgcctaatacatgcaagtagaacgctgaagagaggagcttgctcttcttggatgagttgcgaacgggtgagtaacgcgtaggtaacctgcctggtagcgggggataactattggaaacgatagctaataccgcataaaattgattattgcatgataattaattgaaagatgcaattgcatcactaccagatggacctgcgttgtattagctagttggtgaggtaacggctcaccaaggcgacgatacatagccgacctgagagggtgatcggccacactgggactgagacacggcccagactcctacgggaggcagcagtagggaatcttcggcaatggggggaaccctgaccgagcaacgccgcgtgagtgaagaaggttttcggatcgtaaagctctgttgtaagagaagaacgggtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtatgctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctctgaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtagccgtatcggaaggtgcggctggatcacctcctt

SEQ ID NO: 140 - DNA, 1544 bp, Streptococcus vestibularis
  source 1..1544: mol_type = genomic DNA
aatgagagtttgatcctggctcaggacgaacgctggcggcgtgcctaatacatgcaagtagaacgctgaagagaggagcttgctcttcttggatgagttgcgaacgggtgagtaacgcgtaggtaacctgccttgtagcgggggataactattggaaacgatagctaataccgcataacaatggatgacacatgtcatttatttgaaaggggcaattgctccactacaagatggacctgcgttgtattagctagtaggtgaggtaacggctcacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagtcaagaacgagtgtgagagtggaaagttcacactgtgacggtagcttaccagaaagggacggctaactacgtgccagcagccgcggtaatacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttggatcctttccgggattcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatttctagagatagaaagttacttcggtacatcggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggttggtacaacgagttgcgagtcggtgacggcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatcggaaggtgcggctggatcacctcctt

SEQ ID NO: 141 - DNA, 1106 bp, Streptococcus vestibularis
  source 1..1106: mol_type = genomic DNA
aatgagagtttgatcctggctcaggacgaacgctggcggcgtgcctaatacatgcaagtagaacgctgaagagaggagcttgctcttcttggatgagttgcgaacgggtgagtaacgcgtaggtaacctgccttgtagcgggggataactattggaaacgatagctaataccgcataacaatgggtgacacatgtcatttatttgaaaggggcaattgctccactacaagatggacctgcgttgtattagctagtaggtgaggtaacggctcacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagtcaagaacgagtgtgagagtggaaagttcacactgtgacggtagcttaccagaaagggacggctaactacgtgccagcagccgcggtaatacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttggatcctttccgggattcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatttctagagatagaaagttacttcggtacatcggtgacaggtggtgcatggttgtcgtcagctcgtgtcgtgagatgttgggttaagtcccgcaa

SEQ ID NO: 142 - DNA, 1546 bp, Assigned species1791
  source 1..1546: mol_type = genomic DNA
taatgagagtttgatcctggctcaggatgaacgctggcggcgtgcctaatacatgcaagtagaacgctgaagcttggtgcttgcaccgagcggatgagttgcgaacgggtgagtaacgcgtaggtaacctgcctggtagcgggggataactattggaaacgatagctaataccgcataacagtagatattgcatgatatctgcttgaaaggggcaattgctccactaccagatggacctgcgttgtattagctagttggtgaggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtaacttaccagaaagggacggctaactacgtgccagcagccgcggtaatacgtaggtcccgagcgttatccggatttattgggcgtaaagcgagcgcaggcggttagataagtctgaagttaaaggctgtggcttaaccatagtacgctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttgggtcctttccgggactcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattgagttgggcactctagcgagactgccggtaataaaccggaggaaggtggggatgacgtcaaatcatcatgccccttatgacctgggctacacacgtgctacaatggctggtacaacgagtcgcaagtcggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttaggagccagccgcctaaggtgggatagatgattggggtgaagtcgtaacaaggtagccgtatcggaaggtgcggctggatcacctcctt

SEQ ID NO: 143 - DNA, 1460 bp, Paracoccus aminovorans
  source 1..1460: mol_type = genomic DNA
cttgagagtttgatcctggctcagaacgaacgctggcggcaggcctaacacatgcaagtcgagcgaggacttcggttctagcggcggacgggtgagtaacgcgtgggaatgtgcccttctctacggaatagccctgggaaactgggagtaataccgtatacgccctattggggaaagatttatcggagaaggatcagcccgcgttggattaggtagttggtggggtaatggcctaccaagccgacgatccatagctggtttgagaggatgatcagccacactgggactgagacacggcccagactcctacgggaggcagcagtggggaatcttagacaatgggggcaaccctgatctagccatgccgcgtgagtgatgaaggccttagggttgtaaagctctttcagctgggaagataatgacggtaccagcagaagaagccccggctaactccgtgccagcagccgcggtaatacggagggggctagcgttgttcggaattactgggcgtaaagcgcacgtaggcggaccggaaagtcagaggtgaaatcccagggctcaaccttggaactgcctttgaaactatcggtctggagttcgagagaggtgagtggaattccgagtgtagaggtgaaattcgtagatattcggaggaacaccagtggcgaaggcggctcactggctcgatactgacgctgaggtgcgaaagcgtggggagcaaacaggattagataccctggtagtccacgccgtaaacgatgaatgccagtcgtcgggcagcatgctgttcggtgacacacctaacggattaagcattccgcctggggagtacggtcgcaagattaaaactcaaaggaattgacgggggcccgcacaagcggtggagcatgtggtttaattcgaagcaacgcgcagaaccttaccaacccttgacatcgcaggacagcccgagagatcgggtcttctcgtaagagacctgtggacaggtgctgcatggctgtcgtcagctcgtgtcgtgagatgttcggttaagtccggcaacgagcgcaacccacgtctttagttgccagcattcagttgggcactctaaagaaactgccgatgataagtcggaggaaggtgtggatgacgtcaagtcctcatggcccttacgggttgggctacacacgtgctacaatggtggtgacagtgggttaatccccaaaagccatctcagttcggattggggtctgcaactcgaccccatgaagttggaatcgctagtaatcgcggaacagcatgccgcggtgaatacgttcccgggccttgtacacaccgcccgtcacaccatgggagttgggtctacccgacggccgtgcgccaaccagcaatggaggcagcggaccacggtaggctcagcgactggggtgaagtcgtaacaaggtagccgtaggggaacctgcggctggatcacctccttt

SEQ ID NO: 144 - DNA, 1461 bp, Paracoccus
  source 1..1461: mol_type = genomic DNA
cttgagagtttgatcctggctcagaacgaacgctggcggcaggcctaacacatgcaagtcgagcgaggacttcggttctagcggcggacgggtgagtaacgcgtgggaatgtgcccttctctacggaatagccctgggaaactgggagtaataccgtatacgcccttttggggaaagatttatcggagaaggatcagcccgcgttggattaggtagttggtggggtaatggcctaccaagccgacgatccatagctggtttgagaggatgatcagccacactgggactgagacacggcccagactcctacgggaggcagcagtggggaatcttagacaatgggggcaaccctgatctagccatgccgcgtgagtgatgaaggccttagggttgtaaagctctttcagctgggaagataatgacggtaccagcagaagaagccccggctaactccgtgccagcagccgcggtaatacggagggggctagcgttgttcggaattactgggcgtaaagcgcacgtaggcggaccggaaagtcagaggtgaaatcccagggctcaaccttggaactgcctttgaaactatcggtctggagttcgagagaggtgagtggaattccgagtgtagaggtgaaattcgtagatattcggaggaacaccagtggcgaaggcggctcactggctcgatactgacgctgaggtgcgaaagcgtggggagcaaacaggattagataccctggtagtccacgccgtaaacgatgaatgccagtcgtcgggcagcatgctgttcggtgacacacctaacggattaagcattccgcctggggagtacggtcgcaagattaaaactcaaaggaattgacgggggcccgcacaagcggtggagcatgtggtttaattcgaagcaacgcgcagaaccttaccaacccttgacatcgcaggacagcccgagagatcgggtcttctcgtaagagacctgtggacaggtgctgcatggctgtcgtcagctcgtgtcgtgagatgttcggttaagtccggcaacgagcgcaacccacgtctttagttgccagcattcagttgggcactctaaagaaactgccgatgataagtcggaggaaggtgtggatgacgtcaagtcctcatggcccttacgggttgggctacacacgtgctacaatggtggtgacagtgggttaatccccaaaagccatctcagttcggattggggtctgcaactcgaccccatgaagttggaatcgctagtaatcgcggaacagcatgccgcggtgaatacgttcccgggccttgtacacaccgcccgtcacaccatgggagttgggtctacccgacggccgtgcgccaaccctttacgggaggcagcggaccacggtaggctcagcgactggggtgaagtcgtaacaaggtagccgtaggggaacctgcggctggatcacctccttt

SEQ ID NO: 145 - DNA, 1486 bp, Roseomonas mucosa
  source 1..1486: mol_type = genomic DNA
cttgagagtttgatcctggctcagagcgaacgctggcggcatgcttaacacatgcaagtcgcacgggcagcaatgtcagtggcggacgggtgagtaacgcgtaggaacgtgtcctgaggtgggggacaaccccgggaaactggggctaataccgcatatgggctgaggcccaaagccgagaggcgcctttggagcggcctgcgtccgattaggtagttggtggggtaaaggcctaccaagcctgcgatcggtagctggtctgagaggacgaccagccacactgggactgagacacggcccagactcctacgggaggcagcagtggggaatattggacaatgggcgaaagcctgatccagcaatgccgcgtgggtgaagaaggtcttcggatcgtaaagccctttcgacggggacgatgatgacggtacccgtagaagaagccccggctaacttcgtgccagcagccgcggtaatacgaagggggctagcgttgctcggaattactgggcgtaaagggcgcgtaggcggcggcccaagtcaggcgtgaaattcctgggctcaacctggggactgcgcttgatactgggttgcttgaggatggaagaggctcgtggaattcccagtgtagaggtgaaattcgtagatattgggaagaacaccggtggcgaaggcggcgagctggtccattactgacgctgaggcgcgacagcgtggggagcaaacaggattagataccctggtagtccacgccgtaaacgatgtgcgctggatgttggggcccatagggtctcagtgtcgtagccaacgcggtaagcgcaccgcctggggagtacggccgcaaggttgaaactcaaaggaattgacgggggcccgcacaagcggtggagcatgtggtttaattcgaagcaacgcgcagaaccttaccagcccttgacatggtcacgaccggtccagagatggactttcctagcaataggcgtgatgcacaggtgctgcatggctgtcgtcagctcgtgtcgtgagatgttgggttaagtcccgcaacgagcgcaaccctcgcctctagttgccagcatgttcgggtgggcactctagaggaactgccggtgacaagccggaggaaggtggggatgacgtcaagtcctcatggcccttatgggctgggctacacacgtgctacaatggcggtgacagagggaagccaggtcgcgaggccgagccgatcccgaaaagccgtctcagttcggattgcactctgcaactcgggtgcatgaaggtggaatcgctagtaatcgcggatcagcacgccgcggtgaatacgttcccgggccttgtacacaccgcccgtcacaccatgggagttggttctaccttaagtcgttgcgctaaccagcgatgggggcaggcgaccacggtagggtcagcgactggggtgaagtcgtaacaaggtagccgtaggggaacctgcggctggatcacctccttt

SEQ ID NO: 146 - DNA, 1484 bp, Roseomonas
  source 1..1484: mol_type = genomic DNA
cttgagagtttgatcctggctcagagtgaacgctggcggcatgcttaacacatgcaagtcgcacgggcagcaatgtcagtggcggacgggtgagtaacgcgtagggaagtgtccagaggtgggggacaaccccgggaaactggggctaataccgcatatgagctgaggctcaaagccgtgaggcgcctttggagttacctgcgtccgattagctagttggtggggtaaaggcctaccaaggcgacgatcggtagctggtctgagaggacgaccagccacactgggactgagacacggcccagactcctacgggaggcagcagtggggaatattggacaatgggcgcaagcctgatccagcaatgccgcgtgggtgaagaaggtcttcggatcgtaaagccctttcggcggggacgatgatgacggtacccgcagaagaagccccggctaacttcgtgccagcagccgcggtaatacgaagggggctagcgttactcggaattactgggcgtaaagggcgcgtaggcggcgcaccaagttaggcgtgaaagccctgggctcaacctggggactgcgcttaagactggtgtgcttgaggatggaagaggctcgtggaattcccagtgtagaggtgaaattcgtagatattgggaagaacaccggtggcgaaggcggcgagctggtccattactgacgctgaggcgcgatagcgtggggagcaaacaggattagataccctggtagtccacgccgtaaacgatgtgcgctggatgttgggtgacctagtcactcagtgtcgtagctaacgcggtaagcgcaccgcctggggagtacggccgcaaggttgaaactcaaaggaattgacgggggcccgcacaagcggtggagcatgtggtttaattcgaagcaacgcgcagaaccttaccagctcttgacatggtcaggaccgacgcagagatgcgtttttcccgcaagggacctgatgcacaggtgctgcatggctgtcgtcagctcgtgtcgtgagatgttgggttaagtcccgcaacgagcgcaaccctcgcctttagttgccagcacgtttgggtgggcactctagaggaactgccggtgacaagccggaggaaggtggggatgacgtcaagtcctcatggcccttatgggctgggctacacacgtgctacaatggcggtgacaatgggaagccaggcagcgatgtcgagccgatcccaaaaagccgtctcagttcagatcgcagcctgcaactcggctgcgtgaaggtggaatcgctagtaatcgcggatcagcacgccgcggtgaatacgttcccgggccttgtacacaccgcccgtcacaccatgggagttggttctaccttaagcaggtgcgctaaccgcaagggggtagcctgccacggtagggtcagcgactggggtgaagtcgtaacaaggtagccgtaggggaacctgcggctggatcacctccttt

SEQ ID NO: 147 - DNA, 1484 bp, Sphingomonas desiccabilis
  source 1..1484: mol_type = genomic DNA
cttgagagtttgatcctggctcagaacgaacgctggcggcatgcctaacacatgcaagtcgaacgagaccttcgggtctagtggcgcacgggtgcgtaacgcgtgggaatctgcccttgggttcggaataacagttggaaacgactgctaataccggatgatgacgtaagtccaaagatttatcgcccagggatgagcccgcgtaggattagctagttggtggggtaaaggcctaccaaggcgacgatccttagctggtctgagaggatgatcagccacactgggactgagacacggcccagactcctacgggaggcagcagtggggaatattggacaatgggcgaaagcctgatccagcaatgccgcgtgagtgatgaaggccttagggttgtaaagctcttttacccgggatgataatgacagtaccgggagaataagccccggctaactccgtgccagcagccgcggtaatacggagggggctagcgttgttcggaattactgggcgtaaagcgcacgtaggcggctttgtaagttagaggtgaaagcctggagcttaactccagaactgcctttaagactgcatcgcttgaatccaggagaggtgagtggaattccgagtgtagaggtgaaattcgtagatattcggaagaacaccagtggcgaaggcggctcactggactggtattgacgctgaggtgcgaaagcgtggggagcaaacaggattagataccctggtagtccacgccgtaaacgatgataactagctgtccgggtgcttggcatttgggtggcgcagctaacgcattaagttatccgcctggggagtacggtcgcaagattaaaactcaaaggaattgacgggggcctgcacaagcggtggagcatgtggtttaattcgaagcaacgcgcagaaccttaccagcgtttgacatgtccggacgatttccagagatggatctcttcccttcggggactggaacacaggtgctgcatggctgtcgtcagctcgtgtcgtgagatgttgggttaagtcccgcaacgagcgcaaccctcgcctttagttaccatcatttagttgggtactctaaaggaaccgccggtgataagccggaggaaggtggggatgacgtcaagtcctcatggcccttacgcgctgggctacacacgtgctacaatggcaactacagtgggcagcaatcccgcgagggtgagctaatctccaaaagttgtctcagttcggattgttctctgcaactcgagagcatgaaggcggaatcgctagtaatcgcggatcagcatgccgcggtgaatacgttcccaggccttgtacacaccgcccgtcacaccatgggagttggattcacccgaaggcgttgcgccaacccgcaagggaagcaggcgaccacggtgggttcagcgactggggtgaagtcgtaacaaggtagccgtaggggaacctgcggctggatcacctccttt

SEQ ID NO: 148 - DNA, 1440 bp, Massilia
  source 1..1440: mol_type = genomic DNA
gcgagtggcgaacgggtgagtaatatatcggaacgtacccaagagtgggggataacgtagcgaaagttacgctaataccgcatacgatctatggatgaaagcaggggaccgcaaggccttgtgctcctggagcggccgatatctgattagctagttggtgaggtaaaggctcaccaaggcgacgatcagtagctggtctgagaggacgaccagccacactggaactgagacacggtccagactcctacgggaggcagcagtggggaattttggacaatgggcgcaagcctgatccagcaatgccgcgtgagtgaagaaggccttcgggttgtaaagctcttttgtcagggaagaaacggtagaggctaatatcctttgctaatgacggtacctgaagaataagcaccggctaactacgtgccagcagccgcggtaatacgtagggtgcaagcgttaatcggaattactgggcgtaaagcgtgcgcaggcggttttgtaagtctgacgtgaaagccccgggctcaacctgggaattgcgttggagactgcaaggcttgaatctggcagaggggggtagaattccacgtgtagcagtgaaatgcgtagagatgtggaggaacaccgatggcgaaggcagccccctgggtcaagattgacgctcatgcacgaaagcgtggggagcaaacaggattagataccctggtagtccacgccctaaacgatgtctactagttgtcgggttttaattaacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatgtcaggaagtctggagagatctggatgtgcccgaaagggaacctgaacacaggtgctgcatggctgtcgtcagctcgtgtcgtgagatgttgggttaagtcccgcaacgagcgcaacccttgtcattagttgctacgcaagagcactctaatgagactgccggtgacaaaccggaggaaggtggggatgacgtcaagtcctcatggcccttatgggtagggcttcacacgtcatacaatggtacatacagagggccgccaacccgcgagggggagctaatcccagaaagtgtatcgtagtccggatcgtagtctgcaactcgactacgtgaagttggaatcgctagtaatcgcggatcagcatgccgcggtgaatacgttcccgggtcttgtacacaccgcccgtcacaccatgggagcgggttttaccagaagtaggtagcttaaccgtaaggagggcgcttaccacggtaggattcgtgactggggtgaagtcgtaacaaggtagccgtatcggaaggtgcggctggatcacctccttt

SEQ ID NO: 149 - DNA, 1536 bp, Neisseria macacae
  source 1..1536: mol_type = genomic DNA
taagagtttgatcctggctcagattgaacgctggcggcatgctttacacatgcaagtcggacggcagcacagggaagcttgcttcttgggtggcgagtggcgaacgggtgagtaacatatcggaacgtaccgagcagtgggggataactaatcgaaagattagctaataccgcatattttctgaggaagaaagcaggggacctttgggccttgcgctgtttgagcggccgatatctgattagctagttggtggggtaaaggcctaccaaggcgacgatcagtagcgggtctgagaggatgatccgccacactgggactgagacacggcccagactcctacgggaggcagcagtggggaattttggacaatgggcgcaagcctgatccagccatgccgcgtgtctgaagaaggccttcgggttgtaaaggacttttgtcagggaagaaaagggcggggttaatacccctgtctgatgacggtacctgaagaataagcaccggctaactacgtgccagcagccgcggtaatacgtagggtgcgagcgttaatcggaattactgggcgtaaagcgggcgcagacggttacttaagcaggatgtgaaatccccgggctcaacctgggaactgcgttctgaactgggtgactagagtgtgtcagagggaggtagaattccacgtgtagcagtgaaatgcgtagagatgtggaggaataccgatggcgaaggcagcctcctgggataacactgacgttcatgcccgaaagcgtgggtagcaaacaggattagataccctggtagtccacgccctaaacgatgtcgattagctgttgggcagcatgactgcttagtagcgaagctaacgcgtgaaatcgaccgcctggggagtacggtcgcaagattaaaactcaaaggaattgacggggacccgcacaagcggtggatgatgtggattaattcgatgcaacgcgaagaaccttacctggtcttgacatgtacggaaccctccagagacggaggggtgccttcgggagccgtaacacaggtgctgcatggctgtcgtcagctcgtgtcgtgagatgttgggttaagtcccgcaacgagcgcaacccttgtcattagttgccatcattaagttgggcactctaatgagactgccggtgacaagccggaggaaggtggggatgacgtcaagtcctcatggcccttatgaccagggcttcacacgtcatacaatggtcggtacagagggtagccaagccgcgaggtggagccaatctcacaaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagtaggtagggtaaccgcaaggagcccgcttaccacggtatgcttcatgactggggtgaagtcgtaacaaggtagccgtaggggaacctgcggctggatcacctccttt

SEQ ID NO: 150 - DNA, 1536 bp, Neisseria subflava
  source 1..1536: mol_type = genomic DNA
taagagtttgatcctggctcagattgaacgctggcggcatgctttacacatgcaagtcggacggcagcacagagaagcttgcttcttgggtggcgagtggcgaacgggtgagtaacatatcggaacgtaccgagtaatgggggataactaatcgaaagattagctaataccgcatattctctgaggaggaaagcaggggaccttcgggccttgcgttattcgagcggccgatatctgattagctagttggtggggtaagggcctaccaaggcgacgatcagtagcgggtctgagaggatgatccgccacactgggactgagacacggcccagactcctacgggaggcagcagtggggaattttggacaatgggcgcaagcctgatccagccatgccgcgtgtctgaagaaggccttcgggttgtaaaggacttttgtcagggaagaaaaggctgttgctaatatcgacagctgatgacggtacctgaagaataagcaccggctaactacgtgccagcagccgcggtaatacgtagggtgcgagcgttaatcggaattactgggcgtaaagcgagcgcagacggttacttaagcaggatgtgaaatccccgggcttaacctgggaactgcgttctgaactgggtgactagagtgtgtcagagggaggtagaattccacgtgtagcagtgaaatgcgtagagatgtggaggaataccgatggcgaaggcagcctcctgggataacactgacgttcatgctcgaaagcgtgggtagcaaacaggattagataccctggtagtccacgccctaaacgatgtcaattagctgttgggcaacttgattgcttggtagcgtagctaacgcgtgaaattgaccgcctggggagtacggtcgcaagattaaaactcaaaggaattgacggggacccgcacaagcggtggatgatgtggattaattcgatgcaacgcgaagaaccttacctggtcttgacatgtacggaatcctccagagatggaggagtgccttcgggagccgtaacacaggtgctgcatggctgtcgtcagctcgtgtcgtgagatgttgggttaagtcccgcaacgagcgcaacccttgtcattagttgccatcatttagttgggcactctaatgagactgccggtgacaagccggaggaaggtggggatgacgtcaagtcctcatggcccttatgaccagggcttcacacgtcatacaatggtcggtacagagggtagccaagccgcgaggtggagccaatctcacaaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagtaggtagggtaaccgcaaggagcccgcttaccacggtatgcttcatgactggggtgaagtcgtaacaaggtagccgtaggggaacctgcggctggatcacctccttt

SEQ ID NO: 151 - DNA, 1536 bp, Neisseria subflava
  source 1..1536: mol_type = genomic DNA
taagagtttgatcctggctcagattgaacgctggcggcatgctttacacatgcaagtcggacggcagcacagagaagcttgcttcttgggtggcgagtggcgaacgggtgagtaacatatcggaacgtaccgagtaatgggggataactaatcgaaagattagctaataccgcatattctctgaggaggaaagcaggggaccttcgggccttgcgttattcgagcggccgatatctgattagctagttggtggggtaaaggcctaccaaggcgacgatcagtagcgggtctgagaggatgatccgccacactgggactgagacacggcccagactcctacgggaggcagcagtggggaattttggacaatgggcgcaagcctgatccagccatgccgcgtgtctgaagaaggccttcgggttgtaaaggacttttgtcagggaagaaaaggctgttgctaataccgacagctgatgacggtacctgaagaataagcaccggctaactacgtgccagcagccgcggtaatacgtagggtgcgagcgttaatcggaattactgggcgtaaagcgagcgcagacggttacttaagcaggatgtgaaatccccgggctcaacctgggaactgcgttctgaactgggtgactagagtgtgtcagagggaggtagaattccacgtgtagcagtgaaatgcgtagagatgtggaggaataccgatggcgaaggcagcctcctgggataacactgacgttcatgctcgaaagcgtgggtagcaaacaggattagataccctggtagtccacgccctaaacgatgtcaattagctgttgggcaacttgattgcttagtagcgtagctaacgcgtgaaattgaccgcctggggagtacggtcgcaagattaaaactcaaaggaattgacggggacccgcacaagcggtggatgatgtggattaattcgatgcaacgcgaagaaccttacctggtcttgacatgtacggaatcctccagagacggaggagtgccttcgggagccgtaacacaggtgctgcatggctgtcgtcagctcgtgtcgtgagatgttgggttaagtcccgcaacgagcgcaacccttgtcattagttgccatcatttagttgggcactctaatgagactgccggtgacaagccggaggaaggtggggatgacgtcaagtcctcatggcccttatgaccagggcttcacacgtcatacaatggtcggtacagagggtagccaagccgcgaggtggagccaatctcacaaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagtaggtagggtaaccgcaaggagcccgcttaccacggtatgcttcatgactggggtgaagtcgtaacaaggtagccgtaggggaacctgcggctggatcacctccttt

SEQ ID NO: 152 - DNA, 1536 bp, Enterobacter cloacae
  source 1..1536: mol_type = genomic DNA
ttgaagagtttgatcatggctcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataatgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggtgttgtggttaataaccgcagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctggagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 153 - DNA, 1539 bp, Proteus mirabilis
  source 1..1539: mol_type = genomic DNA
ttgaagagtttgatcatggctcagattgaacgctggcggcaggcctaacacatgcaagtcgagcggtaacaggagaaagcttgctttcttgctgacgagcggcggacgggtgagtaatgtatggggatctgcccgatagagggggataactactggaaacggtggctaataccgcataatgtctacggaccaaagcaggggctcttcggaccttgcactatcggatgaacccatatgggattagctagtaggtggggtaaaggctcacctaggcgacgatctctagctggtctgagaggatgatcagccacactgggactgagacacggcccagactcctacgggaggcagcagtggggaatattgcacaatgggcgcaagcctgatgcagccatgccgcgtgtatgaagaaggccttagggttgtaaagtactttcagcggggaggaaggtgataaggttaatacccttatcaattgacgttacccgcagaagaagcaccggctaactccgtgccagcagccgcggtaatacggagggtgcaagcgttaatcggaattactgggcgtaaagcgcacgcaggcggtcaattaagtcagatgtgaaagccccgagcttaacttgggaattgcatctgaaactggttggctagagtcttgtagaggggggtagaattccatgtgtagcggtgaaatgcgtagagatgtggaggaataccggtggcgaaggcggccccctggacaaagactgacgctcaggtgcgaaagcgtggggagcaaacaggattagataccctggtagtccacgctgtaaacgatgtcgatttagaggttgtggtcttgaaccgtggcttctggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagcgaatcctttagagatagaggagtgccttcgggaacgctgagacaggtgctgcatggctgtcgtcagctcgtgttgtgaaatgttgggttaagtcccgcaacgagcgcaacccttatcctttgttgccagcacgtgatggtgggaactcaaaggagactgccggtgataaaccggaggaaggtggggatgacgtcaagtcatcatggcccttacgagtagggctacacacgtgctacaatggcagatacaaagagaagcgacctcgcgagagcaagcggaactcataaagtctgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 154 - DNA, 1029 bp, Proteus mirabilis
  source 1..1029: mol_type = genomic DNA
cgtgccagcagccgcggtaatacggagggtgcaagcgttaatcggaattactgggcgtaaagcgcacgcaggcggtcaattaagtcagatgtgaaagccccgagcttaacttgggaattgcatctgaaactggttggctagagtcttgtagaggggggtagaattccatgtgtagcggtgaaatgcgtagagatgtggaggaataccggtggcgaaggcggccccctggacaaagactgacgctcaggtgcgaaagcgtggggagcaaacaggattagataccctggtagtccacgctgtaaacgatgtcgatttagaggttgtggtcttgaaccgtggcttctggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagcgaatcctttagagatagaggagtgccttcgggaacgctgagacaggtgctgcatggctgtcgtcagctcgtgttgtgaaatgttgggttaagtcccgcaacgagcgcaacccttatcctttgttgccagcacgtgatggtgggaactcaaaggagactgccggtgataaaccggaggaaggtggggatgacgtcaagtcatcatggcccttacgagtagggctacacacgtgctacaatggcagatacaaagagaagcgacctcgcgagagcaagcggaactcataaagtctgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 155 - DNA, 1539 bp, Proteus mirabilis
  source 1..1539: mol_type = genomic DNA
ttgaagagtttgatcatggctcagattgaacgctggcggcaggcctaacacatgcaagtcgagcggtaacaggagaaagcttgctttcttgctgacgagcggcggacgggtgagtaatgtatggggatctgcccgatagagggggataactactggaaacggtggctaataccgcataatgtctacggaccaaagcaggggctcttcggaccttgcactatcggatgaacccatatgggattagctagtaggtggggtaaaggctcacctaggcgacgatctctagctggtctgagaggatgatcagccacactgggactgagacacggcccagactcctacgggaggcagcagtggggaatattgcacaatgggcgcaagcctgatgcagccatgccgcgtgtatgaagaaggccttagggttgtaaagtactttcagcggggaggaaggtgataaggttaatacccttatcaattgacgttacccgcagaagaagcaccggctaactccgtgccagcagccgcggtaatacggagggtgcaagcgttaatcggaattactgggcgtaaagcgcacgcaggcggtcaattaagtcagatgtgaaagccccgagcttaacttgggaattgcatctgaaactggttggctagagtcttgtagaggggggtagaattccatgtgtagcggtgaaatgcgtagagatgtggaggaataccggtggcgaaggcggccccctggacaaagactgacgctcaggtgcgaaagcgtggggagcaaacaggattagataccctggtagtccacgctgtaaacgatgtcgatttagaggttgtggtcttgaaccgtggcttctggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagcgaatcctttagagatagaggagtgccttcgggaacgctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcagatacaaagagaagcgacctcgcgagagcaagcggaactcataaagtctgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 156 - DNA, 1002 bp, Erwinia
  source 1..1002: mol_type = genomic DNA
ttgaagagtttgatcatggctcagattgaacgctggcggcaggcctaacacatgcaagtcggacggtagcacaggggagcttgctctccgggtgacgagtggcggacgggtgagtaatgtctgggaaactgcccggtggagggggataactactggaaacggtagctaataccgcataacgtcttcggaccaaagtgggggatcttcggacctcacgccaccggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcggtgaggttaataacctcgccgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggctgttcccctgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccagagaa

SEQ ID NO: 157 - DNA, 1539 bp, Erwinia
  source 1..1539: mol_type = genomic DNA
ttgaagagtttgatcatggctcagattgaacgctggcggcaggcctaacacatgcaagtcggacggtagcacaggggagcttgctctccgggtgacgagtggcggacgggtgagtaatgtctgggaaactgcccggtggagggggataactactggaaacggtagctaataccgcataacgtcttcggaccaaagtgggggatcttcggacctcacgccaccggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcggtgaggttaataacctcgccgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggctgttcccctgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccagagaacttagcagagatgcttcggtgccttcgggaactctgagacaggtgctgcatggctgtcgtcagctcgtgttgtgaaatgttgggttaagtcccgcaacgagcgcaacccttatcctttgttgccagcgggtgatgccgggaactcaaaggagactgccggtgataaaccggaggaaggtggggatgacgtcaagtcatcatggcccttacggccagggctacacacgtgctacaatggcgcatacaaagagaagcgacctcgcgagagcaagcggacctcataaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaacctccgggagggcgcttaccactttgtgattcatgactggggtgaagtcgtaacaaggtaaccgtaggggaacctgcggttggatcacctcctt

SEQ ID NO: 158 - DNA, 1002 bp, Erwinia
  source 1..1002: mol_type = genomic DNA
ttgaagagtttgatcatggctcagattgaacgctggcggcaggcctaacacatgcaagtcggacggtagcacaggggagcttgctccccgggtgacgagtggcggacgggtgagtaatgtctgggaaactgcccggtggagggggataactactggaaacggtagctaataccgcataacgtcttcggaccaaagtgggggatcttcggacctcacgccaccggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcggtgaggttaataacctcgccgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggctgttcccctgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccagagaa

SEQ ID NO: 159 - DNA, 1539 bp, Erwinia
  source 1..1539: mol_type = genomic DNA
ttgaagagtttgatcatggctcagattgaacgctggcggcaggcctaacacatgcaagtcggacggtagcacaggggagcttgctccccgggtgacgagtggcggacgggtgagtaatgtctgggaaactgcccggtggagggggataactactggaaacggtagctaataccgcataacgtcttcggaccaaagtgggggatcttcggacctcacgccaccggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcggtgaggttaataacctcgccgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggctgttcccctgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccagagaacttagcagagatgcttcggtgccttcgggaactctgagacaggtgctgcatggctgtcgtcagctcgtgttgtgaaatgttgggttaagtcccgcaacgagcgcaacccttatcctttgttgccagcgggtgatgccgggaactcaaaggagactgccggtgataaaccggaggaaggtggggatgacgtcaagtcatcatggcccttacggccagggctacacacgtgctacaatggcgcatacaaagagaagcgacctcgcgagagcaagcggacctcataaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaacctccgggagggcgcttaccactttgtgattcatgactggggtgaagtcgtaacaaggtaaccgtaggggaacctgcggttggatcacctcctt

SEQ ID NO: 160 - DNA, 1533 bp, Acinetobacter radioresistens
  source 1..1533: mol_type = genomic DNA
ctgaagagtttgatcatggctcagattgaacgctggcggcaggcttaacacatgcaagtcgagcggatgaaggtagcttgctaccggattcagcggcggacgggtgagtaatgcttaggaatctgcctattagtgggggacaacgttccgaaaggagcgctaataccgcatacgtcctacgggagaaagcaggggacctttgggccttgcgctaatagatgagcctaagtcggattagctagttggtagggtaaaggcctaccaaggcgacgatctgtagcgggtctgagaggatgatccgccacactgggactgagacacggcccagactcctacgggaggcagcagtggggaatattggacaatggggggaaccctgatccagccatgccgcgtgtgtgaagaaggccttttggttgtaaagcactttaagcgaggaggaggctacctagattaatactttaggatagtggacgttactcgcagaataagcaccggctaactctgtgccagcagccgcggtaatacagagggtgcgagcgttaatcggatttactgggcgtaaagcgtgcgtaggcggccaattaagtcaaatgtgaaatccccgagcttaacttgggaattgcattcgatactggttggctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcccttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggccttgacatacagagaactttccagagatggattggtgccttcgggaactctgatacaggtgctgcatggctgtcgtcagctcgtgtcgtgagatgttgggttaagtcccgcaacgagcgcaacccttttccttatttgccagcacttcgggtgggaactttaaggatactgccagtgacaaactggaggaaggcggggacgacgtcaagtcatcatggcccttacggccagggctacacacgtgctacaatggtcggtacaaagggttgctacacagcgatgtgatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtaggtagtctaaccgcaaggaggacgcttaccacggtgtggccgatgactggggtgaagtcgtaacaaggtagccgtaggggaacctgcggctggatcacctcctt

SEQ ID NO: 161 - DNA, 1527 bp, Enhydrobacter aerosaccus
  source 1..1527: mol_type = genomic DNA
tgaagagtttgatcatggctcagattgaacgctggcggcaggcttaacacatgcaagtcgaacgatgattatctagcttgctagatatgattagtggcggacgggtgagtaacatttaggaatctgcctagtagtgggggatagctcggggaaactcgaattaataccgcatacgacctacgggtgaaagggggcgcaagctcttgctattagatgagcctaaatcagattagctagttggtggggtaaaggcccaccaaggcgacgatctgtaactggtctgagaggatgatcagtcacaccggaactgagacacggtccggactcctacgggaggcagcagtggggaatattggacaatgggggcaaccctgatccagccatgccgcgtgtgtgaagaaggccttttggttgtaaagcactttaagcagggaggagaggctaatggttaatacccattagattagacgttacctgcagaataagcaccggctaactctgtgccagcagccgcggtaatacagagggtgcgagcgttaatcggaattactgggcgtaaagcgagtgtaggtggctcattaagtcacatgtgaaatccccgggcttaacctgggaactgcatgtgatactggtggtgctagaatatgtgagagggaagtagaattccaggtgtagcggtgaaatgcgtagagatctggaggaataccgatggcgaaggcagcttcctggcataatattgacactgagattcgaaagcgtgggtagcaaacaggattagataccctggtagtccacgccgtaaacgatgtctactagccgttggggtccttgagactttagtggcgcagttaacgcgataagtagaccgcctggggagtacggccgcaaggttaaaactcaaatgaattgacgggggcccgcacaagcggtggagcatgtggtttaattcgatgcaacgcgaagaaccttacctggtcttgacatagtgagaatcctgcagagatgcgggagtgccttcgggaattcacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtaggtacagagggttgctacacagcgatgtgatgctaatctcaaaaagcctatcgtagtccggattggagtctgcaactcgactccatgaagtcggaatcgctagtaatcgcagatcagaatgctgcggtgaatacgttcccgggccttgtacacaccgcccgtcacaccatgggagtctattgcaccagaagtaggtagcctaacgcaagagggcgcttaccacggtgtggtcgatgactggggtgaagtcgtaacaaggtagccgtaggggaacctgcggctggatcacctcctt

SEQ ID NO: 162 - DNA, 1527 bp, Enhydrobacter aerosaccus
  source 1..1527: mol_type = genomic DNA
tgaagagtttgatcatggctcagattgaacgctggcggcaggcttaacacatgcaagtcgaacgatgactctctagcttgctagagaagattagtggcggacgggtgagtaacatttaggaatctgcctagtagtgggggatagctcggggaaactcgaattaataccgcatacgacctacgggtgaaagggggcgcaagctcttgctattagatgagcctaaatcagattagctagttggtggggtaaaggcccaccaaggcgacgatctgtaactggtctgagaggatgatcagtcacaccggaactgagacacggtccggactcctacgggaggcagcagtggggaatattggacaatgggggcaaccctgatccagccatgccgcgtgtgtgaagaaggccttttggttgtaaagcactttaagcagggaggagaggctaatggttaatacccattagattagacgttacctgcagaataagcaccggctaactctgtgccagcagccgcggtaatacagagggtgcgagcgttaatcggaattactgggcgtaaagcgagtgtaggtggctcattaagtcacatgtgaaatccccgggcttaacctgggaactgcatgtgatactggtggtgctagaatatgtgagagggaagtagaattccaggtgtagcggtgaaatgcgtagagatctggaggaataccgatggcgaaggcagcttcctggcataatattgacactgagattcgaaagcgtgggtagcaaacaggattagataccctggtagtccacgccgtaaacgatgtctactagccgttggggtccttgagactttagtggcgcagttaacgcgataagtagaccgcctggggagtacggccgcaaggttaaaactcaaatgaattgacgggggcccgcacaagcggtggagcatgtggtttaattcgatgcaacgcgaagaaccttacctggtcttgacatagtgagaatcctgcagagatgcgggagtgccttcgggaattcacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtaggtacagagggttgctacacagcgatgtgatgctaatctcaaaaagcctatcgtagtccggattggagtctgcaactcgactccatgaagtcggaatcgctagtaatcgcagatcagaatgctgcggtgaatacgttcccgggccttgtacacaccgcccgtcacaccatgggagtctattgcaccagaagtaggtagcctaacgcaagagggcgcttaccacggtgtggtcgatgactggggtgaagtcgtaacaaggtagccgtaggggaacctgcggctggatcacctcctt

SEQ ID NO: 163 - DNA, 1106 bp, Enhydrobacter aerosaccus
  source 1..1106: mol_type = genomic DNA
tgaagagtttgatcatggctcagattgaacgctggcggcaggcttaacacatgcaagtcgaacgatgattatctagcttgctagatatgattagtggcggacgggtgagtaacatttaggaatctgcctagtagtgggggatagctcggggaaactcgaattaataccgcatacgacctacgggtgaaagggggcgcaagctcttgctattagatgagcctaaatcagattagctagttggtggggtaaaggcccaccaaggcgacgatctgtaactggtctgagaggatgatcagtcacaccggaactgagacacggtccggactcctacgggaggcagcagtggggaatattggacaatgggggcaaccctgatccagccatgccgcgtgtgtgaagaaggccttttggttgtaaagcactttaagcagggaggagaggctaatggttaatacccattagattagacgttacctgcagaataagcaccggctaactctgtgccagcagccgcggtaatacagagggtgcgagcgttaatcggaattactgggcgtaaagcgagtgtaggtggctcattaagtcacatgtgaaatccccgggcttaacctgggaactgcatgtgatactggtggtgctagaatatgtgagagggaagtagaattccaggtgtagcggtgaaatgcgtagagatctggaggaataccgatggcgaaggcagcttcctggcataatattgacactgagattcgaaagcgtgggtagcaaacaggattagataccctggtagtccacgccgtaaacgatgtctactagccgttggggtccttgagactttagtggcgcagttaacgcgataagtagaccgcctggggagtacggccgcaaggttaaaactcaaatgaattgacgggggcccgcacaagcggtggagcatgtggtttaattcgatgcaacgcgaagaaccttacctggtcttgacatagtgagaatcctgcagagatgcgggagtgccttcgggaattcacatacaggtgctgcatggctgtcgtcagctcgtgtcgtgagatgttgggttaagtcccgcaacgagcgcaacccttttc

SEQ ID NO: 164 - DNA, 1527 bp, Enhydrobacter
  source 1..1527: mol_type = genomic DNA
tgaagagtttgatcatggctcagattgaacgctggcggcaggcttaacacatgcaagtcgaacgatgactctctagcttgctagagatgattagtggcggacgggtgagtaacatttaggaatctgcctagtagtgggggatagctcggggaaactcgaattaataccgcatacgacctacgggtgaaagggggcgcaagctcttgctattagatgagcctaaatcagattagctagttggtggggtaaaggcccaccaaggcgacgatctgtaactggtctgagaggatgatcagtcacaccggaactgagacacggtccggactcctacgggaggcagcagtggggaatattggacaatgggggcaaccctgatccagccatgccgcgtgtgtgaagaaggccttttggttgtaaagcactttaagcagggaggagaggctaatggttaatacccattagattagacgttacctgcagaataagcaccggctaactctgtgccagcagccgcggtaatacagagggtgcgagcgttaatcggaattactgggcgtaaagcgagtgtaggtggctcattaagtcacatgtgaaatccccgggcttaacctgggaactgcatgtgatactggtggtgctagaatatgtgagagggaagtagaattccaggtgtagcggtgaaatgcgtagagatctggaggaataccgatggcgaaggcagcttcctggcataatattgacactgagattcgaaagcgtgggtagcaaacaggattagataccctggtagtccacgccgtaaacgatgtctactagccgttggggtccttgagactttagtggcgcagttaacgcgataagtagaccgcctggggagtacggccgcaaggttaaaactcaaatgaattgacgggggcccgcacaagcggtggagcatgtggtttaattcgatgcaacgcgaagaaccttacctggtcttgacatagtgagaatctctcagagatgagagagtgccttcgggaactcacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtaggtacagagggttgctacacagcgatgtgatgctaatctcaaaaagcctatcgtagtccggattggagtctgcaactcgactccatgaagtcggaatcgctagtaatcgcggatcagaatgccgcggtgaatacgttcccgggccttgtacacaccgcccgtcacaccatgggagtctattgcaccagaagtaggtagcctaacgaaagagggcgcttaccacggtgtggtcgatgactggggtgaagtcgtaacaaggtagccgtaggggaacctgcggctggatcacctcctt

SEQ ID NO: 165 - DNA, 1532 bp, Pseudomonas
  source 1..1532: mol_type = genomic DNA
tgaagagtttgatcatggctcagattgaacgctggcggcaggcctaacacatgcaagtcgagcggatgaagggagcttgctccctgattcagcggcggacgggtgagtaatgcctaggaatctgcctattagtgggggacaacgtttcgaaaggaacgctaataccgcatacgtcctacgggagaaagcaggggaccttcgggccttgcgctaatagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggtcgttgcctaatacgtgacggttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcatccaaaactggcaagctagagtacggtagagggtggtggaatttcctgtgtagcggtgaaatgcgtagatataggaaggaacatcagtggcgaaggcgaccacctggactgatactgacactgaggtgcgaaagcgtggggagcaaacaggattagataccctggtagtccacgccgtaaacgatgtcaactagccgttgggatccttgagatcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccagtgaactttccagagatggattggtgccttcgggaacactgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatctcacaaaaccgatcgtagtccggatcgcagtctgcaactcgactgcgtgaagtcggaatcgctagtaatcgcgaatcagaacgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 166 - DNA, 1532 bp, Pseudomonas
  source 1..1532: mol_type = genomic DNA
tgaagagtttgatcatggctcagattgaacgctggcggcaggcctaacacatgcaagtcgagcggatgaagggagcttgctccctgattcagcggcggacgggtgagtaatgcctaggaatctgcctattagtgggggacaacgtttcgaaaggaacgctaataccgcatacgtcctacgggagaaagcaggggaccttcgggccttgcgctaatagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggtcgttgcctaatacgtgacggttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcatccaaaactggcaagctagagtacggtagagggtggtggaatttcctgtgtagcggtgaaatgcgtagatataggaaggaacatcagtggcgaaggcgaccacctggactgatactgacactgaggtgcgaaagcgtggggagcaaacaggattagataccctggtagtccacgccgtaaacgatgtcaactagccgttgggatccttgagatcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccagtgaactttccagagatggattggtgccttcgggaacactgagacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatctcacaaaaccgatcgtagtccggatcgcagtctgcaactcgactgcgtgaagtcggaatcgctagtaatcgcgaatcagaacgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 167 - DNA, 1428 bp, Aspergillus kawachii
  source 1..1428: mol_type = genomic DNA
ttgagtttgatgatggctctgattgaacgctgtccaagtacttgacacatgctaatcgaacgactatttattaaaataaaatgagtagtagtggtgtacaggtgagtaaaagataatttggctaccttaaagtaagggaaaaatcccttatataagaaaggaaaataaaaggtccgctttaagatgttaatctttatctcggagagtagtaggaaaggtaatgacttttctagctaatatccgtagtcgtgactgagaggtcgatcgaccacattgggtctgagaaaaccccaatgcgtattagtacagcagtgaggaatattggtcaatggccgaaaggctgaaccagtaacttggaagaatgtaaatgtatttaataaatacaataacgattaactcgtataaaattctaaataggataatgataatgacaatttcctatttataagtcttgaccaaattacgtgccagcagtcgcggtaatacgtagaagactagtgttagtcatctttattaggtttaaagggtacctagacggtaaattaaactctaaatgagtacttttttactagagttttatgtgagaaggaagaacttctggagtagggataaaatacgcaaataccagaaggactgataacggcgaaggcgtccttctatgtaaaaactgacgttgagggacgaaggctcgggtagcgataaggattagataccctagtagtccaagcagacaatgatgaatgtcatagactagatttaatatttagtctataaatgaaagtgtaagcattccacctcaagagtaatatggcaacatataaactgaaatcattagaccgtttctgaaaccagtagtgaagtatgttatttaattcgataacccgcgaaaaaccttaccacagtttgaataacaattaaaaaattgttacaagcgctgcacggctgtctttagttaatgtcgtgagatctggttaactcctttaattaacgaaaaccctcactttatttagatatataaagtggttcgccgctacataggatatagataatagggattaagacaagtcatcatggccttaatactgtgggctatagacgtgccacatacgccttaacaaagggatgcgatattgtgaaatggagctaatccccaaaaaaggatataatatggattgtagtctgtaactcgactacatgaataaggaattactagtaatcgtgtatcaccaacggcacggtgaatttattctcagctaggtactaaccactcgtcaggcgctgaaagaagtatgtgcaataagtttgatttgcttatgtattaatatatataatcaggtatataaatatgtatgtcttattttcgtatgcatgactttgattggtgttaagtcgaaataaggttcgtgtaatggaaattgcacgggatgg

SEQ ID NO: 168 - DNA, 1428 bp, Aspergillus niger; identical to SEQ ID NO: 167
  source 1..1428: mol_type = genomic DNA

SEQ ID NO: 169 - DNA, 1426 bp, Aspergillus pseudoglaucus
  source 1..1426: mol_type = genomic DNA
tttgagtttgatgatggctctgattgaacgctgtccaagtacttgacacatgctaatcgaacgacttcgctccctgcggggagcgacgtagtggtgtacaggtgagtataagataatttggctacctaaaagtaagggaaaaatcccttatataaagaaaggaataaaaggtccgctttaagatgttaatctttatcccggtgagtagtaggaaaggtaatgacttttctagctaataaccgtagtcgtgactgagaggtcgatcgaccacattgggtctgaaaaaaccccaatgcgttttagtacagcagtgaggaatattggtcaatggccgaaaggctgaaccagtaacttggaagaatgaaaaaatgtattaaaataatacattaacgatttactcgtataaagttctaaataggataatgataatgacaatttcctatttataagtcttgaccaaattacgtgccagcagtcgcggtaatacgtaaaagactagtgttagtcatctttattaggtttaaagggtacctagacggtaaattaaactctaaatgagtacttttttactagagttttataagagaaggaagaatttctggagtagtgatagaatattttaataccagaaggactgataacggcgaaggcgtccttctatgtaaaaactgacgttgagggacgaaggcttgggtagcaataaggattagataccctagtagtccaagcagaaaatgatgaatgtcatagactagaaaatatatttagtctataaatgaaagtgtaagcattccacctcaagagtaatatggcaacatataaactgaaatcattagaccgtttctgaaaccagtagtgaagtatgttatttaattcgataacccgcgaaaaaccttaccacagtttgaataacaataaaattgttacaagcgctgcacggctgtctttagttaatgtcgtgagatttggttaactcctcaaattaacgaaaaccctcactttatttatatacatagagtggaacgccgttatattggactggtaatagggattaagacaagtcatcatggcctaaatactgtgggctatagacgtgccacatacgccttaacaaagggatgcaatattgtgaaatggagctaatccccaaaattggatataatatggattgtagtctgtaactcgactacatgaataaggaattactagtaatcgtgaatcaccaacgtcacggtgaatctaatctcagctaggtactaaccactcgtcaggcgctgaaaggagtatgtgcaataagtttgattaatttacgtattaatatatataatcaggtatataaatacgtaggtttagttttcgtatgcatgactctgattggtgttaagtcgaaatacggttcgtgtaatggaaattgcacgggatgaat

SEQ ID NO: 170 - DNA, 1606 bp, Saccharomyces cerevisiae
  source 1..1606: mol_type = genomic DNA
ttgagtttgatgatggctctgagtgaacgctatgtaaatgcttgacacatgctaatcgaacgtcaatatttaatttagataaattatgtatattgaagtggtgtacaggtgagtaagtggtattatggctacctaagagtaaggcaaaataaatcccttataataacaatttattattatgtattaatactagatctaatcacccaatggtcgattatatctaaaaggttaggtataaaggttaataatagtaagaataaatgtgtttaaaaagaaatagtatttcgctcttagttgattattaataaccatcggacaagtagtaagtatggtaatggcatatttagcttatgtccgtagtcaagactgagaggtctattgaccacactgggtctgaaaaaaccccagtgcgtgtagtacagcagtgaggaattttggtcaatggcctaacggctgaaccagcaacttgcaggaatgagagttaatataataagttattaacttgcttgactaggtcttataaagttctggataaagcttattatgaaaactttatatccataggtctcgaccaaatcttgtgccagcagtcgcggtaagacaagggagacgagtgttattcatctttaacaggtatatagggtacctagacggtgtgcaatggcttacataagtacctggtacacttgagtttgatatgtgagaggaatatgtcggaattgttggaggaaagatgaaattttttgataccaataggaccggtaacggcgaaggcaaacctctatgtacatgtacttgctatttctctagtataaagaggaaaagagcaatgtactataactgacgttgagggacgaaggctcagatagtgaagaggattagatacccccgtaacctgggcagacaatgatgagtgccataggttggctttaaggcggaactataaatgaaagtggaagcactccacctcaagagtaatgtggcaacgcaggaactgaaatcattagaccgtttctgaaaccagtagtgaagtatgttatttaattcgataatccgcgaataaccttaccacaatttgaatgacctaaaatattttttaaatttttagaccacatgcgttgcatggctgtcttcagttaatgtcgtgagatttggttaggtccattaaattaacgtaaacccttgttttatttattaagataaagcagctctccgcttacaattggatgtgataatagggactaagacaagtcatcatggccttaatattgtgggctatagacgtgccacatatgcctttacaaagggatgcaataatgcgaattggagctaatccctaaaacaggataaagtatggattgttgtctgaaattcgactgcatgaaaaaggaattactagtaatcgtgagtaatcacatcacggtgaattttatctcagataggtactaaccactcgtcaggcgctgaaaggagtatgtgcaataagtttggtggctgcggattgctagtctaaatgtacaggtttagatcttttggcagaaatatcttcgtatgcgtgactctgattggtgttaagtcgaaatacggttcgggttggggaactagc

SEQ ID NO: 171 - DNA, 1449 bp, Sphingomonas
  source 1..1449: mol_type = genomic DNA
agagtttgattctggctcagaacgaacgctggcggcatgcctaacacatgcaagtcgaacgagatcttcggatctagtggcgcacgggtgcgtaacgcgtgggaatctgccctttggttcggaataacagttggaaacgactgctaataccggatgatgacgtaagtccaaagatttatcgccagaggatgagcccgcgtaggattagctagttggtgtggtaagagcgcaccaaggcgacgatccttagctggtctgagaggatgatcagccacactgggactgagacacggcccagactcctacgggaggcagcagtggggaatattggacaatgggcgaaagcctgatccagcaatgccgcgtgagtgatgaaggccttagggttgtaaagctcttttacccgggatgataatgacagtaccgggagaataagctccggctaactccgtgccagcagccgcggtaatacggagggagctagcgttgttcggaattactgggcgtaaagcgcacgtaggcggctttgtaagttagaggtgaaagcctggagctcaactccagaattgcctttaagactgcatcgcttgaatccaggagaggtgagtggaattctgagtgtagagctgaaattcgtatatattccgaataacaccacgcgcgaaggcggctcactggactggtattgacgttgaggtgcgaaagcgtgggagcaaaccaggattagataccttggtagtccacgccgtaaacgatgataactagctgtccggggacttggtctttgggtggcgcagctaacgcattaagttatccgcctggggagtacggccgcaaggttaaaactcaaatgaattgacgggggcctgcacaagcggtggagcatgtggtttaattcgaagcaacgcgcagaaccttaccagcgtttgacatgtccggacgatttctggagacagatctcttcccttcggggactggaacacaggtgctgcatggctgtcgtcagctcgtgtcgtgagatgttgggttaagtcccgcaacgagcgcaaccctcgcctttagttaccatcatttagttggggactctaaaggaaccgccggtgataagccggaggaaggtggggatgacgtcaagtcctcatggcccttacgcgctgggctacacacgtgctacaatggcggtgacagtgggcagcaaactcgcgagagtgcgctaatctccaaaagccgtctcagttcggattgttctctgcaactcgagagcatgaaggcggaatcgctagtaatcgcggatcagcatgccgcggtgaatacgttcccaggccttgtacacaccgcccgtcacaccatgggagttgggttcacccgaaggcgttgcgctaactcgtaagagaggcaggcgaccacggtgggcttagcgactggggtgaagtcgtaacaaggtaaccg

SEQ ID NO: 172 - DNA, 1457 bp, Tepidimonas
  source 1..1457: mol_type = genomic DNA
ctttacacatgcaagtcgaacggtaacaggccttcgggtgctgacgagtggcgaacgggtgagtaatgcatcggaacgtgcccagaggtgggggataacgcagcgaaagctgtgctaataccgcatgtgatctgaggatgaaagcgggggaccaagcagcaatgtttggcctcgcgcctctggagcggccgatgtcagattaggtagttggtggggtaaaggcctaccaagccgacgatctgtagctggtctgagaggacgaccagccacactgggactgagacacggcccagactcctacgggaggcagcagtggggaattttggacaatgggcgcaagcctgatccagcaatgccgcgtgcgggaagaaggccttcgggttgtaaaccgcttttgtacggaacgaaaaggctctggctaatacctggggctgatgacggtaccgtaagaataagcaccggctaactacgtgccagcagccgcggtaatacgtagggtgcgagcgttaatcggaattactgggcgtaaagcgtgcgcaggcggtcttgtaagacagaggtgaaatccctgggctcaacctaggaatggcctttgtgactgcaaggctggagtgcggcagagggggatagaattccgcgtgtagcagtgaaatgcgtagatatgcggaggaacaccgatggcgaaggcagtcccctgggcctgcactgacgctcatgcacgaaagcgtggggagcaaacaggattagataccctggtagtccacgccctaaacgatgtcgactggttgttgggccttaggtggctcagtaacgaagctaacgcgtgaagtcgaccgcctggggagtacggccgcaaggttgaaactcaaaggaattgacggggacccgcacaagcggtggatgatgtggtttaattcgatgcaacgcgaaaaaccttacctacccttgacatgccaggaatcctgcagagatgtgggagtgctcgcaagagagcctggacacaggtgctgcatggctgtcgtcagctcgtgtcgtgagatgttgggttaagtcccgcaacgagcgcaacccttgccattagttgctacgaaagggcactctaatgggactgccggtgacaaaccggaggaaggtggggatgacgtcaagtcctcatggcccttatgggtagggctacacacgtcatacaatggccggtacagagggctgccaacccgcgagggggagccaatcccgtaaaaccggtcgtagtccggattgcagtctgcaactcgactgcatgaagtcggaatcgctagtaatcgcggatcagcatgtcgcggtgaatacgttcccgggtcttgtacacaccgcccgtcacaccatgggagcgggttctgccagaagtagttagcctaaccgcaaggggggcgattaccacggcagggttcgtgactggggtgaagtcgtaacaaggt

SEQ ID NO: 173 - DNA, 1435 bp, Methylobacterium organophilum
  source 1..1435: mol_type = genomic DNA
cctggctcagagcgaacgctggcggcaggcttaacacatgcaagtcgaacgcatccttcgggatgagtggcagacgggtgagtaacacgtgggaacgtacccttcggttcggaataactcagggaaacttgagctaataccggatacgcccttttggggaaaggtttactgccgaaggatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttgtccgggacgataatgacggtaccggaagaataagccccggctaacttcgtgccagcagccgcggtaatacgaagggggctagcgttgctcggaatcactgggcgtaaagggcgcgtaggcggccatttaagtcgggggtgaaagcctgtggctcaaccacagaattgccttcgatactgggtggcttgagtccggaagaggttggtggaactgcgagtgtagaggtgaaattcgtagatattcgcaagaacaccggtggcgaaggcggccaactggtccggaactgacgctgaggcgcgaaagcgtggggagcaaacaggattagataccctggtagtccacgccgtaaacgatgaatgccagccgttgggcacttgctgctcaatggcgcaaccaacgctttgagcattccgcctggggagtacggtcgcaagattaaaactcaaaggaattgacgggggcccgcacaagcggtggagcatgtggtttaattcgaagcaacgcgcagaaccttaccatcccttgacatggcatgttacccagagagatttggggtcctcttcggaggcgtgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagagggacgcgaaggggcgacctggagcaaatcccgaaaaaccgtctcagttcggattgcactctgcaactcgggtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtaaccgt

SEQ ID NO: 174 - DNA, 1407 bp, Methylobacterium mesophilicum
  source 1..1407: mol_type = genomic DNA
agcgaacgctggcggcaggcttaacacatgcaagtcgagcgggccctttcgggggtcagcggcggacgggtgagtaacgcgtgggaacgtgccctctggttcggaataactcagggaaacttgagctaataccggatacgcccttttggggaaaggcttgctgccggaggatcggcccgcgtctgattagctagttggtgaggtaacggctcaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttatccgggacgataatgacggtaccggaggaataagccccggctaacttcgtgccagcagccgcggtaatacgaagggggctagcgttgctcggaatcactgggcgtaaagggcgcgtaggcggcgttttaagtcgggggtgaaagcctgtggctcaaccacagaatggccttcgatactgggacgcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcacctcagtagcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttatggggagagattcccagtcctcttcggaggcgcgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggggcgacctggagcaaatccccaaaagccgtctcagttcggattgcactctgcaactcgggtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtaggctcagcgactggggtg

SEQ ID NO: 175 - DNA, 1326 bp, unidentified
  misc_feature 1..1326: note = 16S ribosomal RNA of unknown bacteria
  source 1..1326: mol_type = genomic DNA
atctcatggagagttcgatcctggctcaggatgaacgctggcggcatgcttaacacatgcaagtcggacgggaagtggtgtttccagtggcggacgggtgagtaacgcgtaagaacctgcccttgggaggggaacaacagctggaaacggctgctaataccccgtaggctgaggagcaaaaggaggaatccgcccgaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtggaggtagaaggcccacgggtcgtgaacttcttttcccggagaagaagcaatgacggtatctggggaataagcatcggctaactctgtgccagcagccgcggtaatacagaggatgcaagcgttatccggaatgattgggcgtaaagcgtctgtaggtggctttttaagtccgccgtcaaatcccagggctcaaccctggacaggcggtggaaactaccaagctggagtacggtaggggcagagggaatttccggtggagcggtgaaatgcgtagagatcggaaagaacaccaacggcgaaagcactctgctgggccgacactgacactgagagacgaaagctaggggagcgaatgggattagataccccagtagtcctagccgtaaacgatggatactaggcgctgtgcgtatcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccttcgggaacgcggacacaggtggtgcatggctgtcgtcagctcgtgccgtaaggtgttgggttaagtcccgcaacgagcgcaaccctcgtgtttagttgccatcgttgagtttggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaaccccaaaaacccgtcctcagttcggattgcaggctgcaactcgcctgcatgaagccggaatcgctagtaatcgccggtcagccatacggcggtgaat

SEQ ID NO: 176 - DNA, 1348 bp, Finegoldia
  source 1..1348: mol_type = genomic DNA
acgaacgctggcggcgtgcttaacacatgcaagtcgaacgggatttagtagacagaagcctcggtggaagattactaatgagagtggcgaacgggtgagtaacgcgtgagcaacctgcctatgacagtgggatagcctcgggaaaccgggattaataccgcataaaatcgtagaaacacatgttttaacggtcaaagatttatcggtcatagatgggctcgcgtctgattagctagttggtgagataacagcccaccaaggcgacgatcagtagccggtctgagaggatgaacggccacattggaactgagacacggtccaaactcctacgggaggcagcagtggggaatattgcacaatgggggaaaccctgatgcagcgacgccgcgtgaacgaagaaggtattcgtatcgtaaagttctgtcctatgggaagataatgacagtaccatagaagaaagctccggctaaatacgtgccagcagccgcggtaatacgtatggagcgagcgttgtccggaattattgggcgtaaagggtacgcaggcggtttaataagtcgaatgttaaagatcggggctcaaccccgtaaagcattggaaactgataaacttgagtagtggagaggaaagtggaattcctagtgtggtggtgaaatacgtagatattaggaggaataccagtagcgaaggcgactttctggacacaaactgacgctgaggtacgaaagcgtggggagcaaacaggattagataccctggtagtccacgccgtaaacgatgaatgctaggtgttggggatcatacctcggtgccgaagttaacacattaagcattccgcctggggagtacgcacgcaagtgtgaaactcaaaggaattgacggggacccgcacaagcagcggagcatgtggtttaattcgatgcaacgcgaagaaccttaccagggcttgacatgtgggtgaaaggtatagagatatacccctctctttatgagacatccatacaggtggtgcatggttgtcgtcagctcgtgtcgtgagatgttgggttaagtcccgcagcgagcgcaacccctatacttagttaccagcggataatgccggggactctaagtagactgccgatgacaaatcggaggaaggtggggatgacgtcaaatcatcatgccctttatgtcctgggctacacacgtgctacaatggttggtacagagggaagctatatagtgatataatgcaaaactccaaagccaatcccagttcggattgtaggctgcaactcgcctacatgaagtcggagttgctagtaatcgcggatcagaatgtcgcggtgaatgcgttcccgggtct

SEQ ID NO: 177 - DNA, 1333 bp, Leptotrichia shahii
  source 1..1333: mol_type = genomic DNA
gatgaacgctgacagaatgcttaacacatgcaagtctttggcgaagctgcgcttgcgcagacgagccaaggcggacgggtgagtaacgcgtaaggaacttgccctgcagacagggataacagacggaaacgactgacaagacctgatacagttgccggcacgcatgtgcctggcgatgaaaggagacgctgcaggagagccttgcgtcctattagcttgttggcggggtaatggcccaccaaggcgatgataggtagccggcctgagagggtggacggccacaaggggactgagatacggcccttactcctacgggaggcagcagtggggaatattggacaatgggggcaaccctgatccagcaattctgtgtgcacgaagaaggttttcggattgtaaagtgctttcagcagggaagaagaaagtgacggtacctgcagaagaagcgacggctaaatacgtgccagcagccgcggtaatacgtatgtcgcgagcgttatccggaattattgggcataaagggcatctaggcggccctgtaagtctagggtgaaaacctgcggctcaaccgcaggcctgccccggaaactacagggctagagtgctggagaggtggacggaactgcacgagtagaggtgaaattcgtagatatgtgcaggaatgccgatgatgaagatagttcactggacggcaactgacgctgaagtgcgaaagctaggggagcaaacaggattagataccctggtagtcctagctgtaaacgatgattactgggtgtgggcatgaagagtgtccgtgccgaagctaatgcgataagtaatccgcctggggagtacggccgcaaggctgaaactcaaaggaattgacggggacccgcacaagcggtagagcatgtggtttaattcgacgcaacgcgaggaaccttaccagatcttgacatcctgcgaaggcctgcgagagcaggccgtgccttcgggaacgcagagacaggtggtgcatggctgtcgacagctcgtgtcgtgagatgttgggttaagtcccgcaacgagcgcaacccctatcgctagttgccatcattaggttgaggactctagcgagactgcctgcgaagagcaggaggaaggcggggatgacgtcaagtcatcatgccccttatgatctgggctacacacgtgctacaatggccggtacaaagagctgccaggcggcaacgccaagcgaatctttaaagccggtccaagttcggattgaagcctgcaactcggcttcatgaagctggaatcgctagtaatcgcagatcagcaatgctgcggtgaatacgttctcgggtct

SEQ ID NO: 178 - DNA, 1372 bp, Staphylococcus
  source 1..1372: mol_type = genomic DNA
gatgaacgctggcggcgtgcctaatacatgcaagtcgagcgaacagacgaggagcttgctcctctgacgttagcggcggacgggtgagtagcacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtct

SEQ ID NO: 179 - DNA, 1503 bp, Staphylococcus haemolyticus
  source 1..1503: mol_type = genomic DNA
gatgaacgctggcggcgtgcctaatacatgcaagtcgagcgaacagacgaggagcttgcccctctgacgttagcggcggacgggtgagtaacacgtgggtaacctacctataagactgggataactccgggaaaccggagctaataccggataatattttgaaccgcatggttcaaaagtgaaagatggttttgctatcacttatagatggacccgcgccgtattagctagttggtaaggtaacggcttaccaaggcgacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatacgtgtaagtaactgtgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgtagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaagg

SEQ ID NO: 180 - DNA, 1358 bp, Acinetobacter ursingii
  source 1..1358: mol_type = genomic DNA
attgaacgctggcggcaggcttaacacatgcaagtcgagcagagagaggtagcttgctactgatcttagcggcggacgggtgagtaatacttaggaatctggctattagtgggggacaacatttcgaaagggatgctaataccgcatacgtcctacgggagaaagcaggggatcttcggaccttgcgctaatagatgagcctaagtcggattagctagttggtggggtaaaggcctaccaaggcgacgatctgtagcgggtctgagaggatgatccgccacactgggactgagacacggcccagactcctacgggaagcagcagtggggaatattggacaatggggggaaccctgatccagccatgccgcgtgtgtgaagaaggccttatggttgtaaagcactttaagcgaggaggagggtactggtattaatactaccaggtactggacgttactcgcagaataagcaccggctaactctgtgccagcagccgcggtaatacagagggtgcgagcgttaatcggatttactgggcgtaaagcgtgcgtaggcggctaattgagtcggatgtgaaatccccgagcttaacttgggaattgcattcgatactggttagctagagtgtgggagaggatggtagaattccaggtgtagcggtgaaatgcgtagagatctggaggaataccgatggcgaaggcagccatctggcctaacactgacgctgaggtacgaaagcatggggagcaaacaggattagataccctggtagtccatgccgtaaacgatgtctactagccgttggtctctttgagggattagtggcgcagctaacgcgataagtagaccgcctggggagtacggtcgcaagactaaaactcaaatggattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcactttgggtgggaactttaaggatactgccagtgacaaactggaggaaggcggggacgacgtcaagtcatcatggcccttacgaccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggcct

SEQ ID NO: 181 - DNA, 1461 bp, Ralstonia mannitolilytica
  source 1..1461: mol_type = genomic DNA
attgaacgctggcggcatgccttacacatgcaagtcgaacggcagcgggggaaagcttgctttcctgccggcgagtggcgaacgggtgagtaatacatcggaacgtgccctgtagtgggggataactagtcgaaagattagctaataccgcatacgacctgagggtgaaagtgggggaccgcaaggcctcatgctataggagcggccgatgtctgattagctagttggtggggtaaaggcctaccaaggcgacgatcagtagctggtctgagaggacgatcagccacactgggactgagacacggcccagactcctacgggaggcagcagtggggaattttggacaatgggcgaaagcctgatccagcaatgccgcgtgtgtgaagaaggccttcgggttgtaaagcacttttgtccggaaagaaatggctctggttaatacccggagttgatgacggtaccggaagaataaggaccggctaactacgtgccagcagccgtggtaatacgtagggtccaagcgttaatcggaattactgggcgtaaagcgtgcgcaggcggttgtgcaagaccgatgtgaaatccccgggcttaacctgggaattgcattggtgactgcacggctagagtgtgtcagagggaggtagaattccacgtgtagcagtgaaatgcgtagagatgtggaggaataccgatggcgaaggcagcctcctgggataacactgacgctcatgcacgaaagcgtggggagcaaacaggattagataccctggtagtccacgccctaaacgatgtcaactagttgttggggattcatttccttagtaacgtagctaacgcgtgaagttgaccgcctggggagtacggtcgcaagattaaaactcaaaggaattgacggggacccgcacaagcggtggatgatgtggattaattcgatgcaacgcgaaaagccttacctacccttgacatgccactaacgaagcagagatgcattaggtgctcgaaagagaaagtggacacaggtgctgcatggctgtcgtcagctcgtgtcgtgagatgttgggttaagtcccgcaacgagcgcaacccttgtctctagttgctacgaaagggcactctagagagactgccggtgacaaaccggaggaaggtggggatgacgtcaagtcctcatggcccttatgggtagggcttcacacgtcatacaatggtgcatacagagggttgccaagccgcgaggtggagctaatcccagaaaatgcatcgtagtccggatcgtagtctgcaactcgactacgtgaagctggaatcgctagtaatcgcggatcagcatgccgcggtgaatacgttcccgggtcttgtacacaccgcccgtcacaccatgggagtgggctttaccagaagtagttagcctaaccgcaaggagggcgattaccacggtagggttcatgactggggtga

SEQ ID NO: 182 - DNA, 1384 bp, Corynebacterium stationis
  source 1..1384: mol_type = genomic DNA
gagtttgatcctggctcaggatgaacgctggcggcgtgcttaacacatgcaagtcgaacggaaaggccttgtgcttgcacaaggtactcgagtggcgaacgggtgagtaacacgtgggtgatctgccctgcactgtgggataagcctgggaaactgggtctaataccatataggaccgcatcttggatggtgtggtggaaagcttttgcggtgtgggatgagcctgcggcctatcagcttgttggtggggtaatggcctaccaaggcggcgacgggtatccggcctgagagggtgtacggacacattgggactgagatacggcccagactcctacgggaggcagcagtggggaatattgcacaatgggcgcaagcctgatgcagcgacgccgcgtgggggatgaaggccttcgggttgtaaactcctttcgctatcgacgaagccacttgtggtgacggtaggtagataagaagcaccggctaactacgtgccagcagccgcggtaatacgtagggtgcaagcgttgtccggaattactgggcgtaaagagctcgtaagtggttcgtcgcgtcgtctgtgaaatcccggggcttaacttcgggcgtgcaggcgatacgggcataacttgagtgctgtaggggagactggaattcctggtgtaccggtgaaatgcgcagatatcaggaggaacaccgatggcgaaggcaggtctctgggcagttactgacgctgaggagcgaaagcatgggtagcgaacaggattagataccctggtagtccatgccgtaaacggtgggcgctaggtgtagggggcttccacgtcttctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatatacaggatcgggctagagatagtctttcccttgtggtctgtatacaggtggtgcatggttgtcgtcagctcgtgtcgtgagatgttgggttaagtcccgcaacgagcgcaacccttgtcttatgttgccagcacgttatggtgggaactcatgagagactgccggggttaactcggaggaaggtggggatgacgtcaaatcatcatgccccttatgtccagggcttcacacatgctacaatggtcgatacagtgggcagcgacatcgtaaggtggagcgaatccctgaaagtcggccttagttcggattggggtctgcaactcgaccccatgaagtcggagtcgctagtaatcgcagatcagcaacgctgcggtgaatacgttcaccgggccttgtacacaccgcccgtc

SEQ ID NO: 183 - DNA, 1372 bp, Actinomyces oris
  source 1..1372: mol_type = genomic DNA
gacgaacgctggcggcgtgcttaacacatgcaagtcgaacggtgaagcaccttccttttggtgggtgtggatgagtggcgaacgggtgagcaacacgtgagtaacctgcccccttcttctggataaccgcatgaaagtgtggctaatacgggatattctgcgcctgctgcatggtgggtgtgggaaagatattgtttcggtgggggatgggctcgcggcctatcagcttgttggtggggtgatggcctaccaaggcggtgacgggtagccggcctgagagggtggacggtcacactgggactgagacacggcccagactcctacgggaggcagcagtggggaatattgcacaatgggcgcaagcctgatgcagcgacgccgcgtgagggatggaggccttcgggttgtaaacctctttcgccagtgaagcaggcctgtcccttgtgggtgggttgacggtagctggataagaagcgccggctaactacgtgccagcagccgcggtaatacgtagggcgcgagcgttgtccggaattattgggcgtaaagagctcgtaggcggctggtcgcgtctgtcgtgaaatcctctggcttaactgggggcttgcggtgggtacgggccggcttgagtgcggtaggggagactggaactcctggtgtagcggtggaatgcgcagatatcaggaagaacaccggtggcgaaggcgggtctctgggccgttactgacgctgaggagcgaaagcgtggggagcgaacaggattagataccctggtagtccacgccgtaaacgttgggcactaggtgtggggggccttttccgggtcttccgcgccgtagctaacgcattaagtgccccgcctggggagtacggccgcaaggctaagactcaaaggaattgacgggggcccgcacaagcggcggagcatgcggattaattcgatgcaacgcgaagaaccttaccaaggcttgacatgtgccggtcggctccggagacggggtttcctccttgtggggccggttcacaggtggtgcatggttgtcgtcagctcgtgtcgtgagatgttgggttaagtcccgcaacgagcgcaacccttgtcccgtgttgccagcacgttgtggtggggactcgcgggagactgccggggtcaactcggaggaaggtggggatgacgtcaaatcatcatgccccttatgtcttgggcttcacgcatgctacaatggccggtacagagggctgcgataccgtgaggtggagcgaatcccttaaagccggtctcagttcggatcggtgtctgcaactcgacaccgtgaagttggagtcgctagtaatcgcagatcagcaacgctgcggtgaatacgttctcgggcct

SEQ ID NO: 184 - DNA, 1531 bp, Lactobacillus iners
  source 1..1531: mol_type = genomic DNA
ggctcgggacgaacgctggcggcgtgcctaatacatgcaagtcgagcgagtctgccttgaagatcggagtgcttgcactctgtgaaacaagatacaggctagcggcggacgggtgagtaacacgtgggtaacctgcccaagagatcgggataacgcctggaaacagatgctaataccggataacaacagatgatgcctatcaactgtttaaaagatggttctgctatcactcttggatggacctgcggtgcattagctagttggtagggtaacggcctaccaaggcgatgatgcatagccgagttgagagactgatcggccacattgggactgagacacggcccaaactcctacgggaggcagcagtagggaatcttccacaatggacgcaagtctgatggagcaacgccgcgtgagtgaagaagggtttcggctcgtaaagctctgttgttggtgaagaaggacaggggtagtaactgaccttttgtttgacggtaatcaattagaaagtcacggctaactacgtgtcagcagccgcagtaatacgtaggtggcaagcgttgtccggatttattgggcgtaaagcgagtgcaggcggctcgataagtctgatgtgaaagcctttcggctcaaccggaaaattggattcagatactgtcaagccttggagtacagaaagaaggagatgtggaactccatgtgttagccggttcaattgccttattatatggaagaacaccggtggcgaaggcggctctctggtctgttactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccatagccagtctaagagattagatgttcccttcggggactatgagacaggtggtgcatggctgtcgtcagctcgtgtcaagaatgttaggtttaaagtcccgcaacgagcgcaacccttgtcattagttgccagcattaagttgggcactctaatgagactgccggtgacaaaccggaggaaggtggggatgacgtcaagtcatcatgccccttatgacctgggctacacacgtgctacaatggacggtacaacgagaagcgaccctgtgaaggcaagcggatctctgaaagccgttctcagttcggattgcaggctgcaactcgcctgcatgaagctggaatcgctagtaatcgcaaatcagcacgttgcggtgaatacgttcccgggccttgtacacaccgcccgtcacaccatgagagtctgtaacgcccgaagccggcgggaccacccaaaggagtcagccgtctcaggcgggacagatgattagggtgaagtcgtaacaaggtagcc

SEQ ID NO: 185 - DNA, 1375 bp, Abiotrophia defectiva
  source 1..1375: mol_type = genomic DNA
gacgaacgctggcggcgtgcctaatacatgcaagtcgaacgaaccgcgactgagtgcttgcacttggtcaaggtgagtggcgaacgggtgagtaacacgtgggtaacctacctcatagtgggggataacagtcggaaacgactgctaataccgcataggacataggatcacatgatccagtgaggaaaggtggcgcaagctatcgctaagagatggacccgcggtgcattagctagttggtagggtaaaggcctaccaaggcgatgatgcatagccgacctgagagggtgatcggccacattgggactgaggcacggcccaaactcctacgggaggcagcagtagggaatcttccgcaatggacgcaagtctgacggagcaacgccgcgtgagtgaagaaggtcttcggatcgtaaagctctgttgttagagaagaacagcgcatagagtaactgttatgcgtgtgacggtatctaaccagaaagccacggctaactacgtgccagcagccgcggtaatacgtaggtggcgagcgttgtccggatttattgggcgtaaagggagtgtaggcggtcttttaagtctgatgtgaaagcccacggctcaaccgtggagggtcattggaaactgggagacttgagtgcagaagaggagagcggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggaggggttccacccttcagtgctggagttaacgcaataagcactccgcctggggagtacggccgcaaggctgaaactcaaaggaattgacggggacccgcacaagcggtggagcatgtggtttaattcgaagcaacgcgaagaaccttaccaggtcttgacatcccgacgaccgctctagagatagagtttttcttcggaacgtcggtgacaggtggtgcatggttgtcgtcagctcgtgtcgtgagatgttgggttaagtcccgcgacgagcgcaacccctataactagttgccagcattaagatggggactctagttagactgccggtgacaaaccggaggaaggtggggatgacgtcaaatcatcatgccccttatgacctgggctacacacgtgctacaatggatggtacaacgagcagcgaactcgcgagggtaagcgaatctctaaaagccattctcagttcggattgtagtctgcaactcgactacatgaagccggaatcgctagtaatcgcggatcagcacgccgcggtgaatacgttcccgggtct

SEQ ID NO: 186 - DNA, 1367 bp, Streptococcus
  source 1..1367: mol_type = genomic DNA
gacgaacgctggcggcgtgcctaatacatgcaagtagaacgctgaaggaggagcttgctcttctggatgagttgcgaacgggtgagtaacgcgtaggtaacctgcctggtagcgggggataactattggaaacgatagctaataccgcataacagtagatgttgcatgacatttgcttaaaaggtgcaattgcatcactaccagatggacctgcgttgtattagctagttggtggggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgccgtaaacgatgagtgctaggtgttagaccctttccggggtttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgacctctctagagatagagctttccttcgggacagaggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctcttaaagccagtctcagttcggattgtaggctgcaactcgcctacatgaagtcggaatcgctagtaatcgcggatcagcacgccgcggtgaatacgttcccgggcct

SEQ ID NO: 187 - DNA, 1347 bp, Wautersiella
  source 1..1347: mol_type = genomic DNA
gatgaacgctagcgggaggcttaacacatgcaagccgaggggtatagttctttcgggaactagagaccggcgcacgggtgcgtaacgcgtatgcaacttgccctactgaaaaggatagcccttcgaaaggaggattaatactttataatagaaatcatggcatcatgaatttttgaaagatttatcgcagtaggataggcatgcgtaagattagttagttggtgaggtaacggctcaccaagacgatgatctttagggggcctgagagggtgaacccccacactggtactgagacacggaccagactcctacgggaggcagcagtgaggaatattggacaatgggtggaagcctgatccagccatcccgcgtgtaggatgacggccttatgggttgtaaactacttttatctggggataaacctacttacgtgtaagtagctgaaggtaccagaagaataagcaccggctaactccgtgccagcagccgcggtaatacggagggtgcaagcgttatccggatttattgggtttaaagggtccgtaggcggattaatcagtcagtggtgaaatctcatagcttaactatgaaactgccattgatactgttagtcttgagtgatgttgaagttgctggaatgtgtagtgtagcgtggaaatgcttagatattacgcagaacaccaattgcgaaggcaggtgactaaacatcaactgacgctgatggacgaaagcgtggggagcgaacaggattagataccctggtagtccacgccgtaaacgatggatactttctgttggattttcggattcagtggctaagcgaaagttataagtatcccacctggggagtacgttcgcaagaatgaaactcaaaggaattgacgggggcccgcacaagcggtggagcatgtggtttaattcgatgatacgcgaggaaccttaccaaggcttaaatgcaatttgacagatctagaaatagatttttcttcggacagaatgcaaggtgctgcatggctgtcgtcagctcgtgccgtgaggtgttaggttaagtcctgcaacgagcgcaacccctatcattagttgccagcgtttaaagacggggactctaatgagactgccggtgcaaaccgcgaggaaggtggggacgacgtcaagtcatcacggcccttacgtcttgggctacacacgtgctacaatggtaagtacagagggcagctacttggcaacaagatgcgaatctcaaaaacttatctcagttcggattggagtctgcaactcgactctatgaagctggaatcgctagtaatcgcatatcagccatgatgcggtgaatacgttcccgggcct

SEQ ID NO: 188 - DNA, 1367 bp, Streptococcus cristatus
  source 1..1367: mol_type = genomic DNA
gacgaacgctggcggcgtgcctaatacatgcaagtagaacgctgaaggaggagcttgctcttctggatgagttgcgaacgggtgagtaacgcgtaggtaacctgcctggtagcgggggataactattggaaacgatagctaataccgcataatattgactattgcatgatagtcaattgaaaggtgcaaatgcaccactaccagatggacctgcgttgtattagctagttggtggggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtacgctttggaaactgtttaacttgagtgcagaaggggagagtggaattccatgtgtagcggtgagatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttgggtcctttccgggactcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccggtgcccgtcctagagataggattttgcttcggcacaccggtgacaggtggtgcatggttgtcgtcagctcgtgtcgtgagatgttgggttaagtcccgcaacgagcgcaacccttattgttagttgccatcattgagttgggcactctagcgagactgccggtaataaaccggaggaaggtggggatgacgtcaaatcatcatgccccttatgacctgggctacacacgtgctacaatggctggtacaacgagtcgcaagtcggtgacggcaagctaatctcttaaagccagtctcagttcggattgtaggctgcaactcgcctacatgaagtcggaatcgctagtaatcgcggatcagcacgccgcggtgaatacgttcccgggcct

SEQ ID NO: 189 - DNA, 1496 bp, Klebsiella pneumoniae
  source 1..1496: mol_type = genomic DNA
agagtttgatcctggctcagattgaacactggcggcaggcctaacacatgcaagtcgagcggtagcacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctggtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggcggtgaggttaataacctcatcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctggtcttgacatccacagaacttaccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccacttagagattcatgactggggtgaagtcgtaacaagg

SEQ ID NO: 190 - DNA, 1341 bp, Blastococcus
  source 1..1341: mol_type = genomic DNA
gacgaacgctggcggcgtgcttaacacatgcaagtcgagcgaggcctttccttcgggagagtgtcctagcggcgaacgggtgagtaacacgtgggcaacctgccctcagctctgggataactccaagaaattggggctaataccggatatgaccgctgaccgcatggtctggtggtggaaagatttatcggctggggatgggcccgcggcctatcagcttgttggtggggtagtggcctaccaaggcgacgacgggtagccggcctgagagggtgaccggccacactgggactgagacacggcccagactcctacgggaggcagcagtggggaatattgcgcaatgggcgaaagcctgacgcagcgacgccgcgtgggggatgacggccttcgggttgtaaacctctttcagcagggacgaagcgaaagtgacggtacctgcagaagaagcaccggccaactacgtgccagcagccgcggtaatacgtagggtgcaagcgttgtccggaattattgggcgtaaagagctcgtaggcggtttgttgcgtcggctgtgaaatcccgaggctcaacctcgggtctgcagtcgatacgagcaaactagagtgttgcaggggagactggaattcctggtgtggcggtgaaatgcgcagatatcaggaggaacaccggtggcgaaggcgggtctctgggcaacaactgacgctgaggagcgaaagcgtggggagcgaacaggattagataccctggtagtccacgccgtaaacgttgggcgctaggtgtggggctcattccacgagttccgtgccgcagctaacgcattaagcgccccgcctggggagtacggccgcaaggctaaaactcaaaggaattgacgggggcccgcacaagcggcggagcatgttgcttaattcgatgcaacgcgaagaaccttacctaggcttgacatgcacggaaatcctccagagatggtgggtccgtaagggccgtgcacaggtggtgcatggttgtcgtcagctcgtgtcgtgagatgttgggttaagtcccgcaacgagcgcaaccctcgttctatgttgccagcacgtcatggtggggactcataggagactgccggggtcaactcggaggaaggtgggggtgacgtcaaatcatcatgccccttatgtctagggctgcaaacatgctacaatggccggtacaaagggctgcgataccgcgaggtggagcgaatcccaaaaagccggtctcagttcggattggggtctgcaactcgaccccatgaagttggagtcgctagtaatcgcagatcagcaacgctgcggtgaatacgttcccgggcct

SEQ ID NO: 191 - DNA, 1428 bp, Anoxybacillus kestanbolensis
  source 1..1428: mol_type = genomic DNA
agcggacaaatcgaacgcttgcttttgattcgttagcggcggacgggggagtaacacgtgggcaacctgccctgtagacggggataacaccgagaaatctgcgctaataccggataacacgaaatgtcgcatgacgtttcgttgaaagacggcgcaagctgtcgctacaggatgggcccgcggcgcattatctagttggtgaggtaacggctcaccaaggcgacgatgcgtacccgacctgagagggtgatcggccacactggcactgacacacggcccaaactcctacggcaggcagcagtacgcaatcttccgcaatggacgaaagtctgacggagcaacgccgcgtgagcgaagaaggccttcgcgccgtaaagctctgttgttagggaagaacaagtagcgcactaactggcgttaccgtgacgggacctaacgagaaagccacggctaactacgtgccagcagccgcggtaatacgtaggtggcaagcgttgtccggaattattgtgcgtaaagcgcgcgcaggccgttccttaagtctgatgtgaaagtccacggctcaaccgtggagggtcattggaaactgggggacttgagtgcagaagaggagagcggaattccacgtgtagcgtgtgaaatgcgtagagatgtggaggaacaccagtggcgaaggcggctctctggtctgtaactgacgctgaggcgcgaaagcgtggggagcaaacaggattagataccctggtagtccacgccgtaaacgatgagtgctaagtgttagagggtatccaccctttagtgctgtagttaacgcattaagcactccgcctggggagtacgctcgcaagagtgaaactcaaaggaattgacgggggcccgcacaagcggtggagcatgtggtttaattcgaagcaacgcgaagaaccttaccaggtcttgacatcccctgacaacccgagagatcgggcgttcccccttcggggggacagggtgacaggtggtgcatggttgtcgtcagctcgtgtcgtgagatgttgggttaagtcccgcaacgagcgcaaccctcgaccttagttgccagcattcagttgggcactctaaggtgactgccggctaaaagtcggaggaaggtggggatgacgtcaaatcatcatgccccttatgacctgggctacacacgtgctacaatgggcggtacaaagggtcgcgaacccgcgagggggagccaatcccaaaaagccgctctcagttcggattgcaggctgcaactcgcctgcatgaagccggaatcgctagtaatcgcggatcagcatgccgcggtgagtacgttcccgggccttgtacacaccgcccgtcacaccacgagagtttgcaacacccgaagtcggtgaggtaacccttaagggagccagccgccgaaggtggggc

SEQ ID NO: 192 - DNA, 1454 bp, Prevotella tannerae
  source 1..1454: mol_type = genomic DNA
gatgaacgctagctataggcttaacacatgcaagtcgtggggcagcggatgcttagcttgctaagtatgccggcgaccggcgcacgggtgagttaacgcgtaccgaacctgcccatcacacagggataggcttgcgaaagcagaattaatacctgatggtctcagttgtatgcatgtatgattgagtaaagccttcgggcggtgatggatggcggtgcgtcccattaaggaagttggcggggtaacggcccaccaatccttcgatgggtaggggttctgagaggaaggtcccccacattggaactgagatacggtccaaaactctacgggaggcagcagtgaggaatgattggtcaatgggcgagagcctgaaccagccaagtagcgtgcgggatgaaagcccttgtggtcgtaaaccgcttttatcagtgaataaagtggcaccacgtgtggtgtcattgcaggtagctgaagaaaaaggaccggctaattccgtgccagcagccgcggtaatacggaaggtccgggcgttatccggaattattgggtttaaagggagcgcaggcgggagtgtaagtcagctgttaaatatcagggcccaactctgttatgcagttgaaactatatttcttgagtacgcacagggatggcggaattcagggtgtagcggtgaaatgcttagatatcctgaagaactccgatcgcgaaggcagccatccggagcgtaactgacgctgaggctcgaaggtgcgggtatcgaacaggattagataccctggtagtccgcacggtaaacgatgaatactcgcagttcggccgtttaggtcgagttgtccagcgaaagcgttaagtattccacctggggagtacgccggcaacggtgaaactcaaaggaattgacgggggcccgcacaagcggaggaacatgtggtttaattcgatgatacgcgaggaaccttacccgggcttgaaccaagagtgacgagtgcagagatgcgcttattcttcggacacttttggaggtgctgcatggttgtcgtcagctcgtgccgtgaagtgtcggcttaagtgccataacgagcgcaacccttttctacgatgccatcgggtaatgctgggaaccccgtggatactgccgccgcaaggcgaggaaggtgggaatgacgtcaaatcagcacggcccttacgtccggggctacacacgtgttacaatggtcggagcagcaggaagctacctggcgacaggatgctgatcccaaaacccggcctcagttcggactggagtctgcaacccgactccacgaagccggattcgctagtaatcgcgcatcagccatggcgcggtgaatacgttcccgggccttgtacacaccgcccgtcaagccatgaaagccgggggtgcctgaaagccgtgaccgcgagggtcggcctagggtaaaaccggtgattggggc

SEQ ID NO: 193 - DNA, 1501 bp, Neisseria oralis
  source 1..1501: mol_type = genomic DNA
agagtttgatcacggctcagattgaacgctggcggcatgctttacacatgcaagtcggacggcagcggggtagtgcttgcactactgccggcgagtggcgaacgggtgagtaacatatcggaacgtaccgagcagtgggggataactaatcgaaagattagctaataccgcatatattctgaggaagaaagcaggggacctttgggccttgcgctgtttgagcggccgatatctgattagctggttggtggggtaaaggcctaccaaggcgacgatcagtagcgggtctgagaggatgatccgccacactgggactgagacacggcccagactcctacgggaggcagcagtggggaattttggacaatgggggcaaccctgatccagccatgccgcgtgtctgaagaaggccttcgggttgtaaaggacttttgtccgggaagaaaagcgcgatgttaataccattgcgtgctgacggtaccggaagaataagcaccggctaactacgtgccagcagccgcggtaatacgtagggtgcgagcgttaatcggaattactgggcgtaaagcgggcgcagacggttacttaagcaggatgtgaaatccccgggctcaacctgggaattgcgttctgaactgggtggctagagtgtgtcagaggggggtagaattccacgtgtagcagtgaaatgcgtagagatgtggaggaataccgatggcgaaggcagccccctgggatggcactgacgttcatgcccgaaagcgtgggtagcaaacaggattagataccctggtagtccacgccctaaacgatgtcgattagctgttgggcaacttgattgcttggtagcgaagctaacgcgtgaaatcgaccgcctggggagtacggtcgcaagattaaaactcaaaggaattgacggggacccgcacaagcggtggatgatgtggattaattcgatgcaacgcgaagaaccttacctggtcttgacatgtacggaaccttccggagacggaagggtgccttcgggagccgtaacacaggtgctgcatggctgtcgtcagctcgtgtcgtgagatgttgggttaagtcccgcaacgagcgcaacccttgtcattagttgccatcatttggttgggcactctaatgagactgccggtgacaagccggaggaaggtggggatgacgtcaagtcctcatggcccttatgaccagggcttcacacgtcatacaatggtcggtacagagggtagccaagccgcgaggtggagccaatcccaaaaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagcaggtaggctaaccgcaagggggccgcttgccacggtatgcttcatgactggggtgaagtcgtaacaaggtaac

SEQ ID NO: 194 - DNA, 1397 bp, Acinetobacter
  source 1..1397: mol_type = genomic DNA
agagtttgatcctggctcagattgaacgctggcggcaggcttaacacatgcaagtcgagcgggcgaggttgcttcggtaacttagctagcggcggacgggtgagtaatgcttaggaatctgcctattagtgggggacaacattccgaaagggatgctaataccgcatacgtcctacgggagaaagcaggggctctttatgaccttgcgctaatagatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatggggggaaccctgatccagccatgccgcgtgtgtgaagaaggccttttggttgtaaagcactttaagtggggaggaggctctcttggttaatacctgagaagagtggacgttacccacagaataagcaccggctaactctgtgccagcagccgcggtaatacagagggtgcgagcgttaatcggatttactgggcgtaaagcgcgcgtaggtggttaattaagtcaaatgtgaaatccccgagcttaacttgggaattgcattcgatactggttagctagagtatgggagaggatggtagaattccaggtgtagcggtgaaatgcgtagagatctggaggaataccgatggcgaaggcagccatctggcctaatactgacactgaggtgcgaaagcatggggagcaaacaggattagataccctggtagtccacgccgtaaacgatgtctactagccgttgggggatttgatcctttagtggcgcagctaacgcgataagtagaccgcctggggagtacggtcgcaagactaaagctcaaatgaattgacgggggcccgcacaagcggtggagcatgtggtttaattcgatgcaacgcgaagaaccttacctggccttgacatagtagaaactttccagagatggattggtgccttcgggaatctacatacaggtgctgcatggctgtcgtcagctcgtgtcgtgagatgttgggttaagtcccgcaacgagcgcaacccttttccttacttgccagcatttcggatgggaactttaaggatactgccagtgacaaactggaggaaggcggggacgacgtcaagtcatcatggcccttacggccagggctacacacgtgctacaatggtcggtacaaagggttgctacacagcgatgtgatgctaatctcaaaaagccgatcgtagtccggatcgcagtctgcaactcgactgcgtgaagtcggaatcgctagtaatcgcggatcagaatgccgcggtgaatacgttcccgggccttgtactcaccgcccgtca

SEQ ID NO: 195 - DNA, 1466 bp, Faecalibacterium prausnitzii
  source 1..1466: mol_type = genomic DNA
gtttgatcatggctcaggacgaacgctggcggcgcgcctaacacatgcaagtcgaacgagagatgaagagcttgctcttcaaatcgagtggcgaacgggtgagtaacgcgtgaggaacctgcctcaaagagggggacaacagttggaaacgactgctaataccgcataagcccacggctcggcatcgagcagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggagaacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacaccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgatgcaacgcgaagaaccttaccaagtcttgacatcctgcgacgcacatagaaatatgtgtttccttcgggacgcagagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgtggatcagcatgccacggtgaatacgttcccgggccttgtacacaccgcccgtcacaccatgagagccggggggacccgaagtcggtagtctaaccgcaaggaggacgccgccgaaggtaaaactaatgattggggtgaagtcgtaacaaggta

SEQ ID NO: 196 - DNA, 1343 bp, Janibacter
  source 1..1343: mol_type = genomic DNA
gacgaacgctggcggcgtgcttaacacatgcaagtcgaacggtgaagctccagcttgctggagtggatcagtggcgaatgggtgagtaacacgtgagcaacctgcccctgactctggaataagcgctggaaacggcgtctaatactggatatgagaagggtccgcatggacactttctggaaagtttttcggttggggatgggctcgcggcctatcagcttgttggtgaggtaatggctcaccaaggcgacgacgggtagccgacctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcaccggctaactacgtgccagcagccgcggtaatacgtagggtgcgagcgttgtccggaattattgggcgtaaagagcttgtaggcggtttgtcgcgtctgctgtgaaaatccggggctcaaccccggacttgcagtgggtacgggcagactagagtgtggtaggggagactggaattcctggtgtagcggcgaaatgcgcagatatcaggaggaacaccgatggcgaaggcaggtctctgggccactactgacgctgagaagcgaaagcatggggagcgaacaggattagataccctggtagtccatgccgtaaacgttgggcgctaggtgtgggactcattccacgagttccgtgccgcagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatataccggaaacttccagagatggttgccccctttgggtcggtatacaggtggtgcatggttgtcgtcagctcgtgtcgtgagatgttgggttaagtcccgcaacgagcgcaaccctcgttctatgttgccagcacgttatggtggggactcatggaagactgccggggtcaactcggaggaaggtggggatgacgtcaaatcatcatgccccttatgtcttgggcttcacgcatgctacaatggccggtacaaagggctgcgatgccgtaaggtggagcgaatcccaaaaaaccggtctcagttcggattggggtctgcaactcgaccccatgaagtcggagtcgctagtaatcgcagatcagcaacgctgcggtgaatacgttcccgggcct

SEQ ID NO: 197 - DNA, 1492 bp, unidentified
  misc_feature 1..1492: note = 16S ribosomal RNA of unknown bacteria
  source 1..1492: mol_type = genomic DNA
atctcatggagagttcgatcctggctcaggatgaacgctggcggcatgcttaacacatgcaagtcggacgggaagtggtgtttccagtggcggacgggtgagtaacgcgtaagaacctgcccttgggaggggaacaacagctggaaacggctgctaataccccgtaggctgaggagcaaaaggaggaatccgcccgaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtggaggtagaaggcctacgggtcgtgaacttcttttcccggagaagaagcaatgacggtatctggggaataagcatcggctaactctgtgccagcagccgcggtaatacagaggatgcaagcgttatccggaatgattgggcgtaaagcgtctgtaggtggctttttaagtccgccgtcaaatcccagggctcaaccctggacaggcggtggaaactaccaagctggagtacggtaggggcagagggaatttccggtggagcggtgaaatgcgtagagatcggaaagaacaccaacggcgaaagcactctgctgggccgacactgacactgagagacgaaagctaggggagcgaatgggattagataccccagtagtcctagccgtaaacgatggatactaggcgctgtgcgtatcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccttcgggaacgcggacacaggtggtgcatggctgtcgtcagctcgtgccgtaaggtgttgggttaagtcccgcaacgagcgcaaccctcgtgtttagttgccaccgttgagtttggaaccctgagcagactgccggtgataagccggaggaaggtgaggatgacgtcaagtcatcatgccccttatgccctgggcgacacacgtgctacaatggccgggacaaagggtcgcgatcccgcgagggtgagctaactccaaaaacccgtcctcagttcggattgtaggctgcaactcgcctacatgaagccggaatcgctagtaatcgccggtcagccatacggcggtgaattcgttcccgggccttgtacacaccgcccgtcacactatgggagctggccatgcccgaagtcgttaccttaaccgcaaggagggggatgccgaaggcagggctagtgactggagtgaagtcgtaacaaggtagccgtactggaaggtgcggctggatcacctccttt

SEQ ID NO: 198 - DNA, 1362 bp, unidentified
  misc_feature 1..1362: note = 16S ribosomal RNA of unknown bacteria
  source 1..1362: mol_type = genomic DNA
gacgaacgctggcggcgtgcttaacacatgcaagtcgaacgatgaagaaaagaattgaagcttcggcagaaattcatttcggattagtggcagacgggtgagtaacgcgtgagcaacctacctcttacaaagggatagcctcgggaaaccgggattaataccatataacattgcgacgaagcatttcgatgcaatcaaagctacggcggtaagagatgggctcgcgtcccattaaccggatggtgaggtaacggctcaccatagtgacgatgggtaaccggcctgagagggtgaacggtcacactggaactgagacacggtccagactcctacgggaggcagcagtggggaatattgcacaatggagggaactctgatgcagcgacgccgcgtgagtgatgaaggaattcgtttcgtaaaactctgttctatgggaagaaaaggactgtaccataggagaaagctccggctaaatacgtgccagcagccgcggtaatacgtatggagcgagcgttgtccggaattattgggcgtaaagggtgcgcaggcggctttacaagttggatgtgaaatattgtggctcaaccacaaacgtgcatccaaaactgcaaagcttgagttaaggagaggtaagtggaattcctggtgtagcggtggaatgcgtagatatcaggaggaataccggtggcgaaggcgacttactggacttaaactgacgctcaggcacgaaagcgtggggagcaaacaggattagataccctggtagtccacgccgtaaacgatgagtgctaggtgttgggggtcaaacctcggtgccgcaagcaaacgcaataagcactccgcctggggagtacgtgcacaagcatgaaactcaaaggaattgacagggacccgcacaagcagcggagcatgtggtttaattcgtggctacgcgaagaaccttaccagggcttgacatatatatgaggggacatagagatatgaaaccggtaagagcttgctcttaacatatatacaggtggtgcatggttgtcgtcagctcgtgtcgtgagatgttgggttaagtcccgcaacgagcgcaacccttgcaattagttactaacagatagtgctgaggactctaattggactgccgatgataaatcggaggaaggtggggatgacgtcaaatcatcatgccctttgtgtcctgggctacacacgtgctacaatggtcggtacaaagaggagcgaaatggtgacatcaagcaaatctcaaaaaaccgatcccagttcggattgtactctgcaactcgagtacatgaagtcggagttgctagtaatcgcggatcagaatgccgcggtgaatgcgttcccgggtct

SEQ ID NO: 199 - DNA, 1531 bp, Lactobacillus iners; identical to SEQ ID NO: 184
  source 1..1531: mol_type = genomic DNA

SEQ ID NO: 200 - DNA, 1526 bp, Pseudomonas aeruginosa
  source 1..1526: mol_type = genomic DNA
agtttgatcctggctcagattgaacgctggcggcaggcctaacacatgcaagtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaatccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggttaaaactcaaatgaattgacgggggcccgcacaagcggtggagcatgtggtttaattccaagcaacgcgaagaaccttacctggccttgacatgctgagaactttccagagatggattggtgacttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccgcaagggggacggttaccacggagtaattcatgactggggtgaagtcgtaacaaggtagccgtaggggaacctgcggctggatcacctcctt

SEQ ID NO: 201 - DNA, 1339 bp, Propionibacterium granulosum
  source 1..1339: mol_type = genomic DNA
gacgaacgctggcggcgtgcttaacacatgcaagtcgtacggtaaggccctttcgggggtacacgagtggcgaacgggtgagtaacacgtgagtaacctgcccacaactttgggataacgctaggaaactggtgctaatactggatatgtgctcctgctgcatggtgggggttggaaagctccggcggttgtggatggactcgcggcctatcagcttgttggtggggtagtggcctaccaaggcggcgacgggtagccggcctgagagggtgaccggccacattgggactgagatacggcccagactcctacgggaggcagcagtggggaatattgcacaatgggcgcaagcctgatgcagcaacgccgcgtgcgggatgacggccttcgggttgtaaaccgctttcagcagggacgaagctttttgtgacggtacctgcagaagaagcaccggctaactacgtgccagcagccgcggtgatacgtagggtgcgagcgttgtccggatttattgggcgtaaagggctcgtaggcggttgatcgcgtcggaagtggaaacttgatgcttaacgttgagcgtgctttcgatacgggttgacttgaggaaagtaggggagaatggaattcctgtgggagcggtggaatgcgcagatatcaggaggaacaccagtggcgaaggcggtcttctggacctttcctgacgctgaggagcgaaagcgtggggagcgaacaggcttagataccctggtagtccacgctgtaaacggtgggtactaggtgtggggtccattccacggattctgtgccgtagctaacgcattaagtaccccgcctggggagtacggccgcaaggctaaaactcaaaggaattgacggggccccgcacaagcggcggagcatgcggattaattcgatgcaacgcgaagaaccttacctgggtttgacatggatcgggagcttccagagatggttgtgcctcttttggggtcggttcacaggtggtgcatggctgtcgtcagctcgtgtcgtgagatgttgggttaagtcccgcaacgagcgcaaccctcgtccactgttgccagcaattcggttggggactcagtggagaccgccggggtcaactcggaggaaggtggggatgacgtcaagtcatcatgccccttatgtccagggcttcacgcatgctacaatggccggtacagcgagttgcgacatcgtaaggtggagcgaatctcaaaaagccggtctcagttcggattggggtctgcaactcgacctcatgaagtcggagtcgctagtaatcgcagatcagcaacgctgcggtgaatacgttcccggg

SEQ ID NO: 202 - DNA, 1347 bp, Rothia mucilaginosa
  source 1..1347: mol_type = genomic DNA
gacgaacgctggcggcgtgcttaacacatgcaagtcgaacgatgaagcctagcttgctaggtggattagtggcgaacgggtgagtaatacgtgagtaacctacctttaactctgggataagcctgggaaactgggtctaataccggatacgaccaatctccgcatggggtgttggtggaaagcgttatgtagtggttatagatgggctcacggcctatcagcttgttggtgaggtaacggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctgttagcagggaagaagagagattgacggtacctgcagagaaagcgccggctaactacgtgccagcagccgcggtaatacgtaaggcgcgagcgttgtccggaattattgggcgtaaagagcttgtaagccgtttgtcgcgtctgctgtgaaggccggagcttaactccgtgtattgcagtgggtacgggcagactagagtgcagtaggggagactggaattcctggtgtagcggtggaatgcgcagatatcaggaggaacaccgatggcgaaggcaggtctctgggctgtaactgacgctgagaagcgaaagcatggggagcgaacaggattagataccctggtagtccatgccgtaaacgttgggcactaggtgtggggaacattccacgttttccgcgccgtagctaacgcattaagtgccccgcctggggagtacggccgcaaggctaaaactcaaagaaattgacgggggcccgcacaagcggcggagcatgcggattaattcgatgcaacgcgaagaaccttaccaaggcttgacatatactggaccgcatcagagatggtgtttcccttcggggctggtatacaggtggtgcatggttgtcgtcagctcgtgtcgtgagatgttgggttaagtcccgcaacgagcgcaaccctcgttctatgttgccagcacgttatggtggggactcataggagactgccggggtcaactcggaggaaggtggggatgacgtcaaatcatcatgccccttatgtcttgggcttcacgcatgctacaatggccggtacagagggttgcgatactgtgaggtggagctaatccctaaaagccggtctcagttcggattggggtctgcaactcgaccccatgaagtcggagtcgctagtaatcgcagatcagcaacgctgcggtgaatacgttcccgggcct

SEQ ID NO: 203 - DNA, 1531 bp, Lactobacillus iners; identical to SEQ ID NO: 184
  source 1..1531: mol_type = genomic DNA

SEQ ID NO: 204 - DNA, 1367 bp, Streptococcus infantis
  source 1..1367: mol_type = genomic DNA
gacgaacgctggcggcgtgcctaatacatgcaagtagaacgctgaaggaggagcttgctctttccggatgagttgcgaacgggtgagtaacgcgtaggtaacctgcctggtagcgggggataactattggaaacgatagctaataccgtataacagtagatatcgcatgatagctgcttgaaaggtgcaattgcaccactaccagatggacctgcgttgtattagctagttggtgaggtaacggctcaccaaggcaacgatacatagccgacctgagagggtgatcggccacactgggactgagacacggcccagacacctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaagtcccgagcgttgtccggatttaatgggcgtaaagcgagcgcagcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgccgtaaacgatgagtgctaggtgttagaccctttccggggtttagtgccgtagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggttggtacaacgagtcgcaagccggtgacggcaagctaatctcttaaagccaatctcagttcggattgtaggctgcaactcgcctacatgaagtcggaatcgctagtaatcgcggatcagcacgccgcggtgaatacgttcccgggcct

SEQ ID NO: 205 - DNA, 1520 bp, Veillonella
  source 1..1520: mol_type = genomic DNA
tgatcctggctcaggacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaatctcggcatcgaggatagatgaaaggtggcctctacatgtaagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaatagtgagaaggattgacggtaccggaatagaaagccacggctaactacgtgccagcagccgcggtaatacgtaggtggcaagcgttgtccggaattattgggcgtaaagcgcgcgcaggcggataggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaactagagatagttcctcttcttcggaagccagaaaacaggtggtgcacggttgtcgtcagctcgtgtcgtgagatgttgggttaagtcccgcaacgagcgcaacccctatcttatgttgccagcacgtaaaggtgggaactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattgggaaaaagtcgtaacaaggtagccg

SEQ ID NO: 206 - DNA, 1364 bp, Rothia dentocariosa
  source 1..1364: mol_type = genomic DNA
ctggcggcgtgcttaacacatgcaagttcgaacgaagaagcctagctttgctaggtggattagtcggcgaaacgggtgagtaatacgttgagtgacctaccttttgactctgggataagcctgggaaactgggtctaataccggatacgaccaatctccgcatggggtgttggtggaaagcgttatggagtggttttagatgggctcacggcctatcagcttgttggtgaggtaatggcttaccaaggcgacgacgggtagccggcctgagagggtgaccggccacactgggactgagacacggcccagactcctatgggaggcagcagtggggaatattgcacaatgggcgcaagcctgatgcagcgacgccgcgtgagggatgacggccttcgggttgtaaacctctgttagcatcgaagaagcgaaagtgacggtaggtgcagagaaagcgccggctaactacgtgccagcagccgcggtaatacgtagggcgcgagcgttgtccggaattattgggcgtaaagagcttgtaggcggttggtcgcgtctgctgtgaaaggctggggcttaaccctggttttgcagtgggtacgggctaactagagtgcagtaggggagactggaattcctggtgtagcggtggaatgcgcagatatcaggaggaacaccgatggcgaaggcaggtctctgggctgtaactgacgctgagaagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaagaaattgacgggggcccgcacaagcggcggagcatgcggattaattcgatgcaacgcgaagaaccttaccaaggcttgacatatactggactgcgtcagagatggcgtttcccttcggggctggtatacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggatgacgtcaaatcatcatgccccttatgtcttgggcttcacgcatgctacaatggccggtacagagggttgcgatactgtgaggtggagctaatccctaaaagccggtctcagttcggattggggtctgcaactcgaccccatgaagtcggagtcgctagtaatcgcagatcagcaacgctgcggtgaatacgttcccgggcctttgtacacaccgcccgtca

SEQ ID NO: 207 - DNA, 1343 bp, Corynebacterium
  source 1..1343: mol_type = genomic DNA
gacgaacgctggcggcgtgcttaacacatgcaagtcgaacggaaaggctccagcttgctggagtactcgagtggcgaacgggtgagtaacacgtgggtaatctgccctgcactctgggataagcctgggaaactgggtctaatactggatatgacctttctccttatggggtttggtggaaagtttttcggtgtaggatgagcccgcggcctatcagcttgttggtggggtgatggcctaccaaggcgacgacgggtagccggcctgagagggtgatcggccacattgggactgagatacggcccagactcctacgggaggcagcagtggggaatattgcacaatgggcggaagcctgatgcagcgacgccgcgtgagggatgacggccttcgggttgtaaacctctttcagcagggacgaagcgtaagtgacggtacctgcagaagaagcaccggctaactatgtgccagcagccgcggtaatacgtagggtgcgagcgttgtccggaattactgggcgtaaagagctcgtaggcggtttgtcacgtcgtctgtgaaatcctagggctcaaccctggacgtgcaggcgatacgggctgacttgagtactacaggggagactggaatttctggtgtagcggggggaatgcacagatatcaggaagaacaccgatggcgaaggcaggtctctgggtagtaactgacgctgaggagcgaaagcatgggtagcgaacaggattagataccctggtagtccatgccgtaaacggtgggcgctaggtgtggggttccttccacgggctccgcgccgtagctaacgcattaagcgccccgcctggggagtacggccgcaaggctaaaactcaaagaaattgacgggggcccgcacaagcggcggagcatgtggattaattcgatgcaacgcgaagaaccttacctgggcttgacatatagaggatcgctacagagatgtggtttcccttgtggcttctatacaggtggtgcatggttgtcgtcagctcgtgtcgtgagatgttgggttaagtcccgcaacgagcgcaacccttgtcttatgttgccagcacgttatggtggggactcgtgagagactgccggggtcaactcggaggaaggtggggatgacgtcaaatcatcatgccccttatgtccagggcttcacacatgctacaatggccggtacagagggctgcgatgccgtgaggcggagcgaatctcttaaagccggtctcagttcggattggggtctgcaactcgaccccatgaagtcggagtcgctagtaatcgcagatcagcaacgctgcggtgaatacgttcccgggcct

SEQ ID NO: 208 - DNA, 1372 bp, Staphylococcus pasteuri
  source 1..1372: mol_type = genomic DNA
gatgaacgctggcggcgtgcctaatacatgcaagtcgagcgaacagataaggagcttgctcctttgacgttagcggcggacgggcgagtaacacgtggataacctacctataagactgggataacttcgggaaaccggagctaataccggataagattttgaaccgcatggttcaatagtgaaagacggccttgctgtcacttatagatggatccgcgccgtattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttatcagggaagaacaaacgtgtaagtaactgtgcacgtcttgacggtacctgatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgaccgctctagagatagagttttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagctaaaccgcgaggtcaagcaaatcccataaagttgttctcagttcggattgtagtctgcaactcgactacatgaagctggaatcgctagtaatcgtagatcagcatgctacggtgaatacgttcccgggtct

SEQ ID NO: 209 - DNA, 1415 bp, Prevotella melaninogenica
  source 1..1415: mol_type = genomic DNA
agagtttgatcctggggctcaggataaaaacgctagctacatggcttaacacatgcaagtcgaggggaaacggcattgagtgcttgcactctttggacgtcgaccggcgcacgggtgagtaacgcgtatccaaccttcccattactgtgggataacctgccgaaaggcagactaataccgcatagtcttcaatgacggcatcagatttgaagtaaagatttatcggtaatggatggggatgcgtctgattagcttgttggcggggtaacggcccaccaaggcaacgatcagtaggggttctgagaggaaggtcccccacattggaactgagacacggtccaaactcctacgggaggcagcagtgaggaatattggtcaatggacggaagtctgaaccagccaagtagcgtgcaggatgacggccctatgggttgtaaactgcttttgtatggggataaagttagggacgtgtccctatttgcaggtaccatacgaataaggaccggctaattccgtgccagcagccgcggtaatacggaaggtccaggcgttatccggatttattgggtttaaagggagcgtaggctggagattaagtgtgttgtgaaatgtagacgctcaacgtctgaattgcagcgcatactggtttccttgagtacgcacaacgttggcggaattcgtcgtgtagcggtgaaatgcttagatatgacgaagaactccgattgcgaaggcagctgacgggagcgcaactgacgcttaagctcgaaggtgcgggtatcaaacaggattagataccctggtagtccgcacagtaaacgatggatgcccgctgttggtacctggtatcagcggctaagcgaaagcattaagcatcccacctggggagtacgccggcaacggtgaaactcaaaggaattgacgggggcccgcacaagcggaggaacatgtggtttaattcgatgatacgcgaggaaccttacccgggcttgaattgcagaggaaggatttagagataatgacgcccttcggggcctctgtgaaggtgctgcatggttgtcgtcagctcgtgccgtgaggtgtcggcttaagtgccataacgagcgcaacccctctcttcagttgccatcaggtgatgctgggcactctggagacactgccaccgtaaggtgtgaggaaggtggggatgacgtcaaatcagcacggcccttacgtccggggctacacacgtgttacaatggccggtacagagggacggtgtaatgcaaattgcatccaatcttgaaagccggtcccagttcggactggggtctgcaacccgaccccacgaagctggattcgctagtaatcgcgcatcagccatggcgcggtgaatacgttcccgggccttgtacacaccgcccgtcaagccatgaaag

SEQ ID NO: 210 - DNA, 1327 bp, Fusobacterium nucleatum
  source 1..1327: mol_type = genomic DNA
gatgaacgctgacagaatgcttaacacatgcaagtctacttgagcttcggtttgggtggcggacgggtgagtaacgcgtaaagaacttgcctcacagatagggacaacatttggaaacgaatgctaatacctgatattatgattttagggcatcctaggattatgaaagctatatgcgctgtgagagagctttgcgtcccattagctagttggagaggtaacggctcaccaaggcgatgatgggtagccggcctgagagggtgatcggccacgaggggactgagacacggcccttactcctacgggaggcagcagtggggaatattggacaatggaccaagagtctgatccagcaattctgtgtgcacgatgaagtttttcggaatgtaaagtgctttcagttgggaagaaaaaaatgacggtaccaacagaagaagtgacggctaaatacgtgccagcagccgcggtaatacgtatgtcacaagcgttatccggatttattgggcgtaaagcgcgtctaggtggttatgtaagtctgatgtgaaaatgcagggctcaactctgtattgcgttggaaactgtgtaactagagtactggagaggtaagcggaactacaagtgtagaggtgaaattcgtagatatttgtaggaatgccgatggggaagccagcttactggacagatactgacgctgaagcgcgaaagcgtgggtagcaaacaggattagataccctggtagtccacgccgtaaacgatgattactaggtgttgggggtcgaacctcagcgcccaagcaaacgcgataagtaatccgcctggggagtacgtacgcaagtatgaaactcaaaggaattgacgaggacccgcacaagcggtggagcatgtggtttaattcgacgcaacgcgaggaaccttaccagcgtttgacatcttaggaatgagacagagatgtttcagtgtcccttcggggaaacctaaagacaggtggtgcatggctgtcgtcagctcgtgtcgtgagatgttgggttaagtcccgcaacgagcgcaacccctttcgtatgttaccatcattaagttggggactcatgcgatactgcctgcgatgagcaggaggaaggtggggatgacgtcaagtcatcatgccccttatacgctgggctacacacgtgctacaatgggtagtacagagagtcgcaaagccgtgaggtggagctaatctcagaaaactattcttagttcggattgtactctgcaactcgagtacatgaagttggaatcgctagtaatcgcgaatcagcaatgtcgcggtgaatacgttctcgggtct

SEQ ID NO: 211 - DNA, 1326 bp, unidentified; identical to SEQ ID NO: 175
  misc_feature 1..1326: note = 16S ribosomal RNA of unknown bacteria
  source 1..1326: mol_type = genomic DNA

SEQ ID NO: 212 - DNA, 1348 bp, Finegoldia; identical to SEQ ID NO: 176
  source 1..1348: mol_type = genomic DNA

SEQ ID NO: 213 - DNA, 1358 bp, Acinetobacter ursingii; identical to SEQ ID NO: 180
  source 1..1358: mol_type = genomic DNA

SEQ ID NO: 214 - DNA, 1385 bp, Streptococcus pneumoniae
  source 1..1385: mol_type = genomic DNA
taatacatgcaagtagaacgctgaaggaggagcttgctaatcaggatgagttgcgaacgggtgagtaacgcgtaggtaacctgcctggtagcgggggataactattggaaacgatagctaataccgcataatagtagatgttgcatgacatttgcttaaaaggtgcaattgcatcactaccagatggacctgcgttgtattagctagttggtggggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgccgtaaacgatgagtgctaggtgttagaccctttccggggtttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacac

SEQ ID NO: 215 - DNA, 1353 bp, Prevotella Unknown
  source 1..1353: mol_type = genomic DNA
gatgaacgctagctacaggcttaacacatgcaagtcgaggggcagcatggaagaagcttgcttcttctgatggcgaccggcgcacgggtgcgtaacgcgtatcaaacctgcctcatactcggggatagccttgcgaaagtaagattaatacccgatgttgttatgttaccgcatggtgatataaccaaagatttatcggtatgagatggtgatgcgtccgattaggtagtaggcggggtaacggcccacctagccttcgatcggtaggggttctgagaggaaggtcccccacactggaactgagacacggtccagactcctacgggaggcagcagtgaggaatattggtcaatggacgaaagtctgaaccagccaagtagcgtgcaggatgacggccctctgggttgtaaactgcttttagttgggaataaaaaagaggacgtgtcctctattgtatgtaccttcagaaaaaggaccggctaattccgtgccagcagccgcggtaatacggaaggtccaggcgttatccggatttattgggtttaaagggagcgtaggcggattattaagtcagtggtgaaagacggtggctcaaccatcgttagccattgaaactggtagtcttgagtgcagacagggatgctggaactcgtggtgtagcggtgaaatgcttagatatcacgatgaactccgatcgcgaaggcaggtgtccgggctgcaactgacgctgaggctcgaaagtgtgggtatcaaacaggattagataccctggtagtccacacagtaaacgatgtatactcgcggtttgcgatacaatgtaagccgccaagcgaaagcattaagtataccacctggggagtacgccggcaacggtgaaactcaaaggaattgacgggggcccgcacaagcggaggaacatgtggtttaattcgatgatacgcgaggaaccttacccgggcttgaactaggagtgacagatttagagatagatctttcttcggacactcttggaggtgctgcatggttgtcgtcagctcgtgccgtgaggtgtcggcttaagtgccataacgagcgcaacccttttccttggttgccatcaggtaatgctgggcactccagggatactgccatcgtaagatgtgaggaaggtggggatgacgtcaaatcagcacggcccttacgtccggggctacacacgtgttacaatggggggtacagaaggtcgctacctggtgacaggatgctaatctcaaaaacctctctcagttcggattggagtctgcaacccgactccatgaagctggattcgctagtaatcgcgcatcagccatggcgcggtgaatacgttcccgg

SEQ ID NO: 216 - DNA, 1372 bp, Staphylococcus
  source 1..1372: mol_type = genomic DNA
gatgaacgctggcggcgtgcctaatacatgcaagtcgagcgaacagataaggagcttgctcctttgacgttagcggcggacgggtgagtaacacgtggataacctacctataagactgggataacttcgggaaaccggagctaataccggataacatattgaaccgcatggttcaatagtgaaaggcggctttgctgtcacttatagatggatccgcgccgtattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttatcagggaagaacaaatgtgtaagtaactgtgcacatcttgacggtacctgatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggactatacaaagggcagcgaaaccgcgaggtcaagcaaatcccataaagttgttctcagttcggattgtagtctgcaactcgactacatgaagctggaatcgctagtaatcgtagatcagcatgctacggtgaaaaggttcccgggtct

SEQ ID NO: 217 - DNA, 1543 bp, Veillonella
  source 1..1543: mol_type = genomic DNA
gagagtttgattctggctcatgatgaaggctgacataatgcttaacacatgcatttcaacttgaatttgggtttttaactttcgtttgggtggcggacgggtgagtgacgcgtaaagaacttgcctcgcacctggggacaagagttggaaacgactgctaataccgcataccatctaatctcggcatcaaggaaagatgaaaggtggcctctatgtataagctatcactgaaggaggggattgcgtctgattagctagttggaggggtaacggcccaccaaggcgatgatcatggtccggtctgagaggatgaacggccacattgggactgagacactgcccacactcctacgggaggcatcaatggggaatcctcctcaatggactaaagtctgacggatcaacgccgcgtgagtgatgacggccttcgggttgtaaagctctgttaatcaggacgaaaggccttcttgcgaatattgagagtgattgacggtaccggaatacaaggccacggctaactacgtgccagcagccgcggtaatacgtaagtggcaagcgttgtccggaattattgggcgtaaagcgcgcgcaggcggatcggtcagtctgtcattaaagttcggggcttaaccccgtgatgggatgaaactgctgatctagagtatcggagcgaaagtggaattcctagtgtagcggtgaatgcgtagatattaggaagaacaccagtggcgaaggcgactttctggacgaaaactgacgctgaggcgcgaaagcccatgggagcgaacgggattagataccccggtagtcctggccgtaaacgatgggtactaggtgtaggaggtatcgaccccttctgtgccggagttaacgcaataagtaccccgcctgggagtacgaccgcaaggttgaaactcaaaggaattgacgggggcccgcacaagcggtggagtatgtggtttaattcgacgcaacgcgaagaaccttaccaggtcttgacattgatggacagaaccagagatggttcctcttcttcggaagccagaaaacaggtggtgcacggttgtcgtcagctcgtgtcgtgagatgttgggttaagtcccgcaacgagcgcaacccctatcttatgttgccagcacttcgggtggggactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaaggtgcggctggaacacct

SEQ ID NO: 218 - DNA, 1388 bp, Paracoccus chinensis
  source 1..1388: mol_type = genomic DNA
aacgaacgctggcggcaggcctaacacatgcaagtcgagcgcgcccttaggggtgagcggcggacgggtgagtaacgcgtgggaacgtgcccttctctgcggaatagcctcgggaaactgagagtaataccgcatacgccctttgggggaaagatttatcggagaaggatcggcccgcgttggattaggtagttggtggggtaatggcccaccaagccgacgatccatagctggtttgagaggatgatcagccacactgggactgagacacggcccagactcctacgggaggcagcagtggggaatcttagacaatgggggcaaccctgatctagccatgccgcgtgagtgatgaaggccttagggttgtaaagctctttcagctgggaagataatgacggtaccagcagaagaagccccggctaactccgtgccagcagccgcggtaatacggagggggctagcgttgttcggaattactgggcgtaaagcgcacgtaggcggaccagaaagttgggggtgaaatcccggggctcaacctcggaactgccttcaaaactattggtctggagttcgagagaggtgagtggaattccgagtgtagaggtgaaattcgtagatattcggaggaacaccagtggcgaaggcggctcactggctcgatactgacgctgaggtgcgaaagcgtggggagcaaacaggattagataccctggtagtccacgccgtaaacgatgaatgccagacgtcgggcagcatgctgttcggtgtcacacctaacggattaagcattccgcctggggagtacggtcgcaagattaaaactcaaaggaattgacgggggcccgcacaagcggtggagcatgtggtttaattcgaagcaacgcgcagaaccttaccaacccttgacatccctggacagcccgagagatcgggtctccacttcggtggccaggtgacaggtgctgcatggctgtcgtcagctcgtgtcgtgagatgttcggttaagtccggcaacgagcgcaacccacgtccccagttgccagcattcagttgggcactctgtggaaactgccgatgataagtcggaggaaggtgtggatgacgtcaagtcctcatggcccttacgggttgggctacacacgtgctacaatggtggtgacagtgggttaatccccaaaagccatctcagttcggattggggtctgcaactcgaccccatgaagttggaatcgctagtaatcgcgtaacagcacgacgcggtgaatacgttcccgggccttgtacacaccgcccgtcacaccatgggagttgggtctacccgacggccgtgcgctaacccttacgggaggcagcggaccacggtaggctcagcgactggggtgaag

SEQ ID NO: 219 - DNA, 1432 bp, Massilia timonae
  source 1..1432: mol_type = genomic DNA
tcgaacggtaacagggagcttgctccgctgacgagtggcgaacgggtgagtaatatatcggaacgtacccaagagtgggggataacgtagcgaaagttacgctaataccgcatacgatctatggatgaaagcaggggaccttcgggccttgtgctcctggagcggccgatatctgattagctagttggtgaggtaaaggctcaccaaggcgacgatcagtagctggtctgagaggacgaccagccacactggaactgagacacggtccagactcctacgggaggcagcagtggggaattttggacaatgggcgcaagcctgatccagcaatgccgcgtgagtgaagaaggccttcgggttgtaaagctcttttgtcagggaagaaacggctgtggctaatatccacggctaatgacggtacctgaagaataagcaccggctaactacgtgccagcagccgcggtaatacgtagggtgcaagcgttaatcggaattactgggcgtaaagcgtgcgcaggcggttttgtaagtctgtcgtgaaatccccgggctcaacctgggaattgcgatggagactgcaaggctagaatctggcagagggggggtagaattccacgtgtagcagtgaaatgcgtagagatgtggaggaacaccgatggcgaaagcagccccctgggtcaagattgacgctcatgcacgaaagcgtgggaagcaaacagaattagataccctgatagtccacgccctaaacgatgtctactagttgtcgggttttaattaacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatgtcaggaagaccggagagatttggttgtgcccgaaagggaacctgaacacaggtgctgcatggctgtcgtcagctcgtgtcgtgagatgttgggttaagtcccgcaacgagcgcaacccttgtcattagttgctacgcaagagcactctaatgagactgccggtgacaaaccggaggaaggtggggatgacgtcaagtcctcatggcccttatgggtagggcttcacacgtcatacaatggtacatacagagggccgccaacccgcgagggggagctaatcccagaaagtgtatcgtagtccggatcgtagtctgcaactcgactacgtgaagttggaatcgctagtaatcgcggatcagcatgccgcggtgaatacgttcccgggtcttgtacacaccgcccgtcacaccatgggagcgggttttaccagaagtaggtagcttaaccgcagggagggcgcttaccacggtaggattcgtgactggggtgaagtcgtaacaa

SEQ ID NO: 220 - DNA, 1377 bp, Paracoccus marcusii
  source 1..1377: mol_type = genomic DNA
acgctggcggcaggcttacacatgcaagtcgagcgagaccttcgggtctagcggcggacgggtgagtaacgcgtgggaacgtgcccttctctacggaatagccccgggaaactgggagtaataccgtatacgccctttgggggaaagatttatcggagaaggatcggcccgcgttggattaggtagttggtggggtaatggcccaccaagccgacgatccatagctggtttgagaggatgatcagccacactgggactgagacacggcccagactcctacgggaggcagcagtggggaatcttagacaatgggggcaaccctgatctagccatgccgcgtgagtgatgaaggccttagggttgtaaagctctttcagctgggaagataatgacggtaccagcagaagaagccccggctaactccgtgccagcagccgcggtaatacggagggggctagcgttgttcggaattactggagcgtaaagcgcacgtaagcggactggaaagtcagaggtgaaatcccagggctcaaccttgggaactgccttttgaaactatcagactggagttccaagagagggtgagtggaattccgagtgtagaggtgaaattcgtagatattcggaggaacaccagtggcgaaggcggctcactggctcgatactgacgctgaggtgcgaaagcgtggggagcaaacaggattagataccctggtagtccacgccgtaaacgatgaatgccagacgtcggcaagcatgcttgtcggtgtcacacctaacggattaagcattccgcctggggagtacggtcgcaagattaaaactcaaaggaattgacgggggcccgcacaagcggtggagcatgtggtttaattcgaagcaacgcgcagaaccttaccaacccttgacatggcaggaccgctggagagattcagctttctcgtaagagacctgcacacaggtgctgcatggctgtcgtcagctcgcgtcgtgagatgttcggttaagtccggcaacgagcgcaacccacgtccctagttgccagcattcagttgggcactctatggaaactgccgatgataagtcggaggaaggtgtggatgacgtcaagtcctcatggcccttacgggttgggctacacacgtgctacaatggtggtgacagtgggttaatccccaaaagccatctcagttcggattgtcctctgcaactcgagggcatgaagttggaatcgctagtaatcgcggaacagcatgccgcggtgaatacgttcccgggccttgtacacaccgcccgtcacactatgggagttggttctacccgacgacgctgcgctaaccttcggggggcaggcggccacggagatcagcgacgggg

SEQ ID NO: 221 - DNA, 1339 bp, Propionibacterium granulosum; identical to SEQ ID NO: 201
  source 1..1339: mol_type = genomic DNA

SEQ ID NO: 222 - DNA, 1410 bp, Kaistobacter Unknown
  source 1..1410: mol_type = genomic DNA
aacgaacgctggcggcatgcctaacacatgcaagtcgaacgagaccttcgggtctagtggcgcacgggtgcgtaacgcgtgggaatctgcccttgggttcggaacaacagttggaaacgactgctaataccggatgatgacttcggtccaaagatttatcgcccaaggatgagcccgcgtaggattagcttgttggtgaggtaaaagctcaccaaggcgacgatccttagctggtctgagaggatgatcagccacactgggactgagacacggcccagactcctacgggaggcagcagtggggaatattggacaatgggcgaaagcctgatccagcaatgccgcgtgagtgatgaaggccttagggttgtaaagctcttttacccgggatgataatgacagtaccgggagaataagccccggctaactccgtgccagcagccgcggtaatacggagggggctagcgttgttcggaatcactgggcgtaaagcgcacgtaggcggctttgtaagttagaggtgaaagcccggagctcaactccggaattgcctttaagactgcatcgcttgaatcatggagaggtgagtggaattccgagtgtagaggtgaaatttcgtagatatccggaagaacaccagtggcgaaggcgactcactggacatgttttgacgcttaggtgcgaaagcgtggggagcaaacaggattagataccctggtagtccacgccgtaaacgatgatgactagctgtcggggcgcttggcgtttcggtggcgcagctaacgcgttaagtcatccgcctggggagtacggccgcaaggttaaaactcaaagaaattgacgggggcctgcacaagcggtggagcatgtggtttaattcgaagcaacgcgcagaaccttaccagcgtttgacatggtaggacggtttccagagatggattccttcccttacgggacctacacacaggtgctgcatggctgtcgtcagctcgtgtcgtgagatgttgggttaagtcccgcaacgagcgcaaccctcgtctttagttgctaccatttagttgggcactctaaagaaactgccggtgataagccggaggaaggtggggatgacgtcaagtcctcatggcccttacgcgctgggctacacacgtgctacaatggcggtgacagagggctgcaaacccgcgagggtgagctaatctccaaaagccgtctcagttcggattgttctctgcaactcgagagcatgaaggcggaatcgctagtaatcgcggatcagcatgccgcggtgaatacgttcccaggccttgtacacaccgcccgtcacaccatgggagttggattgacccgaaggtggtgagctaacccgcaagggaggcagccaaccacggtcggttcagcgactggggtg

SEQ ID NO: 223 - DNA, 1520 bp, Veillonella; identical to SEQ ID NO: 205
  source 1..1520: mol_type = genomic DNA

SEQ ID NO: 224 - DNA, 1343 bp, Corynebacterium; identical to SEQ ID NO: 207
  source 1..1343: mol_type = genomic DNA

SEQ ID NO: 225 - DNA, 1347 bp, Rothia mucilaginosa; identical to SEQ ID NO: 202
  source 1..1347: mol_type = genomic DNA

SEQ ID NO: 226 - DNA, 1353 bp, Prevotella Unknown; identical to SEQ ID NO: 215
  source 1..1353: mol_type = genomic DNA

SEQ ID NO: 227 - DNA, 1531 bp, Lactobacillus iners; identical to SEQ ID NO: 184
  source 1..1531: mol_type = genomic DNA

SEQ ID NO: 228 - DNA, 1365 bp, Sphingomonas yunnanensis
  source 1..1365: mol_type = genomic DNA
acacatgcaagtcgaacgaaggcttcggccttagtggcgcacgggtgcgtaacgcgtgggaatctgcccttgggttcggaataacagttggaaacgactgctaataccggatgatgtcgcgagaccaaagatttatcgcccagggatgagcccgcgtcggattaggtagttggtggggtaaaggcctaccaagccgacgatccgtagctggtctgagaggatgatcagccacactgggactgagacacggcccagactcctacgggaggcagcagtggggaatattggacaatgggcgaaagcctgatccagcaatgccgcgtgagtgatgaaggccttagggttgtaaagctcttttacccgggatgataatgacagtaccgggagaataagctccggctaactccgtgccagcagccgcggtaatacggagggagctagcgttgttcggaattactgggcgtaaagcgcacgtaggcggctttgtaagtcaggggtgaaagcctggagctcaactccagaactgcctttgagactgcatcgcttgaatccgggagaggtgagtggaattccgagtgtagaggtgaaattcgtagatattcggaagaacaccagtggcgaaggcggctcactggaccggtattgacgctgaggtgcgaaagcgtggggagcaaacaggattagataccctggtagtccacgccgtaaacgatgataactagctgtccgggcacttggtgcttgggtggcgcagctaacgcattaagttatccgcctggggagtacggccgcaaggttaaaactcaaaggaattgacgggggcctgcacaagcggtggagcatgtggtttaattcgaagcaacgcgcagaaccttaccagcgtttgacatgtccggacgattcccagagatgggtctcttcccttcggggactggaacacaggtgctgcatggctgtcgtcagctcgtgtcgtgagatgttgggttaagtcccgcaacgagcgcaaccctcgcctttagttaccatcattcagttgggtactctaaaggaaccgccggtgataagccggaggaaggtggggatgacgtcaagtcctcatggcccttacgcgctgggctacacacgtgctacaatggcgactacagtgggctgcaatcccgcgagggtgagctaatctccaaaagtcgtctcagttcggattgctctctgcaactcgagagcatgaaggcggaatcgctagtaatcgcggatcagcatgccgcggtgaatacgttcccaggccttgtacacaccgcccgtcacaccatgggagttgggttcacccgaaggcgttgcgctaactcgcaagagaggcaggcgaccacg

SEQ ID NO: 229 - DNA, 1380 bp, unidentified
  misc_feature 1..1380: note = 16S ribosomal RNA of unknown bacteria
  source 1..1380: mol_type = genomic DNA
agagtttgatctggctcaggacgaacgctggcggcgtgcttaacacatgcaagtcgaacggtgacgacggagcttgctctgtctgatcagtggcgaacgggtgagtaacacgtgagtaacctgcccccgactctggaataaccgtgggaaacgacggctaataccggatatgagacgcacaggcatctgtgggtctggaaagatttatcggtcagggatggactcgcggcctatcagcttgctggtggggtaatggcccaccaaggcgacgacgggtagccggcctgagagggcgaccggccacactgggactgagatacggcccagactcctacgggaggcagcagtggggaatattgcacaatgggcgcaagcctgatgcagcgacgccgcgtgagggatgaaggccttcgggttgtaaacctctttcagcagggaagaagcgaaagtgacggtacctgcagaagaagcgccggctaactacgtgccagcagccgcggtgatacgtagggtgcgagcgttgtccggaattattgggcgtaaagagcttgtaggcggtctgttgcgtcggaagtgaaaactcagggcttaaccctgagcctgctttcgatacgggcagacttgagggatgtaggggagaacggaattcctggtggagcggtggaatgcgcagatatcaggaggaacaccagtggcgaaggcggttctctggacatttcctgacgctgagaagcgaaagcgtggggagcaaacaggcttagataccctggtagtccacgccgtaaacggtgggcactaggtgtgggttccattccacggaatccgtgccgaagctaacgcattaagtgccccgcctggggagtacggccgcaaggctaaaactcaaaggaattgacgggggcccgcacaagcggcgggagcatgcggattaattcgatgcaacgcgaagaaccttacctgggtttgacatatgccggaaacacctagagataggtgccccgcaaggccggtttacaggtggtgcatggctgtcgtcagctcgtgtcgtgagatgttgggttaagtcccgcaacgagcgcaaccctcgttctatgttgccagcgggtaatgccgggagcactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgacctcatgaagtcggagtcgctagtaatcgcagatcagcaacgctgcggtgaatacgttcccgggccttgtacacaccgcccgt

SEQ ID NO: 230 - DNA, 1377 bp, Paracoccus marcusii; identical to SEQ ID NO: 220
  source 1..1377: mol_type = genomic DNA

SEQ ID NO: 231 - DNA, 1310 bp, Roseomonas mucosa
  source 1..1310: mol_type = genomic DNA
agcgaacgctggcggcatgcttaacacatgcaagtcgcacgggcagcaatgtcagtggcggacgggtgagtaacgcgtaggaacgtgtccagaggtgggggacaaccccgggaaactggggctaataccgcataggggctgaggcccaaagccgaaaggcgcctttggagcggcctgcgtccgattaggtagttggtggggtaaaggcctaccaagcctgcgatcggtagctggtctgagaggacgaccagccacactgggactgagacacggcccagactcctacgggaggcagcagtggggaatattggacaatgggcgaaagcctgatccagcaatgccgcgtgggtgaagaaggtcttcggatcgtaaagccctttcgacggggacgatgatgacggtacccgtagaagaagccccggctaacttcgtgccagcagccccggtaatactaagggggctagcgatgctcggaattactgggcgtaaagggcgcgtaagcggcggcccaagtcaggcgtgaaattcctgggctcaacctggggactgcgcttgatactgggttgcttgaggatggaagaggctcgtggaattcccagtgtagaggtgaaattcctagatattgggaagaaccccggtggcgaaggcggcgagctggtcccattactgacgctgaggcgcgacagcgtggggagccaaacaggattagataccctggtagtccacgccgtaaacgatgtgcgctggatgttggggcccatagggtctcagtgtcgtagccaacgcggtaagcgcaccgcctggggagtacggccgcaaggttgaaactcaaaggaattgacgggggcccgcacaagcggtggagcatgtggtttaattcgaagcaacgcgcagaaccttaccagcccttgacatggtcacgaccggtccagagatgggctttccccgcaaggggcgtgatgcacaggtgctgcatggctgtcgtcagctcgtgtcgtgagatgttgggttaagccccgcaacgagcgcaaccctcgcctctagttgccagcatgttcgggtgggcactctagaggaactgccggtgacaagccggaggaaggtggggatgacgtcaagtcctcatggcccttatgggctgggctacacacgtgctacaatggcggtgacagagggaagccaggtcgcgaggccgagccgatcccgaaaagccgtctcagttcggattgcactctgcaactcgggtgcatgaaggtggaatcgctagtaatcgcggatcagcacgccgcggtgaatacgttcccgggcct

SEQ ID NO: 232 - DNA, 1358 bp, Pseudomonas baetica
  source 1..1358: mol_type = genomic DNA
attgaacgctggcggcaggcctaacacatgcaagtcgagcggatgagaggagcttgctccttaattcagcggcggacgggtgactaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaaattaatactttgctgttttgacgttaccgacagaataagcaccggctaactctgtgccagcagccgcggtaatacagagggtgcaagcgttaatcggaattactgggcgtaaagcgcgcgtaggtggttcgttaagttggatgtgaaatccccgggctcaacctgggaactgcattcaaaactgtcgagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggagcattgagacaggtgctgcatggctgtcgtcagctcgtgtcgtgagatgttgggttaagtcccgtaacgagcgcaacccttgtccttagttaccagcacgtcatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggcct

SEQ ID NO: 233 - DNA, 1340 bp, Propionibacterium
  source 1..1340: mol_type = genomic DNA
gacgaacgctggcggcgtgcttaacacatgcaagtcgtacggtaaggccctttcgggggtacacgagtggcgaacgggtgagtaacacgtgagtaacctgcccacaactttgagataacgctaggaaactggtgctaatactggatatgtgctcctgctgcatggtgggggttggaaagctccggcggttgtggatggacttgcggcctatcagcttgttggtggggtagtggcctaccaaggcggcgacgggtagccggcctgagagggtgaccggccacattgggactgagatacggcccagactcctacgggaggcagcagtggggaatattgcacaatgggcgcaagcctggtgcagcaacgccgcgtgcgggatgacggccttcgggttgtaaaccgctttcagcagggacgaagctttttgtgacggtacctgcagaagaagcaccggctaactacgtgccagcagccgcggtgatacgtagggtgcgagcgttgtccggatttattgggcgtaaagggctcgtaggtggttgatcgcgtcggaagtgtaatcttggggcttaaccctgagcgtgctttcgatacgggttgacttgaggaaggtaggggagaatggaattcctggtggagcggtggaatgcgcagatatcaggaggaacaccagtggcgaaggcggttctctgggcctttcctgacgctgaggagcgaaagcgtggggagcgaacaggcttagataccctggtagtccacgctgtaaacggtgggtactaggtgtggggtccattccacgggttccgcgccgtagctaacgctttaagtaccccgcctggggagtacggccgcaaggctaaaactcaaaggaattgacggggccccgcacaagcggcggagcatgcggattaattcgatgcaacgcgtagaaccttacctgggtttgacatggatcgggagtgctcagagatgggtgtgcctcttttggggtcggttcacaggtggtgcatggctgtcgtcagctcgcgtcgtgagatgttgggttaagtcccgcaacgagcgcaacccttgttcactgttgccagcacgttatggtggggactcagtggagaccgccggggtcaactcggaggaaggtggggatgacgtcaagtcatcatgccccttatgtccagggcttcacgcatgctacaatggctggtacagagagtggcgagcctgtgagggtgagcgaatctcggaaagccggtctcagttcggattggggtctgcaactcgacctcatgaagtcggagtcgctagtaatcgcagatcagcaacgctgcggtgaatacgttcccggg

SEQ ID NO: 234 - DNA, 1310 bp, Sphingomonas
  source 1..1310: mol_type = genomic DNA
aacgaacgctggcggcatgcctaacacatgcaagtcgaacgaggtgccttcgggtatctagcggcgcacgggtgcgtaacgcgtgggaatctgccctggggtctgggataaccgttggaaacgacggcaaataccggatgatgatgtaaatccaaagatttatcgccctgggatgagcccgcgtaggattagctagttggtggggtaaaggcctaccaaggcgacgatccttagctggtctgagaggatgatcagccacactgggactgagacacggcccagactcctacgggaggcagcagtggggaatattggacaatgggcgaaagcctgatccagcaatgccgcgtgagtgatgaaggccttagggttgtaaagctcttttacccgggatgataatgacagtaccgggagaataagccccggctaactccgtgccagcagccgcggtaatacggagggggctagcgttgttcggaattactgggcgtaaagcgcacgtaggcggccgtttaagttagaggtgaaagcccggggctcaaccccggaattgcctttgatactggacggcttgaatccgggagaggtgagtggaattccgagtgtagaggtgaaattcgtagatattcggaagaacaccagtggcgaaggcggctcactggaccggcattgacgctgaggtgcgaaagcgtggggagcaaacaggattagataccctggtagtccacgccgtaaacgatgataactagctgtcgggccgcttagcgggtcggtggcgcagctaacgcattaagttatccgcctggggagtacggccgcaaggttaaaactcaaatgaattgacgggggcctgcacaagcggtggagcatgtggtttaattcgaagcaacgcgcagaaccttaccagcgtttgacatgtccggacgatttccagagatggatctcttcccttcggggactggaacacaggtgctgcatggctgtcgtcagctcgtgtcgtgagatgttgggttaagtcccgcaacgagcgcaaccctcgaccttagttgccatcatttagttgggcactttaaggtaaccgccggtgataagccgggggaaggtggggatgacgtcaagtcctcatggcccttacgcgctgggctacacacgtgctacaatggcggtgacagtgggcggcaaaccggcgacggtgagctaatctccaaaagccgtctcagttcggattgttctctgcaactcgagagcatgaaggcggaatcgctagtaatcgcggatcagcatgccgcggtgaatacgttcccaggcct

SEQ ID NO: 235 - DNA, 1521 bp, Staphylococcus warneri
  source 1..1521: mol_type = genomic DNA
gtttgatcatggctcaggatgaacgctggcggcgtgcctaatacatgcaagtcgagcgaacagataaggagcttgctcctttgacgttagcggcggacgggtgggtaacacgtggataacctacctataagactgggataacttcgggaaaccggagctaataccggataacatattgaaccgcatggttcaatagtgaaaggcggctttgctgtcacttatagatggatccgcgccgtattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttatcagggaagaacaaatgtgtaagtaactgtgcacatcttgacggtacctgatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgaaaacgatgagtgctaagtgttagggggtttccgccccttagtgctgcagctaacgcattaagcactccgcctggggagtacgaccgcaaggttgaaaactcaaaggaattgacggggacccgcacaagcggtggagcatgtggtttgattcgaagcaacgcgaagaaccttaccaaatcttgacatcctttgaccgctctagagatagagtcttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagctaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccatttatggagctagccgtcgaaggtgggacaaatgattggggtgaagtcgtaacaaggtagccgtatcggaa

SEQ ID NO: 236 - DNA, 1462 bp, Alcaligenes faecalis
  source 1..1462: mol_type = genomic DNA
attgaacgctagcgggatgctttacacatgcaagtcgaacggcagcgcgagagagcttgctctcttggcggcgagtggcggacgggtgagtaatatatcggaacgtgcccagtagcgggggataactactcgaaagagtggctaataccgcatacgccctacgggggaaagggggggatcgcaagacctctcactattggagcggccgatatcggattagctagttggtggggtaaaggctcaccaaggcaacgatccgtagctggtttgagaggacgaccagccacactgggactgagacacggcccagactcctacgggaggcagcagtggggaattttggacaatgggggaaaccctgatccagccatcccgcgtgtatgatgaaggccttcgggttgtaaagtacttttggcagagaagaaaaggtaactcctaatacgagatactgctgacggtatctgcagaataagcaccggctaactacgtgccagcagccgcggtaatacgtagggtgcaagcgttaatcggaattactgggcgtaaagcgtgtgtaggcggttcggaaagaaagatgtgaaatcccagggctcaaccttggaactgcatttttaactgccgagctagagtatgtcagaggggggtagaattccacgtgtagcagtgaaatgcgtagatatgtggaggaataccgatggcgaaggcagccccctgggataatactgacgctcagacacgaaagcgtggggagcaaacaggattagataccctggtagtccacgccctaaacgatgtcaactagctgttggggccgttaggccttagtagcgcagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtctggaaagccgaagagatttggccgtgctcgcaagagaaccggaacacaggtgctgcatggctgtcgtcagctcgtgtcgtgagatgttgggttaagtcccgcaacgagcgcaacccttgtcattagttgctacgcaagagcactctaatgagactgccggtgacaaaccggaggaaggtggggatgacgtcaagtcctcatggcccttatgggtagggcttcacacgtcatacaatggtcgggacagagggtcgccaacccgcgagggggagccaatctcagaaacccgatcgtagtccggatcgcagtctgcaactcgactgcgtgaagtcggaatcgctagtaatcgcggatcagaatgtcgcggtgaatacgttcccgggtcttgtacacaccgcccgtcacaccatgggagtgggtttcaccagaagtaggtagcctaaccgtaaggagggcgcttaccacggtgggattcatgactggggtgaag

SEQ ID NO: 237 - DNA, 1437 bp, Acidovorax temperans
  source 1..1437: mol_type = genomic DNA
atgccttacacatgcaagtcgaacggtaacaggtcttcggatgctgacgagtggcgaacgggtgagtaatacatcggaacgtgcccgatcgtgggggataacgaagcgaaagctttgccaataccgcataagatctacggatgaaagcaggggaccgcaaggccttgcgcgaacggagcggccgatggcagattaggtagttggtggggtaaaagcttaccaagccgacgatctgtagctggtctgagaggacgaccagccacactggggactgagacacggcccagactcctacgggaggcagcagtggggaattttggacaatgggcgaaagcctgatccagccatgccgcgtgcaggatgaaggccttcgggttgtaaactgcttttgtacggaacgaaaagactctggttaatacctggggtccatgacggtaccgtaagaataagcaccggctaactacgtgccagcagccgcggtaatacgtaaggtgcaagcgttaatcggaattactgggcgtaaagcgtgcgcaggcggttatataagacagatgtgaaatccccgggctcaacctgggaactgcatttgtgactgtatagctagagtacggcagagggggatggaattccgcgtgtagcagtgaaatgcgtagatatgcggaggaacaccgatggcgaaggcaatcccctgggcctgtactgacgctcatgcacgaaagcgtggggagcaaacaggattagataccctggtagtccacgccctaaacgatgtcaactgagttgttgggtcttcactgactcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtacggaatcctttagagatagaggagtgctcgaaagagagccgtaacacaggtgctgcatggctgtcgtcagctcgtgtcgtgagatgttgggttaagtcccgcaacgagcgcaacccttgccattagttgctacgaaagggcactctaatgggactgccggtgacaaaccggaggaaggtggggatgacgtcaagtcctcatggcccttataggtggggctacacacgtcatacaatggctggtacagagggttgccaacccgcgagggggagctaatcccataaagccagtcgtagtccggatcgcagtctgcaactcgactgcgtgaagtcggaatcgctagtaatcgcggatcagaatgtcgcggtgaatacgttcccgggtcttgtacacaccgcccgtcacaccatgggagcgggttctgccagaagtaggtagcctaaccgtaaggagggcgcttaccacggcagggttcgtgactggggtg

SEQ ID NO: 238 - DNA, 1342 bp, Corynebacterium
  source 1..1342: mol_type = genomic DNA
gacgaacgctggcggcgtgcttaacacatgcaagtcgaacggaaaggctccagcttgctggagtactcgagtggcgaacgggtgagtaacacgtgggtaatccgccctgcactctgggataagcctgggaaactgggtctaatactggatatgacctttctccttatggggtttggtggaaagtttttcggtgtaggatgagcccgcggcctatcagcttgttggtggggtgatggcctaccaaggcgacgacgggtagccggcctgagagggtgatcggccacattgggactgagatacggcccagactcctacgggaggcagcagtggggaatattgcacaatgggcggaagcctgatgcagcgacgccgcgtgagggatgacggccttcgggttgtaaacctctttcagcagggacgaagcgtaagtgacggtacctgcagaagaagcaccggctaactacgtgccagcagccgcggtaatacgtagggtgcgagcgttgtccggaattactgggcgtaaagagctcgtaggcggtttgtcacgtcgtctgtgaaatcctagggcttaaccctggacgtgcaggcgatacgggctgacttgagtactacaggggagactggaatttctggtgtagcggtggaatgcacagatatcaggaagaacaccgatggcgaaggcaggtctctgggtagtaactgacgctgaggagcgaaagcatggggagcgaacaggcttagataccctggtagtccacgctgtaaacggtgggtactaggtgtggggtccattccacgggttccgtgccgtagctaacgctttaagtaccccgcctggggagtacggccgcaaggctaaaactcaaaggaattgacggggccccgcacaagcggcggagcatgcggattaattcgatgcaacgcgtagaaccttacctgggtttgacatggatcgggagcgctcagagatgggtgtgcctcttttggggtcggttcacaggtggtgcatggctgtcgtcagctcgtgtcgtgagatgttgggttaagtcccgcaacgagcgcaacccttgttcactgttgccagcacgttatggtggggactcagtggagaccgccggggtcaactcggaggaaggtggggatgacgtcaagtcatcatgccccttatgtccagggcttcacgcatgctacaatggctggtacagagagtggcgagcctgtgagggtgagcgaatctcggaaagccggtctcagttcggattggggtctgcaactcgacctcatgaagtcggagtcgctagtaatcgcagatcagcaacgctgcggtgaatacgttcccggg

SEQ ID NO: 239 - DNA, 1368 bp, Streptococcus
  source 1..1368: mol_type = genomic DNA
gacgaacgctggcggcgtgcctaatgcatgcaagtagaacgctgaagagaggagcttgctcttcttggatgagttgcgaacgggtgagtaacgcgtaggtaacctgccttgtagcgggggataactattggaaacgatagctaataccgcataacaatggatgacacatgtcatttatttgaaaggggcaattgctccactacaagatggacctgcgttgtattagctagtaggtgaggtgatggctcacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgatagtcaagaacgggtgtgagagtggaaagttcacactgtgacggtagcttaccagaaagggacggctaactacgtgccagcagccgcggttatacgtaagtcccgagcgttgtccggatttattgggcgtaaagcgagcgcaggcggtttgataagtctgaagttaaggctgtggctcaaccatagttcgctttggaaactgtcaaacttgagtgcaggaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttggatcctttccgggattcagtgccgcagctaacgcattaagcactccgcctggggagtacgaccgcaaggttgaaactcaaaggaatcgacgggggcccgcacaagcggtggagcatgtggtttaattcgaagcaacgcgaagaaccttaccaggtcttgacatcccgatgctatttctagagatagaaagttacttcggtacatcggtgacaggtggtgcatggttgtcgtcagctcgtgtcgtgagatgttgggttatgtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggttggtacaacgagttgcgagtcggtgacggcgagctaatctcttaaagccaatctcagttcggattgtaggctgcaactcgcctacatgaagtcggaatcgctagtaatcgcggatcagcacgccgcggtgaatacgttcccgggcct

SEQ ID NO: 240 - DNA, 1348 bp, Finegoldia; identical to SEQ ID NO: 176
  source 1..1348: mol_type = genomic DNA

SEQ ID NO: 241 - DNA, 1377 bp, Paracoccus marcusii; identical to SEQ ID NO: 220
  source 1..1377: mol_type = genomic DNA

SEQ ID NO: 242 - DNA, 1504 bp, Staphylococcus aureus
  source 1..1504: mol_type = genomic DNA
gatgaacgctggcggcgtgcctaatacatgcaagtcaagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattaactagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcggggaacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaagg

SEQ ID NO: 243 - DNA, 1490 bp, Bacteroides dorei
  source 1..1490: mol_type = genomic DNA
gtttgaaattggctcaggatgaacgctagctacaggcttaacacatgcaagtcgaggggcagcatggtcttagcttgctaaggctgacggcgaccggcgcacgggtgagtaacacgtatccaacctgccgtctactcttggccagccttctgaaaggaagattaatccaggatgggatcatgagttcacatgtccgcatgattaaaggtattttccggtagacgatggggatgcgttccattagatagtaggcggggtaacggcccacctagtcaacgatggataggggttctgagaggaaggtcccccacattggaactgagacacggtccaaactcctacgggaggcagcagtgaggaatattggtcaatgggcgatggcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcactcgaatgatccggaaacggttcagctagcaatagcgagtgtgaaggtgctgcatggttgtcgtcagctcgtgccgtgaggtgtcggcttaagtgccataacgagcgcaacccttgttgtcagttactaacaggtgatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccctaaaacccctctcagttcggactggagtctgcaacccgactccacgaagctggattcgctagtaatcgcgcatcagccacggcgcggtgaatacgttcccgggccttgtacacatcgcccgtcaagccatgggagccgggggtacctgaagtgcgtaaccgcgaggatcgccctagggtaaaactggtgactggggctaagtcgtaaaaaggtaacc

SEQ ID NO: 244 - DNA, 1394 bp, Pseudomonas
  source 1..1394: mol_type = genomic DNA
gcagacgggtgaattatgcctaagaatctgcctgggagggggagatcacgtccagaaacgggctcttataccgcatacctcctgagagagagagtgggagatcttcccacctcacgctatcagatgagcctaggtcgtattatctagttggtggggtaaaggcctacctaggcgaccatccgttactggtgctgagaggatgatcagtcacgctggaactgagacacggtccatactcctactggaggcagcagtggggaatgttggacaatggacgaaagcctgatccagccatgccgcgtgagtgaagaacgtcttcggattgtaaagcactttaagttggaaggaagggcagtaagttaataccttgctggtttgacgttaccaacagaataagcaccgcttaacttccagccacagccgcggtaattcgaaaggggcaagcgttaatctaaattacttggcgtaaagctcgccgtaggtggttcatcaaatttgaatgtgaattccccgggctcagcatgggaactgcatccaaaacctattgagctagaagtacggtagagggtggtggaattttcccgtgtagcggtgaaatggcgtagatatagaaaggaacaccagtggcgaaggcgcccacctggactgatactgacactgaggtgcgaaagcgtggggagcaaacaggattagataccctggtagtccacgccgtaaacgatgtcgactagccgttgggatccctgagatcttagtggcgcagctaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccgcaagggggacggttaccacggagtgattcatgactggggtga

SEQ ID NO: 245 - DNA, 1418 bp, Selenomonas
  source 1..1418: mol_type = genomic DNA
gagtttgatcatggctcaggacgaacgctggcggcgtgcttaacacatgcaagtcgaacggagcgaatgaaagcttgcttttatgagcttagtggcaaacgggtgagtaacacgtagacaacctgccgacaggatggggacaacattccgaaaggaatgctaataccgaatgaagcggaggagaggcatctctcctccgtgaaagatggcctctatttataagctatcacctgtcgatgggtctgcgtctgattagctagttggtgaggtaacggctcaccaaggcgacgatcagtagccggtctgagaggatgaacggccacattgggactgagacacggcccagactcctacgagaggcagcagtggggaatcttccgcaatgggcgcaagcctgacggagcaacgccgcgtgagtgaagaaggtcttcggatcgtaaagctctgttgatggggacgaacgtgcgaagggtgaataatcctttgcaatgacggtacctatcgaggaagccacggctaactacgtgccagcagccgcggtaatacgtaggtggcgagcgttgtccggaatcattgggcgtaaagggagcgcaggcgggcatgtaagtctttcttaaaagttcggggctcaaccccgtgatgggaaagaaactacatgtcttgagtacaggagaggaaagcggaattcccagtgtagcggtgaaatgcgtagatattgggaggaacaccagtggcgaaggcggctttctggactgcaactgacgctgaggctcgaaagccaggggagcgaacgggattagataccccggtagtcctggccgtaaacgatggatactaggtgtgggaggtatcgacccctaccgtgccggagttaacgcaataagtatcccgcctggggagtacggtcgcaagactgaaactcaaaggaattgacggggacccgcacaagcggtggagtatgtggtttaattcgaagcaacgcgaagaaccttaccaggccttgacattgactgtaagagttagagataactccctctcttcggagacaggaaaacaggtggtgcatggctgtcgtcagctcgtgtcgtgagatgttgggttaagtcccgcaacgagcgcaacccctgttctttgttgccatcaggtaaagctgggcactcaaaggagactgccgcggagaacgcggaggaaggcggggatgacgtcaagtcatcatgccccttatggtctgggctacacacgtactacaatggaacggacagagagcagcgaacccgcgagggcaagcgaacctcaaaaaccgtttcccagttcggattgcaggctgcaactcgcctgcatgaagtcggaatcgctagtaatcgcaggtcagcatactgcggtgaatacgttcccgggtcttgtacacaccgcccgtc

SEQ ID NO: 246 - DNA, 1505 bp, Pseudomonas viridiflava
  source 1..1505: mol_type = genomic DNA
attgaacgctggcggcaggcctaacacatgcaagtcgggcggcagcacaggtacttgtaccgggtggcgagcggcggacgggtgagtaatgcctaggaatctgcctggtagtgggggataacgctcggaaacggacgctaataccgcatacgtcctacgggagaaagcaggggaccttcgggccttgcgctatcagatgagcctaggtcggattagctagttggtgaggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggtcgttacctaatacgtgacgattttgacgttaccgacagaataagcaccggctaactctgtgccagcagccgcggtaatacagagggtgcaagcgttaatcggaattactgggcgtaaagcgcgcgtaggtggttcgttaagttggatgtgaaatccccgggctcaacctgggaactgcatccaaaactggcgagctagagtagggcagagggtggtggaatttcctgtgtagcggtgaaatgcgtagatataggaaggaacaccagtggcgaaggcgaccacctgggctcatactgacactgaggtgcgaaagcgtggggagcaaacaggattagataccctggtagtccacgccgtaaacgatgtcaactagccgttggaatccttgagattttagtggcgcaaataacgcattaagttgaccgcctggggagtacggccgcaaggttaaaactcaaatgaattgacgggggcccgcacaagcggtggagcatgtggtttaattcgaagcaacgcgaagaaccttaccaggccttgacatccagtgaacttaccagagatggtttggtgccttcgggaacactgagacaggtgctgcatggctgtcgtcagctcgtgtcgtgagatgttgggttaagtcccgtaacgagcgcaacccttgtccttagttaccagcacgtgatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatctcacaaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcggggggacggttaccacggtgtgattcatgactggggtgaagtcgtaacaaggtagccgtaggggaacctgcggctggatcac

SEQ ID NO: 247 - DNA, 1463 bp, Geobacillus
  source 1..1463: mol_type = genomic DNA
ggacgaacgctggcggcgtgcctaatacatgcaagtcgagcggaccaaatcggagcttgctctgatttggtcagcggcggacgggtgagtaacacgtgggcaacctgcccgcaagaccgggataactccgggaaaccggagctaataccggataacaccgaagaccgcatggtctttggttgaaaggcggcctttggctgtcacttgcggatgggcccgcggcgcattagctagttggggaggtaacggctcaccaaggcgacgatgcgtaaccggcctgagagggtgaccggccacactgggactgacacacggcccagactcctacgggaggcaacagtagggaatcttccgcaatgggcgaaagcctgacggagcgacgccacgtgagcgaagaaagccttcgggtcgtaaagctctgttgagagggacgaaggagcgccgtttgaacaaggcggcgcggtgacggtacctcacgaggaagccccggctaactacgtgccagcagccgcggtaatacgtagggggcgagcgttgtccggaattattgggcgtaaagcgcgcgcaggcggtctcttaagtctgatgtgaaagcccacggctcaaccgtggagggtcattggaaactgggggacttgagtgcaggagaggagagcggaattccacgtgtagcggtgaaatgcgtagagatgtggaggaacaccagtggcgaaggcggctctctggcctgcaactgacgctgacgcgcgaaagcgtggggagcaaacaggttttgataccctgggtgtcggcgccgtaacgatgaggtgttaagtggttaggggggtcacacccttttagtgctgcagttaacgcgataagcactcccggctggggagaaccgcccgcaaggctgaaactcaaaggaattgtcgggggcccgcacaagcgggtggagcatgtggtttaattggaagcaacgcgaagaaccttaccaggtcttgacatcccctgacaacccaagagattgggcgttcccccttcggggggacagggtgacaggtggtgcatggttgtcgtcagctcgtgtcgtgagatgttgggttaagtcccgcaacgagcgcaaccctcgcctctagttgccagcacgaaggtgggcactctagagggactgccggcgacaagtcggaggaaggtggggatgacgtcaaatcatcatgccccttatgacctgggctacacacgtgctacaatgggcggtacaaagggctgcgaacccgcgagggggagcgaatcccaaaaagccgctctcagttcggattgcaggctgcaactcgcctgcatgaagccggaatcgctagtaatcgcggatcagcatgccgcggtgaatacgttcccgggccttgtacacaccgcccgtcacaccacgagagcttgcaacacccgaagtcggtaaagtaacccttacgggagccagccgccga

SEQ ID NO: 248 - DNA, 1360 bp, Klebsiella
  source 1..1360: mol_type = genomic DNA
attgaacgctggcggcaggcctaacacatgcaagtcgaacggtagcacagagagcttgctctcgggtgacgagtggcggacgggtgagtaatgtctgggaaactgcccgatggagggggataactaccggaaacggtagctaataccgcataatgtcgcaagaccaaagagggggaccttcgggcctcttgccatcggatgtgcccagatgggattagcttgtaggtgaggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtgaggttaataaccttattcattgacgttacccgcagaagaagcaccggctaactccgtgccagcagccgcggtaatgcggagggtgcaagcgttaatcggaaatactgggcgtaagcgcacgcaggcggtctgtcaagtcggatgtgaaatccccgggctcaacctgggaactgcattcgaaactggcaggctggagtcttgtagaggggggtagaattccaggtgtaccggtgaaatgcgtagagatctggaggaataccggtggcgaaggcggccccctggacaaagactgacgctcacgtgcgaaagcgtggggagcaaacaggattagataccctggtagtccacgctgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggttttattcgatgcaacgcgaagaaccttacctactcttgacatccacggaatttggcagagatgccttagtgccttcgggaaccgtgagacaggtgctgcatggctgtcgtcagctcgtgttgtgaaatgttgggttaagtcccgcaacgagcgcaacccttatcctttgttgccagcgattcggtcgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatggagtcggaatcgctagtaatcgtggatcagaatgccacggtgaatacgttcccgggcct

SEQ ID NO: 249 - DNA, 1482 bp, Pseudomonas
  source 1..1482: mol_type = genomic DNA
agagtttgatcatggctcagattgaacgctggcggcaggcctaacacatgcaagtcgagcggatgaagggagcttgctcctggattcagcagcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccacggattatgcaccggctaacttaatgccagcagctatgttcataccaacgccgcggcaataaataggatgcactcgttaatcgatattactgagcgtagtgcgcgcgtaggtggttcatcatgctggatgtgacatccgcaggctcaccctgcgaactgcatcaaagaatacgtgaactagtggtacggaagggtggtggaattctggttagcgtgaaatgcgtagatataggaaggacaccagtggcgatgcgatcacctggactgatactgacactgaggtgcgaaagcgtggggagcaaacaggattagataccctggtagtacacgccgtaaacgatgtcgactagccgttgggatccttgagatcttagtggcgcagctaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggagctcagacacaggtgctgcatggctgtcgtcagctcgtgtcgtgagatgttgggttaagtcccgtaacgagcgcaacccttgtccttagttaccagcacctcgggtgggcactctaaggggactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagagctagtctaaccgcaagggggacggttaccacggaggattcatgactg

SEQ ID NO: 250 - DNA, 1309 bp, unidentified
  misc_feature 1..1309: note = 16S ribosomal RNA of unknown bacteria
  source 1..1309: mol_type = genomic DNA
gatgaacgctggcggcatgcttaacacatgcaagtcgaacgggaagtggtgtttccagtggcggacgggtgagtaacgcgtaagaacctgcccttgggaggggaacaacaactggaaacggttgctaataccccgtaggctgaggagcaaaaggagaaatccgcccaaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtggaggtggaaggcctacgggtcgtcaacttcttttctcggagaagaaacaatgacggtatctgaggaataagcatcggctaactctgtgccagcagccgcggtaagacagaggatgcaagcgttatccggaatgattgggcgtaaagcgtctgtaggtggcttttcaagtccgccgtcaaatcccagggctcaaccctggacagacggtggaaactaccaagctggagtacggtaggggcagagggaatttccggtggagcggtgaaatgcattgagatcggaaagaacaccaacggcgaaagcactctgctgggccgacactgacactgagagacgaaagctaggggagcaaatgggattagagaccccagtagtcctagccgtaaacgatggatactaggtgctgtgcgactcgacccgtgcagtgctgtagctaacgcgttaagtatcccgcctgggagtacgttcgcaagaatgaagctcaaaggaattgacgggggcccgcacaagcggtggagcatgtggtttaattcgatgcaaagcgaagaaccttaccagggcttgacatgccgcgaatcctcttgaaagaggggggtgcccttgggaacgcggacacaggtggtgcatggctgtcgtcagctcgtgccgtaaggtgttgggttaagtctcgcaacgagcgcaaccctcgtgtttagttgccactatgagtttggaaccctgaacagaccgccggtgttaagccggaggaaggagaggatgaggccaagtcatcatgccccttatgccctgggcgacacacgtgctacaatgggcgggacaaagggtcgcgatctcgcgagggtgagctaactccaaaaacccgtcctcagttcggattgcaggctgcaactcgcctgcatgaagcaggaatcgctagtaatcgccggtcagccatacggcggtgaatccgttcccgggcct

SEQ ID NO: 251 - DNA, 1428 bp, Xanthomonas
  source 1..1428: mol_type = genomic DNA
acacatgcaagtcgaacggcagcacagtaagagcttgctcttatgggtggcgagtggcggacgggtgaggaatacatcggaatctactctttcgtgggggataacgtagggaaacttacgctaataccgcatacgacctacgggtgaaagcggaggaccttcgggcttcgcgcgattgaatgagccgatgtcggattagctagttggcggggtaaaggcccaccaaggcgacgatccgtagctggtctgagaggatgatcagccacactggaactgagacacggtccagactcctacgggaggcagcagtggggaatattggacaatgggcgcaagcctgatccagccatgccgcgtgggtgaagaaggccttcgggttgtaaagcccttttgttgggaaagaaaagcagtcggttaatacccgattgttctgacggtacccaaagaataagcaccggctaacttcgtgccagcagccgcggtaatacgaagggtgcaagcgttactcggaattactgggcgtaaagcgtgcgtaggtggtggtttaagtctgttgtgaaagccctgggctcaacctgggaattgcagtggatactgggtcactagagtgtggtagagggtagcggaattcccggtgtagcagtgaaatgcgtagagatcgggaggagcatccgtggcgaaggcggctacctggacggacactgacactgaggcacgaaagcgtggggagcaaacaggattagataccctggtagtccacgccctaaacgatgcgaactggatgttgggtgcaatttggcacgcagtatcgaagctaacgcgttaagttcgccgcctggcggagtacggtcgcaagactgaaactcaaaggaattgacggggggcccggcacaacgcggtggagtatgtgggtttaattcgatgcaacgcgaagaaccttacctggtcttgacatccacggaactttccagagatggattggtgccttcgggaaccgtgagacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtggggatgacgtcaagtcatcatggcccttacgaccagggctacacacgtactacaatggtagggacagagggctgcaaacccgcgagggtaagccaatcccagaaaccctatctcagtccggattggagtctgcaactcgactccatgaagtcggaatcgctagtaatcgcagatcagcattgctgcggtgaatacgttcccgggccttgtacacaccgcccgtcacacctatgggagtttgttgcacctagaagcaggtagcttaaccttcgggagggcgctgcc

SEQ ID NO: 252 - DNA, 1343 bp, Corynebacterium
  source 1..1343: mol_type = genomic DNA
gacgaacgctggcggcgtgcttaacacatgcaagtcgaacggaaaggctccagctcgctggagtactcgagtggcgaacgggtgagtaacacgtgggtaatctgccctgcactctgggataagcctgggaaactgggtctaatactggatatgacctttctccttatggggtttggtggaaagtttttcggtgtaggatgagcccgcggcctatcagcttgttggtggggtgatggcctaccaaggcgacgacgggtagccggcctgagagggtgatcggccacattgggactgagatacggcccagactcctacgggaggcagcagtggggaatattgcacaatgggcggaagcctgatgcagcgacgccgcgtgagggatgacggccttcgggtttgtaacctctttcagcagggacgaagcgtaagtgacggtacctgcagaagaagcaccggctaactacgtgccagcagcccgcgtaataccgtaggtgcgagcgttgtccggaattactgggcgtaaagagctcgtaggcggtttgtcacgtcgtctgtgaaatccctagggcttaaccctggacgtgcaggcgatacgggctgacttgaatactacaggggagactggaatttctgtgtagcgggtggaatgcacagatatcaggaagaacaccgatggcgaaggcaggtctctgggtagtaactgacgctgaggagcgaaagcatgggtagcgaacaggattagataccctggtagtccatgccgtaaacggtgggcgctaggtgtggggttccttccacgggctccgcgccgtagctaacgcattaagcgccccgcctggggagtacggccgcaaggctaaaactcaaagaaattgacgggggcccgcacaagcggcggagcatgtggattaattcgatgcaacgcgaagaaccttacctgggcttgacatatagaggatcgctacagagatgtggtttcccttgtggcttctatacaggtggtgcatggttgtcgtcagctcgtgtcgtgagatgttgggttaagtcccgcaacgagcgcaacccttgtcttatgttgccagcacgttatggtggggactcgtgagagactgccggggtcaactcggaggaaggtggggatgacgtcaaatcatcatgccccttatgtccagggcttcacacatgctacaatggccggtacagagggctgcgatgccgtgaggcggagcgaatctcttaaagccggtctcagttcggattggggtctgcaactcgaccccatgaagtcggagtcgctagtaatcgcagatcagcaacgctgcggtgaatacgttcccgggcct

SEQ ID NO: 253 - DNA, 1355 bp, Clostridium
  source 1..1355: mol_type = genomic DNA
gatgaacgctggcggcgtgcttaacacatgcaagttgaacgaaactttcagaaacagattcttcggatgacgattctgaaacgtttagtggcggatgggcgagtaacacgtgaggaacctacctttcacaggggaataacactgggaaaccagtgctaataccgcataatgtattaaagtggcatcacattaataccaaagattttatcggtgaaagatggcctcgcgcctgattagctagtaggtgaggtaacggctcacctaggtgatgatcagtagccggactgagaggttgatcggccgcattgggactgagacacggcccagactcctacgggaggcagcagtggggaatattgggcaatgggcgcaagcctgacccagcaacgccgcgtgaaggaagacggtcttcggattgtaaacttctttagacagggacgaaacaaatgacggtacctgtagaataagccacggctaactacgtgccagcagccgcggtaatacgtaggtggcgagcgttatccggaattactgggtgtaaagggcgtgtaggcggcactgtaagtcagatgtgaaatctcccggctcaaccgggagcgtgcatctgatactgcaatgcttgagtgatagaggggaaagcggaattcctagtgtagcggtaaaatgcgtagatattaggaggaacaccagtggcgaaggcggctttctggctattaactgacgctgaggcgcgaaagcgtggggagcaaacaggattagataccctggtagtccacgccgtaaacgatgaatgctagatgtgggaggtatcgaccccttccgtgtcgcagctaacgcaataagcattccacctggggagtacggccgcaaggctgaaactcaaaggaattgacggggacccgcacaagcagtggagtatgtggtttaattcgaggctacgcgaagaaccttaccaaggcttgacatcttctgacagctctagagatagagttttcctacgggacagaatgacaggtggtgcatggctgtcgtcagctcgtgtcgtgagatgttcggttaagtccggcaacgagcgcaacccctacggtatattgccatcattcagttgggcacttttactggactgccgttaacaaaacggaggaaggtggggatgacgtcaagtcatcatgccccttatgtcttgggctacacacgtactacaatggtaatgacagagggcagcgacatagcgatatgaagcgaatcccaaaacgttatctcagttcggattggaggctgcaactcgcctccatgaagccggaattgctagtaatggcaggtcagcatactgccgtgaatacgttctcgggtct

SEQ ID NO: 254 - DNA, 1359 bp, Acinetobacter
  source 1..1359: mol_type = genomic DNA
attgaacgctggcggcaggcttaacacatgcaagtcgagcggggatagggtgcttgcacctgattcctagcggcggacgggtgagtaatgcttaggaatctgcctattagtgggggacaacgttccgaaagggacgctaataccgcatacgtcctacgggagaaagcaggggatcttcggaccttgcgctaatagatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatggggggaaccctgatccagccatgccgcgtgtgtgaagaaggccttttggttgtaaagcactttaagcgaggaggaggcttacctggttaatacctgggataagtggacgttactcgcagaataagcaccggctaactctgtgccagcagccgcggtaatacagagggtgcaagcgttaatcggatttactgggcgtaaagcgcgcgtaggtggctaattaagtcaaatgtgaaatccccgagcttaacttgggaattgcattcgatactggttagctagagtatgggagaggatggtagaattccaggtgtagcggtgaaatgcgtagagatctggaggaataccgatggcgaaggcagccatctggcctaatactgacactgaggtgcgaaagcatggggagcaaacaggattagataccctggtagtccatgccgtaaacgatgtctactagccgttggggcccttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggccttgacatactaagaactttccagagatggattggtgccttcgggaacttagatacaggtgctgcatggctgtcgtcagctcgtgtcgtgagatgttgggttaagtcccgcaacgagcgcaacccttttccttatttgccagcacttcgggtgggaactttaaggatactgccagtgacaaactggaggaaggcggggacgacgtcaagtcatcatggcccttacggccagggctacacacgtgctacaatggtcggtacaaagggttgctactgcgcgagcagatgctaatctcaaaaagccgatcgtagtccggatcgcagtctgcaactcgactgcgtgaagtgggaatcgctagtaatcgcggatcagaatgccgcggtgaatacgttcccgggcct

SEQ ID NO: 255 - DNA, 1483 bp, Massilia
  source 1..1483: mol_type = genomic DNA
ggctcagattgaacgctggcggcatgctttacacatgcaagtcgaacggtaacagggagcttgctccgctgacgagtggcgaacgggtgagtaatatatcggaacgtacccaagagtgggggataacgtagcgaaagttacgctaataccgcatacgatccaaggatgaaagcaggggaccgcaaggccttgtgctcctggagcggccgatatctgattagctagttggtgaggtaaaagctcaccaaggcgacgatcagtagctggtctgagaggacgaccagccacactggaactgagacacggtccagactcctacgggaggcagcagtggggaattttggacaatgggcgcaagcctgatccagcaatgccgcgtgagtgaagaaggccttcgggttgtaaagctcttttgtcagggaagaaacggtaggggctaatatcctttgctaatgacggtacctgaagaataagcaccggctaactacgtgccagcagcccggtaatacgtaggggcaagcgttaatcggattactgggcgtaagcgtgcccaggcgtttttgtaagtctgtcgtgaaatccccgggctcaacctgggaattgcgatggagactgcaaggctagaatctgggagaggggggtagaattccacgtgtagcagtgaaatgcgtagagatgtggaggaacaccgatggcgaaggcggccccctgggtcaagattgacgctcatgcacgaaagcgtggggagcaaacaggattagatacccctggtagtccacgcctaaacgatgtctactagttgtcgggttttaattaacttggtaacgcaggtaacgcgtgaagtagaaccgcctgggggagtacggtccgcaagattaaaactcaaaggaattgacgaggaccccgcacaagcggtggatgatgtggattaattcgatgcaacgcgaaaaaccttacctacccttgacatgtcaggaaggccggagagatttggctgtgcccgaaagggaacctgaacacaggtgctgcatggctgtcgtcagctcgtgtcgtgagatgttgggttaagtcccgcaacgagcgcaacccttgtcattagttgctacattcagttgagcactctaatgagactgccggtgacaaaccggaggaaggtggggatgacgtcaagtcctcatggcccttatgggtagggcttcacacgtcatacaatggtacatacagagggccgccaacccgcgagggggagctaatcccagaaagtgtatcgtagtccggatcgcagtctgcaactcgactgcgtgaagttggaatcgctagtaatcgcggatcagcatgtcgcggtgaatacgttcccgggtcttgtacacaccgcccgtcacaccatgggagcgggttttaccagaagtaggtagcttaaccgcaaggggggcgcttaccacggtaggattcgtgactggggtgaagtcgtaacaaggtaacc

SEQ ID NO: 256 - DNA, 1391 bp, unidentified
  misc_feature 1..1391: note = 16S ribosomal RNA of unknown bacteria
  source 1..1391: mol_type = genomic DNA
agagtttgatcctggctcaggctgaacgctggcggcgtgcttaacacatgcaagtcgaacggagtgaataaaacggaagttttcggatggaacatttatttacttagtggcggacgggtgagtaacgcgtgggtaacctgccgtatacagggggataacacttagaaataggtgctaataccgcataagcgcgcagcttcgcatgaagcagtgtgaaaagccttgacggtatacgatggacccgcgtctgattagctagttggtgaggtagaggctcaccaaggcgacgatcagtagccggcctgagagggtgaccggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtgatgcaagtctgaagtgaaagcccggtgctcaacgccgggactgctttggaaactgtgttactagagtgcaggagaggtaagtggaattcctagtgtagcggtgaaatgcgtagatattaagaggaccaccagtggcgaaggcggcttactggactgtaactgacagttgaggctcgaagcgtggggagcaacaggattagataccttgctagtccactccgtaaacgatgattagtaggtgctggttgtaagagcctatcggtgcactcgccaatgcagtacgttatccacgtggggagtacgctcgctagaatgaacctcaaaggaattgacggggacccgcacaagcggtggagcatgtggtttcattcgaatccacgcgaagaaccttaacatatcttgacatcttttcgacctagtatgttaagtactatctgttcggagcaggagtgacagatggtgcatggttgtcttcagctcgagtcgtgacatgttggcattagtcacgcgacgagtcccacccttatctctagtagccatcattacgatggggagtctagagagcatcccggggataacccggaggaaggtggggataacctctcatcatcatgcccattatgacatgggatacacacgtgttacaagggagtacacaaagggaaaagacccatcgagggtgagcaaatcatcataaataacgtctcatcacggatcgtactctgcaattagaaaacatgcatctggaatcgttagtaatagtgaatcagcatgtcgcgcagaatacgttcaccgggtccagtacacaccgcccgtca

SEQ ID NO: 257 - DNA, 1451 bp, Eikenella corrodens
  source 1..1451: mol_type = genomic DNA
cggcatgctttacacatgcaagtcgaacggcagcggggtagtgcttgcactactgccggcgagtggcgaacgggtgagtaatatatcggaacgtaccgagtaatgggggataaccaatcgaaagattggctaataccgcatacgtcctgagggagaaagcgggggatcgcaagacctcgcgttattcgagcggccgataactgattagctagttggtggggtaaaggcctaccaaggcgacgatcagtagcgggtctgagaggacgatccgccacactgggactgagacacggcccagactcctacgggaggcagcagtggggaattttggacaatgggggcaaccctgatccagccatgccgcgtgtatgaagaaggccttcgggttgtaaagtacttttgttagggaagaaaagggaagtgctaataccactttttgctgacggtacctaaagaataagcaccggctaactacgtgccagcagctgcggtaatacgtagggtgcgagcgttaatcggaattactgggcgtaaagcgagcgcagacggttatttaagcaggatgtgaaatccccgggcttaacctgggaactgcgttctgaactggatggctagagtgtgtcagaggggggtagaattccacgtgtagcagtgaaatgcgtagagatgtggaggaataccgatggcgaaggcagccccctgggataacactgacgttcatgctcgaaagcgtgggtagcaaacaggattagataccctggtagtccacgccctaaacgatgtcgattagctgttgggcaacttgattgcttagtagcgtagctaacgcgtgaaatcgaccgcctggggagtacggtcgcaagattaaaactcaaaggaattgacggggacccgcacaagcggtggatgatgtggattaattcgatgcaacgcgaagaaccttacctggtcttgacatgtacggaaccttccagagacggaagggtgccttcgggagccgtaacacaggtgctgcatggctgtcgtcagctcgtgtcgtgagatgttgggttaagtcccgcaacgagcgcaacccttgtcattagttgccatcatttagttgggcactctaatgagactgccggtgacaaaccggaggaaggtggggatgacgtcaagtcctcatggcccttatgaccagggcttcacacgtcatacaatggtcggtacagagggtagccaagccgcgaggtggagccaatcccagaaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagcaggtagggtaaccgcaaggagcccgcttgccacggtatgcttcatgactggggtg

SEQ ID NO: 258 - DNA, 1443 bp, Bacteroides
  source 1..1443: mol_type = genomic DNA
caggcttaacacatgcaagtcgaggggcagcatggtcttagcttgctaaggctgatggcgaccggcgcacgggtgagtaacacgtatccaacctgccgtctactcttggccagccttctgaaaggaagattaatccaggatgggatcatgagttcacatgtccgcatgattaaaggtattttccggtagacgatggggatgcgttccattagatagtaggcggggtaacggcccacctagtcaacgatggataggggttctgagaggaaggtcccccacattggaactgagacacggtccaaactcctacgggaggcagcagtgaggaatattggtcaatgggcgatggcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgccaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcactcgaatgatccggaaacggttcagctagcaatagcgagtgtgaaggtgctgcatggttgtcgtcagctcgcgccgtgaggtgtcggcttaagtgccataacgagcgcaacccttgttgtcagttactaacaggtgatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccctaaaacccctctcagttcggactggagtctgcaacccgactccacgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagt

SEQ ID NO: 259 - DNA, 1413 bp, Selenomonas
  source 1..1413: mol_type = genomic DNA
gagtttgatcatggctcaggacgaacgctggcggcgtgcttaacacatgcaagtcgaacgggaaactttatttcggtaaaggtgagagtggcaaacgggtgagtaacacgtaggcaacctgccgacaggatggggacaacattccgaaaggaatgctaataccgaatggagtccggagatggcatcatccccggataaaagatggcctctgaatatgctatcgcctgtcgatgggcctgcgtctgattagccagttggcggggtaacggcccaccaaagcgacgatcagtagccggtctgagaggatgaacggccacattgggactgagacacggcccagacccctacgggaggcagcagtggggaatcttccacaatgggcgaaagcctgatggagcaacgccgcgtgagtgaagaaggtcttcggatcgtaaagctctgttgcacgggacgaaaaccgggtttgagaatattgaacccgggtgacggtaccgagcgaggaagccacggctaactacgtgccagcagccgcggtaatacgtaggtggcgagcgttgtccggaattattgggcgtaaagggagcgcaggcggacatataagtccatcttaaaagtgcggggctcaaccccgtgaggggatggaaactgtatgccttgagtgcaggagaggaaagcggaattcccagtgtagcggtgaaatgcgtagatattgggaggaacaccagtggcgaaggcggctttctggactgcaactgacgctgaggctcgaaagccaggggagcgaacgggattagataccccggtagtcctggccgtaaacgatgaatgctaggtgtaggaggtatcgacccctcctgtgccggagttaacgcaataagcattccgcctggggagtacggtcgcaagactgaaactcaaaggaattgacgggggcccgcacaagcggtggagtatgtggtttaattcgacgcaacgcgaagaaccttaccagggcttgacattgagtgaaagagctagagatagcttcctcccttcggggacacgaaaacaggtggtgcatggctgtcgtcagctcgtgtcgtgagatgttgggttaagtcccgcaacgagcgcaacccctgtcctttgttgccagcgcgtaatggcgggaactcaaaggagactgccgcggagaacgcggaggaaggcggggatgacgtcaagtcatcatgccccttatgtcctgggctacacacgtactacaatgggatggacagagggaagcgaaggcgcgagccggagcggaccccacaaaccgtcccccagttcggattgcaggctgcaacccgcctgcatgaagtcggaatcgctagtaatcgcaggtcagcatactgcggtgaatacgttcccgggccttgtacacaccgcccgtca

SEQ ID NO: 260 - DNA, 1391 bp, Eubacterium saburreum
  source 1..1391: mol_type = genomic DNA
agagtttgatcatggctcaggatgaacgctggcggcgtgcttaacacatgcaagtcgaacgaagctgttaaaaggaagtcttcggatggaattttaacagacttagtggcggacgggtgagtaacgcgtggataacctgccttatacagggggataacggagagaaatttccgctaagaccgcataagaccacagcaccgcatggtgcaggggtaaaatattaataggtataagatggatccgcgtccgattagctggttggtgaggtagaggctcaccaaggcgacgatcggtagccggcctgagagggtgaacggccacattgggactgagacacggcccaaactcctacgggaggcagcagtggggaatattggacaatgggggaaaccctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgcagacggccaagcaagtctgaagtgaaatgcatgggctcaacccatggattgctttggaaactgttaggcttgagtgtcggaggggtaagcggaattcctagtgtagcggtgaaatgcgtagatattaggaggaacaccggaggcgaaggcggcttactggacgacaactgacgttgaggctcgaaggcgtggggagcaaacaggattagataccctggtagtccacgcagtaaacgatgaatacttggtgtcggggagataaactcttcggtgccgcaagcaaacgcattaagtattccacctggggagtacgttcgcaagaatgaaactcaaaggaattgacggggacccgcacaagcggtggagcatgtggtttaattcgaagcaacgcgaagaaccttaccaaatcttgacatactcttgaataatcttgtaatgagattagtctttcgggacaagggatacaggtggtgcatggttgtcgtcagctcgtgtcgtgagatgttgggttaagtcccgcaacgagcgcaacccttgtcgtcagtagccagcagtaagatgggaactctgacgagacagccggagataatccggaggaaggtggggatgacgtcaaatcatcatgccccttatgatttgggctacacacgtgctacaatggcgtaaacaaagtgaagcaagaccgcgaggttaagcaaatcacagaaataacgtctcagttcggattgtagtctgcaactcgactatatgaagccggaatcgctagtaatcgcagatcagaatgctgcggtgaatacgttcccgggtcttgtacacaccgcccgtca

SEQ ID NO: 261 - DNA, 1414 bp, Selenomonas
  source 1..1414: mol_type = genomic DNA
gagtttgattcatggctcaggacgaacgctggcggcgtgcttaacacatgcaagtcgaacgggaacctttatttcggtaaaggtgagagtggcaaacgggtgagtaacacgtaggcaacctgccgacaggatggggacaacattccgaaaggaatgctaataccgaatggagtccggggatggcatcatcctcggataaaagatggcctctgaatatgctatcgcctgtcgatgggcctgcgtctgattagccagttggcggggtaacggcccaccaaagcgacgatcagtagccggtctgagaggatgaacggccacattgggactgagacacggcccagactcctacgggaggcagcagtggggaatcttccacaatgggcgaaagcctgatggagcaacgccgcgtgagtgaagaaggtcttcggatcgtaaagctctgttgcacgggacgaaaaccgggtttgagaatattgaacccgggtgacggtaccgagtgaggaagccacggctaactacgtgccagcagccgcggcaatacgtaggtggcgagcgttgtccggaattattgggcgtaaagggagcgcaggcggacatataagtccatcttaaaagtgcggggctcaaccccgtgaggggatggaaactgtatgccttgagtgcaggagaggaaagcggaattcccagtgtagcggtgaaatgcgtagatattgggaggaacaccagtggcgaaggcggctttctggactgcaactgacgctgaggctcgaaagccaggggagcgaacgggattagataccccggtagtcctggccgtaaacgatgaatgctaggtgtaggaggtatcgacccctcctgtgccggagttaacgcaataagcattccgcctggggagtacggccgcaaggctgaaactcaaaggaattgacgggggcccgcacaagcggtggagtatgtggtttaattcgacgcaacgcgaagaaccttaccagggcttgacattgagtgaaagagctggagacagctccctcccttcggggacacgaaaacaggtggtgcatggctgtcgtcagctcgtgtcgtgagatgttgggttaagtcccgcaacgagcgcaacccctgtcctttgttgccagcgcgtaatggcgggaactcaaaggagactgccgcggagaacgcggaggaaggcggggatgacgtcaagtcatcatgccccttatgtcctgggctacacacgtactacaatgggatggacagagggaagcgaaggcgcgagccggagcggaccccacaaaccatcccccagttcggattgcaggctgcaacccgcctgcatgaagtcggaatcgctagtaatcgcaggtcagcatactgcggtgaaatacgttcccgggccttgtacacaccgcccgtc

SEQ ID NO: 262 - DNA, 1467 bp, Citrobacter freundii
  source 1..1467: mol_type = genomic DNA
attgaacgctggcggcaggcctaacacatgcaagtcgaacggtagcacagaggagcttgctccttgggtgacgagtggcggacgggtgagtaatgtctgggaaactgcccgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcgttgtggttaataaccgcagcgattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcgattaggacgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaag

SEQ ID NO: 263 - DNA, 1496 bp, Klebsiella pneumoniae; identical to SEQ ID NO: 189
  source 1..1496: mol_type = genomic DNA

SEQ ID NO: 264 - DNA, 1425 bp, Enterobacter asburiae
  source 1..1425: mol_type = genomic DNA
agtcgaacggtaacaggaagcagcttgctgcttcgctgacgagtggcggacgaatgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggtgttgaggttaataacctcagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgac

SEQ ID NO: 265 - DNA, 1520 bp, Veillonella; identical to SEQ ID NO: 205
  source 1..1520: mol_type = genomic DNA

SEQ ID NO: 266 - DNA, 1327 bp, Fusobacterium nucleatum; identical to SEQ ID NO: 210
  source 1..1327: mol_type = genomic DNA

SEQ ID NO: 267 - DNA, 1396 bp, Enterobacter cloacae
  source 1..1396: mol_type = genomic DNA
agagtttgatcaggctcagattgaacgctggcggcaggcctaacacatgcaagtcgaacggtagcgcagagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaatggctcacctaggcgacgatccctagctggtctgagaggatgaccagccacactggaactgagacacggtccagactcctatgggaggcagcagtggggaatattgcacaatgggcgcaagcctgatgcagccatgccgcgtgtatgaagaaggccttcgggttgtaaagtactttcagcggggaggaaggtgttgtggttaataaccacagcaattgacgttacccgcagaagaagcaccggctaactccgtgccagcagccgcggtaatacggagggtgcaagcgttaatcggaattactgggcgtaaagcgcacacaggcggtctgtcaagtcggatgtgaaatccccgggctcaacctgggaactgcattcgaaactggcaggctagagtctagtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccgtgtacacaccgcccgt

SEQ ID NO: 268 - DNA, 1407 bp, Klebsiella oxytoca
  source 1..1407: mol_type = genomic DNA
agtcgaacggtagcacagaagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgataaggttaataaccttgtcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctggagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcgatacggacgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaacccttcgggagggcgctacc

SEQ ID NO: 269 - DNA, 1471 bp, Enterobacter aerogenes
  source 1..1471: mol_type = genomic DNA
gagtttgaatcctggctcagattgaacgctggcggcaggcctaacacatgcaagtcgagcggtaacacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcgttaaggttaataaccttggcgattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcggtcaggccgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtggttgcaaagaataatagcttacatcggaggcgctacacttgtgatcatgactggggtga

SEQ ID NO: 270 - DNA, 1524 bp, Streptococcus anginosus
  source 1..1524: mol_type = genomic DNA
aacgctggcggcgtgcctaatacatgcaagtaggacgcacagtttataccgtagcttgctacaccatagactgtgagttgcgaacgggtgagtaacgcgtaggtaacctgcctattagagggggataactattggaaacgatagctaataccgcataacagtatgtaacacatgttagatgcttgaaagatgcaattgcatcgctagtagatggacctgcgttgtattagctagtaggtagggtaaaggcctacctaggcaacgatacatagccgacctgagagggtgatcggccacactgggactgagacacggcccagactcctacgggaggcagcagtagggaatcttcggcaatggggggaaccctgaccgagcaacgccgcgtgagtgaagaaggttttcggatcgtaaagctctgttgttaaggaagaacgagtgtgagaatggaaagttcatgctgtgacggtacttaaccagaaagggacggctaactacgtgccagcagccgcggtaatacgtaggtcccgagcgttgtccggatttattgggcgtaaagcgagcgcaggcggttagaaaagtctgaagtgaaaggcagtggctcaaccattgtaggctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttaggtcctttccgggacttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccagtgaccgccctagagatagggtttcccttcggggcactggtgacaggtggtgcatggttgtcgtcagctcgtgtcgtgagatgttgggttaagtcccgcaacgagcgcaacccttattgatagttgccatcattaagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctctgaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtagccgtatcggaaggtgcggctggatcacctcctt

SEQ ID NO: 271 - DNA, 1357 bp, Pseudomonas mendocina
  source 1..1357: mol_type = genomic DNA
attgaacgctggttgcaggcctaacacatgcaagtcgagcggatgacgggggcttgctccctaatttagcggcggacgggtgagtaatgcctaggaatctgcctggtagtgggggataacgttccgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtacggtagagggtggtggaatttcctgtgcagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggcct

SEQ ID NO: 272 - DNA, 1347 bp, Rothia mucilaginosa; identical to SEQ ID NO: 202
  source 1..1347: mol_type = genomic DNA

SEQ ID NO: 273 - DNA, 1326 bp, Shewanella decolorationis
  source 1..1326: mol_type = genomic DNA
ttcgcgattggatgacctaggtgggattagctagttggtgaggtaatggctcaccaaggcgacgatccctagctgttctgagaggatgatcagccacactgggactgagacacggcccagactcctacgggaggcagcagtggggaatattgcacaatgggggaaaccctgatgcagccatgccgcgtgtgtgaagaaggccttcgggttgtaaagcactttcagtagggaggaaaggttgtaagttaataccttgcagctgtgacgttacctacagaagaaggaccggctaactccgtgccagcagccgcggtaatacggagggtccaagcgttaatcggaattactgggcgtaaagcgtgcgcaggcggtttgttaagcgagatgtgaaagccccgggctcaacctgggaattgcatttcgaactggcaaactagagtcttgtagaggggggtagaattccaggtgtagcggtgaaatgcgtagagatctggaggaataccggtggcgaaggcggccccctggacaaagactgacgctcaggcacgaaagcgtggggagcaaacaggattagataccctggtagtccacgccgtaaacgatgtctactcggagtttggtgtcttgaacactgggctctcaagctaacgcattaagtagaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccctatccttatttgccagcacgtaatggtgggaactctagggagactgccggtgataaaccggaggaaggtggggacgacgtcaagtcatcatggcccttacgagtagggctacacacgtgctacaatggcgagtacagagggttgcaaagccgcgaggtggagctaatctcacaaagctcgtcgtagtccggattggagtctgcaactcgactccatgaagtcggaatcgctagtaatcgtggatcagaatgccacggtgaatacgttcccgggccttgtacacaccgcccgtcacaccatgggagtgggctgcaaaagaagtgggtagcttaaccttcgggagggcgctcaccactttgtggttcatgactggggtgaagtcgtaacaaggtagccctaggggaacctggggctggatcacctcctta

SEQ ID NO: 274 - DNA, 1507 bp, Enterococcus gallinarum
  source 1..1507: mol_type = genomic DNA
catgcaagtcgaacgctttttctttcaccggagcttgctccaccgaaagaaaaagagtggcgaacgggtgagtaacacgtgggtaacctgcccatcagaaggggataacacttggaaacaggtgctaataccgtataacactattttccgcatggaagaaagttgaaaggcgcttttgcgtaacactgatggatggacccgcggtgcattagctagttggtgaggtaacggctcaccaaggccacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggatgagagtagaacgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcgaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaaggaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtatgcgaagtcgccaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgattggggtgaagtcgtaacaaggtagccgtatcggaaggtgcggctggatcacctc

SEQ ID NO: 275 - DNA, 1511 bp, Granulicatella adiacens
  source 1..1511: mol_type = genomic DNA
gcgtgcctaatacatgcaagtcgaacgagagcgaccggtgcttgcactggtcaatctagtggcgaacgggtgagtaacacgtgggtaacctgcccatcagagggggataacattcggaaacggatgctaaaaccgcataggtcttcgaaccgcatggtttgaagaggaaaagaggcgcaagcttctgctgatggatggacccgcggtgcattagctagttggtgaggtaacggctcaccaaggccgtgatgcatagccgacctgagagggtgatcggccacattgggactgagacacggcccaaactcctacgggaggcagcagtagggaatcttccgcaatggacgcaagtctgacggagcaacgccgcgtgagtgaagaaggttttcggatcgtaaaactctgttgttagagaagaacaagtgctagagtaactgttagcgccttgacggtatctaaccagaaagccacggctaactacgtgccagcagccgcggtaatacgtaggtggcaagcgttgtccggatttattgggcgtaaagcgagcgcaggcggttccttaagtctgatgtgaaagcccccggctcaaccggggagggtcattggaaactggggaacttgagtgcagaagaggagagtggaattccatgtgtagcggtgaaatgcgtagatatatggaggaacaccagtggcgaaggcgactctctggtctgtaactgacgctgaggctcgaaagcgtgggtagcaaacaggattagataccctggtagtccacgccgtaaacgatgagtgctaagtgttggagggtttccgcccttcagtgcttcagttaacgcattaagcactccgcctggggagtacgaccgcaaggttgaaactccaaaggaaattgacggggacccgcacaagcggtggagcatgtggtttaattcgaagcaacgcgaagaaccttaccaagtcttgacatcctttgaccactctagagatagagctttcccttcggggacaaagtgacaggtggtgcatggttgtcgtcagctcgtgtcgtgagatgttgggttaagtcccgcggcgagcgcaacccttattactagttgccagcattcagttgggcactctagtgagactgccggtgacaaaccggaggaaggtggggatgacgtcaaatcatcatgccccttatgacttgggctacacacgtgctacaatggatggtacaacgagcagcgaactcgcgagggtaagcgaatctcttaaagccattctcagttcggattgtaggctgcaactcgcctacatgaagccggaatcgctagtaatcacggatcagcacgccgcggtgaatacgttcccgggtcttgtacacaccgcccgtcacaccacgagagtttgtaacacccaaagtcggtgaggtaaccttttggagccagccgcctaaggtgggatagatgattggggtgaagtcgtaacaaggtagccgtatcggaaggtgcggctgagaacacctcctt

SEQ ID NO: 276 - DNA, 1513 bp, Brachybacterium conglomeratum
  source 1..1513: mol_type = genomic DNA
agagtttgataatggctcaggacgaacgctggcggcgtgcttaacacatgcaagtcgaacgatgacggtggtgcttgcaccgcctgattagtggcgaacgggtgagtaacacgtgagtaacctgccctccacttcgggataacctcgggaaatcgtggctaataccggatatgagcactcatcgcatggtgggtgttggaaaaatttatcggtgggggatggactcgcggcctatcagtttgttggtgaggtgatggctcaccaagacgatgacgggtagccggcctgagagggcgaccggccacactgggactgagacacggcccagactcctacgggaggcagcagtggggaatattgcacaatgggcgaaagcctgatgcagcgacgccgcgtgggggatgacggccttcgggttgtaaacccctttcagtagggaagaagcgagagtgacggtacctgcagaagaagcgccggctaactacgtgccagcagccgcggtaattcgtagggcgcaagcgttgtccggaattattgggcgtaaagagcttgtaggtggcttgtcgcgtctgccgtgaaaacccgaggctcaacctcgggcgtgcggtgggtacgggcaggctagagtgtggtaggggagactggaactcctggtgtagcggtgaaatgcgcagatatcaggaagaacaccgatggcgaaggcaggtctctgggccattactgacactgagaagcgaaagcatgggtagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgctgattaattcgatgcaacgcgaagaaccttaccaaggtttgacatgcactggacggctgcagagatgtggctttttttggactggtgcacaggtggtgcatggttgtcgtcagctcgtgtagtgagatgttgggttaagtcccgcaacgagcgcaaccctcgttctatgttgccagcacgtgatggtggggactcataggagactgccggggtcaactcggaggaaggtggggacgacgtcaaatcatcatgccccttatgtcttgggcttcaagcatgctacaatggtcggtacaatgggttgcgaaactgtgaggtggagcgaatcccaaaaagccggcctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccagtggcccatcctcgtgagggagctgtctaaggtgggatcggtgattgggactaagtcgtaacaaggtagccgtaccggaaggtgcggctggatcacctccttt

SEQ ID NO: 277 - DNA, 1362 bp, Neisseria subflava
  source 1..1362: mol_type = genomic DNA
attgaacgctggcggcatgctttacacatgcaagtcggacggcagcacaaagaagcttgcttcttgggtggcgagtggcgaacgggtgagtagcatatcggaacgtaccgagtaatgggggataactaatcgaaagattagctaataccgcatattctctgaggaggaaagcaggggaccttcgggccttgcgttattcgagcggccgatatctgattagctagttggtggggtaaaggcctaccaaggcgacgatcagtagcgggtctgagaggatgatccgccacactgggactgagacacggcccagactcctacgggaggcagcagtggggaattttggacaatgggcgcaagcctgatccagccatgccgcgtgtctgaagaaggccttcgggttgtaaaggacttttgtcagggaagaaaaggctgttgctaatatcgacagctgatgacggtacctgaagaataagcaccggctaactacgtgccagcagccgcggtaatacgtagggtgcgagcgttaatcggaattactgggcgtaaagcgagcgcagacggttacttaagcaggatgtgaaatccccgggctcaacctgggaactgcgttctgaactgggtgactagagtgtgtcagagggaggtagaattccacgtgtagcagtgaaatgcgtagagatgtggaggaataccgatggcgaaggcagcctcctgggataacactgacgttcatgctcgaaagcgtgggtagcaaacaggattagataccctggtagtccacgccctaaacgatgtcaattagctgttgggcaacttgattgcttagtagcgtagctaacgcgtgaaattgaccgcctggggagtacggtcgcaagattaaaactcaaaggaattgacggggacccgcacaagcggtgaatgatgtggattaattcgatgcaacgcgaagaaccttacctggtcttgacatgcacggaatcctccagagacggaggagtgccttcgggagccgtaacacaggtgctgcatggctgtcgtcagctcgtgtcgtgagatgttgggttaagtcccgcaacgagcgcaacccttgtcattagttgccatcatttagttgggcactctaatgagactgccggtgacaagccggaggaaggtggggatgacgtcaagtcctcatggcccttatgaccagggcttcacacgtcatacaatggtcggtacaaagggtagccaagccgcgaggtggagccaatctcacaaaaccgatcgtagtccggattgcactctgcaactcgagtgcatgaagtcggaatcgctagtaatcgcaggtcagcatactgcggtgaatacgttcccgggtct

SEQ ID NO: 278 - DNA, 1353 bp, Prevotella Unknown; identical to SEQ ID NO: 215
  source 1..1353: mol_type = genomic DNA

SEQ ID NO: 279 - DNA, 1506 bp, Enterococcus faecium
  source 1..1506: mol_type = genomic DNA
ggctcaggacgaacgctggcggcgtgcctaatacatgcaagtcgtacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgcgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcagcccttattgttagttgccatcattcagttgggcactctagcaagactgccggtgacaaacaggaggaaggtggggatgacgtcaaatcatcatgccccttatgacctgggctacacacgtgctacaatgggaagtacaacgagttgcgaagtcgcgaggctaagctaatctcttaaggcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgattggggtgaagtcataacatggta

SEQ ID NO: 280 - DNA, 1364 bp, Rothia dentocariosa; identical to SEQ ID NO: 206
  source 1..1364: mol_type = genomic DNA

SEQ ID NO: 281 - DNA, 1333 bp, Leptotrichia
  source 1..1333: mol_type = genomic DNA
gatgaacgctgacagaatgcttaacacatgcaagtctttggcgaagctgtgcttgcacagcctagccaaggcggacgggtgagtaacgcgtaaggaacttgccctgcagtcagggataacagacggaaacgactgataagacctggtaaagtcaggcggactcatgtccagcctgatgaaaagagatgctgcaggagagccttgcgtcctattagcttgttggtggggtaacggcccaccaaggcgatgataggtagccggcctgagagggtggacggccacaaggggactgagatacggcccttactcctacgggaggcagcagtggggaatattggacaatgggggcaaccctgatccagcaattctgtgtgcacgatgaaggttttcggattgtaaagtgctttcagcagggaagaaaaaaatgacggtacctgcagaagaagcgacggctaaatacgtgccagcagccgcggtaatacgtatgtcgcaagcgttatccggaattattgggcataaagggcatctaggcggccagataagtctggggtgaaaacttgcggctcaaccgcaagcctgccctggaaactatgtggctagagtactggagaggtggacggaactgcacgagtagaggtgaaattcgtagatatgtgcaggaatgccgatgatgaagatagttcactggacggtaactgacgctgaagtgcgaaagctaggggagcaaacaggattagataccctggtagtcctagctgtaaacgatgattactgggtgtgggcatgaagagtgtccgtgccgaagctaatgcgataagtaatccgcctggggagtacggccgcaaggctgaaactcaaaggaattgacggggacccgcacaagcggtggagcatgtggtttaattcgacgcaacgcgaggaaccttaccagatcttgacatcctgcgaaggcctgcgagagcaggctgtgcctccgggaacgcagagacaggtggtgcatggctgtcgacagctcgtgtcgtgagatgttgggttaagtcccgcaacgagcgcaacccctattgctagttgccatcattaggttgggaactctagcaagactgcctgcgaagagcaggaggaaggtggggatgacgtcaagtcatcatgccccttatgatctgggctacacacgtgctacaatggccggtacagagagccgcaaggcggcaacgccaagccaatctttaaagccggtccaagttcggattgaagtctgcaactcgacttcatgaagctggaatcgctagtaatcgcagatcagcaatgctgcggtgaatacgttctcgggtct

SEQ ID NO: 282 - DNA, 1310 bp, Roseomonas mucosa; identical to SEQ ID NO: 231
  source 1..1310: mol_type = genomic DNA

SEQ ID NO: 283 - DNA, 1449 bp, Sphingomonas; identical to SEQ ID NO: 171
  source 1..1449: mol_type = genomic DNA

SEQ ID NO: 284 - DNA, 1372 bp, Staphylococcus cohnii
  source 1..1372: mol_type = genomic DNA
gatgaacgctggcggcgtgcctaatacatgcaagtcgagcgaacagataaggagcttgctcctttgacgttagcggcggacgggtgagtatcacgtgggtaacctacctataagactggaataactccgggaaaccggggctaatgccggataacatttagaaccgcatggttctaaagtgaaagatggttttgctatcacttatagatggacccgcgccgtattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggtttcttaagtctgatgtgaaagcccacggctcaaccgtggagggtcattggaaactggggaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaatgagcgcaacccttaaacttagttgccagcatttagttgggcactctaagttgactgccggtgacaaaccggaggaaggtggggatgacgtcaaatcatcatgccccttatgatttgggctacacacgtgctacaatggacaatacaaagggcagctaaaccgcgaggtcatgcaaatcccataaagttgttctcagttcggattgtagtctgcaactcgactacatgaagctggaatcgctagtaatcgtagatcagcatgctacggtgaatacgttcccgggtct

SEQ ID NO: 285 - DNA, 1456 bp, Deinococcus
  source 1..1456: mol_type = genomic DNA
ggtgaacgctggcggcgtgcttaagacatgcaagtcgaacgcagtcttcggactgagtggcgcacgggtgagtaacacgtaactgacctacccccaagtcgcgaataaccagccgaaaggatggctaatacgtgatgtgatgatccgctgtggcggaccattaaagacttgatcgcttggggatggggttgcgttccatcagctagttggtaaggtaaaggcttaccaaggcaacgacggatagccggcctgagagggtggccggccacaggggcactgagacacgggtcccactcctacgggaggcagcagttaggaatcttccacaatgggcgcaagcctgatggagcgacgccgcgtgagggatgaaggttctcggatcgtaaacctctgaaccaacgacgaaagacccgacaagggagatgacggtagttgggtaatagcaccggctaactccgtgccagcagccgcggtaatacggagggtgcaagcgttacccggaatcactgggcgtaaagggcgtgtaggcggttacctaagtccgattttaaagaccgaagctcaacttcgggagtggattggatactgagtgacttgacctctggagaggaaacctgaattcctggtgtagcggtggaatgcgtagataccaggaggaacaccaatggcgaaggcaggtttctggacagaaggtgacgctgaggcgcgaaagtgtggggagcgaaccggattagatacccgggtagtccacaccctaaacaatgtacgttggctaaccgccggatgcggtggttggcgaagctaacgcgataaacgtaccgcctgggaagtacggccgcaaggttgaaactcaaagaaattgacgggggcccgcacaagcggtggagtatgtggtttaattcgaagcaacgcgaagaaccttaccaggtcttgacatccaaggaaccttccggaaatggaagggtgcccctcggggaaccttgagacaggtgctgcatggctgtcgtcagctcgtgtcgtgagatgttgggttaagtcccgcaacgagcgcaacccctacctttagttgctagcattgagttgagcactctagagggactgcctatgaaagtaggaggaaggcggggatgacgtctagtcagcatggtccttacgacctgggctacacacgtactacaatggccaagacaacgcgcagccaacccgcgagggtcagcgaatcgcttaaacttggccccagttcagatcggagtctgcaactcgactccgtgaagttggaatcgctagtaatcgcaggtcagcatactgcggtgaatacgttcccgggccttgtacacaccgcccgtcacaccatgggagtacgttgcagttgaaaccgccgggagccgcaaggcaggcgtctagactgtggcgcatgactggggtgaagtcgtaacaaggtaactgtaccggaagg

SEQ ID NO: 286 - DNA, 1437 bp, Lactobacillus
  source 1..1437: mol_type = genomic DNA
agagtttgatcctggctcaggacgaacgctggcggcgtgcctaatacatgcaagtcgaacgcactctcgtttagattgaaggagcttgctcctgattgataaacatttgagtgagtggcggacgggtgagtaacacgtgggtaacctgccctaaagtgggggataacatttggaaacagatgctaataccgcataaaacctaacaccgcatggtgtagggttgaaagatggtttcggctatcactttaggatggacccgcggtgcattagttagttggtgaggtaaaggctcaccaagaccgtgatgcatagccgacctgagagggtaatcggccacactgggactgagacacggcccagactcctacgggaggcagcagtagggaatcttccacaatggacgaaagtctgatggagcaacgccgcgtgagtgaagaaggttttcggatcgtaaaactctgttgttggagaagaatgtatctgatagtaactgatcaggtagtgacggtatccaaccagaaagccacggctaactacgtgccagcagccgcggtaatacgtaggtggcaagcgttgtccggatttattgggcgtaaagcgagcgcaggcggtttcttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcaaacaggattagataccctggtagtccatgccgtaaacgatgagtgctaggtgttggagggtttccgcccttcagtgccgcagctaacgcattaagcactccgcctggggagtacgaccggcaagttgaaaattcaaaggaattgacggggggcccgcacaagcggaggagcatgaggtttaatcaggagcaacgttaaggaactttaccaggtcttgacatccttttgaccattctggagatagaggctttcccatcgggggcaaagtgacaggtggtgcaaggtagtcgtcagctacgtgtcgtgagaagttgggttaagtcccgcaacgagcgcaacccttattactagttgccagcatttagttgggcactctagtgagactgccggtgacaaaccggaggaaggtgggggcgacgtcaaatcatcatgccccttatgacctgggctacacacgtgctacaatggatggtacaacgagttgcgagaccgcgaggtttagctaatctcttaaaaccattctcagttcggattgtaggctgcaactcgcctacatgaagccggaatcgctagtaatcgcggatcagcatgccgcggtgaatacgagggcgggccttgcactcaccgcccgtca

SEQ ID NO: 287 - DNA, 1405 bp, Sphingomonas yanoikuyae
  source 1..1405: mol_type = genomic DNA
agtttgatcctggctcagaacgaacgctggcggcatgcctaatacatgcaagtcgaacgagatcttcggatcgagtggcccacgggtgcgtaacgcgtgggaatctgcccttgggttcggaataacttctggaaacggaagctaataccggatgatgacgtaggtccaaagatttatcgcccaaggatgagcccgcgtaggattagctagttggtgaggtaaaggctcaccaaggcgacgatccttagctggtctgagaggatgatcagccacactgggactgagacacggcccagactcctacgggaggcagcagtggggaatattggacaatgggcgaaagcctgatccagcaatgccgcgtgagtgatgaaggccttagggttgtaaagctcttttacccgggatgataatgacagtaccgggagaataagctccggctaactccgtgccagcagccgcggtaatacggagggagctagcgttgttcggaattactgggcgtaaagcgcacgtaggcggctattcaagtcagaggtgaaagcccggggctcaaccccggaactgcctttgaaactagatagcttgaatccaggagaggtgagtggaattccgagtgtagaggtgaaattcgtagatattcggaagaacaccagttgcgaaggcggctcactggactggtattgacgctgaggtgcgaaagcgtggggagcaaacaggattagataccctggtagtccacgccgtaaacgatgataactagctgtcagggcacatggtgttttggtggcgcacgtaacgcattaagttatccgcctggggagtacggtcgcaagattaaaactcaaaggaattgacgggggcctgcacaagcggtggagcatgtggtttaattcgaagcaacgcgcagaaccttaccaacgtttgacatccctatcgcggatcgtggagacactttccttcagttcggctggataggtgacaggtgctgcatggctgtcgtcagctcgtgtcgtgagatgttgggttaagtcccgcaacgagcgcaaccctcgcctttagttgcctgcatttagttgggtactctaaaggaaccgccggtgataagccggaggaaggtggggatgacgtcaagtcctcatggcccttacgcgttgggctacacacgtgctacaatggcgactacagtgggcagccacctcgcgagagggagctaatctccaaaagtcgtctcagttcggatcgttctctgcaactcgagagcgtgaaggcggaatcgctagtaatcgcggatcagcatgccgcggtgaatacgttcccaggccttgtacacaccgcccgtcacaccatgggagttggattcactcgaaggcgttgagctaaccgtaaggaggcaggcgaccac

SEQ ID NO: 288 - DNA, 1339 bp, Propionibacterium granulosum; identical to SEQ ID NO: 201
  source 1..1339: mol_type = genomic DNA

SEQ ID NO: 289 - DNA, 1387 bp, Actinomyces massiliensis
  source 1..1387: mol_type = genomic DNA
gacgaacgctggcggcgtgcttaacacatgcaagtcgaacgggccttgtcctcctttttttgggggtggggttagtggcgaacgggtgagtaacacgtgagtaacctgcccctcacttctggataaccgcttgaaagggtggctaatacggggtgttctggcctgctcgcatgggtgggtttggaaagattcgaccggttttggttgttttggtgggggatgggctcgcggcctatcagcttgttggtggggtgatggcctgccaaggctttgacgggtagccggcctgagagggtgggcggtcacactgggactgagacacggcccagactcctacgggaggcagcagtggggaatattgcacaatgggcgcaagcctgatgcagcgacgccgcgtgagggatggaggccttcgggttgtgaacctctttcgccagtgaagaaggtcctgctctttgtggtggggttgacggtagctgggttaatgaagcgccggctaactacgtgccagcagccgcggtaatacgtagggcgcgagcgttgtccggaattattgggcgtaaagggcttgtaagcggctggtcgcgtctgccgtgaaatcctctggctcagctgggggcgtgcggtgggtacgggctggcttgagtgcggtaggggagactggaactcctggtgtagcggtggaatgcgcagatatcaggaggaacaccggtggcgaaggcgggtctctgggccgttactgacgctgaggagcgaaagcgtggggagcgaacaggattagataccctggtagtccacgctgtaaacgttgggcactaggtgtggggggccttttccgggttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgggctggattgcttcggagacggggtttcccttttttgggctggttcacaggtggtgcatggttgtcgtcagctcgtgtcgtgagatgttgggttaagtcccgcaacgagcgcaacccttgtctcgtgttgccagcacgttgtggtggggactcgcgggagactgccggggttaactcggaggaaggtggggatgacgtcaaatcatcatgccccttatgtcttgggcttcacgcatgctacaatggccggtacagtgggttgcgatgctgtgaggcggagcgaatccctgaaagccggtctcagttcggatcggtgtctgcaactcgacaccgtgaagttggagtcgctagtaatcgcagatcagcagcgctgcggtgaatacgttctcgggcct

SEQ ID NO: 290 - DNA, 1348 bp, Pseudomonas argentinensis
  source 1..1348: mol_type = genomic DNA
tcagcggcggacgggtgagttaatgcctaggaatctgcctattagtgggggacaacgtttcgaaaggaacgctaataccgcatacgtcctacgggagaaagcaggggaccttcgggccttgcgctaatagatgagcctaggtcggattagctagttggtggggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggtttgttaagttggatgtgaaagccccgggctcaacctgggaactgcatccaaaactggcaagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggaactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgcaccagaagtagctagtct

SEQ ID NO: 291 - DNA, 1425 bp, Enterobacter asburiae; identical to SEQ ID NO: 264
  source 1..1425: mol_type = genomic DNA

SEQ ID NO: 292 - DNA, 1498 bp, Pseudomonas
  source 1..1498: mol_type = genomic DNA
agagtttgatcctggctcagattgaacgctggcggcaggcctaacacatgcaagtcgagcggatgaaggtagcttgctactggattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaactctgtgccagcagccgcggtaatacagagggtgcaagcgttaatcggaattactgggcgtaaagcgcgcgtaggtggtttgttaagttggatgtgaaagccccgggctcaacctgggaactgcatccaaaactggcaagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggattccttgaggatttagtggcgcagctaacgcattaagttgaccgcctggggagtacggccgcaaggttaaaactcgaatgaattgacgggggcccgcacaagcggtggagcatgtggtttaattcgaagcaacgcgaagaaccttacctggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccgcaagggggacggttaccacggagtgattcatgactggggtgaagtcgtaacaaggtaacc

SEQ ID NO: 293 - DNA, 1367 bp, Streptococcus
  source 1..1367: mol_type = genomic DNA
gacgaacgctggcggcgtgcctaatacatgcaagtagaacgctgaaggaggagcttgcttctctggatgagttgcgaacgggtgagtaacgcgtaggtaacctgcccggtagcgggggataactattggaaacgatagctaataccgcataacagtagatgttgcatgacatttgcttaaaaggtgcaattgcaccactaccagatggacctgcgttgtattagctagttggtggggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgccgtaaacgatgagtgctaggtgttagaccctttccggggtttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaagtcttgacatcctttgaccactctagagatagagctttcccttcggggacaaagtgacaggtggtgcatggttgtcgtcagctcgtgtcgtgagatgttgggttaagtcccgcaacgagcgcaacccttattactagttgccagcattcggttgggcactctagtgagactgccggtgacaaaccggaggaaggtggggatgacgtcaaatcatcatgccccttatgacttgggctacacacgtgctacaatggatggtacaacgagcagcgaactcgcgagggtaagcgaatctcttaaagccattctcagttcggattgtaggctgcaactcgcctacatgaagccggaatcgctagtaatcgcggatcagcacgccgcggtgaatacgttcccgggtct

SEQ ID NO: 294 - DNA, 1362 bp, unidentified
  misc_feature 1..1362: note = 16S ribosomal RNA of unknown bacteria
  source 1..1362: mol_type = genomic DNA
ggcgagcggcggacggctgagtaacgcgtgggaacgtaccccaaagtgagggataacgcatcgaaaggtgtgctaataccgcatatggtcttcggattaaagccttcgggcgctttgggaacggcctgcgtaagattagatagttggtggggtaatggcctaccaagtcgacgatctttaactggtttgagaggatgatcagtcagactggaactgagacacggtccagactcctacgggaggcagcagtgaggaatcttccacaatgggcgaaagcctgatggagcaacgccgcgtgcaggatgaaggccttcgggttgtaaactgcttttataagtgaggattatgacagtaacttatgaataaggatcggctaactacgtgccagcagccgcggtcatacgtaggatccgagcgttatccggagtgactgggcgtaaagggttgcgtaggtggttcgataagcgaatagtgaaatctggtggctcaaccattcagactattattcgaactgttggactcgagagcagaagaggtagctggaatttctagtgtaggagtgaaatccgtagatattagaaggaacaccgatggcgtaggcaggctactgggctgtttctgacactgaggcacgaaagcgtggggagcgaaccggattagatacccgggtagtccacgccgtgaacgatggatactagctgtttggggtatcgaccccctgagtagcgaagctaacgcgttaagtatcccgcctgtggagtacgatcgcaagattaaaacataaaggaattgacggggacccgcacaagcggtggatcatgttctttaattcgatgataaccgataaaccttaccaggtcttgacatccttggaatctttcagaaatgagagagtgctttttaagagccaagtgacaggtgatgcatggccgtcgtcagctcgtgtcgtgagatgtttggttaagtccatcaacgagcgcaacccttgtcagtagttgtatttttctactgagactgccccggcaacggggaggaaggaggggatgacgtcaggtcagtatttcccttacgtcctgggctagaaacgtgatacaatggctagtacaatgcgcagcgaagccgcgaggtgaagcaaatcgcatcaaagctagtcccagttcggattgtaggctgaaactcgcctgcatgaagtcggaatcgctagtaatcgcagatcagcatgctgcggtgaatacgttcccgggtcttgtacacaccgcccgtcaaaccatgaaagtcactaacacccgaagtccgattcgtcggcctaaggtgggagggatgattggggttaagtcgtaacaaggtatccgtaccggaagg

SEQ ID NO: 295 - DNA, 1407 bp, Treponema socranskii
  source 1..1407: mol_type = genomic DNA
catgcaagtcgagcggcaggcagcaatgccgagagcggcggactggtgagtaacacgtggataacgtaccccgatgtccgggacagcctgtagaaatagagggtaataccggatagatcactgtttacggaaggtagacagcgggaaaggagcttcggctccgcattgggcacggtctgcggcccatcagctagacggcggggtaagggcccgccgtggcgaggacgggtatccggcctgagagggcggacggacacattgggactgagatacggcccagactcctacgggaggcagcaggtaagaatattccgcaatggggggaaccctgacggagcgacgccgcgtgaacgaagaaggccggaaggttgtaaagttcttttctgtccgaggaataagtgtaggaggaaatgcctgcatggtgacggtagggcaggaataagcaccggctaattacgtgccagcagccgcggtaacacgtaaggtgcgagcgttgttcggaattattgggcgtaaagggcatgcaggcgggtcgccaagcttggtaagaaataccggggctcaactccggagctatattgagaactggcgagctagagttgccgaagggtatccggaattccgcgtgaaggggtgaaatctgtagatatgcggaagaacaccgatggcgaaggcaggataccggcggacgactgacgctgaggtgcgaaggtgcggggagcaaacaggattagataccctggtagtccgcacagtcaacgatgtacactgggcgtgtgcgcaagagcgtgcgtgccgaagcaaacgcgataagtgtaccgcctggggagtatgcccgcaagggtgaaactcaaaggaattgacgggggcccgcacaagcggtggagcatgtggtttaattcgatggtacgcgaggaaccttacctgggtttgacatcaagagcgatcatatagagatatgtgagcgtagcaatacggctcttgacaggtgctgcatggctgtcgtcagctcgtgccgtgaggtgttgggttaagtcccgcaacgagcgcaacccctactgccggttactaacaggtaaagctgaggactcaggcggaactgcctgcgacaagcaggaggaaggcggggacgacgtcaagtcatcatggcccttatgtccagggctacacacgtgctacaatggccgccacagagcggggcgaagccgagaggaggagcagaacgcagaaaagcggtcgtagtccggattgaagtctgaaactcgacttcatgaagctggaatcgctagtaaccgcacatcagcacggcgcggtgaatacgttcccgggccttgtacacaccgcccgtcacaccatccgagcagggggtacccgaagccggcagtccaaccgcaagggggacgctgt

SEQ ID NO: 296 - DNA, 1474 bp, Bacillus clausii
  source 1..1474: mol_type = genomic DNA
gacgaacgctggcggcgtgcctaatacatgcaagtcgagcggacagaagggagcttgctcccggacgttagcggcggacgggtgagtaacacgtgggcaacctgccccttagactgggataactccgggaaaccggagctaataccggataatccctttctccacctggagagagggtgaaagatggcttcggctatcactaggggatgggcccgcggcgcattagctagttggtaaggtaacggcttaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgaggaaggccttcgggtcgtaaagctctgttgtgagggaagaagcggtaccgttcgaatagggcggtaccttgacggtacctcaccagaaagccacggctaactacgtgccagcagccgcggtaatacgtaggtggcaagcgttgtccggaattattgggcgtaaagcgcgcgcaggcggcttcttaagtctgatgtgaaatctcggggctcaaccccgagcggccattggaaactggggagcttgagtgcagaagaggagagtggaattccacgtgtagcggtgaaatgcgtagagatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccgtagtgccgaagttaacacattaagcactccgcctggggagtacggccgcaaggctgaaactcaaaggaattgacggggacccgcacaagcagtggagcatgtggtttaattcgaagcaacgcgaagaaccttaccaggtcttgacatcctttgaccacccaagagattgggcttccccttcgggggcaaagtgacaggtggtgcatggttgtcgtcagctcgtgtcgtgagatgttgggttaagtcccgcaacgagcgcaacccttgatcttagttgccagcattaagttgggcactctaaggtgactgccggtgacaaaccggaggaaggtggggatgacgtcaaatcatcatgccccttatgacctgggctacacacgtgctacaatggatggtacaaagggcagcgaaaccgcgaggtgaagccaatcccataaagccattctcagttcggattgcaggctgcaactcgcctgcatgaagccggaattgctagtaatcgcggatcagcatgccgcggtgaatacgttcccgggtcttgtacacaccgcccgtcacaccacgagagtttgtaacacccgaagtcggtgaggcaaccttttggagccagccgcctaaggtgggacaaatgattggggtgaag

SEQ ID NO: 297 - DNA, 1340 bp, Corynebacterium
  source 1..1340: mol_type = genomic DNA
gacgaacgctggcggcgtgcttaacacatgcaagtcgaacggaaaggccctgcttgcagggtactcgagtggcgaacgggtgagtaacacgtgggtgatctgccctgcacttcgggataagcctgggaaactgggtctaataccggataggagccatttttagtgtgatggttggaaagttttttctgtgtaggatgagctcgcggcctatcaacttgatggtggggtaatggcctaccaaggcggcgacgggtatccggcctgagagggtggactgccaccttgggactgatataccgcccatactcctacgggagggagcaatggggaatattgcacgatgggctctggcctgatgcaacaactcctcgtggaggatgacggccttcgggttgaaaagctcctttctctaggcgctaagctttttgtgacgatacctagataagagtgcgcggctacctacgtgccccttagcagctgtaatacgtagggcgccaccgttgtccggaagtactgggcgtaaacggctcgtagtggtttgtcgcgtcgtctgtgaattccggggcttaactccgggcgtgcaggcgatacgggcataacttgagtactgtaggggtaactggaattcctggtgtagcggtgaaatgcgcagatatcaggaggaacaccgatggcgaaggcaggttactgggcagttactgacgctgaggagcgaaagcatgggtagcgaacaggattagataccctggtagtccatgccgtaaacggtgggcgctaggtgtgagggtcttttcacgactttcgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatacactggatcgctgcagagatgtagtttccctttgtggctggtgtacaggtggtgcatggttgtcgtcagctcgtgtcgtgagatgttgggttaagtcccgcaacgagcgcaacccttgtcttatgttgccagcatttggttggggactcatgagagactgccggggtcaactcggaggaaggtggggatgacgtcaaatcatcatgccccttatgtccagggcttcacacatgctacaatggtcggtacaacgcgcagcgacactgtgaggtggagcgaatcgctgaaagccggccttagttcggattggggtctgcaactcgaccccatgaagtcggagtcgctagtaatcgcagatcagcaatgctgcggtgaatacgttcccgggcct

SEQ ID NO: 298 - DNA, 1396 bp, Enterobacter cloacae; identical to SEQ ID NO: 267
  source 1..1396: mol_type = genomic DNA

SEQ ID NO: 299 - DNA, 1499 bp, Neisseria macacae
  source 1..1499: mol_type = genomic DNA
attgaacgctggcggcatgctttacacatgcaagtcggacggcagcacagagaagcttgcttcttgggtggcgagtggcgaacgggtgagtaacatatcggaacgtaccgagcagtgggggataactaatcgaaagattagctaataccgcatattttctgaggaagaaagcaggggaccatttggccttgcgctgtttgagcggccgatatctgattagctagttggtggggtaaaggcctaccaaggcgacgatcagtagcgggtctgagaggatgatccgccacactgggactgagacacggcccagactcctacgggaggcagcagtggggaattttggacaatgggcgcaagcctgatccagccatgccgcgtgtctgaagaaggccttcgggttgtaaaggacttttgtcagggaagaaaagggcggggttaatacccctgtctgatgacggtacctgaagaataagcaccggctaactacgtgccagcagccgcggtaatacgtagggtgcgagcgttaatcggaattactgggcgtaaagcgggcgcagacggttacttaagcaggatgtgaaatccccgggctcaacctgggaactgcgttctgaactgggtgactagagtgtgtcagagggaggtagaattccacgtgtagcagtgaaatgcgtagagatgtggaggaataccgatggcgaaggcagcctcctgggataacactgacgttcatgcccgaaagcgtgggtagcaaacaggattagataccctggtagtccacgccctaaacgatgtcgattagctgttgggcagcatgactgcttagtagcgaagctaacgcgtgaaatcgaccgcctggggagtacggtcgcaagattaaaactcaaaggaaattgacggggaacccgcacaagcggtggatgatgtggattaattcgatgcaacgcgaagaaccttacctggtcttgacatgtacggaaccctccagagacggaggggtgccttcgggagccagtaacacaggtgctgcatggctgtcgtcagctcgtgtcgtgagatgttgggttaagtcccgcaacgaggcgcaacccttgtccattagttgccatcattaagttgggcactctaatgaggactgccggtgacaagccggaggaaggtggggatgacgtcaagtcctcatggcccttatgaccagggcttcacacgtcatacaatggtcggtacagagggtagccaagccgcgaggcggagccaatctcataaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagtaggtagggtaaccgcaaggagcccgcttaccacggtatgcttcatgactggggtgaagtcgtaacaaggtagccgtaggggaacc

SEQ ID NO: 300 - DNA, 1348 bp, Kocuria atrinae
  source 1..1348: mol_type = genomic DNA
gacgaacgctggcggcgtgcttaacacatgcaagtcgaacgatgaagctccagcttgctggggtggattagtggcgaacgggtgagtaatacgtgagtaacctgcccttgactctgggataagcctgggaaactgggtctaatactggatactacacattctcgcatgggggtgtgtggaaagggttttactggttttggatgggctcacggcctatcagcttgttggtggggtaatggcctaccaaggcgacgacgggtagccggcctgagagggtgaccggccacactgggactgagacgcggcccagactcctacgggaggcagcagtggggaatattgcacaatgggcgaaagcctgatgcagcgacgccgcgtgagggatgacggccttcgggttgtaaacctctttcagcacggaagaagcgaaagtgacggtacgtgcagaagaagcgccggctaactacgtgccagcagccgcggtaatacgtagggcgcaagcgttgtccggaattattgggcgtaaagagctcgtaggcggtttgtcgcgtctgctgtgaaagcccggggcttaaccccgtggtgtgcagtgggtacgggcagactagagtgcagtaggggagactggaattcctggtgtagcggtgaagtgcgcagatatcaggaggaacaccgatggcgaaggcaggtctctgggctgttactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatacaccagaccggtccagagatggtccttcccctttggggttggtgtacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggatgacgtcaaatcatcatgccccttatgtcttgggcttcacgcatgctacaatggccggtacaatgggttgcgataccgtgaggtggagctaatcccaaaaagctggtctcagttcggatcgtggtctgcaactcgaccacgtgaagtcggagtcgctagtaatcgcagatcagcaacgctgcggtgaatacgttcccgggcct

SEQ ID NO: 301 - DNA, 1505 bp, Acinetobacter
  source 1..1505: mol_type = genomic DNA
ttagagtttgatcatggctcagattgaacgctggcggcaggcttaacacatgcaagtcgagcggagtgatggtgcttgcaccttatcttagcggcggacgggtgagtaatgcttaggaatctgcctattagtgggggacaacattccgaaaggaatgctaataccgcatacgtcctacgggagaaagcaggggatcttcggaccttgcgctaatagatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatggggggaaccctgatccagccatgccgcgtgtgtgaagaaggccttatggttgtaaagcactttaagcgaggaggaggctactgagactaatactcttggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccccgagcttaacttgggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtaggtagtctaaccgtaaggaggacgcttaccacggtgtggccgatgactggggtgaagtcgtaacaaggtaaccgtag

SEQ ID NO: 302 - DNA, 1540 bp, Escherichia/Shigella
  source 1..1540: mol_type = genomic DNA
ttaaattgaagagtttgatcatggctcagattgaacgctggcggcaggcctaacacatgcaagtcgaacggtaacaggaaacagcttgctgtttcgctgacgagtggcggacgggtgagtaatgtctgggaaactgcctgatggagggggataacgtactggaacggtagctataccgcataacgtcgcaagaccaaagagggggaccttcgggcctcttgccatcggatgtgccagatgggattagcttgttggtgggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagagggggtagaattccaggtgtagcggtgaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaaatggaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaagtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 303 - DNA, 1501 bp, Enterobacter
  source 1..1501: mol_type = genomic DNA
gtttgatcatggctcagattgaacgctggcggcaggcctaacacatgcaagtcgagcggcagcgggaagtagcttgctactttgccggcgagcggcggacgggtgagtaatgtctgggaaactgcctgatggagggggataactactggaaacggtagctaacaccgcataacgtcgcaagaccaaagagggggaccttcgggcctcttgccatcagatgtgcccagatgggattagctagtaggtggggtaacggctcacctaggcgacaatccctagctggtctgagaggatgaccagccacactggaactgagacacggtccagactcctactggaggcagcagtggggaatattgcacaatgggcgcaagcctgatgcagccatgccgcgtgtatgaagaaggccttcgggttgtaaagtactttcagcggggaggaaggtgttgtggttaataaccacagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggcttgactcttgtacaggggggtagaattccaggtgtagcggtgaaatgcctaaagatctggaggaataccggtggcgaaagcgggccccctgtacaaagactgacgctccaggtgccaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaatttggcagagatgccttagtgccttcgggaaccgtgagacaggtgctgcatggctgtcgtcagctcgtgttgtgaaatgttgggttaagtcccgcaacgagcgcaacccttatcctttgttgccagcgattcggtcgggaactcaaaggagactgccagtgataaactggaggaaggtgggatgacgtcaagtcatcatggcccttacgagtagggctacacacgtgctacaatggcatatacaaagagaagcgacctcgcgagagcaagcggacctcataaagtatgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaacc

SEQ ID NO: 304 - DNA, 1466 bp, Agromyces mediolanus
  source 1..1466: mol_type = genomic DNA
gacgaacgctgtcggcgtgcttaacacatacaagtcgaacgatgaacttggagcttgctctgggggattagtgacgaacgggtgagtaacacgtgagtaacctcccctggactctgggataacttcgagaaatcggagctaataccggataggaccttgcaccgcatggtgtggggtggaaagtttttcggtttgggatggactcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgcaagcctgatgcaccaaccccgcgtgcgggatgacggccttcgggttgtaaaccgcttttagtagggaagaagccttcgggtgacggtacctgcagaaaaaggaccggctaactacgtgccagcaaccgcggtaatacgtagggtccgagcgttgtccggaattattgggcgtaaagagctcgtaggcggtttgtcgcgtctcctgtgtaaactagaggctcaacctctatcctccggtgggtacgggcagacttgagtggtgtaggggagactggaattcctggtgtatcggtggaatgcgcagatatcaggaggaacaccgatgtcgaaggcaggtctctgggcacttactgacgctgaggagcgaaagcgtggggagcgaacaggattagataccctggtagtccacgccgtaaacgttgggcgctagatgtggggacctttccacggtttccgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacacgagaacgggccagaaatggtcaactctttggacacttgtgaacaggtggtgcatggttgtcgtcagctcgtgtcgtgagatgttgggttaagtcccgcaacgagcgcaaccctcgtcgcatgttgccagcacgttatggtggggactcatgtgagactgccggggtcaactcggaggaaggtggggatgacgtcaaatcatcatgccccttatgtcttgggcttcacgcatgctacaatggccggtacaaagggctgcgatgtcgtaaggcggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaacccttgtggagggagccgtcgaaggtgggatcggtgattaggactaagtcgtaacaaggtaacc

SEQ ID NO: 305 - DNA, 1469 bp, Agrobacterium
  source 1..1469: mol_type = genomic DNA
gagtttgatcctggctcagaacgaacgctggcggcaggcttaacacatgcaagtcgagcgccccgcaaggggagcggcagacgggtgagtaacgcgtgggaacgtaccctttactacggaacaactccgggaaactggagctaataccgtatgtgccctttgggggaaagatttatcggtaaaggatcggcccgcgttggattagctagttggtggggtaaaggcctaccaaggcgacgatccatagctggtctgagaggatgatcagccacattgggactgagacacggcccaaactcctacggcaggcagcagtggggaatattggacaatgggcgcaagcctgacccagccatgccgcgtgaatgatgaaggccttagggttgtaaagttctttcaccggagaagataatgacggtatccggagaagaagccccggctaacttcgtgccagcagccgcggtaatacgaagggggctagcgttgttcggaattactgggcgtaaagcgcacgtaggcggacatttaagtcaggggtgaaatcccagagctcaactctggaactgcctttgatactgggtgtcttgagtgtggaagaagtcagtggaattgcgagtgtagaggtgaaattcgtagatattcgcaggaacaccagtggcgaaggcggctgactggtccacaactgacgctgaggtgcgaaagcgtggggagcaaacaggattagataccctggtagtccacgccgtaaacgatgaatgttagtcgtcgggcagtttactgttcggtgacgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacatgcccggctacatccagagatggatggttcccttcggggaccgggacacaggtgctgcatggctgtcgtcagctcgtgtcgtgagatgttgggttaagtcccgcaacgagcgcaaccctcgcccttagttgccatcattcagttgggcactctaaggggactgccggtgataagccgagaggaaggtggggatgacgtcaagtcctcatggcccttacgggctgggctacacacgtgctacaatggtggtgacagtgggcagcaagcacgcgagtgtgagctaatctccaaaagccatctcagttcggattgcactctgcaactcgagtgcatgaagttggaatcgctagtaatcgcggatcagcacgccgcggtgaatacgttcccgggccttgtacacaccgcccgtcacaccatgggagttggttttacccgaaggcgctgcgctaacccgcaagggaggcaggcgaccacggtagggtcagcgactggggtgaagtcgtaacaaggtagccgtaggggaacctgcggctggatcacc

SEQ ID NO: 306 - DNA, 1366 bp, Luteimonas
  source 1..1366: mol_type = genomic DNA
agtgaacgctggtggcaggcctaacacatgcaagtcgaacggcagcacagaggagcttgctccttgggtggcgagtggcggacgggtgaggaatacatcggaatctgcctatttgtgggggataacgtagggaaacttacgctaataccgcatacgacctacgggtgaaagcggaggaccttcgggcttcgcgcagatagatgagccgatgtcggattagctagttggcggggtaaaggcccaccaaggcgacgatccgtagctggtctgagaggatgatcagccacactggaactgagacacggtccagactcctacgggaggcagcagtggggaatattggacaatgggcgcaagcctgatccagccatgccgcgtgggtgaagaaggccttcgggttgtaaagcccttttgttgggaaagaaaagcagtcggttaatacccgattgttctgacggtacccaaagaataagcaccggctaacttcgtgccagcagccgcggtaatacgaagggtgcaagcgttactcggaattactgggcgtaaagcgtgcgtaggtggtttgttaagtctgatgtgaaagccctgggctcaacctgggaattgcattggatactggcaggctagagtgcggtagagggtagtggaattcccggtgtagcagtgaaatgcgtagagatcgggaggaacatccgtggcgaaggcgactacctggaccagcactgacactgaggcacgaaagcgtggggagcaaacaggattagataccctggtagtccacgccctaaacgatgcgaactggatgttgggttcaactaggaactcagtatcgaagctaacgcgttaagttcgccgcctggggagtacggtcgcaagactgaaactcaaaggaattgacgggggcccgcacaagcggtggagtatgtggtttaattcgatgcaacgcgaagaaccttacctggccttgacatgcacggaactttccagagatggattggtgccttcgggaaccgtgacacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtgggaatgacgtcaagtcatcatggcccttacggccagggctacacacgtactacaatgggaaggacagagggctgcgaacccgcgagggcaagccaatcccagaaaccttctctcagtccggatcggagtctgcaactcgactccgtgaagtcggaatcgctagtaatcgcagatcagcattgctgcggtgaatacgttcccgggcct

SEQ ID NO: 307 - DNA, 1412 bp, Lysinibacillus boronitolerans
  source 1..1412: mol_type = genomic DNA
cgaacgctggcggcgtgcctaatacatgcaagtcgagcgaacagagaaggagcttgcaccttagacgttagcggcggacgggtgagtaacacgtgggcaacctaccttatagtttgggataacaccgggaaaccggggctaataccgaataatctgtttcacctcatggtgaaacactgaaagacggtttcggctgtcgctataggatgggcccgcggcgcattagctagttggtgaggtaacggctcaccaaggcgacgatgcgtagccgacctgagagggtgatcggccacactgggactgagacacggcccagactcctacgggaggcagcagtagggaatcttccacaatgggcgaaagcctgatggagcaacgccgcgtgagtgaagaaggatttcggttcgtaaaactctgttgtaagggaagaacaagtacagtagtaactggctgtaccttgacggtaccttattagaaagccacggctaactacgtgccagcagccgcggtaatacgtaggtggcaagcgttgtccggaattattgggcgtaaagcgcgcgcagctggtttcttaagtctgatgtgaaagcccacggctcaaccgtggagggtcattggaaactgggagacttgagtgcagaagaggatagtggaattccaagtgtagcggtgaaatgcgtagagatttggaggaacaccagtggcgaaggcgactatctggtctgtaactgacactgaggcgcgaaagcgtggggagcaaacaggattagataccctggtagtccaggccgtaaacgatgagtgctaagtgttagggggtttccgccccttagtgctgcagctaacgcattaagcactccgcctggggagtacggtcgcaagactgaaactcaaaggaattgacggcggcccgcacaagccgtggagcatcttgtttaattcgaagcaacgcgaagaaccttaccaggtcttgacatcccgttgaccactgtagagatatcgtttccccttcgggggcaacggtgacaggtggtgcatggttgtcgtcagctcgtgtcgtgagatgttgggttaagtcccgcaacgagcgcaacccttgatcttagttgccatcatttagttgggcactctaaggtgactgccggtgacaaaccggaggaaggtggggatgacgtcaaatcatcatgccccttatgacctgggctacacacgtgctacaatggacgatacaaacggttgccaactcgcgagagggagctaatccgataaagtcgttctcagttcggattgtagactgcaactcgcctacatgaagccggaatcgctagtaatcgcggatcagcatgccgcggtgaatacgttcccgggccgagtacacaccgcccgtcacaccacgagagtttgtaacaccc

SEQ ID NO: 308 - DNA, 1525 bp, Exiguobacterium
  source 1..1525: mol_type = genomic DNA
agagtttgatcatggctcaggacgaacgctggcggcgtgcctaatacatgcaagtcgagcgcaggaagccgttccgaacccttcggggggacgaacggtggaatgagcggcggacgggtgagtaacacgtaaagaacctgcccataggtctgggataaccacgagaaatcggggctaataccggatgtgtcatcggaccgcatggtccgctgatgaaaggcgcttcggcgtcgcccatggatggctttgcggtgcattagctagttggtggggtaatggcccaccaaggcgacgatgcatagccgacctgagagggtgatcggccacactgggactgagacacggcccagactcctacgggaggccagcagtagggaatcttccacaatggacgaaagtctgatggagcaacgccgcgtgaacgatgaaggctttcgggtcgtaaagttctgttgtaagggaagaacaagtgccacaggcaatggcggcaccttgacggtaccttgcgagaaagccacggctaactacgtgccagcagccgcggtaatacgtaggtggcaagcgttgtccggaattattgggcgtaaagcgcgcgcaggcggcctcttaagtctgatgtgaaagcccccggctcaaccggggagggccattggaaactgggaggcttgagtataggagagaagagtggaattccacgtgtagcggtgaaatgcgtagagatgtggaggaacaccagtggcgaaggcgactctttggcctataactgacgctgaggcgcgaaagcgtggggagcaaacaggattagataccctggtagtccacgccgtaaacgatgagtgctaggtgttggagggtttccgcccttcagtgctgaagctaacgcattaagcactccgcctggggagtacggtcgcaaaggctgaaactcaaaggaatttgacggggacccgcacaagcggtggagcatgtggtttaattcgaagcaacgcgaagaaccttaccaactcttgacatccccctgaccggtacagagatgtaccttccccttcgggggcaggggtgacaggtggtgcatggttgtcgtcagctcgtgtcgtgagatgttgggttaagtcccgcaacgagcgcaacccttgtccttagttgccagcattcagttgggcactctagggagactgccggtgacaaaccggaggaaggtggggatgacgtcaaatcatcatgccccttatgagttgggctacacacgtgctacaatggacggtacaaagggcagcgaagccgcgaggtggagccaatcccagaaagccgttctcagttcggattgcaggctgcaactcgcctgcatgaagtcggaatcgctagtaatcgcaggtcagcatactgcggtgaatacgttcccgggtcttgtacacaccgcccgtcacaccacgagagtttgcaacacccgaagtcggtgaggtaaccgtaaggagccagccgccgaaggtggggcagatgattggggtgaagtcgtaacaaggta

SEQ ID NO: 309 - DNA, 1358 bp, Acinetobacter
  source 1..1358: mol_type = genomic DNA
attgaacgctggcggcaggcttaacacatgcaagtcgagcggagatggggtgcttgcaccttatcttagcggcggacgggtgagtaatgcttaggaatctgcctattagtgggggacaacattccgaaagggatgctaataccgcatacgtcctacgggagaaagcaggggatcttcggaccttgcgctaatagatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatggggggaaccctgatccagccatgccgcgtgtgtgaagaaggccttatggttgtaaagcactttaagcgaggaggaggctactgagactaatactcttggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccccgagcttaacttgggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggccttgacatactagaaactttccagagatggattggtgccttcgggaatctagatacaggtgctgcatggctgtcgtcagctcgtgtcgtgagatgttgggttaagtcccgcaacgagcgcaacccttttccttacttgccagcatttcggatgggaactttaaggatactgccagtgacaaactggaggaaggcggggacgacgtcaagtcatcatggcccttacggccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggcct

SEQ ID NO: 310 - DNA, 1432 bp, Psychrobacter
  source 1..1432: mol_type = genomic DNA
agtcgagcggaacgatgatagcttgctatcaggcgtcgagcggcggacgggtgagtaatacttaggaatctacctagtagtgggggatagctcggggaaactcgaattaataccgcatacgacctacgggagaaagggggcaacttgttgctctcgctattagatgaacctaagtcggattagctagatggtggggtaaaggcctaccatggcaacgatctgtagctggtctgaaaggatgatcagccacaccgggactgagacacggcccggactcctacgggagcgcagcattaatttcagaattatttgagacaactgagagacaccttgatccaggccatgcggagtgcggcgacgaactgctttttggttgctaaagcactttatagcagtgaagaagactccgtggttaatacccacggacgattgacattagctgcagaactaagcaccggttaactctgtgccagcagccgcggtaatacagagggtgcaagcgttaatcggaattactgggcgtaaagcgagccgtaggtggcttgataagtcatatgtgaaatccccgggcttaaccagggaactcgcatctgatactgttaggctagaataggtgagaggaagggtataattccatgtgtggggggtgaaatgcgtattttatttggaggaatacggatagcgaaggcatcttttcttgcatcataccgacactgaggttcgaaagcgtgggtagcaaacaggattagataccctggtcgtccacgccgtaaacgatgtctacttgtcgttgggtcccttgaggacttagtgacgcagcccacccaataaatacaccgccaggggagtacgagccgccgggttagaactcaaaatgaatttattggggccggcacacccagtggaacgtgtgggtttattttcatacaacgcaaagaaccttacctggtcttgttgtgatctacagtccggcagaaaaagaaggagtgccggtggggtaaatacaagaaaagtaccgcgctggctgtcgtcagctcgtgtcgtgagatgttgggttaagtcccgcaacgagcgcaaccacttgtccttatttactaccgggtgaagcagggggaggtcgcgccgatactgccagtgacaaactggaggaaggcggggacgacgtcaagtcatcatggcccttacgaccagggctacacacgtgctacaatggtaggtacagagggcagctacacagcgatgtgatgcgaatctcaaaaagcctatcgtagtccagattggagtctgcaactcgactccatgaagtaggaatcgctagtaatcgcggatcagaatgccgcggtgaatacgttcccgggccttgtacacaccgcccgtcacaccatgggagttgattgcaccagaagtggatagcttaaccttaaggaaggcg

SEQ ID NO: 311 - DNA, 1520 bp, Veillonella dispar; identical to SEQ ID NO: 205
  source 1..1520: mol_type = genomic DNA

SEQ ID NO: 312 - DNA, 1415 bp, Acinetobacter
  source 1..1415: mol_type = genomic DNA
agtcgagcgggggaaggtagcttgctaccggacctagcggcggacgggtgagtaatgcttaggaatctgcctattagtgggggacaacgtttcgaaaggaacgctaataccgcatacgtcctacgggagaaagcaggggatcttcggaccttgcgctaatagatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatggggggaaccctgatccagccatgccgcgtgtgtgaagaaggccttttggttgtaaagcactttaagcgaggaggaggctactagtattaatactaactggatagtggacgttagactcgcaagaaataagcaccggctaactctggtgccaggcaggccgcggtaataacagagggtgcgagcgttaattcggatttactgggcgtaaaggcgtgcgtaggcggctttttaagtcggatgtgaaatccctgagcttaaacttaggaaattgcattcgatactgggaagctagagtatgggagaggatggtagaattccaggtgtaagcgggtgaaatgcgtagagatcttggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcggttcggccgggaactttaaggatactgccagtgacaaactggaggaaggcggggacgacgtcaagtcatcatggcccttacgaccagggctacacacgtgctacaatggtcggtacaaagggttgctacctcgcgagaggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtaggtagtctaaccgtaaggaggacgct

SEQ ID NO: 313 - DNA, 1372 bp, Staphylococcus
  source 1..1372: mol_type = genomic DNA
gatgaacgctggcggcgtgcctaatacatgcaagtcgagcgaacagacgaggagcttgctcctttgacgttagcggcggacgggtgagtaacacgtaggtaacctacctataagactgggataacttcgggaaaccggagctaataccggataatatttcgaaccgcatggttcgatagtgaaagatggctttgctatcacttatagatggacctgcgccgtattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaacgtgtaagtaactgtgcacgtcttgacggtacctaatcagaaagccacggctaactacgtgccagcagcccgcgtgatacgtaagtgggcagcgttatccggaatttatggaacgtaagcgcgcgtaggcggttttttaagtctgatgtgaaagcccacggctcaaccgtggagggtcattggaaactggaaaacttgagtgcagaagaggaaagtggaattccatgtgtagtggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacccttctagagatagaagtttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtct

SEQ ID NO: 314 - DNA, 1365 bp, Sphingomonas yunnanensis; identical to SEQ ID NO: 228
  source 1..1365: mol_type = genomic DNA

SEQ ID NO: 315 - DNA, 1481 bp, Ralstonia
  source 1..1481: mol_type = genomic DNA
agagtttgatcctggctcagattgaacgctggcggcatgccttacacatgcaagtcgaacggcagcatgatctagcttgctagattgatggcgagtggcgaacgggtgagtaatacatcggaacgtgccctgtagtgggggatgactagtcgaaagattagctaataccgcatacgacctgagggtgaaagtgggggaccgcaaggcctcatgctataggagcggccgatgtttgtttagcttgtttgtgaggtaaagtctcaccaaggcgacgatcagtagctgttctgagaggacgattagccacactgtgactgtgacacgtcccagattcctacgggaggcagcagtgtggcattttggacaattggcgaaagcctgattcagcaatttcgcgtgtgtgaagaagtctttcgggttgtaaagcacttttgtccggaaagaaatggctcttgttaatacctgggttcgatgacggtaccggaagaataaggaccggctaactacgtgccagcagccgcggttatacgtagggtccaagcgttaatcggatttactgggcgtaaagcgtgcgcaggcggttgtgcaagaccgatgtgaaatccccgagcttaacttgggaattgcattggtgactgcacggctagagtgtgtcagaggggggtagaattccacgtgtagcagcgaaatgcgtagagatgtggaggaataccgatggcgaaggcagccccctgggataacactgacgctcatgcacgaaagcgtggggagcaaacaggattagataccctggtagtccacgccctaaacgatgtcaactagttgttggggattcatttccttagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgccactaacgaagcagagatgcattaggtgctcgaaagagaaagtggacacaggtgctgcatggctttcgtcagctcgtgtcgtgagatgttgggttaagtcccgcaacgagcgcaacccttgtctctagttgctacgaaagggcactctagagagactgccggtgacaaaccggaggaaggtggggatgacgtcaggtcctcatggcccttatgggtagggcttcacacgccatacaatggtgcatagagagggttgccacgccgcgaggtggagctaatcccagaaaatgcatcgtagtccggatcgtagtctgcaactcgactacgtgaagctggagtcgctagtaatcgcggatcagcatgccgcggtgaatacgttcccgggtcttgttcacaccgcccgtcacaccatgggagtgggctttaccagaagtagttagcctaaccgcaaggagggcgattaccacggtagggttcatgactggggtga

SEQ ID NO: 316 - DNA, 1545 bp, unidentified
  misc_feature 1..1545: note = 16S ribosomal RNA of unknown bacteria
  source 1..1545: mol_type = genomic DNA
agagtttgatcatggctcagattgaacgctggcggcaggcctaacacatgcaagtcgaacggtaacaggaagcagcttgctgcttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggtgttgaggttaataacctcagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctccaggtgcgaaagcgtggggagcaaacagggattagataccctggtagtcccacgccgtaaacgatgtcgacttggaggttgtgcccttgaggcgtggcttccggagctaacgcgttaagtcgaccgcctgggggagtacggccgcaaggttaaaactccaaatgaattgacgggggcccgcacaagcggtggagcatgtggtttaattccgatgcaacgcgaagaaccttacctactcttgacatcccagagaacttagcagagatgctttggtgccttcgggaactctggagacaggtgctgcatggctgtcgtccagctcgtgttgtgaaatgttgggttaagtccagcaacgtttagcctgctaagcccttatccattggtagccagcggtccggccgggaactcaaagcagactgccagtgataaactgtgaggtcacaggtggggatgacgccaagtcatcatggcccttacgatgtagggctacacacgtgctacaatggcttgcatacaaagtactaaccgaccgcgcgcagagcaaacggacctcataaagtgcgtcatagaccagattggagtcttgcaactcgacttccatgaagtcagaatcgctagataatcgtggatcaacaacgccacgctgaatacgttcaccgggccttgttacgacaccgcccgtcacaccatggggagtggggttgcaaaagaagtaggtagcttaaaccttcgggagggcgctttaccactttgtgattgcatgactggggtgaaatcctaaccaaggtaaccgt

SEQ ID NO: 317 - DNA, 1386 bp, unidentified
  misc_feature 1..1386: note = 16S ribosomal RNA of unknown bacteria
  source 1..1386: mol_type = genomic DNA
agagtttgatcctggctcagagtgaacgctggcggtaggcctaacacatgcaagtcgaacggcagcacagtaagagcttgctcttatgggtggcgagtggcggacgggtgaggaatacatcggaatctactttttcgtgggggataacgtagggaaacttacgctaataccgcatacgacctacgggtgaaagcaggggatcttcggaccttgcgcgattgaatgagccgatgtcggattagctagttggcggggtaaaggcccaccaaggcgacgatccgtagctggtctgagaggatgatcagccacactggaactgagacacggtccagactcctacgggaggcagcagtggggaatattggacaatgggcgcaagcctgatccagccataccgcgtgggtgaagaaggccttcgggttgtaaagcccttttgttgggaaagaaatccagccggctaatacctggttgggatgacggtacccaaagaataagcaccggctaacttcgtgccagcagccgcggtaatacgaagggtgcaagcgttactcggaattactgggcgtaaagcgcgcgtaggtggttagttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccagaaaaccgatcgtagtccggatcgcagtctgcaactcgactgcgtgaagtcggaatcgctagtaatcgcgaatcagaatgtcgcggtgaatacgttcccgggcct

SEQ ID NO: 318 - DNA, 1399 bp, Pseudomonas libanensis
  source 1..1399: mol_type = genomic DNA
catgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtggggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccagaaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcggagg

SEQ ID NO: 319 - DNA, 1444 bp, Rubrivivax
  source 1..1444: mol_type = genomic DNA
gctggcggcatgccttacacatgcaagtcgaacggtaacgcggggcaacctggcgacgagtggcgaacgggtgagtaatgcatcggaacgtgcccagaagtgggggatagcccggcgaaagctggattaataccgcatgagacctgagggtgaaagcgggggatcgcaagacctcgcgcttttggagcggccgatgtcagattagctagttagtggggtaaaggcctaccaaggcgacgatctgtagctggtctgagaggacgaccagccacactgggattgagacacggcccagactcctacgggaggcagcagtggggaattttggacaatgggcgcaagcctgatccagccatgccgcgtgcgggaagaaggccttcgggttgtaaaccgcttttgtcagggaagaaacgctctgggctaataccctggggtaatgacggtacctgaagaataagcaccggctaactacgtgctagcagccgcggtaatacgtagggtgcaagcgttaatcggaattactgggcgtaaagcgtgcgcaggcggttatgcaagacagatgtgaaatccccgggctcaacctgggaactgcatttgtgactgcatggctagagtgcggcagagggggatggaattccgcgtgtagcagtgaaatgcgtagatatgcggaggaacaccgatggcgaaggcaatcccctgggcctgcactgacgctcatgcacgaaagcgtggggagcaaacaggattagataccctggtagtccacgccctaaacgatgtcaactggttgttgggagggtttcttctcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacctacccttgacatgtctggaatcctgaagagatttgggagtgctcgaaagagagccagaacacaggtgctgcatggccgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaggtcctcatggcccttatgggtagggctacacacgtcatacaatggccggtacagagggctgccaacccgcgagggggagccaatcccagaaaaccggtcgtagtccggatcgcagtctgcaactcgactgcgtgaagtcggaatcgctagtaatcgcggatcagcttgccgcggtgaatacgttcccgggtcttgtacacaccgcccgtcacaccatgggagcgggttctgccagaagtagttagcctaaccgcaaggagggcgattaccacggcagggttcgtgactggggtg

SEQ ID NO: 320 - DNA, 1309 bp, unidentified; identical to SEQ ID NO: 250
  misc_feature 1..1309: note = 16S ribosomal RNA of unknown bacteria
  source 1..1309: mol_type = genomic DNA

SEQ ID NO: 321 - DNA, 1372 bp, Staphylococcus caprae
  source 1..1372: mol_type = genomic DNA
gatgaacgctggcggcgtgcctaatacatgcaagtcgagcgaacagacgaggagcttgctcctctgacgttagcggtggacgggcgagtaacacgtggataacctacctataagactgggataacttcgggaaaccggagctaataccggataacatgttgaaccgcatggttcaacagtgaaagacggtcttgctgtcacttatagatggatccgcgccgcattagctagttggtgaggtaacggcttaccaaggcaacgatgcgtagccgacctgagagggtgatcgaccacactggaactgagacacggtccagactcctacgggaggcagcagtagggaatcttccgcaatgggcgaaagcctgacggagcaacgccgcgtgagtgaagaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgaccgctctagagatagagtcttccccttcgggggacaaagtgacaggtggtgcatggttgtcgtcagctcgtgtcgtgagatgttgggttaagtcccgcaacgagcgcaacccttaagcttagttgccatcattcagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtct

SEQ ID NO: 322 - DNA, 1514 bp, Streptococcus parasanguinis
  source 1..1514: mol_type = genomic DNA
agagtttgatcctggctcaggatgaacgctggcggcgtgcctaatacatgcaagtagaacgctgaagcttggtgcttgcaccgagcggatgagttgcgaacgggtgagtaacgcgtaggtaacctgcctcttagcgggggataactattggaaacgatagctaataccgcataaaagtcgacatcgcatgatgttgacttgaaaggtgcaattgcatcactaagagatggacctgcgctgtattagctagttggtgaggtaacggctcaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgagtgtgagagtggaaagttcacactgtgacggtaacttaccagaaagggacggctaactacgtgccagcagccgcggtaatacgtaggtcccgagcgttatccggatttattgggcgtaaagcgagcgcaggcggttagataagtctgaagttaaaggctgtggcttaaccatagtacgctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttgggtcctttccagggactcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagactttccttcgggacagaggtgacaggtggtgcatggttgtcgtcagctcgtgtcgtgagatgttgggttaagtcccgcaacgagcgcaacccctattgttagttgccatcattgagttgggcactctagcgagactgccggtaataaaccggaggaaggtggggatgacgtcaaatcatcatgccccttatgacctgggctacacacgtgctacaatggctggcacaacgagtcgcgagtcggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtaaccgta

SEQ ID NO: 323 - DNA, 1456 bp, unidentified
  misc_feature 1..1456: note = 16S ribosomal RNA of unknown bacteria
  source 1..1456: mol_type = genomic DNA
cggcaggcctaacacatgcaagtcgaacggtaacaggaagaagcttgcttctttgctgacgagtggcagacgggtgagtaatgtctgggaaactgcctgatgaagggggataactactgaaaacggtacctaataccgcttaccttctcaagaccaaggagggggaccttcgggcctcttgccatcggatgtgcccagatgtgattagtttgtaggtggggtaacggctcacctaggcgacgatccttagctgctctgagaggatgtccacccccactggaattgagccacgttccagattcttacgggagccaccagtggggattattgcccaatgggggcaaccttgatgcacccatccggggtgtatgaagaagtctttgggtttgtagagtactttcagcggggaggacgggagtaaagttaattcctttggtcattgacgttccccgccgaagaagcaccggttaattccgtcccaccagccgcgttaatacggagggtccaagcttttctcggaatttctgggggtaaagcgcactcaggcgttttgttaattcagatgtgaaatccccgggttcaccctgggaggtccatgtgatattggcaagcttgagtctggtggaggggggtagatttccaggggtagcgttgaaatgcataggagattgggagaactacgggtggcgaagcgggccccttgtacgaaaactgccgttcagttgcgaaggcgtggggagcaaccaggattagataccttggtagtccgcgcggtaaacgaagtcgactagatgttgtgcctttgaggcgtggcttcctcagctaacgcgttaagtagtccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagt

SEQ ID NO: 324 - DNA, 1465 bp, Gardnerella vaginalis
  source 1..1465: mol_type = genomic DNA
gatgaacgctggcggcgtgcttaacacatgcaagtcgaacgggatctgaccagcttgctggttggtgagagtggcgaacgggtgagtaatgcgtgaccaacctgccccatgctccagaatagctcttggaaacgggtggtaatgctggatgctccaacttgacgcatgtcttgttgggaaagtgtttagtggcatgggatggggtcgcgtcctatcagcttgtaggcggggtaatggcccacctaggcttcgacgggtagccggcctgagagggcggacggccacattgggactgagatacggcccagactcctacgggaggcagcagtggggaatattgcgcaatgggggaaaccctgacgcagcgacgccgcgtgcgggatgaaggccttcgggttgtaaaccgcttttgattgggagcaagctttcgggtgagtgtacctttcgaataagcgccggctaactacgtgccagcagccgcggtaatacgtagggcgcaagcgttatccggaattattgggcgtaaagagcttgtaggcggttcgtcgcgtctggtgtgaaagcccatcgcttaacggtgggtctgcgccgggtacgggcgggctagagtgcagtaggggagactggaattcccggtgtaacggtggaatgtgtagatatcgggaagaacaccaatggcgaaggcaggtctctgggctgttactgacgctgagaagcgaaagcgtggggagcgaacaggattagataccctggtagtccacgccgtaaacggtggacgccggatgtggggcccattccacgggttccgtgtcggagctaacgcgttaagcgtcccgcctggggagtacggccgcaaggctaaaactcaaagaaattgacgggggcccgcacaagcggcggagcatgcggattaattcgatgcaacgcgaagaaccttacctgggcttgacatgtgcctgtcgactgcagagatgtggtttcccttcggggcaggttcacaggtggtgcatggtcgtcgtcagctcgtgtcgtgagatgttgggttaagtcccgcaacgagcgcaaccctcgccctgtgttgccagcgggttatgccgggaactcacgggggaccgccggggttaactcggaggaaggtggggatgacgtcagatcatcatgccccttacgtccagggcttcacgcatgctacaatggccggtacaacgggatgcgacatggtgacatggagcggatcccttaaaaccggtctcagttcggatcgtagtctgcaactcgactacgtgaaggcggagtcgctagtaatcgcgaatcagcaacgtcgcggtgaatgcgttcccgggccttgtacacaccgcccgtcaagtcatgaaagtgggcagcacccgaagccggtggcctaacccttttgggatggagccgtctaaggtgaggctcgtgattgggactaagtcgtaacaaggta

SEQ ID NO: 325 - DNA, 1377 bp, Paracoccus marcusii; identical to SEQ ID NO: 220
  source 1..1377: mol_type = genomic DNA